(19) United States
(12) Patent Application Publication
(10) Pub. No.: US 2019/0218614 A1
(43) Pub. Date: Jul. 18, 2019

(54) CLONAL HEMATOPOIESIS AND CYTOKINE TARGETS

(71) Applicant: TRUSTEES OF BOSTON UNIVERSITY, Boston, MA (US)

(72) Inventors: Kenneth WALSH, Carlisle, MA (US); Soichi SANO, Charlottesville, VA (US); Kosei OSHIMA, Kagawa (JP)

(73) Assignee: TRUSTEES OF BOSTON UNIVERSITY, Boston, MA (US)

(21) Appl. No.: 16/249,104

(22) Filed: Jan. 16, 2019

Related U.S. Application Data
(60) Provisional application No. 62/617,875, filed on Jan. 16, 2018.

(51) Int. Cl. C12Q 1/6883

(52) U.S. Cl. CPC C12Q 1/6883; C12Q 2600/118; C12Q 2600/156

(57) ABSTRACT

As demonstrated herein, a preferential and progressive expansion of a subset of hematopoietic cells bearing somatic mutations in one or more HSC cardiometabolic driver genes leads to pro-inflammatory signaling at multiple levels. Accordingly, provided herein are compositions, methods, and assays for modulating a HSC cardiometabolic driver gene mutation-mediated proinflammatory activity.

### CROSS-REFERENCE TO RELATED APPLICATIONS

This application claims benefit under 35 U.S.C. § 119(e) of U.S. Provisional Application No. 62/617,875 filed Jan. 16, 2018, the content of which is incorporated herein by reference in its entirety.

### GOVERNMENT SUPPORT

This invention was made with Government Support under Contract Nos. HL131006 and HL138014 awarded by the National Institutes of Health (NIH). The Government has certain rights in the invention.

### SEQUENCE LISTING

The instant application contains a Sequence Listing which has been submitted electronically in ASCII format and is hereby incorporated by reference in its entirety. Said ASCII copy, created on Jan. 15, 2019, is named 701586-090850USPT_SL.txt and is 1,127,419 bytes in size.

### TECHNICAL FIELD

The technical field relates to compositions, methods, and assays for the treatment, prevention and diagnosis of cardio-metabolic diseases, chronic kidney disease, and other age-dependent chronic diseases that involve pathological inflammatory responses.

### BACKGROUND

Macrovascular diseases, such as cardiovascular disease (CVD) and stroke, steeply increase with age and account for >85% of chronic disease deaths in those >70 years of age (from Belsky et al. Proc. Natl. Acad. Sci. USA 2015). While cardiovascular disease is the leading cause of death in the elderly, almost 60% of elderly patients with atherosclerotic CVD have either no or just one conventional CV risk factors (e.g., hypertension, hypercholesterolemia, etc.). As can be seen in Khot et al. (JAMA 2003), more than 60% of patients with CVD display either zero or one conventional risk factors. Similarly, retrospective analysis of CVD patients revealed that up to 50% of these patients were classified as low-risk when applying predictive scales based on traditional risk factors (Akosah et al. JACC 2003). Consistent with these findings, the recent PESA and AWHS studies have reported that subclinical atherosclerosis can be detected in more than 57% of asymptomatic adults categorized as “low CV risk” on the basis of conventional 10-year risk prediction algorithms (Fernandez-Friera et al. Circulation 2015; Laclaustra et al. JACC 2016). However, much of this amounts to a desire—without identifying what the risk factors are. These clinical studies only demonstrate that there are as-yet-unidentified causal risk factors that drive cardiovascular disease in the human population.

### SUMMARY

The present invention is directed, in part, to a new paradigm of causal risk for cardiovascular and related diseases and provides novel compositions, methods, and assays for treating, preventing, and/or diagnosing the same. Epidemiological studies show that hematopoietic stem cells (HSCs) develop mutations that promote their clonal expansion at a relatively high frequency in the aging population. While very few of the HSCs acquire subsequent mutations in oncogenes that lead to blood cancers, the mechanistic findings of the studies described herein show that a single gene mutation that occurs frequently can predispose an individual to CVD and stroke that are common in the elderly (>50% of individuals). Accordingly, the findings described herein demonstrate that there is a common mechanistic basis between age-associated CVD and clonal expansion of HSCs having somatic mutations. These data provide experimental evidence supporting a mechanism whereby somatic mutations in HSCs represent a new causal risk factor for CVD, potentially adding to the predictive capabilities of the conventional risk factors (hyperlipidemia, hypertension, diabetes and smoking) that were deduced approximately 50 years ago. These data also provide the first mechanistic evidence for how somatic mutations in different genes in HSCs, such as TP53, JAK2, ASXL1, PPM1D/WIP1, TET2 and DNMT3A, can lead to chronic non-cancerous diseases, providing novel personalized therapies or preventive strategies for individuals carrying somatic mutations in blood cells. As shown herein, somatic mutations in HSCs leads, in part, to increases in inflammatory cytokines such as IL-1β, IL-6, and/or TNF-α. Accordingly, in some embodiments of the aspects described herein, neutralizing antibodies against IL-1β, IL-6, and/or TNF-α cytokines, or NLRP3 inflammasome inhibition can be particularly effective for the prevention/treatment of CVD and other metabolic diseases in individuals carrying somatic mutations in these genes.

Accordingly, provided herein, in some aspects, are methods for treating a subject having, or at risk for, a HSC (hematopoietic stem cell) cardiometabolic driver gene mutation-mediated proinflammatory disease comprising: administering a therapeutically effective amount of a pharmaceutical composition comprising an inhibitor of HSC cardiometabolic driver gene mutation-mediated proinflammatory activity and a pharmaceutically acceptable carrier to a subject having one or more somatic mutations in one or more HSC cardiometabolic driver gene in a sub-population of peripheral blood hematopoietic cells.

In some embodiments of these methods and all such methods described herein, the one or more somatic mutations in the one or more HSC cardiometabolic driver genes in the sub-population of peripheral blood hematopoietic cells cause clonal hematopoiesis in the subject.

In some embodiments of these methods and all such methods described herein, at least 2% of the peripheral blood hematopoietic cells have the one or more somatic mutations in the one or more HSC cardiometabolic driver genes.

In some embodiments of these methods and all such methods described herein, the one or more HSC cardiometabolic driver genes are selected from TP53, JAK2, DNMT3A, ASXL1, TET2 and PPM1D/WIP1.

In some embodiments of these methods and all such methods described herein, the one or more somatic mutations are in TP53 and are selected from a G743A mutation in SEQ ID NO:2 and a A659G mutation in SEQ ID NO: 2.

In some embodiments of these methods and all such methods described herein, the one or more somatic mutations are in JAK2 and is a G1849T in SEQ ID NO: 56.

In some embodiments of these methods and all such methods described herein, the one or more somatic mutations are in DNMT3A and are selected from a T1115C mutation in SEQ ID NO: 37; a C2711T mutation in SEQ ID NO: 37; a C1837G mutation in SEQ ID NO: 37; a A1666G mutation in SEQ ID NO: 37; a C1789T mutation in SEQ ID NO: 37; a G2719A mutation in SEQ ID NO: 37; a G1627T mutation in SEQ ID NO: 37; an A2723G mutation in SEQ ID NO: 37; a G1797T mutation in SEQ ID NO: 37; a T2252G mutation in SEQ ID NO: 37; a C1560A mutation in SEQ ID NO: 37; a T1031C mutation in SEQ ID NO: 37; a G2645A mutation in SEQ ID NO: 37; a C2043G mutation in SEQ ID NO: 37; a C2446T mutation in SEQ ID NO: 37; a A2198G mutation in SEQ ID NO: 37; A2281G mutation in SEQ ID NO: 37; a C920G mutation in SEQ ID NO: 37; a A2204G mutation in SEQ ID NO: 37; and a frameshift mutation in DNMT3A.

In some embodiments of these methods and all such methods described herein, the one or more somatic mutations are in ASXL1 and are selected from a C2407T mutation in SEQ ID NO: 61; a C2893T mutation in SEQ ID NO: 61; a 1926_1926delinsAG mutation in SEQ ID NO: 61; and a frameshift mutation in ASXL1.

In some embodiments of these methods and all such methods described herein, the one or more somatic mutations are in PPM1D and are selected from a G1618T mutation in SEQ ID NO: 64; a C1372T mutation in SEQ ID NO: 64; and a frameshift mutation in PPM1D.

In some embodiments of these methods and all such methods described herein, the subject further has one or more somatic mutations in TET2 in a sub-population of peripheral blood hematopoietic cells selected from an S282F mutation in SEQ ID NO: 68, an N312S mutation in SEQ ID NO: 68, an L346P mutation in SEQ ID NO: 68, an S460F mutation in SEQ ID NO: 68, a D666G mutation in SEQ ID NO: 68, a P941S mutation in SEQ ID NO: 68, and a C1135Y mutation in SEQ ID NO: 68.

In some embodiments of these methods and all such methods described herein, the inhibitor of HSC cardiometabolic driver gene mutation-mediated proinflammatory activity is an IL-1β inhibitor.

In some embodiments of these methods and all such methods described herein, the IL-1β inhibitor is an IL-1β inhibitor antibody or antigen-binding fragment thereof that binds to IL-1β and reduces IL-1β binding to its receptor(s).

In some embodiments of these methods and all such methods described herein, the IL-1β inhibitor antibody or antigen-binding fragment thereof is selected from ABT981, an anti-interleukin-1β inhibitor antibody by ABZYME, APX002, Canakinumab/Ilaris, CDP48, immunereszumab, LY2189102, MED18968, and XOMA052.

In some embodiments of these methods and all such methods described herein, the IL-1β inhibitor is an IL-1 receptor antagonist.

In some embodiments of these methods and all such methods described herein, the IL-1 receptor antagonist is selected from CDP484, CP412245, CYT013 IL1bQb, XL 130, AMG108, HL 2351, IL1Hyl, AXXO, orthokine, PRT 1000, anakinra, and rilonacept.

In some embodiments of these methods and all such methods described herein, the IL-1β inhibitor is a small molecule or microRNA inhibitor that inhibits IL-1-mediated pro-inflammatory activity.

In some embodiments of these methods and all such methods described herein, the small molecule or microRNA inhibitor is selected from AC201, CP412245, MCC950 or CRID3, inflabion, inflammasome modulator OPSONA, PGE3935199, PGE527667, TRK530, β-hydroxybutyrate (BHB), and microRNA-223. In some embodiments of these methods and all such methods described herein, the small molecule inhibitor is MCC950.

In some embodiments of these methods and all such methods described herein, the inhibitor of HSC cardiometabolic driver gene mutation-mediated proinflammatory activity is an IL-6 inhibitor.

In some embodiments of these methods and all such methods described herein, the IL-6 inhibitor is an IL-6 inhibitor antibody or antigen-binding fragment thereof that binds to IL-6 and reduces IL-6 binding to its receptor(s).

In some embodiments of these methods and all such methods described herein, the IL-6 inhibitor antibody or antigen-binding fragment thereof is selected from Siltuximab, Olokizumab, Elsilimomab, mAb 1339, Sirukumab, Clazakizumab, ARGX-109, FM101, and C326.

In some embodiments of these methods and all such methods described herein, the IL-6 inhibitor is an IL-6 receptor antagonist.

In some embodiments of these methods and all such methods described herein, the IL-6 receptor antagonist is selected from tocilizumab, sarilumab, REGN88, FE301, and LMT-28.

In some embodiments of these methods and all such methods described herein, the IL-6 inhibitor is a small molecule or microRNA inhibitor.

In some embodiments of these methods and all such methods described herein, the small molecule IL-6 inhibitor is ALX-0061 or LMT-28.

In some embodiments of these methods and all such methods described herein, the IL-6 inhibitor is a JAK-STAT inhibitor.

In some embodiments of these methods and all such methods described herein, the JAK-STAT inhibitor is selected from baricitinib, decernotinid, filgotinib, INCB-039110, ruxolitinib, tofacitinib, Oclacitinib, Gandotinib, Lestaurtinib, Momelotinib, Pacritinib, PF-04965842, Upadacitinib, and Peficitinib.

In some embodiments of these methods and all such methods described herein, the inhibitor of HSC cardiometabolic driver gene mutation-mediated proinflammatory activity is a TNFα inhibitor.

In some embodiments of these methods and all such methods described herein, the TNFα inhibitor is a TNFα inhibitor antibody or antigen-binding fragment thereof that binds to TNFα and reduces TNFα binding to its receptor(s).

In some embodiments of these methods and all such methods described herein, the TNFα inhibitor antibody or antigen-binding fragment thereof is selected from adalimumab, Adalimumab-atto, certolizumab pegol, golimumab, infliximab,

In some embodiments of these methods and all such methods described herein, the TNFα inhibitor is a TNFα receptor antagonist.

In some embodiments of these methods and all such methods described herein, the TNFα receptor antagonist is etanercept.

In some embodiments of these methods and all such methods described herein, the TNFα inhibitor is a small molecule or microRNA inhibitor.

In some embodiments of these methods and all such methods described herein, the method further comprises monitoring hematopoietic cell clonality, IL-1β proinflammatory activity, IL-6 proinflammatory activity, TNFα proinflammatory activity or any combination thereof following the administration of the inhibitor of HSC cardiometabolic driver gene mutation-mediated proinflammatory activity.

In some embodiments of these methods and all such methods described herein, the method further comprises decreasing the number or percentage of hematopoietic cells comprising the one or more somatic mutations in the one or more HSC cardiometabolic driver genes in the subject by performing therapeutic cytapheresis on the subject.

In some embodiments of these methods and all such methods described herein, the method further comprises administering one or more additional therapeutic agents to the subject.

Also provided herein, in some aspects, are methods for treating a subject having, or at risk for, a HSC cardiometabolic driver gene mutation-mediated proinflammatory disease comprising:
(a) sequencing a hematopoietic cell sample from a subject to identify one or more somatic mutations in one or more HSC cardiometabolic driver genes in the hematopoietic cell sample; and
(b) administering a therapeutically effective amount of a pharmaceutical composition comprising an inhibitor of HSC cardiometabolic driver gene mutation-mediated proinflammatory activity and a pharmaceutically acceptable carrier if one or more somatic mutations in one or more HSC cardiometabolic driver genes are identified in the hematopoietic cell sample.

In some embodiments of these methods and all such methods described herein, the hematopoietic cell sample is a peripheral blood hematopoietic cell sample.

In some embodiments of these methods and all such methods described herein, the hematopoietic cell sample is enriched for myeloid-derived cells.

In some embodiments of these methods and all such methods described herein, the one or more somatic mutations in the one or more HSC cardiometabolic driver genes identified in the hematopoietic cell sample cause clonal hematopoiesis in the subject.

In some embodiments of these methods and all such methods described herein, at least 2% of the hematopoietic cells are identified as having one or more HSC cardiometabolic driver gene mutations.

In some embodiments of these methods and all such methods described herein, the one or more HSC cardiometabolic driver genes are selected from TP53, JAK2, DNMT3A, ASXL1, and PPM 1D/WIP1.

In some embodiments of these methods and all such methods described herein, the one or more somatic mutations are in TP53 and are selected from a G743A mutation in SEQ ID NO:2 and a A659G mutation in SEQ ID NO: 2.

In some embodiments of these methods and all such methods described herein, the one or more somatic mutations are in JAK2 and is a G1849T in SEQ ID NO: 56.

In some embodiments of these methods and all such methods described herein, the one or more somatic mutations are in DNMT3A and are selected from a T1115C mutation in SEQ ID NO: 37; a C2711T mutation in SEQ ID NO: 37; a C1837G mutation in SEQ ID NO: 37; a A1666G mutation in SEQ ID NO: 37; a C1789T mutation in SEQ ID NO: 37; a G2719A mutation in SEQ ID NO: 37; a G1627T mutation in SEQ ID NO: 37; an A2723G mutation in SEQ ID NO: 37; a G1797T mutation in SEQ ID NO: 37; a T2252G mutation in SEQ ID NO: 37; a C1560A mutation in SEQ ID NO: 37; a T1031C mutation in SEQ ID NO: 37; a G2645A mutation in SEQ ID NO: 37; a C2043G mutation in SEQ ID NO: 37; a C2446T mutation in SEQ ID NO: 37; a A2198G mutation in SEQ ID NO: 37; A2281G mutation in SEQ ID NO: 37; a C920G mutation in SEQ ID NO: 37; a A2204G mutation in SEQ ID NO: 37; and a frameshift mutation in DNMT3A.

In some embodiments of these methods and all such methods described herein, the one or more somatic mutations are in ASXL1 and are selected from a C2407T mutation in SEQ ID NO: 61; a C2893T mutation in SEQ ID NO: 61; a 1926_1926delinsAG mutation in SEQ ID NO: 61; and a frameshift mutation in ASXL1.

In some embodiments of these methods and all such methods described herein, the one or more somatic mutations are in PPM1D and are selected from a G1618T mutation in SEQ ID NO: 64; a C1372T mutation in SEQ ID NO: 64; and a frameshift mutation in PPM1D.

In some embodiments of these methods and all such methods described herein, the subject further has one or more somatic mutations in TET2 in a sub-population of peripheral blood hematopoietic cells selected from an S282F mutation in SEQ ID NO: 68, an N312S mutation in SEQ ID NO: 68, an L346P mutation in SEQ ID NO: 68, an S460F mutation in SEQ ID NO: 68, a D666G mutation in SEQ ID NO: 68, a P941S mutation in SEQ ID NO: 68, and a C1135Y mutation in SEQ ID NO: 68.

In some embodiments of these methods and all such methods described herein, the inhibitor of HSC cardiometabolic driver gene mutation-mediated proinflammatory activity is an IL-1β inhibitor.

In some embodiments of these methods and all such methods described herein, the IL-1β inhibitor is an IL-1β inhibitor antibody or antigen-binding fragment thereof that binds to IL-1β and reduces IL-1β binding to its receptor(s).

In some embodiments of these methods and all such methods described herein, the IL-1β inhibitor antibody or antigen-binding fragment thereof is selected from ABT981, an anti-interleukin-1β inhibitor antibody by ABZYME, APX002, Canakinumab/Ilaris, CDP48, immunereszumab, LY2189102, MEDI8968, and XOMA052.

In some embodiments of these methods and all such methods described herein, the IL-1β inhibitor is an IL-1 receptor antagonist.

In some embodiments of these methods and all such methods described herein, the IL-1 receptor antagonist is selected from CDP484, CP412245, CYT013 IL1bQb, XL 130, AMG108, HL 2351, IL1Hyl, AXXO, orthokine, PRT 1000, anakinra, and rilonacept.

In some embodiments of these methods and all such methods described herein, the IL-1β inhibitor is a small molecule or microRNA inhibitor that inhibits IL-1β-mediated pro-inflammatory activity.

In some embodiments of these methods and all such methods described herein, the small molecule or microRNA inhibitor is selected from AC201, CP412245, MCC950 or CRID3, inflabion, inflammasome modulator OPSONA, PGE3935199, PGE527667, TRK530, j-hydroxybutyrate (BHB), and microRNA-223. In some embodiments of these methods and all such methods described herein, the small molecule inhibitor is MCC950.

In some embodiments of these methods and all such methods described herein, the inhibitor of HSC cardiometabolic driver gene mutation-mediated proinflammatory activity is an IL-6 inhibitor.

In some embodiments of these methods and all such methods described herein, the IL-6 inhibitor is an IL-6 inhibitor antibody or antigen-binding fragment thereof that binds to IL-6 and reduces IL-6 binding to its receptor(s).

In some embodiments of these methods and all such methods described herein, the IL-6 inhibitor antibody or antigen-binding fragment thereof is selected from Siltuximab, Olokizumab, Elsilimomab, mAb 1339, Sirukumab, Clazakizumab, ARGX-109, FM101, and C326.

In some embodiments of these methods and all such methods described herein, the IL-6 inhibitor is an IL-6 receptor antagonist.

In some embodiments of these methods and all such methods described herein, the IL-6 receptor antagonist is selected from tocilizumab, sarilumab, REGN88, FE301, and LMT-28.

In some embodiments of these methods and all such methods described herein, the IL-6 inhibitor is a small molecule or microRNA inhibitor.

In some embodiments of these methods and all such methods described herein, the small molecule IL-6 inhibitor is ALX-0061 or LMT-28.

In some embodiments of these methods and all such methods described herein, the IL-6 inhibitor is a JAK-STAT inhibitor.

In some embodiments of these methods and all such methods described herein, the JAK-STAT inhibitor is selected from baricitinib, decernotinid, filgotinib, INCB-039110, ruxolitinib, tofacitinib, Oclacitinib, Gandotinib, Lestaurtinib, Momelotinib, Pacritinib, PF-04965842, Upadacitinib, and Peficitinib.

In some embodiments of these methods and all such methods described herein, the inhibitor of HSC cardiometabolic driver gene mutation-mediated proinflammatory activity is a TNFα inhibitor.

In some embodiments of these methods and all such methods described herein, the TNFα inhibitor is a TNFα inhibitor antibody or antigen-binding fragment thereof that binds to TNFα and reduces TNFα binding to its receptor(s).

In some embodiments of these methods and all such methods described herein, the TNFα inhibitor antibody or antigen-binding fragment thereof is selected from adalimumab, Adalimumab-atto, certolizumab pegol, golimumab, infliximab,

In some embodiments of these methods and all such methods described herein, the TNFα inhibitor is a TNFα receptor antagonist.

In some embodiments of these methods and all such methods described herein, the TNFα receptor antagonist is etanercept.

In some embodiments of these methods and all such methods described herein, the TNFα inhibitor is a small molecule or microRNA inhibitor.

In some embodiments of these methods and all such methods described herein, the method further comprises monitoring hematopoietic cell clonality, IL-1β proinflammatory activity, IL-6 proinflammatory activity, TNFα proinflammatory activity or any combination thereof following the administration of the inhibitor of HSC cardiometabolic driver gene mutation-mediated proinflammatory activity.

In some embodiments of these methods and all such methods described herein, the method further comprises decreasing the number or percentage of hematopoietic cells comprising the one or more somatic mutations in the one or more HSC cardiometabolic driver genes in the subject by performing therapeutic cytapheresis on the subject.

In some embodiments of these methods and all such methods described herein, the method further comprises administering one or more additional therapeutic agents to the subject.

In some embodiments of these methods and all such methods described herein, the HSC cardiometabolic driver gene mutation-mediated proinflammatory disease is a cardiometabolic disease or disorder.

In some embodiments of these methods and all such methods described herein, the HSC cardiometabolic driver gene mutation-mediated proinflammatory disease is a chronic kidney disease or disorder.

Also provided here, in some aspects, are pharmaceutical compositions comprising an inhibitor of HSC cardiometabolic driver gene mutation-mediated proinflammatory activity and a pharmaceutically acceptable carrier for use in a subject having one or more somatic mutations in one or more HSC cardiometabolic driver genes in a sub-population of hematopoietic cells.

In some embodiments of these compositions and all such compositions described herein, the one or more somatic mutations in the one or more HSC cardiometabolic driver genes in the sub-population of hematopoietic cells cause clonal hematopoiesis in the subject.

In some embodiments of these compositions and all such compositions described herein, at least 2% of the hematopoietic cells in the subject have the one or more mutations in one or more HSC cardiometabolic driver genes.

In some embodiments of these compositions and all such compositions described herein, the one or more HSC cardiometabolic driver genes are selected from TP53, JAK2, DNMT3A, ASXL1, and PPM1D/WIP1.

In some embodiments of these compositions and all such compositions described herein, the one or more somatic mutations are in TP53 and are selected from a G743A mutation in SEQ ID NO:2 and a A659G mutation in SEQ ID NO: 2.

In some embodiments of these compositions and all such compositions described herein, the one or more somatic mutations are in JAK2 and is a G1849T in SEQ ID NO: 56.

In some embodiments of these compositions and all such compositions described herein, the one or more somatic mutations are in DNMT3A and are selected from a T1115C mutation in SEQ ID NO: 37; a C2711T mutation in SEQ ID NO: 37; a C1837G mutation in SEQ ID NO: 37; a A1666G mutation in SEQ ID NO: 37; a C1789T mutation in SEQ ID NO: 37; a G2719A mutation in SEQ ID NO: 37; a G1627T mutation in SEQ ID NO: 37; an A2723G mutation in SEQ ID NO: 37; a G1797T mutation in SEQ ID NO: 37; a T2252G mutation in SEQ ID NO: 37; a C1560A mutation in SEQ ID NO: 37; a T1031C mutation in SEQ ID NO: 37; a G2645A mutation in SEQ ID NO: 37; a C2043G mutation in SEQ ID NO: 37; a C2446T mutation in SEQ ID NO: 37; a A2198G mutation in SEQ ID NO: 37; A2281G mutation in SEQ ID NO: 37; a C920G mutation in SEQ ID NO: 37; a A2204G mutation in SEQ ID NO: 37; and a frameshift mutation in DNMT3A.

In some embodiments of these compositions and all such compositions described herein, the one or more somatic mutations are in ASXL1 and are selected from a C2407T mutation in SEQ ID NO: 61; a C2893T mutation in SEQ ID NO: 61; a 1926_1926delinsAG mutation in SEQ ID NO: 61; and a frameshift mutation in ASXL1.

In some embodiments of these compositions and all such compositions described herein, the one or more somatic mutations are in PPM1D and are selected from a G1618T mutation in SEQ ID NO: 64; a C1372T mutation in SEQ ID NO: 64; and a frameshift mutation in PPM1D.

In some embodiments of these compositions and all such compositions described herein, the subject further has one or more somatic mutations in TET2 in a sub-population of peripheral blood hematopoietic cells selected from an S282F mutation in SEQ ID NO: 68, an N312S mutation in SEQ ID NO: 68, an L346P mutation in SEQ ID NO: 68, an S460F mutation in SEQ ID NO: 68, a D666G mutation in SEQ ID NO: 68, a P941S mutation in SEQ ID NO: 68, and a C1135Y mutation in SEQ ID NO: 68.

In some embodiments of these compositions and all such compositions described herein, the inhibitor of HSC cardiometabolic driver gene mutation-mediated proinflammatory activity is an IL-1β inhibitor.

In some embodiments of these compositions and all such compositions described herein, the IL-1β inhibitor is an IL-1β inhibitor antibody or antigen-binding fragment thereof that binds to IL-1β and reduces IL-1β binding to its receptor(s).

In some embodiments of these compositions and all such compositions described herein, the IL-1β inhibitor antibody or antigen-binding fragment thereof is selected from ABT981, an anti-interleukin-1β inhibitor antibody by ABZYME, APX002, Canakinumab/Ilaris, CDP48, immunereszumab, LY2189102, MED18968, and XOMA052.

In some embodiments of these compositions and all such compositions described herein, the IL-1β inhibitor is an IL-1 receptor antagonist.

In some embodiments of these compositions and all such compositions described herein, the IL-1 receptor antagonist is selected from CDP484, CP412245, CYT013 IL1bQb, XL 130, AMG108, HL 2351, IL1Hyl, AXXO, orthokine, PRT 1000, anakinra, and rilonacept.

In some embodiments of these compositions and all such compositions described herein, the IL-1β inhibitor is a small molecule or microRNA inhibitor that inhibits IL-1β-mediated pro-inflammatory activity.

In some embodiments of these compositions and all such compositions described herein, the small molecule or microRNA inhibitor is selected from AC201, CP412245, MCC950 or CRID3, inflabion, inflammasome modulator OPSONA, PGE3935199, PGE527667, TRK530, β-hydroxybutyrate (BHB), and microRNA-223. In some embodiments of these compositions and all such compositions described herein, the small molecule inhibitor is MCC950.

In some embodiments of these compositions and all such compositions described herein, the inhibitor of HSC cardiometabolic driver gene mutation-mediated proinflammatory activity is an IL-6 inhibitor.

In some embodiments of these compositions and all such compositions described herein, the IL-6 inhibitor is an IL-6 inhibitor antibody or antigen-binding fragment thereof that binds to IL-6 and reduces IL-6 binding to its receptor(s).

In some embodiments of these compositions and all such compositions described herein, the IL-6 inhibitor antibody or antigen-binding fragment thereof is selected from Siltuximab, Olokizumab, Elsilimomab, mAb 1339, Sirukumab, Clazakizumab, ARGX-109, FM101, and C326.

In some embodiments of these compositions and all such compositions described herein, the IL-6 inhibitor is an IL-6 receptor antagonist.

In some embodiments of these compositions and all such compositions described herein, the IL-6 receptor antagonist is selected from tocilizumab, sarilumab, REGN88, FE301, and LMT-28.

In some embodiments of these compositions and all such compositions described herein, the IL-6 inhibitor is a small molecule or microRNA inhibitor.

In some embodiments of these compositions and all such compositions described herein, the small molecule IL-6 inhibitor is ALX-0061 or LMT-28.

In some embodiments of these compositions and all such compositions described herein, the IL-6 inhibitor is a JAK-STAT inhibitor.

In some embodiments of these compositions and all such compositions described herein, the JAK-STAT inhibitor is selected from baricitinib, decernotinid, filgotinib, INCB-039110, ruxolitinib, tofacitinib, Oclacitinib, Gandotinib, Lestaurtinib, Momelotinib, Pacritinib, PF-04965842, Upadacitinib, and Peficitinib.

In some embodiments of these compositions and all such compositions described herein, the inhibitor of HSC cardiometabolic driver gene mutation-mediated proinflammatory activity is a TNFα inhibitor.

In some embodiments of these compositions and all such compositions described herein, the TNFα inhibitor is a TNFα inhibitor antibody or antigen-binding fragment thereof that binds to TNFα and reduces TNFα binding to its receptor(s).

In some embodiments of these compositions and all such compositions described herein, the TNFα inhibitor antibody or antigen-binding fragment thereof is selected from adalimumab, Adalimumab-atto, certolizumab pegol, golimumab, infliximab,

In some embodiments of these compositions and all such compositions described herein, the TNFα inhibitor is a TNFα receptor antagonist.

In some embodiments of these compositions and all such compositions described herein, the TNFα receptor antagonist is etanercept.

In some embodiments of these compositions and all such compositions described herein, the TNFα inhibitor is a small molecule or microRNA inhibitor.

In some embodiments of these compositions and all such compositions described herein, the HSC cardiometabolic driver gene mutation-mediated proinflammatory disease is a cardiometabolic disease or disorder, e.g., CVD, myocardial infarction, heart failure, cardiac remodeling, or the like.

In some embodiments of these compositions and all such compositions described herein, the HSC cardiometabolic driver gene mutation-mediated proinflammatory disease is a chronic kidney disease or disorder.

Provided herein, in some aspects, are methods for detecting a subject having, or at risk for, a cardiometabolic driver gene mutation-mediated proinflammatory disease comprising: (i) obtaining a hematopoietic cell sample from a subject, and (ii) sequencing the hematopoietic cell sample from the subject to detect one or more somatic mutations in one or more HSC cardiometabolic driver genes in the hematopoietic cell sample.

In some embodiments of these methods and all such methods described herein, the hematopoietic cell sample is a peripheral blood hematopoietic cell sample.

In some embodiments of these methods and all such methods described herein, the hematopoietic cell sample is enriched for myeloid-derived cells.

In some embodiments of these methods and all such methods described herein, the one or more somatic mutations in the one or more HSC cardiometabolic driver genes identified in the hematopoietic cell sample cause clonal hematopoiesis in the subject.

In some embodiments of these methods and all such methods described herein, at least 2% of the hematopoietic cells are identified as having one or more HSC cardiometabolic driver gene mutations. In some embodiments of these methods and all such methods described herein, the one or more HSC cardiometabolic driver genes are selected from TP53, JAK2, DNMT3A, ASXL1, and PPM1D/WIP1.

In some embodiments of these methods and all such methods described herein, the one or more somatic mutations are in TP53 and are selected from a G743A mutation in SEQ ID NO:2 and a A659G mutation in SEQ ID NO: 2.

In some embodiments of these methods and all such methods described herein, the one or more somatic mutations are in JAK2 and is a G1849T in SEQ ID NO: 56.

In some embodiments of these methods and all such methods described herein, the one or more somatic mutations are in DNMT3A and are selected from a T1115C mutation in SEQ ID NO: 37; a C2711T mutation in SEQ ID NO: 37; a C1837G mutation in SEQ ID NO: 37; a A1666G mutation in SEQ ID NO: 37; a C1789T mutation in SEQ ID NO: 37; a G2719A mutation in SEQ ID NO: 37; a G1627T mutation in SEQ ID NO: 37; an A2723G mutation in SEQ ID NO: 37; a G1797T mutation in SEQ ID NO: 37; a T2252G mutation in SEQ ID NO: 37; a C1560A mutation in SEQ ID NO: 37; a T1031C mutation in SEQ ID NO: 37; a G2645A mutation in SEQ ID NO: 37; a C2043G mutation in SEQ ID NO: 37; a C2446T mutation in SEQ ID NO: 37; a A2198G mutation in SEQ ID NO: 37; A2281G mutation in SEQ ID NO: 37; a C920G mutation in SEQ ID NO: 37; a A2204G mutation in SEQ ID NO: 37; and a frameshift mutation in DNMT3A.

In some embodiments of these methods and all such methods described herein, the one or more somatic mutations are in ASXL1 and are selected from a C2407T mutation in SEQ ID NO: 61; a C2893T mutation in SEQ ID NO: 61; a 1926_1926delinsAG mutation in SEQ ID NO: 61; and a frameshift mutation in ASXL1.

In some embodiments of these methods and all such methods described herein, the one or more somatic mutations are in PPM1D and are selected from a G1618T mutation in SEQ ID NO: 64; a C1372T mutation in SEQ ID NO: 64; and a frameshift mutation in PPM1D.

In some embodiments of these methods and all such methods described herein, the subject further has one or more somatic mutations in TET2 in a sub-population of peripheral blood hematopoietic cells selected from an S282F mutation in SEQ ID NO: 68, an N312S mutation in SEQ ID NO: 68, an L346P mutation in SEQ ID NO: 68, an S460F mutation in SEQ ID NO: 68, a D666G mutation in SEQ ID NO: 68, a P941S mutation in SEQ ID NO: 68, and a C1135Y mutation in SEQ ID NO: 68.

In some embodiments of these methods and all such methods described herein, the method further comprises administering a therapeutically effective amount of a pharmaceutical method comprising an inhibitor of HSC cardiometabolic driver gene mutation-mediated proinflammatory activity and a pharmaceutically acceptable carrier if one or more somatic mutations in one or more HSC cardiometabolic driver genes are identified in the hematopoietic cell sample.

In some embodiments of these methods and all such methods described herein, the inhibitor of HSC cardiometabolic driver gene mutation-mediated proinflammatory activity is an IL-1β inhibitor.

In some embodiments of these methods and all such methods described herein, the IL-1β inhibitor is an IL-1β inhibitor antibody or antigen-binding fragment thereof that binds to IL-1β and reduces IL-1β binding to its receptor(s).

In some embodiments of these methods and all such methods described herein, the IL-1β inhibitor antibody or antigen-binding fragment thereof is selected from ABT981, an anti-interleukin-1β inhibitor antibody by ABZYME, APX002, Canakinumab/Ilaris, CDP48, immunereszumab, LY2189102, MEDI8968, and XOMA052.

In some embodiments of these methods and all such methods described herein, the IL-1β inhibitor is an IL-1 receptor antagonist.

In some embodiments of these methods and all such methods described herein, the IL-1 receptor antagonist is selected from CDP484, CP412245, CYT013 IL1bQb, XL 130, AMG108, HL 2351, IL1Hyl, AXXO, orthokine, PRT 1000, anakinra, and rilonacept.

In some embodiments of these methods and all such methods described herein, the IL-1β inhibitor is a small molecule or microRNA inhibitor that inhibits IL-1β-mediated pro-inflammatory activity.

In some embodiments of these methods and all such methods described herein, the small molecule or microRNA inhibitor is selected from AC201, CP412245, MCC950 or CRID3, inflabion, inflammasome modulator OPSONA, PGE3935199, PGE527667, TRK530, β-hydroxybutyrate (BHB), and microRNA-223.

In some embodiments of these methods and all such methods described herein, the small molecule inhibitor is MCC950.

In some embodiments of these methods and all such methods described herein, the inhibitor of HSC cardiometabolic driver gene mutation-mediated proinflammatory activity is an IL-6 inhibitor.

In some embodiments of these methods and all such methods described herein, the IL-6 inhibitor is an IL-6 inhibitor antibody or antigen-binding fragment thereof that binds to IL-6 and reduces IL-6 binding to its receptor(s).

In some embodiments of these methods and all such methods described herein, the IL-6 inhibitor antibody or antigen-binding fragment thereof is selected from Siltuximab, Olokizumab, Elsilimomab, mAb 1339, Sirukumab, Clazakizumab, ARGX-109, FM101, and C326.

In some embodiments of these methods and all such methods described herein, the IL-6 inhibitor is an IL-6 receptor antagonist.

In some embodiments of these methods and all such methods described herein, the IL-6 receptor antagonist is selected from tocilizumab, sarilumab, REGN88, FE301, and LMT-28.

In some embodiments of these methods and all such methods described herein, the IL-6 inhibitor is a small molecule or microRNA inhibitor.

In some embodiments of these methods and all such methods described herein, the small molecule IL-6 inhibitor is ALX-0061 or LMT-28.

In some embodiments of these methods and all such methods described herein, the IL-6 inhibitor is a JAK-STAT inhibitor.

In some embodiments of these methods and all such methods described herein, the JAK-STAT inhibitor is selected from baricitinib, decernotinid, filgotinib, INCB-039110, ruxolitinib, tofacitinib, Oclacitinib, Gandotinib, Lestaurtinib, Momelotinib, Pacritinib, PF-04965842, Upadacitinib, and Peficitinib.

In some embodiments of these methods and all such methods described herein, the inhibitor of HSC cardiometabolic driver gene mutation-mediated proinflammatory activity is a TNFα inhibitor.

In some embodiments of these methods and all such methods described herein, the TNFα inhibitor is a TNFα inhibitor antibody or antigen-binding fragment thereof that binds to TNFα and reduces TNFα binding to its receptor(s).

In some embodiments of these methods and all such methods described herein, the TNFα inhibitor antibody or antigen-binding fragment thereof is selected from adalimumab, Adalimumab-atto, certolizumab pegol, golimumab, infliximab,

In some embodiments of these methods and all such methods described herein, the TNFα inhibitor is a TNFα receptor antagonist.

In some embodiments of these methods and all such methods described herein, the TNFα receptor antagonist is etanercept.

In some embodiments of these methods and all such methods described herein, the TNFα inhibitor is a small molecule or microRNA inhibitor.

In some embodiments of these methods and all such methods described herein, the method further comprises monitoring hematopoietic cell clonality, IL-1β proinflammatory activity, IL-6 proinflammatory activity, TNFα proinflammatory activity or any combination thereof following the administration of the inhibitor of HSC cardiometabolic driver gene mutation-mediated proinflammatory activity.

In some embodiments of these methods and all such methods described herein, the method further comprises decreasing the number or percentage of hematopoietic cells comprising the one or more somatic mutations in the one or more HSC cardiometabolic driver genes in the subject by performing therapeutic cytapheresis on the subject.

In some embodiments of these methods and all such methods described herein, the method further comprises administering one or more additional therapeutic agents to the subject.

In some embodiments of these methods and all such methods described herein, the HSC cardiometabolic driver gene mutation-mediated proinflammatory disease is a cardiometabolic disease or disorder.

In some embodiments of these methods and all such methods described herein, the HSC cardiometabolic driver gene mutation-mediated proinflammatory disease is a chronic kidney disease or disorder.

### Definitions

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by werner Luttmann, published by Elsevier, 2006; Janeway's Immunobiology, Kenneth Murphy, Allan Mowat, Casey weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, an “inhibitor of an HSC cardiometabolic driver gene mutation-mediated proinflammatory activity” refers to any agent or molecule that significantly blocks, inhibits, reduces, or interferes with the downstream effects of one or more somatic mutations in an HSC cardiometabolic driver gene, such as TP53, JAK2, DNMT3A, ASXL1, and/or PPM1D/WIP1, that leads to increased pro-inflammatory IL-1β signaling, increased pro-inflammatory IL-6 signaling, and/or increased pro-inflammatory TNFα signaling. Such increased pro-inflammatory IL-1β signaling, increased pro-inflammatory IL-6 signaling, and/or increased pro-inflammatory TNFα signaling includes, but is not limited to, increased IL-13, IL-6, and/or TNFα transcription, increased NLRP3 inflammasome-mediated IL-1β secretion, increased IL-1-Receptor 1-mediated IL-1β signaling, increased IL-6-Receptor α-mediated IL-6 signaling, increased gp130-mediated IL-6 signaling, increased JAK1/JAK2-mediated IL-6 signaling, increased STAT3/STAT1-mediated IL-6 signaling, increased TNFR1-mediated TNFα signaling, increased TNFR2-mediated TNFα signaling, and/or increased TRAF2/TRAF3-mediated TNFα signaling.

As used herein, the terms reduce(s)/reduced/reducing/reduction, inhibit(s)/inhibiting/inhibited or decrease(s)/decreasing/decreased generally means either a reduction or inhibition of at least 5%, at least 10%, at least 25%, at least 50%, at least 60%, at least 70%, at least 80%, at least 90%, at least 95%, at least 98%, or more, compared to the level of: IL-1β, IL-6, and/or TNFα transcription, IL-1β, IL-6, and/or TNFα translation, NLRP3 inflammasome-mediated IL-1β secretion, IL-1β binding to IL-1 receptor, IL-6 binding to IL-6-Receptor, IL-6 binding to gp130, and/or TNFα binding to TNFR 1 and/or TNFR2 under the same conditions but without the presence of inhibitors of HSC cardiometabolic driver gene-mediated proinflammatory activity described herein.

A disease or medical condition is considered to be mediated by “IL-1β, IL-6, and/or TNFα proinflammatory activity” if the spontaneous or experimental disease or medical condition is associated with, or mediated by, for example, elevated levels of IL-1β, IL-6, and/or TNFα in bodily fluids or tissue, or if cells or tissues taken from the body produce elevated levels of IL-1β, IL-6, and/or TNFα in culture.

As used herein, the phrase “cardiovascular condition, disease or disorder” is intended to include all disorders characterized by insufficient, undesired or abnormal blood vessel or cardiac function, e.g. hypertension, ischemic heart disease, hypertensive heart disease and pulmonary hypertensive heart disease, valvular disease, cardiac arrhythmia, vascular disease, myocardial infarction, congestive heart failure, peripheral vascular disease, myocarditis, atherosclerosis, restenosis, and any condition which leads to congestive heart failure in a subject, particularly a human subject.

As used herein, an “IL-1β inhibitory compound” or “IL-1β inhibitor” or “inhibitor of IL-1β” refers to a compound or agent capable of specifically inhibiting or specifically preventing activation of cellular receptors to IL-1β and consequent downstream effects of IL-1β signaling.

As used herein, an “interleukin-1 receptor antagonist” (“IL-1ra”) is any agent or molecule, including small molecules and antibody or antigen-binding fragments thereof, that binds to an interleukin-1 receptor thereby preventing binding of IL-1β to the receptor and thereby inhibiting IL-1β-mediated pro-inflammatory activity.

As used herein, an “IL-6 inhibitory compound” or “IL-6 inhibitor” or “inhibitor of IL-6” refers to a compound or agent capable of specifically inhibiting or specifically preventing activation of cellular receptors to IL-1β and consequent downstream effects of IL-1β signaling.

As used herein, a “TNFα inhibitory compound” or “TNFα inhibitor” or “inhibitor of TNFα” refers to a compound or agent capable of specifically inhibiting or specifically preventing activation of cellular receptors to TNFα and consequent downstream effects of TNFα signaling.

As used herein, “antibodies” or “antigen-binding fragments” thereof include monoclonal, human, humanized or chimeric antibodies, single chain antibodies, Fab fragments, F(ab′) fragments, fragments produced by a Fab expression library, and/or binding fragments of any of the above. Antibodies also refer to immunoglobulin molecules and immunologically active portions of immunoglobulin molecules, i.e., molecules that contain antigen or target binding sites or “antigen-binding fragments.” The immunoglobulin molecules described herein can be of any type (e.g., IgG, IgE, IgM, IgD, IgA and IgY), class (e.g., IgG1, IgG2, IgG3, IgG4, IgA1 and IgA2) or subclass of immunoglobulin molecule, as is understood by one of skill in the art.

The terms “antibody fragment” or “antigen-binding fragment” include: (i) the Fab fragment, having V_{L}, C_{L}, V_{H} and C_{H}1 domains; (ii) the Fab′ fragment, which is a Fab fragment having one or more cysteine residues at the C-terminus of the C_{H}1 domain; (iii) the Fd fragment having V_{H} and C_{H}1 domains; (iv) the Fd′ fragment having V_{H} and C_{H}1 domains and one or more cysteine residues at the C-terminus of the CH1 domain; (v) the Fv fragment having the V_{L} and V_{H} domains of a single arm of an antibody; (vi) a dAb fragment (Ward et al., Nature 341, 544-546 (1989)) which consists of a V_{H} domain or a V_{L} domain; (vii) isolated CDR regions; (viii) F(ab′)₂ fragments, a bivalent fragment including two Fab′ fragments linked by a disulphide bridge at the hinge region; (ix) single chain antibody molecules (e.g. single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; and Hollinger et al., Proc. Natl. Acad. Sci. USA, 90:6444-6448 (1993)); (xi) “linear antibodies” comprising a pair of tandem Fd segments (V_{H}-C_{H,} 1-V_{H}-C_{H}1) which, together with complementary light chain polypeptides, form a pair of antigen binding regions (Zapata et al. Protein Eng. 8(10): 1057-1062 (1995); and U.S. Pat. No. 5,641,870); and modified versions of any of the foregoing (e.g., modified by the covalent attachment of polyalkylene glycol (e.g., polyethylene glycol, polypropylene glycol, polybutylene glycol) or other suitable polymer).

As used herein, “small molecule inhibitors” include, but are not limited to, small peptides or peptide-like molecules, soluble peptides, and synthetic non-peptidyl organic or inorganic compounds. A small molecule inhibitor or antagonist can have a molecular weight of any of about 100 to about 20,000 daltons (Da), about 500 to about 15,000 Da, about 1000 to about 10,000 Da.

As used herein, the terms “HSC cardiometabolic driver gene activating compound” or “HSC cardiometabolic driver gene potentiatior” or “HSC cardiometabolic driver gene activator” or “HSC cardiometabolic driver gene agonist” refer to a molecule or agent that mimics or up-regulates (e.g., increases, potentiates or supplements) the biological activity of a given HSC cardiometabolic driver gene, thereby decreasing or inhibiting IL-1β, IL-6, and/or TNFα proinflammatory activity caused by deficient and/or reduced activity of the HSC cardiometabolic driver gene.

The terms “biological sample” or “sample” as used herein refers to a cell or population of cells or a quantity of tissue or fluid from a subject comprising one or more hematopoietic cells. Most often, the biological sample has been removed from a subject, but the term “biological sample” can also refer to cells or tissue analyzed in vivo, i.e., without removal from the subject.

As used herein, the term “population of hematopoietic cells” encompasses a heterogeneous or homogeneous population of hematopoietic cells and/or hematopoietic progenitor cells.

The terms “isolate” and “methods of isolation,” as used herein, refer to any process whereby a cell or population of cells, such as a population of hematopoietic cells, is removed from a subject or sample in which it was originally found, or a descendant of such a cell or cells.

### BRIEF DESCRIPTION OF THE DRAWINGS

FIG. 1 shows a summary of cytokine expression effects of different clonal hematopoiesis genes.

FIG. 2 shows effects of Tet2 inactivation on LPS-induced cytokine production. Murine J774.1 cells (macrophage cell line) were stably transformed with a lentivirus vector that inactivates the expression of Tet2 (closed bars) or a control vector (open bars). Cytokine transcript levels were measured at the indicated time points after stimulation with 10 ng/ml LPS.

FIG. 3 shows effects of Dnmt3a inactivation on LPS-induced cytokine production. Murine J774.1 cells (macrophage cell line) were stably transformed with a lentivirus vector that inactivates the expression of Dnmt3a (closed bars) or a control vector (open bars). Cytokine transcript levels were measured at the indicated time points after stimulation with 10 ng/ml LPS.

FIG. 4 shows effects of Asxl1 inactivation on LPS-induced cytokine production. Murine J774.1 cells (macrophage cell line) were stably transformed with a lentivirus vector that inactivates the expression of Asxl1 (closed bars) or a control vector (open bars). Cytokine transcript levels were measured at the indicated time points after stimulation with 10 ng/ml LPS.

FIGS. 5A-5F demonstrate the effect of TP53 loss of function on LPS-induced cytokine and chemokine production. Murine neutrophils were isolated from bone marrow of TP53-heterozygous mice or WT mice. FIGS. 5A-5F. Cytokine and chemokine transcript levels were measured at indicated time points after stimulation with 10 ng/ml LPS. Analysis of transcripts revealed that IL6 (FIG. 5A), IL1β (FIG. 5A.), TNFα (FIG. 5C), CCL3 (FIG. 5D), CXCL2 (FIG. 5E), and CXCL3 (FIG. 5F) were upregulated.

FIG. 6 shows effects of overexpressing a mutant form of Ppm1d/Wip1 on LPS-induced cytokine production. Murine J774.1 cells (macrophage cell line) were stably transformed with a lentivirus vector that overexpresses a mutant form of Ppm1d/Wip1 (closed bars) or a control vector (open bars). Cytokine transcript levels were measured at the indicated time points after stimulation with 10 ng/ml LPS.

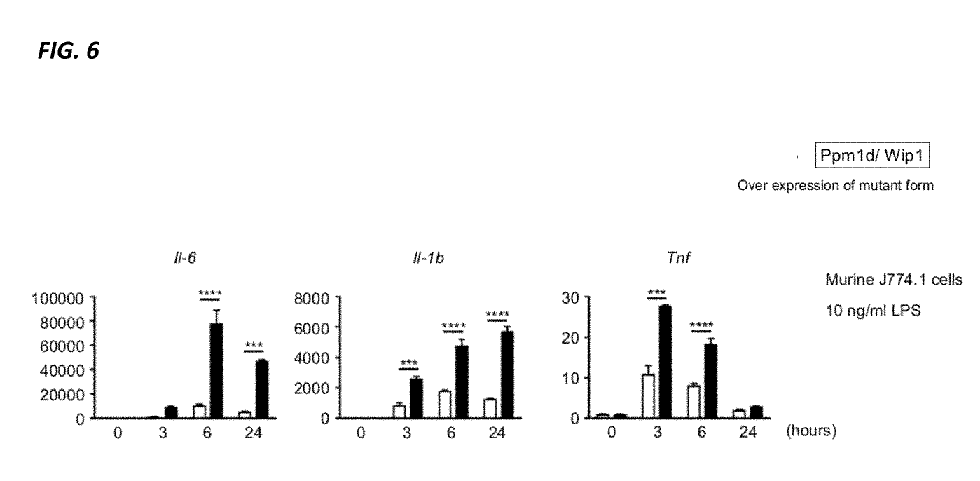

FIGS. 7A-7D show effects of overexpressing a mutant form of JAK2 (JAK2V617F) on cytokine production from unstimulated human THP-1 cells. Human THP-1 cells (macrophage cell line) were stably transformed with a lentivirus vector that overexpresses human JAK2V617F, wild-type JAK2 or a control vector. Cytokine and chemokine transcript levels were measured. FIG. 7A. Depiction of a lentiviral vector for bi-cistronic expression of the human JAK2 (WT or V617F) under CD146 enhancer and gp47 promoter with a Venus from a IRES sequence. FIG. 7B. THP-1 cells are transduced with lentiviral vector encoding JAK2-WT/sGFP, JAK2-V617F/sGFP display modest increase of JAK2, detected by western blotting. Lentiviral vector encoding Venus was used as control. FIG. 7C. THP-1 cells expressing JAK2-V617F shows enhanced STAT1 phosphorylation. No obvious increase in pSTAT3 and pSTAT5 was seen. FIG. 7D. Analysis of transcript expression in the THP-1 cells expressing sGFP, JAK2-WT/sGFP, JAK2-V617F/sGFP after 72h of differentiation. THP-1 monocytes are differentiated into macrophages by 24 h incubation with 100 nM of PMA followed by 24 h incubation in RPMI medium. Gene expression was analyzed by qPCR analysis. LTR: long terminal repeat, sGFP: superfolder green fluorescent protein, PMA: phorbol 12-myristate β-acetate.

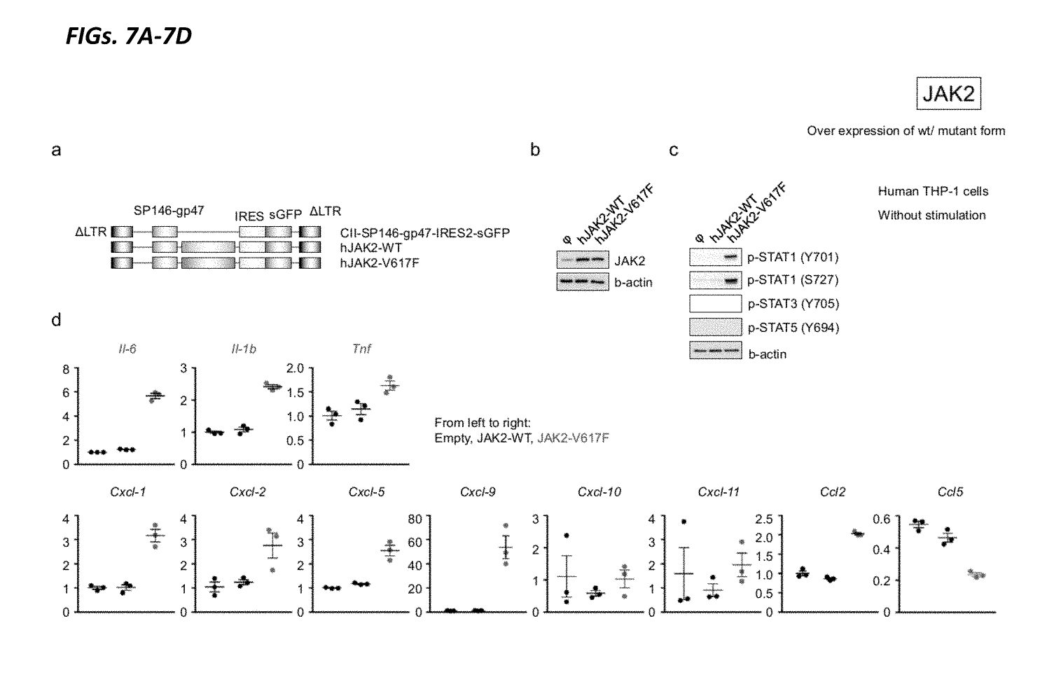

FIGS. 8A-8C demonstrate that competitive bone marrow transplantation studies in mice revealed the selective expansion TP53-deficient cells into multiple blood cell lineages and hematopoietic progenitor cells. FIG. 8A. Mice underwent partial (30%) bone marrow reconstitution with p53-deficient cells (30% KO-BMT) or wild-type cells (30% WT-BMT) following lethal irradiation. FIG. 8B. Flow cytometry analysis of peripheral blood was performed as indicated time points. FIG. 8C. Flow cytometry analysis of peripheral blood over time course to show p53-deficient cells have a greater repopulating ability. Flow cytometry analysis of bone marrow cells to show increase of hematopoietic stem/progenitor cells in mice reconstituted 30% of p53-deficient cells compared to 30% WT-BMT mice. BMT: bone marrow transfer, WBC: white blood cell, Mono: monocyte, Neut: neutrophil, B: B cell, CD4: CD4+ T cell, CD8: CD8+ T cell, LSK: lineage−, Sca1+, c-Kit+ cell, CMP: common myeloid progenitor, GMP: granulocyte and macrophage progenitor, MDP: macrophage and dendritic cell progenitor, MEP: megakaryocyte and erythroid progenitor.

FIGS. 9A-9C demonstrate greater pathological cardiac remodeling and lung congestion following permanent LAD ligation in mice that undergo an expansion of TP53-deficient hematopoietic cells. FIG. 9A. Mice underwent partial (30%) bone marrow reconstitution with p53-deficient cells (30% KO-BMT) or wild-type cells (30% WT-BMT) following lethal irradiation. 8 weeks after recover, mice underwent LAD ligation. Echocardiography was performed at the end of study (8 weeks after LAD ligation). FIG. 9B. Echocardiographic analysis showed that 30% KO-BMT mice display worsening cardiac remodeling after LAD ligation compared to 30% WT-BMT mice. FIG. 9C. LW adjusted by TL, showing that 30%-KO mice display the increase of lung mass after LAD ligation suggesting worsening of lung congestion. BMT: bone marrow transfer, LAD: left anterior descending artery, EF: ejection fraction, LW: lung weight, TL: tibia length.

FIGS. 10A-10B demonstrate human JAK2V617F transgenic hematopoietic stem and progenitor cells preferentially expand into the neutrophil and monocyte lineage. FIG. 10A. Mice underwent partial (20%) bone marrow reconstitution with human JAK2-V617F transgenic cells (20% V617F-BMT) or wild-type cells (20% WT-BMT) following lethal irradiation. Flow cytometry analysis of peripheral blood was performed as indicated time points. FIG. 10B. Flow cytometry analysis of peripheral blood over time course to show that JAK2V617F-expressing cells have a greater competitive advantage in myeloid populations. BMT: bone marrow transfer, Neut: neutrophil, Mono: monocyte.

FIGS. 11A-11F demonstrate validation of the targeted lentivirus vector to express human JAK2V617F in myeloid lineage cells with no impact on hemoglobin or platelet levels. FIG. 11A. Depiction of a lentiviral vector for bi-cistronic expression of the human JAK2 (WT or V617F) under CD146 enhancer and gp91 promoter with a Venus from a IRES sequence. FIG. 11B. Lineage-negative cells were harvested from bone marrow of 8-week old male C57B6/J mice. Lentivirus transduction was performed ex vivo for 16-24 hours. Transduced lineage-negative cells were transplanted to the lethally irradiated male C57B6/J mice (5×105 cells/mouse). FIG. 11C. Representative flow cytometry data of peripheral blood to show myeloid specific expression of lentivirus-transduced gene in vivo. Data is obtained 8 weeks after bone marrow reconstitution. FIG. 11D. Summary of data shown in FIG. 10C. FIG. 11E. Absolute numbers of peripheral blood of Hb and Plt from WT and V617F mice to show there is no significant changes in those hematological parameters. FIG. 11F. Flow cytometry analysis of the cardiac immune cells 7 days after myocardial infarction to show the expression of lentivirally transduced Venus gene in each population. IRES: internal ribosome entry site, LTR: long terminal repeat, BM: bone marrow, PB: peripheral blood, Mono: monocyte, Neut: neutrophil, B: B cell, T: T cell, Hb: hemoglobin, Plt: platelet, Mac: macrophage.

FIGS. 12A-12D demonstrate myeloid-specific expression of JAK2V617F expression promotes pathological remodeling and broad cytokine expression in hearts subjected to permanent LAD ligation. FIG. 12A. Mice underwent partial bone marrow reconstitution with lentivirus-transduced cells (JAK2-WT or JAK2-V617F) following lethal irradiation. 8 weeks after recover, mice underwent LAD ligation. Echocardiography was performed at indicated time points. FIG. 12B. Echocardiographic analysis showed that JAK2-V617F-BMT mice display reduced cardiac function after LAD ligation compared to JAK2-WT-BMT mice. FIG. 12C. Masson-Trichrome staining to show JAK2-V617F mice display greater infarct area 2 weeks after LAD ligation. FIG. 12D. Analysis of transcript expression in the infarct zone obtained from JAK2-V617F- and JAK2-WT-BMT mice. Gene expression was analyzed by qPCR analysis. BMT: bone marrow transfer, LAD: left anterior descending artery, MI: myocardial infarction, qPCR: quantitative polymerase chain reaction.

FIGS. 13A-13D demonstrate myeloid-specific expression of JAK2V617F expression promotes pathological remodeling in hearts subjected to transverse aortic constriction (TAC). FIG. 13A. Mice underwent partial bone marrow reconstitution with lentivirus-transduced cells (JAK2-WT or JAK2-V617F) following lethal irradiation. 8 weeks after recover, mice underwent TAC surgery. Echocardiography was performed at indicated time points. FIG. 13B. Echocardiographic analysis shows that JAK2-V617F-BMT mice display worsening cardiac hypertrophy and systolic function after TAC compared with JAK2-WT-BMTmice. FIG. 13C. HW and LW adjusted by TL, showing that mice underwent bone marrow reconstitution with JAK2-V617F cells display increase of cardiac and lung mass after TAC compared to control mice transplanted with JAK2-WT cells. FIG. 13D. Cardiac fibrosis at 8 weeks after TAC surgery detected by Picrosirius staining was more severe in JAK2-V617F-BMT mice than JAK2-WT-BMT mice. BMT: bone marrow transfer, TAC: transverse aortic constriction, PWd: posterior wall thickness at diastole, FS: fractional shortening, HW: heart weight, LW: lung weight, TL: tibia length.

FIGS. 14A-14D demonstrate lentivirus-CRISPR-mediated mutation of Dnmt3a in hematopoietic cells. FIG. 14A. Lineage-negative cells were harvested from bone marrow of 8-week old male C57B6/J mice. Lentivirus transduction was performed ex vivo for 16-24 hours. Transduced lineage-negative cells were transplanted to the lethally irradiated male C57B6/J mice (5×10⁵ cells/mouse). FIG. 14B. Guide targeting sequence of Dnmt3a gene (SEQ ID NO: 105). FIG. 14C. Flow cytometry analysis of peripheral blood over time course to show the stable modification of hematopoietic cell populations. FIG. 14D. Sequencing analysis revealed deletions and insertions in Dnmt3a gene. BM: bone marrow, BMT: bone marrow transfer, WBC: white blood cells, Mono: monocytes, Neut: neutrophils. Insertions underlined. FIG. 14D discloses SEQ ID NOS 106-108, respectively, in order of appearance.

FIGS. 15A-15E demonstrate lentivirus-CRISPR-mediated mutation of Dnmt3a in hematopoietic cells accelerates heart failure in mice infused with angiotensin II. FIG. 15A. Mice underwent bone marrow reconstitution with transduced lineage-negative cells following lethal irradiation. 8 weeks after recovery, mice underwent angiotensin-II infusion (1 μg/min/kg). Echocardiography was performed at the indicated time points. FIG. 15B. Echocardiographic analysis showed adverse cardiac remodeling in mice reconstituted with Dnmt3a-targeted bone marrow cells (hematopoietic Dnmt3a-KO). FIG. 15C. HW and LW adjusted by TL, showing that hematopoietic Dnmt3a-KO mice present increased cardiac and lung mass after angiotensin-II infusion. FIG. 15D. Representative images and measurements of CSA stained with WGA shows that hematopoietic Dnmt3a-KO mice display greater hypertrophy of the myocytes. Bar indicates 25 μm. FIG. 15E. Representative images and quantitative analysis of cardiac sections stained with Picrosirius red. Hematopoietic Dnmt3a-KO mice exhibit greater cardiac fibrosis after angiotensin-II infusion. Bar indicates 1 mm. BMT: bone marrow transfer, Ang-II: angiotensin-II, PWd: posterior wall thickness at diastole, FS: fractional shortening, HW: heart weight, LW: lung weight, TL: tibia length, PBS: phowphate-buffered saline, WGA: wheat germ agglutinin, CSA: cross-sectional area of myocytes.

FIGS. 16A-16G demonstrate that hematopoietic Tet2-KO mice show greater post-infarction remodeling. FIG. 16A. Scheme of the experimental study. Mice underwent partial (10%) bone marrow reconstitution with Tet2-deficient cells (10% KO-BMT) or wild type cells (10% WT-BMT) following lethal irradiation. After 8 weeks of recovery, mice underwent permanent LAD ligation. Echocardiography was performed at the indicated time points. FIG. 16B. Tet2-KO bone marrow cells (Cd45.2+) display a competitive advantage over wild type competitor cells (Cd45.1+) in their ability to expand into multiple blood cell lineages in vivo. Peripheral blood was obtained 8 weeks and 12 weeks after BMT (before (Pre) and 4 weeks after (Post) MI, respectively) from 10% WT-BMT (n=1) mice and 10% KO-BMT mice (n=10). Statistical analysis was evaluated by evaluated by two-way ANOVA with Tukey's multiple comparison tests. FIG. 16C. Absolute numbers of WBC before (Pre) and 4 weeks after (Post) LAD ligation of 10% KO-BMT mice (n=10) and 10% WT-BMT mice (n=11). Statistical analysis was evaluated by evaluated by two-way ANOVA with Tukey's multiple comparison tests. FIG. 16D Echocardiographic analysis shows that 10% KO-BMT mice (n=10) display worsening cardiac remodeling after LAD ligation compared to 10% WT-BMT mice (n=1). Statistical analysis was evaluated by two-way repeated measure ANOVA with Sidak's multiple comparison tests. FIG. 16E. Representative images and analysis of infarct size in myocardial tissue sections from 10% WT-BMT (n=3) mice and 10% KO-BMT mice (n=3) stained with TTC 2 days after LAD ligation, showing there is no statistical significance between both groups. Hearts were sliced at 2 mm below from the ligation site. Statistical analysis was evaluated by two-tailed unpaired Student's t test. f. Representative images and analysis of fibrosis in marginal zone of myocardial tissue sections from 10% WT-BMT (n=7) mice and 10% KO-BMT mice (n=7) stained with Masson's Trichrome dye at 4 weeks after ligation, showing worsening fibrosis in 10% KO-BMT mice. The percentage of the fibrotic area was calculated with the image-J software. Statistical analysis was evaluated by two-tailed unpaired Student's t test. Scale bars indicate 100 μm. FIG. 16G. Representative images and analysis of WGA staining of the heart sections from hearts 10% WT-BMT (n=7) mice and 10% KO-BMT mice (n=7) isolated at 4 weeks after LAD ligation. Staining shows that the non-infarcted, remote area of the heart displays greater hypertrophy of the cardiac myocytes. Statistical analysis was evaluated by two-way ANOVA with Tukey's multiple comparison test. Scale bars indicate 50 μm. **p<0.01, ***p<0.001, ****p<0.0001, NS: not significant. WT: wild type, KO: knockout, BMT: bone marrow transfer, LAD: left anterior ascending artery. WBC: white blood cells, Mono: monocytes, Neut: neutrophils, LV: left ventricle, EF: ejection fraction, TTC: 2,3,5-triphenyl-tetrazolium chloride, CSA: cross-sectional area of myocytes. From left to right at each timepoint/condition on the x-axes of FIGS. 16B, 16C, 16D, 16E, 16F, and 16G is presented 10% Wt and 10% KO.

FIGS. 17A-17H demonstrate that conditional myeloid Tet2-deficiency in mice leads to worsening of cardiac remodeling in hearts subjected to LAD ligation. FIG. 17A. Scheme of the study. Control and conditional myeloid Tet2-knockout (Myelo-KO) mice underwent LAD ligation. Mice underwent permanent LAD ligation, and echocardiography was performed at the indicated time points. FIG. 17B. The efficiency of Tet2 ablation was analyzed by qPCR in BMDM at 7 days after in vitro differentiation from conditional Tet2-Myelo-KO mice and control mice (3 mice per genotype). Two-tailed Student's t test was performed for statistical analysis. FIG. 17C. Flow cytometry representative data and analysis of peripheral blood from Tet2-Myelo-KO mice (n=6) and control mice (n=6) to show there are no detectable changes in myeloid populations. Statistical significance of difference was evaluated by multiple t test. FIG. 17D. Mice survival curve after LAD ligation. The mortality of the conditional KO mice and control mice after surgery was 37.5% and 40.0%, respectively. Log-rank test was used for statistical analysis (n=20 for control mice and n=16 for conditional Tet2-KO mice). FIG. 17E. Echocardiographic evaluation shows that surviving mice with conditional Tet2 ablation in myeloid cells (n=10) display worsening cardiac remodeling after LAD ligation surgery compared to control mice (n=12). Statistical analysis was evaluated by two-way repeated measure ANOVA with Sidak's multiple comparison tests. FIG. 17F. Representative images and analysis of infarct size in myocardial tissue sections from conditional KO mice (n=3) and control (n=3) mice stained with TTC 2 days after LAD ligation. Hearts were sliced at 2 mm below from the ligation site, showing that there was no difference in initial infarct size. Statistical analysis was evaluated by Mann-Whitney U test. FIG. 17G. Representative images and analysis of fibrosis in the marginal zone of myocardial tissue sections from conditional KO mice (n=6) and control (n=6) mice stained with Masson's Trichrome dye at 4 weeks after ligation, showing worsening fibrosis in conditional KO mice. The percentage of the fibrotic area was calculated with the image-J software. Statistical analysis was evaluated by two-tailed unpaired Student's t test. Scale bars indicate 100 μm. FIG. 17H. WGA staining of the heart sections from hearts control (n=6) mice and conditional KO mice (n=6) isolated at 4 weeks after LAD ligation. Analysis of CSA shows that the non-infarcted, remote area of the heart of conditional KO mice display greater hypertrophy of the cardiac myocytes. Statistical analysis was evaluated by two-way ANOVA followed with Tukey's multiple comparison tests. Scale bars indicate 50 μm. *p<0.05, **p<0.01, ****p<0.0001, NS: not significant. WT: wild type, KO: knockout, LAD: left anterior ascending artery, qPCR: quantitative polymerase chain reaction, BMDM: bone marrow-derived macrophages, LAD: left anterior ascending artery, Mono: monocytes, Neut: neutrophils, LV: left ventricle, EF: ejection fraction, TTC: 2,3,5-triphenyl-tetrazolium chloride, WGA: wheat germ agglutinin, CSA: cross-sectional area of myocytes. From left to right at each timepoint/condition on the x-axes of FIGS. 17B, 17C, 17E, 17F, 17G, and 17G: control, myelo-KO

FIGS. 18A-18F show the effect of Tet2-deficient hematopoietic cells on the expression of pro-inflammatory cytokines and chemokines in the remodeling heart tissue. FIG. 18A. Analysis of transcript expression in the non-infarcted marginal zone obtained from 10% KO-BMT mice (n=10) and 10% WT-BMT (n=1) mice. Gene expression was analyzed by qPCR analysis. Statistical significance was evaluated by two-tailed unpaired Student's t tests with Welch's Correction when variance was unequal or by Mann Whitney U tests for data which failed to pass the Shapiro-Wilk normality test. FIG. 18B. Flow cytometry analysis of cardiac remote area from 10% KO-BMT mice (n=7) and 10% WT-BMT (n=7) mice to show the absolute number of total CD45⁺ immune cells are increased in the myocardial tissue from 10% KO-BMT mice. Data is expressed as number of cells per 100 mg wet weight. Statistical analysis was evaluated by two-tailed unpaired Student's t test. FIG. 18C. Flow cytometry analysis of cardiac remote area from 10% KO-BMT mice (n=7) and 10% WT-BMT (n=7) mice to show the absolute number of each immune cell populations. Statistical significance of difference was evaluated by multiple t tests. FIG. 18D. IL-1β immunofluorescence staining in Mac3-positive macrophage-enriched marginal zone of 10% KO-BMT (n=5) mice and 10% WT-BMT mice (n=5) showing IL-1β signal is higher in 10% KO-BMT mice. Scale bars=20 μm. Images were quantified for integrated fluorescence intensity with Image J software. Statistical analysis was performed by two-tailed unpaired Student's t tests. FIG. 18E. Remote area samples were obtained from conditional myeloid-specific KO mice and control mice, and gene expression was analyzed by qPCR at the indicated time points (n=3 for sham and n=10 at 4 weeks after LAD ligation, per genotype). Statistical significance was evaluated by two-way ANOVA with Tukey's multiple comparison tests. FIG. 18F. Bone marrow-derived macrophages 2 days after in vitro differentiation obtained from Tet2-null mice (n=6) and wild type (n=7) were obtained in vitro and gene expression was analyzed by qPCR analysis. Statistical significance of difference was evaluated by two-tailed unpaired Student's t tests with Welch's Correction when variance was unequal or by Mann Whitney U tests for data which failed to pass the Shapiro-Wilk normality test. *p<0.05, **p<0.01, ****p<0.0001. WT: wild type, KO: knockout, BMT: bone marrow transfer, LAD: left anterior ascending artery, qPCR: quantitative polymerase chain reaction, RM: remote area, Mac: macrophages, Mono: monocytes, Neut: neutrophils, B: B cells, T: T cells, ND: not detected. From left to right at each timepoint/condition on the x-axes of FIG. 18A-18F: WT, KO.

FIGS. 19A-19D demonstrate that inflammasome inhibition reverses post-infarction remodeling associated with hematopoietic Tet2-deficiency. FIG. 19A. Scheme of the experimental study. Mice underwent partial (10%) bone marrow reconstitution with Tet2-deficient cells (10% KO-BMT mice) or wild type cells (10% WT-BMT mice) following lethal irradiation. After 8 weeks of recovery, mice underwent permanent LAD ligation. 1 week after LAD ligation, MCC950 and PBS was continuously infused with osmotic pumps for 4 weeks. Echocardiography was performed at the indicated time points. FIG. 19B. Echocardiographic analysis reveals that treatment with the NLRP3 inflammasome inhibitor MCC950 protects against adverse cardiac remodeling in 10% KO-BMT and 10% WT-BMT mice, and eliminates the differences in cardiac parameters between Tet2-deficient and WT conditions at the post-LAD ligation time point of 5 weeks. Sample sizes were n=12 for 10% WT-BMT with PBS, n=12 for 10% KO-BMT with PBS, n=14 for 10% WT-BMT with MCC950, n=14 for 10% KO-BMT with MCC950. Statistical significance was evaluated by two-way repeated measure ANOVA with Tukey's multiple comparison tests. FIG. 19C. Representative images and analysis of fibrosis in marginal zone of myocardial tissue sections stained with Masson's Trichrome dye at 5 weeks after ligation. MCC950 inhibits the development of cardiac fibrosis after LAD ligation in mice reconstituted with Tet2-KO or WT bone marrow, and eliminates the differences in cell size between Tet2-deficient and WT genotypes. Statistical significances of differences among groups of 10% WT/10% KO with PBS or MCC950 were evaluated by two-way ANOVA with Tukey's multiple comparison tests. Scale bars indicate 100 μm. FIG. 19D. Representative images and analysis of WGA staining of the heart sections of hearts at 5 weeks after LAD ligation. MCC950 inhibits the development of cardiac myocyte hypertrophy after LAD ligation in mice reconstituted with Tet2-KO or WT bone marrow, and eliminates the differences in cell size between Tet2-deficient and WT genotypes. For c and d, sham-operated mice without any pump infusion were used as control (n=3 per genotype). Sample sizes were n=6 for 10% WT-BMT with PBS, n=6 for 10% KO-BMT with PBS, n=8 for 10% WT-BMT with MCC950, n=8 for 10% KO-BMT with MCC950. Statistical significances of differences among groups of 10% WT/10% KO with PBS or MCC950 were evaluated by two-way ANOVA with Tukey's multiple comparison tests. Scale bars indicate 50 μm. **p<0.01, ***p<0.001, ****p<0.0001. WT: wild type, KO: knockout, BMT: bone marrow transfer, LAD: left anterior ascending artery, PBS: phosphate-buffered saline, WGA: wheat germ agglutinin, CSA: cross-sectional area of myocytes.

FIGS. 20A-20G demonstrate that inflammasome inhibition reverses pressure overload-induced cardiac remodeling associated with hematopoietic Tet2-deficiency. FIG. 20A. Scheme of the experimental study. Mice underwent partial (10%) bone marrow reconstitution with Tet2-deficient cells (10% KO-BMT mice) or WT cells (10% WT-BMT) following lethal irradiation. After 8 weeks of recovery, mice underwent permanent TAC surgery to produce pressure overload on the heart. MCC950 or PBS were infused from 1 week after TAC. FIG. 20B. IL-1β transcripts were determined in heart samples from 10% WT-BMT (n=7) mice and 10% KO-BMT mice (n=7) after pressure overload were by qPCR analysis. Statistical significance was evaluated by Mann-Whitney U test. FIG. 20C. Representative images of Picrosirius red staining to show the heart from 10% KO-BMT mice is larger compared to the heart from 10% WT-BMT mice 5 weeks after TAC. Scale bar indicates 1 mm. FIG. 20D. HW adjusted by TL, showing that MCC950 ameliorates the increase of cardiac mass after pressure overload in both strains of mice and eliminates the differences in these parameters between the Tet2-deficient and WT conditions (n=7 for TAC with PBS and n=8 for TAC with MCC950 per genotype). Sham operated mice without any infusion were used as control (n=3 per genotype). Statistical significances of differences among groups of 10% WT/10% KO with PBS or MCC950 were evaluated by two-way ANOVA with Tukey's multiple comparison tests. FIG. 20E. Echocardiographic analysis shows that infusion with MCC950 protects against adverse cardiac remodeling in mice reconstituted with Tet2-KO and wild-type bone marrow, and eliminates the differences in cardiac parameters between Tet2-deficient and WT genotypes at the 5 weeks after TAC surgery. Echocardiography was performed at the indicated time points. Sample sizes were n=7 for 10% WT-BMT with PBS, n=7 for 10% KO-BMT with PBS, n=8 for 10% WT-BMT with MCC950, n=8 for 10% KO-BMT with MCC950. Statistical significance of difference was evaluated by two-way repeated measure ANOVA with Tukey's multiple comparison tests. FIG. 20F. Quantitative analysis of cardiac sections stained with Picro sirius red as presented in FIG. 20C. shows that mice reconstituted with Tet2-knockout bone marrow exhibit greater cardiac fibrosis after pressure overload that can be reversed by treatment with MCC950. The MCC950 treatment eliminates the difference in this parameter between the Tet2-deficient and WT conditions. Sample sizes were n=7 for 10% WT-BMT with PBS, n=7 for 10% KO-BMT with PBS, n=8 for 10% WT-BMT with MCC950, n=8 for 10% KO-BMT with MCC950. Sham mice without any infusion were used as control (n=5 per genotype). Statistical significances of differences among groups of 10% WT/10% KO with PBS or MCC950 were evaluated by two-way ANOVA with Tukey's multiple comparison tests. FIG. 20G. Representative images and measurement of CSA stained with WGA shows that MCC950 inhibits hypertrophy after pressure overload both in wild type and hematopoietic Tet2-KO mice and eliminates the difference in parameters between the Tet2-deficient and WT conditions. Sample sizes were n=7 for 10% WT-BMT with PBS, n=7 for 10% KO-BMT with PBS, n=8 for 10% WT-BMT with MCC950, n=8 for 10% KO-BMT with MCC950. Sham mice without any infusion were used as control (n=5 per genotype). Statistical significances of differences among groups of 10% WT/10% KO with PBS or MCC950 were evaluated by two-way ANOVA with Tukey's multiple comparison tests. *p<0.05, ****p<0.0001; NS: not significant. WT: wild type, KO: knockout, BMT: bone marrow transfer, TAC: transverse aortic constriction, PBS: phosphate-buffered saline, qPCR: quantitative polymerase chain reaction, HW: heart weight, TL: tibia length, WGA: wheat germ agglutinin, WGA: wheat germ agglutinin, CSA: cross-sectional area of myocytes.

FIG. 21 shows a schematic illustration of how clonal hematopoiesis promotes heart failure. The illustration shows that tomatic Tet2 mutations within hematopoietic stem and progenitor cells (HSPC) will lead to their clonal amplification and these HSPC give rise to myeloid cell progeny that promote cardiac remodeling through excessive production of interleukin-1beta (IL-1J3).

FIG. 22 shows the flow cytometry gating strategy of peripheral blood after competitive BMT. Cells were defined as: (i) total white blood cells (CD45⁺), (ii) Ly6C^{hi} monocytes (CD115^{high} Ly6G⁻, CD43^{low}, Ly6C^{high}), (iii) Ly6C^{lo} monocytes (CD115^{high}, Ly6G⁻, CD43^{high}, Ly6C^{low}), (iv) neutrophils (CD115^{low}, Ly6G⁺), (v) B cells (CD3e⁻, B220⁺), (vi) T cells (CD3e⁺, B220⁻, CD4/8⁺). CD45.1 and CD45.2 were used to determine the chimerism in each population. BMT: bone marrow transfer. WBC: white blood cells, Neut: neutrophils, Mono: monocytes, B: B cells, T: T cells.

FIGS. 23A-23C show the flow cytometry gating strategy of cardiac tissues. FIG. 23A. scheme of the sampling of heart tissue after LAD ligation. Hearts were divided into remote area and infarct area with marginal zone. FIG. 23B. Myeloid panel of infarct area (6 days after MI). FIG. 23C. Lymphoid panel of infarct area (14 days after MI). Cells were defined as: (i) total white blood cells (CD45⁺), (ii) neutrophils (CD11b⁺, Ly6G⁺), (iii) Ly6C^{hi} monocytes (CD11b⁺, Ly6G⁻, Ly6C^{hi}, F4/80^{lo}), (iv) macrophages (CD11b⁺, Ly6G⁻, Ly6C^{lo}, F4/80^{hi}), (v) B cells (CD11b⁻, CD3e⁻, B220⁺, CD19⁺), (vi) T cells (CD11b⁻, CD3e⁺, B220⁻, CD4/8⁺). RM: remote area, IA infarct area, MZ: marginal zone, MI: myocardial infarction, Neut: neutrophils, Mac: macrophages, Mono: monocytes, B: B cells, T: T cells.

FIGS. 24A-24D shows an increase in LSK cells in the bone marrow of donor 6-8 week old Tet2-deficient mice. FIG. 24A. Flow cytometry gating strategy of bone marrow hematopoietic stem/progenitor cells. FIG. 24B. Flow cytometry gating strategy of bone marrow myeloid cells. Cells were defined as: (i) LSK cells (Lin⁻, c-Kit⁺, Sca1⁺), (ii) GMP (Lin⁻, c-Kit⁺, Sca1⁻, CD34⁺, CD16/32^{hi}, CD115⁻), (iii) GMP (Lin, c-Kit⁺, Sca1⁻, CD34⁺, CD16/32^{hi}, CD115⁺), (iv) monocytes (CD11b⁺, CD115⁺), (v) neutrophils (CD11b⁺, CD115⁻, Ly6G⁺, MHC-II⁻). FIG. 24C. The number of each populations in 2.5×10⁶ bone marrow cells from Tet2-deficient mice (n=7) and wild type mice (n=7). All mice are 6-8 weeks old. Statistical significance of difference was evaluated by two-tailed unpaired Student's t tests or by Mann Whitney U tests for data which failed to pass the Shapiro-Wilk normality test. From left to right on each x-axis is shown WT and KO. FIG. 24D. The weight of the spleen from Tet2-deficient mice (n=5) and wild type mice (n=5) to show there is no significant difference between both genotypes at this age. Statistical analysis was evaluated by two-tailed unpaired Student's t test with Welch's correction. GMP: granulocyte-macrophage progenitors, MDP: monocyte-dendritic cell progenitors, Neut: neutrophils, Mono: monocytes.

FIG. 25 demonstrates that Tet2-deficient hematopoietic stem cells display a greater repopulating ability in vivo. Tet2-KO bone marrow cells (Cd45.2⁺) display a competitive advantage over wild type competitor cells (Cd45.1⁺) in their ability to expand into multiple blood cell lineages in vivo. Peripheral blood was obtained 8 weeks after BMT from 10% WT-BMT (n=1) mice and 10% KO-BMT mice (n=10). Statistical analysis was evaluated by multiple t tests. **p<0.01, ****p<0.0001. WBC: white blood cells, Mono: monocytes, Neut: neutrophils, B: B cells, T: T cells.

FIGS. 26A-26B demonstrate that LAD ligation does not affect Tet2-deficient peripheral blood chimerism. FIG. 26A. Scheme of the experimental study. Mice underwent partial (10%) bone marrow reconstitution with Tet2-deficient cells or WT cells following lethal irradiation. After 8 weeks of recovery, mice underwent permanent LAD ligation (MI). Blood chimerism was analyzed by flow cytometry at 4 weeks after ligation. FIG. 26B. The percentage of the CD45.2⁺ cells in different peripheral blood lineages. Sample sizes were n=5 for 10% WT-BMT/sham, n=4 for 10% KO-BMT/sham, n=5 for 10% WT-BMT/MI, n=5 for 10% KO-BMT/MI. From left to right the series for each condition are: 10% WT-BMT/sham, 10% WT-BMT/MI, 10% KO-BMT/sham, 10% KO-BMT/MI. Statistical significance was evaluated by two-way ANOVA with Tukey's multiple comparison tests. NS: not significant. HSPC: hematopoietic stem progenitor cells, WT: wild type, KO: knockout, LAD: left anterior ascending artery, MI: myocardial infarction, WBC: white blood cells, Neut: neutrophils, Mono: monocytes, B: B cells, T: T cells.

FIG. 27 shows survival curves of Tet-2 deficient mice with partial BMT after LAD ligation. WT: wild type, KO: knockout, NS: not significant. The statistical analysis of Kaplan-Meier Curve was evaluated by log-rank test.

FIGS. 28A-28C demonstrate a dose-dependent impact of HSPC Tet2-deficiency on post-MI cardiac remodeling. FIG. 28A. Scheme of the experimental study. Mice underwent partial (10%) bone marrow reconstitution with Tet2-homozygous null cells or Tet2-heterogyzous cells or WT cells following lethal irradiation. After 8 weeks of recovery, mice underwent permanent LAD ligation (MI). FIG. 28B. Tet2-homozygous null cells or Tet2-heterogyzous bone marrow cells (Cd45.2⁺) display a competitive advantage over wild type competitor cells (Cd45.1⁺) in their ability to expand into multiple blood cell lineages in vivo. Peripheral blood was obtained 4 weeks and 8 weeks after BMT. Sample sizes were 5 per genotype. Statistical analysis was evaluated by two-way repeated measure ANOVA with Sidak's multiple comparison tests. FIG. 28C. Echocardiographic evaluation of the mice 4 weeks after BMT. Sample sizes were 5 per genotype. Statistical significance was evaluated by one-way ANOVA with Tukey's multiple comparison tests. *p<0.05, **p<0.01, ***p<0.001, ****p<0.0001; NS: not significant. HSPC: hematopoietic stem progenitor cells, WT: wild type, Het: heterozygote, KO: knockout, BMT: bone marrow transfer, LAD: left anterior ascending artery, MI: myocardial infarction, LV: left ventricle, EF: ejection fraction.

FIGS. 29A-29B show myeloid-specific Tet2-deficient mice do not show obvious changes in hematological parameters. FIG. 29A. Absolute numbers of peripheral blood of Hb, WBC, and Plt from Tet2-Myelo-KO mice (n=6) and control mice (n=6) to show there is no significant changes in those hematological parameters. Statistical significance was evaluated by two-tailed unpaired Student's t tests or by Mann Whitney U tests for data which failed to pass the Shapiro-Wilk normality test. FIG. 29B. Flow cytometry representative data and analysis of peripheral blood from Tet2-Myelo-KO mice (n=6) and control mice (n=6) to show there is no significant changes in lymphoid populations. Statistical significance of difference was evaluated by multiple t test. Hb: hemoglobin, WBC: white blood cells, Plt: platelets, B: B cells, T: T cells, NS: not significant.

FIGS. 30A-30B show the flow cytometry analysis of cardiac remote area and infarct area in hematopoietic Tet2-deficient mice. FIG. 30A. Flow cytometry analysis of cardiac remote area from 10% KO-BMT mice (n=7) and 10% WT-BMT (n=7) mice to show the relative proportion of immune cell populations. Statistical analysis was evaluated by multiple t tests. FIG. 30B. Flow cytometry analysis of cardiac infarct area with marginal zone from the same mice of a. to show the absolute number of CD45⁺ cells (left panel) and macrophages (right panel). Statistical significance of difference was evaluated by two-tailed unpaired Student's t test or by Mann Whitney U tests for data which failed to pass the Shapiro-Wilk normality test. *p<0.05, **p<0.01. WT: wild type, Het: heterozygote, KO: knockout, BMT: bone marrow transfer, RM: remote area, IA: infarct area, MZ: marginal zone, Mac: macrophages, Mono: monocytes, Neut: neutrophils, B: B cells, T: T cells.

FIGS. 31A-31B demonstrate that hematopoietic Tet2-KO mice do not show obvious change of the macrophages proliferation. FIG. 31A. Representative data of the Ki67 (green) and Mac3 (red) immunofluorescence staining of the marginal zone from 10% KO-BMT mice (n=6) and 10% WT-BMT (n=6). DAPI is used to detect nuclei (blue). Heart tissue samples are obtained 4 weeks after LAD ligation. Scale bars indicate 20 μm. FIG. 31B. The number of Mac3⁺ cells (left panel) and the ratio of Ki67⁺ cells over Mac3⁺ cells (right) are shown. Unpaired two-tailed Student's t test was performed for statistical analysis. Scale bar, 100 μm. WT: wild type, KO: knockout, LAD: left anterior ascending artery.

FIGS. 32A-32B demonstrate that MCC950 does not impact the peripheral blood chimerism. FIG. 32A. Scheme of the experimental study. Mice underwent partial (10%) bone marrow reconstitution with Tet2-deficient cells or WT cells following lethal irradiation. After 4 weeks of recovery, mice underwent MCC950 or PBS infusion. Blood chimerism was analyzed by flow cytometry at 0 and 2 weeks after infusion. FIG. 32B. The percentage of the CD45.2⁺ cells in different peripheral blood lineages before and after MCC950/PBS infusion. Sample sizes were n=3 for 10% WT-BMT/PBS, n=3 for 10% KO-BMT/PBS, n=4 for 10% WT-BMT/MCC950, n=4 for 10% KO-BMT/MCC950. Statistical significance was evaluated by two-way ANOVA with Tukey's multiple comparison tests. *p<0.05, ***p<0.001; NS: not significant. HSPC: hematopoietic stem progenitor cells, WT: wild type, KO: knockout, BMT: bone marrow transfer, PBS: phosphate-buffered saline, WBC: white blood cells, Mono: monocytes.

FIGS. 33A-33I demonstrate that conditional myeloid Tet2-deficiency in mice leads worse cardiac remodeling during pressure overloaded hypertrophy. FIG. 33A. Scheme of the experimental study. Conditional myeloid Tet2-knockout mice and control mice underwent TAC and echocardiography was performed at the indicated time points. FIG. 33B. Echocardiographic evaluation shows that mice with conditional Tet2 ablation in myeloid cells (n=9) display worsening cardiac remodeling after TAC surgery compared to control mice (n=9). The echocardiographic measurement time points are indicated. Statistical analysis was evaluated by two-way repeated measure ANOVA with Sidak's multiple comparison tests. FIG. 33C. Representative images of Picrosirius Red stained cardiac sections. Hearts from conditional Tet2-KO mice appear larger compared to the hearts from control mice at 8 weeks after TAC. FIG. 33D. Measurements of HW and LW normalized to TL. Statistical significance was evaluated by two-way ANOVA with Tukey's multiple comparison tests. Sample sizes were n=3 for control/sham, n=3 for conditional Tet2-KO/sham, n=9 for control/TAC, n=9 for conditional Tet2-KO/TAC. FIG. 33E. Quantitative analysis of cardiac sections stained with Picrosirius red as presented in FIG. 33C. shows that conditional Tet2-KO mice (n=8) exhibit greater cardiac fibrosis after pressure overload then control mice (n=8) 8 weeks after TAC. For the 0 time point, 3 sham-treated mice were also used as used per genotype. The percentage of the fibrotic area was calculated with the image-J software. Statistical analysis was evaluated by two-way ANOVA with Tukey's multiple comparison tests. FIG. 33F. WGA staining of the heart sections from hearts from conditional Tet2-KO mice (n=8) and control mice (n=8) isolated at 8 weeks after TAC showing that conditional KO mice display greater hypertrophy of the cardiac myocytes. 3 sham mice were used as used per genotype. Statistical analysis was evaluated by two-way ANOVA with Tukey's multiple comparison tests. FIG. 33G. Hearts from conditional myeloid Tet2-deficient mice upregulate of IL-1β transcript after pressure overload. Remodeling heart tissue samples were obtained from control (n=8) mice and conditional KO mice (n=8) and gene expression was analyzed by qPCR analysis. For the 0 time point, sham-treated mice were used (n=8 per genotype). Statistical significance was evaluated by two-way ANOVA with Tukey's multiple comparison tests. FIG. 33H. Flow cytometry analysis of hypertrophied myocardium from Tet2-Myelo-KO mice (n=7) and control (n=7) mice to show the absolute number of total CD45⁺ immune cells are increased in the tissue from Tet2-Myelo-KO mice. Data is expressed as number of cells per 100 mg wet weight. Statistical analysis was evaluated by two-tailed unpaired Student's t test. FIG. 33I. Flow cytometry analysis of hypertrophied myocardium from Tet2-Myelo-KO mice (n=7) and control (n=7) mice to show the relative proportion (left panel) and absolute number (right panel) of each immune cell populations. Statistical significance of difference was evaluated by multiple t tests. *p<0.05, **p<0.01, ***p<0.001, ****p<0.0001; NS: not significant. WT: wild type, Myelo-KO: myeloid-specific knockout, TAC: transverse aortic constriction, HW: heart weight, LW: lung weight, TL: tibia length, LVPWTd: left ventricular posterior wall thickness at end diastole, FS: fractional shortening, qPCR: quantitative polymerase chain reaction, CSA: cross-sectional area of myocytes, Mac: macrophages, Mono: monocytes, Neut: neutrophils, B: B cells, T: T cells. In all graphs, the first series is control, and the second series is myelo-KO.

FIGS. 34A-34D demonstrate that CRISPR/Cas9 (clustered regularly interspaced short palindromic repeats/clustered regularly interspaced short palindromic repeat-associated 9)-mediated Tet2 gene disruption confers a competitive advantage to the hematopoietic stem/progenitor cells (HSPCs). FIG. 34A. Bone marrow lineage-negative cells from wild-type mice were transduced with lentivirus particles expressing Cas9/eGFP (enhanced green fluorescent protein) and delivered to lethally irradiated wild-type mice. FIG. 34B. Flow cytometry analysis of HSPC transduction by lentivirus. Cells are defined as LSK cells (lineage⁻, c-kit⁺, Sca-1⁺) and HSC (hematopoietic stem cell; CD48⁻, CD150⁺ in LSK cells). Transduced cells are GFP positive (n=4). FIG. 34C. Flow cytometry analysis of the peripheral blood at 4 and 16 wk after reconstitution with bone marrow transduced with Tet2 (ten-eleven translocation-2)-targeted and control (no Tet2 guide RNA) lentivirus vectors. The percentage of GFP⁺ cells in both experimental groups is shown (n=6 in both Tet2-indel [insertion and deletion] mice and control mice). Statistical analysis was evaluated by 2-way repeated measure ANOVA with Sidak multiple comparison tests. FIG. 34D. Results of the TA cloning procedure showing that GFP peripheral white blood cells harbor edited Tet2 genes. The wild-type Tet2 sequence is shown for reference. *P<0.05, **P<0.01, ****P<0.0001. BM indicates bone marrow; Mono, monocyte; Neut, neutrophil; Sca-1, stem cells antigen-1; SSC, side scatter; and WBC, white blood cell. FIG. 34D discloses SEQ ID NOS 109-113, respectively, in order of appearance.

FIGS. 35A-35H show a phenotype comparison of Ang II (angiotensin-II) infusion-induced cardiac dysfunction between conventional competitive bone marrow transplant (BMT) model and lenti-CRISPR/Cas9 (clustered regularly interspaced short palindromic repeats/clustered regularly interspaced short palindromic repeat-associated 9) model. FIGS. 35A-35D shows the experimental results obtained from the conventional competitive BMT model. FIGS. 35E-35H, Data from lenti-CRISPR/Cas9 model. FIG. 35A. Echocardiographic analysis at the indicated time points after Ang II infusion (12 mice per group). Statistical analysis was evaluated by 2-way repeated measures ANOVA with Sidak multiple comparison tests. The first series is 10% WT and the second series is 10% Tet2-KO. FIG. 35B. Heart weight (HW) adjusted by TL at the end of the study (8 wk). Statistical analysis was evaluated by 2-way ANOVA with Sidak multiple comparison tests. FIG. 35C. Representative images and analysis of WGA (wheat germ agglutinin) staining of the heart sections from hearts of 10% knockout (KO)-BMT mice and 10% WT (wild type)-BMT mice at the end of the study. Statistical analysis was evaluated by 2-way ANOVA with Sidak multiple comparison tests (scale bar=25 μm). FIG. 35D. Representative images and analysis of Picrosirius red staining of the heart sections from hearts of 10% KO-BMT mice and 10% WT-BMT mice at the end of the study. Statistical analysis was evaluated by 2-way ANOVA with Sidak multiple comparison tests (scale bar=1 mm). The first series is WT and the second series is Tet2-indel. For FIGS. 35B-35D, n=12 for Ang II groups and n=5 for PBS groups were analyzed. FIG. 35E. Echocardiographic analysis at indicated time points after Ang II infusion (6 mice per group). Statistical analysis was evaluated by 2-way repeated measures ANOVA with Sidak multiple comparison tests. FIG. 35F. HW adjusted by TL at the end of the study (8 wk). Statistical analysis was evaluated by Mann-Whitney U test. FIG. 35G Representative images and analysis of WGA staining of the heart sections from hearts of Tet2 (ten-eleven translocation-2)-indel (insertion and deletion) mice and control mice at the end of the study (scale bar=1 mm). Statistical analysis was evaluated by 2-tailed unpaired Student t test. FIG. 35H Representative images and analysis of Picrosirius staining of the heart sections from hearts of Tet2-indel mice and control mice at the end of the study (scale bar=25 μm). Statistical analysis was evaluated by 2-tailed unpaired Student t test. For FIGS. 35E-35H, n=6 per group were analyzed. **P<0.01, ***P<0.001, ****P<0.0001. CSA indicates cross-sectional area; FS, fractional shortening; HW, heart weight; and TL, tibia length.

FIGS. 36-36F demonstrate that CRISPR/Cas9 (clustered regularly interspaced short palindromic repeats/clustered regularly interspaced short palindromic repeat-associated 9)-mediated hematopoietic Dnmt3a gene disruption promotes cardiac dysfunction after Ang II (angiotensin II) infusion. FIG. 36A. Flow cytometry analysis of the peripheral blood at 4 and 16 wk after bone marrow reconstitution. The percentages of GFP⁺ (green fluorescent protein) cells in both experimental groups are shown (n=13 in both Dnmt3a [DNA (cytosine-5)-methyltransferase 3a]-indel [insertion and deletion] mice and control mice). Statistical analysis was evaluated by 2-way repeated measures ANOVA with Sidak multiple comparison tests. FIG. 36B. The result of the TA cloning procedure showing that GFP⁺ peripheral white blood cells harbor edited Dnmt3a genes. The wild-type Dnmt3a sequence is shown for reference. FIG. 36B discloses SEQ ID NOS 114 and 106-108, respectively, in order of appearance. FIG. 36C. Echocardiographic analysis at indicated time points after Ang II infusion (10 mice per group). Statistical analysis was evaluated by 2-way repeated measures ANOVA with Tukey multiple comparison tests. FIG. 36D. Heart weight (HW) adjusted by TL at the end of the study (2 mo). Statistical analysis was evaluated by 2-way ANOVA with Tukey multiple comparison tests. FIG. 36E. Representative images and analysis of Picrosirius staining of the heart sections from hearts of Dnmt3a-indel mice and control mice at the end of the study (scale bar=1 mm). Statistical analysis was evaluated by 2-way ANOVA with Tukey multiple comparison tests. FIG. 36F. Representative images and analysis of WGA (wheat germ agglutinin) staining of the heart sections from hearts of Dnmt3a-indel mice and control mice at the end of the study (scale bar=25 μm). Statistical analysis was evaluated by 2-way ANOVA with Tukey multiple comparison tests. For FIGS. 36D-36F, n=10 for Ang II groups and n=7 for PBS groups were analyzed. NS indicates nonsignificant. ***P<0.001, ****P<0.0001. CSA indicates cross-sectional area; FS, fractional shortening; Mono, monocyte; Neut, neutrophil; TL, tibia length; and WBC, white blood cell. For the graphs in FIGS. 36C-36F, the series are, from left to right: PBS Control, PBS Dnmt3a-Indel, AngII Control, AngII Dnmt3a-Indel.

FIGS. 37A-37D demonstrate that hematopoietic Dnmt3a (DNA [cytosine-5]-methyltransferase 3a) loss of function enhances cardiac inflammation. FIG. 37A. Western blot analysis revealing decrease in Dnmt3a expression in J774.1 cells treated with lentivirus-mediated Dnmt3a knockout. The lentivirus without sgRNA (single guide RNA) was used as control. FIG. 37B. Gene expression analysis of WT (wild type), Tet2 (ten-eleven translocation-2)-indel (insertion and deletion), and Dnmt3a-indel J774.1 cells at 6 h after stimulation with 10 ng/mL lipopolysaccharide (LPS). Statistical analysis was evaluated by 2-way ANOVA with Tukey multiple comparison tests. FIG. 37C. Representative images and analysis of Mac2 staining of the sections of hearts from Dnmt3a-indel mice and control mice at 8 wk after Ang II (angiotensin II) infusion (n=6 per group; scale bar=100 μm). Statistical analysis was evaluated by Mann-Whitney U test. FIG. 37D. Gene expression analysis of heart from Dnmt3a-indel and control mice (n=10 per group) 8 wk after Ang II infusion. Statistical analysis was evaluated by 2-tailed unpaired Student t test or Mann-Whitney U test. NS indicates nonsignificant. *P<0.05, **P<0.01, ***P<0.001, ****P<0.0001. Mac2 indicates macrophage-2 antigen; and NT, nontreated.

FIGS. 38A-38E show the validation of gRNA targeting Tet2 and Dnmt3a. FIG. 38A. Depiction of a lentiviral vector containing the gRNA targeting Tet2 or Dnmt3a under the control of the U6 promoter (U6) and Cas9 under the control of the short EF1a promoter (EFS). eGFP is bicistronically expressed using picorna virus-derived 2A auto-cleavage site (P2A) system. FIG. 38A discloses SEQ ID NOS 115 and 103, respectively, in order of appearance. FIG. 38B. Schema of the experimental study. PX459 plasmid encoding gRNA targeting Tet2 or Dnmt3a under the control of U6 promoter and Cas9 under the control of the CMV promoter is transfected to Nl H-3T3 cells. After 48 hours selection with puromycin (2 mg/ml), followed by 1 week for recovery, cells are collected for analysis. FIG. 38C. Result of T7 endonuclease 1 mismatch cleavage assay performed with genomic DNA from Nl H-3T3 cells transfected with 3 sequences of gRNA targeting Tet2. FIG. 38D Result of T7 endonuclease 1 mismatch cleavage assay performed with genomic DNA from Nl H-3T3 cells transfected with gRNA targeting Dnmt3a. FIG. 38E. Western immunoblot analysis to show that Dnmt3a is ablated in Nl H-3T3 cells.

FIGS. 39A-39B shows the schema of this study. FIG. 39A. Mice underwent partial (10%) bone marrow reconstitution with Tet2-deficient cells (10% knockout-bone marrow transfer) or wild-type cells (10% WT-BMT) following lethal irradiation. After 8 weeks of recovery, mice underwent Angll infusion. FIG. 39B. Bone marrow lineage-negative cells from wild type mice were transduced with lentivirus particles harboring gRNA and Cas9/eGFP into lethally irradiated wild type mice, followed by Angll infusion for 8 weeks. Echocardiography was performed at the indicated time points.

FIG. 40 shows the flow cytometry gating strategy of peripheral blood. Cells were defined as: (i) total white blood cell (CD45+), (ii) monocytes (CD115⁺, Ly6G⁻), (iii) neutrophils (Ly6G⁺, CD115⁻), (iv) B cells (CD115⁻, Ly6G⁻, CD3e⁻, B220⁺), (v) T cells (CD115⁻, Ly6G⁻, CD3e⁺, B220⁻). The percentage of GFP⁺ cells in these populations were measured by using the negative control (GFP⁻) as a reference.

FIG. 41 demonstrates that Tet2-KO bone marrow cells to preferentially expand into multiple blood cells in vivo. Mice underwent partial (10%) bone marrow reconstitution with Tet2-deficient cells (10% KO) or wild-type cells (10% WT) following lethal irradiation. After 8 weeks of recovery, flow cytometry analysis of peripheral blood was performed. Statistical significance was evaluated by two-tailed unpaired Student's t test or Mann-Whitney U test. *p<0.01, ***p<0.001.

FIGS. 42A-42C show a higher magnification of cardiac fibrosis. FIG. 42A. Representative images and analysis of Picro sirius staining. Heart sections from mouse reconstituted with Tet2-deficient bone marrow cells (10% KO) or wild-type bone marrow cells (10% WT). FIG. 42B. Heart sections from Tet2-indel mice. FIG. 42 C. Heart sections from Dnmt3a-indel mice. Scale bar: 100 mm.

FIGS. 43A-43C demonstrate that Myeloid-specific Tet2 gene disruption promotes cardiac dysfunction after Ang-II infusion. FIG. 43A. Echocardiographic analysis of myeloid-specific (LysM-cre) myelo-Tet2-KO mice and control mice at indicated time points after Angll infusion (10 mice per group). Statistical analysis was evaluated by two-way ANOVA with Sidak's multiple comparison tests. FIG. 43B. Analysis of WGA staining of the heart sections from hearts of myelo-Tet2-KO mice and control mice at the end of the study. Statistical analysis was evaluated by two-way ANOVA with Tukey's multiple comparison tests. FIG. 43C. Analysis of picrosirius staining of the heart sections from hearts of myelo-Tet2-KO mice and control mice at the end of the study. Statistical analysis was evaluated by two-way ANOVA with Tukey's multiple comparison tests. p<0.05, **p<0.01, ***p<0.001, ****p<0.0001.

FIGS. 44A-44B demonstrate that CRISPR/Cas9-mediated mutation of Tet2 and Dnmt3a in HSPC promotes AngII-induced renal fibrosis. FIG. 44A. Representative images and analysis of Masson's trichrome staining of the kidney sections from kidney of Tet2-indel mice and control mice at the end of the study. Statistical analysis was evaluated by two-tailed unpaired Student's t test. FIG. 44B. Representative images and analysis of Masson's trichrome staining of the kidney sections from kidney of Dnmt3a-indel mice and control mice at the end of the study. Statistical analysis was evaluated by two-way ANOVA with Tukey's multiple comparison tests. Scale bar: 100 mm. *p<0.05, ****p<0.0001.

### DETAILED DESCRIPTION

Advances in DNA sequencing have revealed that aging is associated with an increased frequency of somatic mutations in proliferative tissues, particularly in the hematopoietic system. Recently, large exome sequencing studies in humans have shown that aging is associated with an increased frequency of somatic mutations in the hematopoietic system which provide a competitive growth advantage to the mutant cell and therefore allow its clonal expansion, referred to herein as “clonal hematopoiesis” (Jaiswal et al, Genovese et al. NEJM 2014; Xi et al, Nat Med 2014). Furthermore, recent studies employing ultra-deep sequencing demonstrate that somatic mutations in blood cells are much more prevalent than previously recognized (McKerrell, Cell Reports 2015). However, while recent human studies demonstrate that somatic mutations can be associated with a broad spectrum of human disease, there is a lack of experimental evidence supporting their causal contribution to age-associated disorders other than cancer (Science special issues on “Mutation and Human Disease” (September 2015) and “Why we Age” (December 2015)). In contrast, experimental evidence is provided herein that mechanistically links clinically relevant somatic mutations in cells of hematopoietic origin to cardiovascular disease (CVD), metabolic, renal, and other chronic diseases that have a large inflammatory component. The experimental demonstrations described herein provide novel evidence of the causal contribution of a scenario of genome mosaicism in the hematopoietic system and subsequent clonal hematopoiesis to a non-hematological disorder.

Epidemiological studies show that HSPCs develop mutations that promote their clonal expansion at a relatively high frequency in the aging population. While very few of the HSCs acquire subsequent mutations in oncogenes that lead to blood cancers, the mechanistic findings of the studies described herein, using TP53, JAK2, and DNMT3A as examples, show that a single gene mutation that occurs frequently can predispose an individual to CVD and stroke that are common in the elderly (>50% of individuals). The studies described herein demonstrate that somatic mutations in genes such as TP53, JAK2, DNMT3A, ASXL1, and PPM1D/WIP1 in HSCs, termed herein as “HSC cardiometabolic driver genes,” that lead to increased production of inflammatory cytokines, such as IL-1β, IL-6, and TNF, can lead to various signs and symptoms of cardiovascular disease and pathological remodeling. As demonstrated herein, TP53 mediated hematopoietic cell expansion contributes to pathological remodeling in heart failure. Competitive bone marrow transplantation studies in mice revealed the selective expansion TP53-deficient cells into multiple blood cell lineages and features consistent with an exacerbated heart failure phenotype. Further, as demonstrated herein, a somatic activating mutation V617F in JAK2 in hematopoietic cells led to greater pathological remodeling of the heart following injury, and was accompanied by the broad over-activation of cytokines, including IL-6, IL-1β and TNFα, in the heart. In addition, hematopoietic cell mutation of Dnmt3a was demonstrated herein to lead to diminished cardiac function, increased cardiac hypertrophy, increased myocyte hypertrophy, and increased cardiac fibrosis. Thus, the data provided herein show that somatic mutations in HSC cardiometabolic driver genes, such as TP53, JAK2, DNMT3A, ASXL1, and PPM1D/WIP1, and consequent clonal hematopoiesis, can contribute to pathological cardiac remodeling following injury and facilitate heart failure.

“TP53” or “tumor protein 53,” which is encoded on human chromosome 17, is a tumor suppressor protein containing transcriptional activation, DNA binding, and oligomerization domains. The encoded protein is known to respond to diverse cellular stresses to regulate expression of target genes, thereby inducing cell cycle arrest, apoptosis, senescence, DNA repair, or changes in metabolism. Mutations in this gene are associated with a variety of human cancers, including hereditary cancers such as Li-Fraumeni syndrome.

Accordingly, the term “TP53” as used herein, refers to the genomic sequence of NG_017013.2 (SEQ ID NO: 1) encoding: the mRNA sequence of NM_000546.5 (isoform a, SEQ ID NO: 2), which encodes the polypeptide having the amino acid sequence of NP_000537.3 (isoform a, SEQ ID NO: 3); the mRNA sequence of NM_001126112.2 (isoform a2, SEQ ID NO: 4) encoding the polypeptide having the amino acid sequence of NP_001119584.1 (isoform a2, SEQ ID NO: 5); the mRNA sequence of NM_001126113.2 (isoform c, SEQ ID NO: 6) encoding the polypeptide having the amino acid sequence of NP_001119585.1 (isoform c, SEQ ID NO: 7); the mRNA sequence of NM_001126114.2 (isoform b, SEQ ID NO: 8) encoding the polypeptide having the amino acid sequence of NP_001119586.1 (isoform b, SEQ ID NO: 9); the mRNA sequence of NM_001126115.1 (isoform d, SEQ ID NO: 10) encoding the polypeptide having the amino acid sequence of NP_001119587.1 (isoform d, SEQ ID NO: 11); the mRNA sequence of NM_001126116.1 (isoform e, SEQ ID NO: 12) encoding the polypeptide having the amino acid sequence of NP_001119588.1 (isoform e, SEQ ID NO: 13); the mRNA sequence of NM_001126117.1 (isoform f, SEQ ID NO: 14) encoding the polypeptide having the amino acid sequence of NP_001119589.1 1 (isoform f, SEQ ID NO: 15); the mRNA sequence of NM_001126118.1 (isoform g, SEQ ID NO: 16) encoding the polypeptide having the amino acid sequence of NP_001119590.1 (isoform g, SEQ ID NO: 17); the mRNA sequence of NM_001276695.1 (isoform h, SEQ ID NO: 18) encoding the polypeptide having the amino acid sequence of NP_001263624.1 (isoform h, SEQ ID NO: 19); the mRNA sequence of NM_001276696.1 (isoform i, SEQ ID NO: 20) encoding the polypeptide having the amino acid sequence of NP_001263625.1 (isoform i, SEQ ID NO: 21); the mRNA sequence of NM_001276697.1 (isoform j, SEQ ID NO: 22) encoding the polypeptide having the amino acid sequence of NP_001263626.1 (isoform j, SEQ ID NO: 23); the mRNA sequence of NM_001276698.1 (isoform k, SEQ ID NO: 24) encoding the polypeptide having the amino acid sequence of NP_001263627.1 (isoform k, SEQ ID NO: 25); the mRNA sequence of NM_001276699.1 (isoform 1, SEQ ID NO: 26) encoding the polypeptide having the amino acid sequence of NP_001263628.1 (isoform 1, SEQ ID NO: 27); the mRNA sequence of NM_001276760.1 (isoform g1, SEQ ID NO: 28) encoding the polypeptide having the amino acid sequence of NP_001263689.1 (isoform g 1, SEQ ID NO: 29); and the mRNA sequence of NM_001276761.1 (isoform g2, SEQ ID NO: 30) encoding the polypeptide having the amino acid sequence of NP_001263690.1 (isoform g2, SEQ ID NO: 31), together with any additional naturally occurring allelic, splice variants, and processed forms thereof. Typically, as used herein, TP53 refers to human TP53. Reference to specific sub-fragments or sub-sequences of TP53 can be identified in the application, e.g., by “nucleic acids 211-402 of TP53 of SEQ ID NO: 1.” Specific nucleic acid or amino acid residues of TP53 can be referred to as, for example, “A282 of TP53 of SEQ ID NO: 1” or A282 of SEQ ID NO: 1.”

“DNMT3A” or “DNA (cytosine-5-1-methyltransferase 3 alpha,” which is encoded on human chromosome 2, encodes a DNA methyltransferase that is believed to function in de novo methylation, rather than maintenance methylation. DNMT3A localizes to the cytoplasm and nucleus and its expression is developmentally regulated.

Accordingly, the term “DNMT3A” as used herein, refers to the genomic sequence of NG_029465.2 (SEQ ID NO: 32) encoding: the mRNA sequence of NM_001320892.1 (isoform c, SEQ ID NO: 33), which encodes the polypeptide having the amino acid sequence of NP_001307821.1 (isoform c, SEQ ID NO: 34); the mRNA sequence of NM_001320893.1 (isoform d, SEQ ID NO: 35) encoding the polypeptide having the amino acid sequence of NP_001307822.1 (isoform d, SEQ ID NO: 36); the mRNA sequence of NM_022552.4 (isoform a, SEQ ID NO: 37) encoding the polypeptide having the amino acid sequence of NP_072046.2 (isoform a, SEQ ID NO: 38); the mRNA sequence of NM_153759.3 (isoform b, SEQ ID NO: 39) encoding the polypeptide having the amino acid sequence of NP_715640.2 (isoform b, SEQ ID NO: 40); the mRNA sequence of NM_175629.2 (isoform a, SEQ ID NO: 41) encoding the polypeptide having the amino acid sequence of NP_783328.1 (isoform a, SEQ ID NO: 42); and the mRNA sequence of NM_175630.1 (isoform c2, SEQ ID NO: 43) encoding the polypeptide having the amino acid sequence of NP_783329.1 (isoform e, SEQ ID NO: 44); together with any additional naturally occurring allelic, splice variants, and processed forms thereof. Typically, as used herein, DNMT3A refers to human DNMT3A. Reference to specific sub-fragments or sub-sequences of DNMT3A can be identified in the application, e.g., by “nucleic acids 211-402 of DNMT3A of SEQ ID NO: 32.” Specific nucleic acid or amino acid residues of DNMT3A can be referred to as, for example, “A282 of DNMT3A of SEQ ID NO: 32” or A282 of SEQ ID NO: 32.”

“JAK2” or “janus kinase 2,” which is encoded on chromosome 9, is a protein tyrosine kinase involved in a specific subset of cytokine receptor signaling pathways. It has been found to be constitutively associated with the prolactin receptor and is required for responses to gamma interferon. Mice that do not express an active protein for this gene exhibit embryonic lethality associated with the absence of definitive erythropoiesis.

Accordingly, the term “JAK2” as used herein, refers to the genomic sequence of NG_009904.1 (SEQ ID NO: 45) encoding: the mRNA sequence of NM_001322194.1 (isoform a2, SEQ ID NO: 46), which encodes the polypeptide having the amino acid sequence of NP_001309123.1 (isoform a2, SEQ ID NO: 47); the mRNA sequence of NM_001322195.1 (isoform a3, SEQ ID NO: 48) encoding the polypeptide having the amino acid sequence of NP_001309124.1 (isoform a3, SEQ ID NO: 49); the mRNA sequence of NM_001322196.1 (isoform a4, SEQ ID NO: 50) encoding the polypeptide having the amino acid sequence of NP_001309125.1 (isoform a4, SEQ ID NO: 51); the mRNA sequence of NM_001322198.1 (isoform c, SEQ ID NO: 52) encoding the polypeptide having the amino acid sequence of NP_001309128.1 (isoform c, SEQ ID NO: 53); the mRNA sequence of NM_001322204.1 (isoform b, SEQ ID NO: 54) encoding the polypeptide having the amino acid sequence of NP_001309133.1 (isoform b, SEQ ID NO: 55); and the mRNA sequence of NM_004972.3 (isoform a1, SEQ ID NO: 56) encoding the polypeptide having the amino acid sequence of NP_004963.1 (isoform a1, SEQ ID NO: 57); together with any additional naturally occurring allelic, splice variants, and processed forms thereof. Typically, as used herein, JAK2 refers to human JAK2. Reference to specific sub-fragments or sub-sequences of JAK2 can be identified in the application, e.g., by “nucleic acids 211-402 of JAK2 of SEQ ID NO: 45.” Specific nucleic acid or amino acid residues of JAK2 can be referred to as, for example, “A282 of JAK2 of SEQ ID NO: 45” or A282 of SEQ ID NO: 45.”

“ASXL1” or “additional sex combs like transcriptional regulator 1,” which is encoded on chromosome 20, is similar to the Drosophila additional sex combs gene, which encodes a chromatin-binding protein required for normal determination of segment identity in the developing embryo. The protein is a member of the Polycomb group of proteins, which are necessary for the maintenance of stable repression of homeotic and other loci. The protein is thought to disrupt chromatin in localized areas, enhancing transcription of certain genes while repressing the transcription of other genes. The protein encoded by this gene functions as a ligand-dependent co-activator for retinoic acid receptor in cooperation with nuclear receptor coactivator 1. Mutations in this gene are associated with myelodysplastic syndromes and chronic myelomonocytic leukemia. Alternative splicing results in multiple transcript variants.

Accordingly, the term “ASXL1” as used herein, refers to the genomic sequence of NG_027868.1 (SEQ ID NO: 58) encoding: the mRNA sequence of NM_001164603.1 (isoform 2, SEQ ID NO: 59), which encodes the polypeptide having the amino acid sequence of NP_001158075.1 (isoform 2, SEQ ID NO: 60); and the mRNA sequence of NM_015338.5 (isoform 1, SEQ ID NO: 61) encoding the polypeptide having the amino acid sequence of NP_056153.2 (isoform 1, SEQ ID NO: 62); together with any additional naturally occurring allelic, splice variants, and processed forms thereof. Typically, as used herein, ASXL1 refers to human ASXL1. Reference to specific sub-fragments or sub-sequences of ASXL1 can be identified in the application, e.g., by “nucleic acids 211-402 of ASXL1 of SEQ ID NO: 56.” Specific nucleic acid or amino acid residues of ASXL1 can be referred to as, for example, “A282 of ASXL1 of SEQ ID NO: 56” or A282 of SEQ ID NO: 56.”

“PPM1D” or “protein phosphatase, Mg2+/Mn2+ dependent, 1D,” which is encoded on human chromosome 17, is a member of the PP2C family of Ser/Thr protein phosphatases. PP2C family members are known to be negative regulators of cell stress response pathways. The expression of this gene is induced in a p53-dependent manner in response to various environmental stresses. While being induced by tumor suppressor protein TP53/p53, this phosphatase negatively regulates the activity of p38 MAP kinase, MAPK/p38, through which it reduces the phosphorylation of p53, and in turn suppresses p53-mediated transcription and apoptosis. This phosphatase thus mediates a feedback regulation of p38-p53 signaling that contributes to growth inhibition and the suppression of stress induced apoptosis. This gene is located in a chromosomal region known to be amplified in breast cancer. The amplification of this gene has been detected in both breast cancer cell line and primary breast tumors, indicating a role of this gene in cancer development.

Accordingly, the term “PPM1D” as used herein, refers to the genomic sequence of NG_023265.1 (SEQ ID NO: 63) encoding: the mRNA sequence of NM_003620.3 (SEQ ID NO: 64), which encodes the polypeptide having the amino acid sequence of NP_003611.1 (SEQ ID NO: 65); together with any additional naturally occurring allelic, splice variants, and processed forms thereof. Typically, as used herein, PPM1D refers to human PPM1D. Reference to specific sub-fragments or sub-sequences of PPM1D can be identified in the application, e.g., by “nucleic acids 211-402 of PPM1D of SEQ ID NO: 56.” Specific nucleic acid or amino acid residues of PPM1D can be referred to as, for example, “A282 of PPM1D of SEQ ID NO: 56” or A282 of SEQ ID NO: 56.”

As described herein, somatic mutations or deficiencies in HSC cardiometabolic driver genes, such as TP53, JAK2, DNMT3A, ASXL1, and PPM1D/WIP1, in HSCs can lead to increased production of inflammatory cytokines, such as IL-1β, IL-6, and TNF, resulting in various signs and symptoms of cardiovascular disease and pathological remodeling. The studies described herein, using competitive bone marrow transplantation experiments in mice, demonstrate for the first time that selective expansion of HSCs lacking or having somatic mutations in a subset of genes, including TP53, JAK2, and DNMT3A, results in features consistent with heart failure phenotypes, including diminished cardiac function, increased cardiac hypertrophy, increased myocyte hypertrophy, and increased cardiac fibrosis. Accordingly, the studies described herein support a new paradigm of causal risk for cardiovascular diseases, metabolic diseases, and other inflammation-mediated diseases, whereby somatic mutations in HSCs, and consequent HSC clonal hematopoiesis, result in increased inflammatory cytokine production.

Hematopoietic stem cells (HSCs) and hematopoietic progenitor cells (HPCs) divide to produce blood cells by a continuous regeneration process. As the cells divide, they are prone to accumulating mutations, including deletions, insertions, and substitutions, that generally do not affect function. However, some mutations confer advantages in self-renewal, proliferation or both, resulting in clonal expansion of the cells comprising the mutations in question. The frequency of such somatic mutation events increases with age. The studies described herein demonstrate that preferential and progressive expansion of a subset of hematopoietic cells bearing somatic mutations in HSC cardiometabolic driver genes, such as TP53, JAK2, DNMT3A, ASXL1, and PPM1D/WIP1, leads to increased production of inflammatory cytokines, such as IL-1β, IL-6, and/or TNFα.

Compositions and Theranostic Methods for Treating IL-1β, IL-6, and/or TNFα-Mediated Proinflammatory Activity

As demonstrated herein, somatic mutations in HSC cardiometabolic driver genes, such as TP53, JAK2, DNMT3A, ASXL1, and/or PPM1D/WIP, causes selective expansion of hematopoietic cells leading to IL-1β (interleukin-1β), IL-6, and/or TNFα proinflammatory activity. Accordingly, provided herein are compositions, methods, and assays for modulating IL-1β (interleukin-1β), IL-6, and/or TNFα proinflammatory activity mediated by selective expansion of hematopoietic cells bearing somatic mutations in HSC cardiometabolic driver genes, such as TP53, JAK2, DNMT3A, ASXL1, and/or PPM 1D/WIP1.

Accordingly, in some aspects, provided herein are pharmaceutical compositions comprising an inhibitor of an HSC cardiometabolic driver gene-mediated proinflammatory activity and a pharmaceutically acceptable carrier for use in a subject having one or more somatic mutations in one or more HSC cardiometabolic driver genes in a sub-population of hematopoietic cells.

Also provided herein, in some aspects, are methods for treating a subject having, or at risk for, a HSC cardiometabolic driver gene-mediated proinflammatory disease comprising: administering a therapeutically effective amount of a pharmaceutical composition comprising an inhibitor of HSC cardiometabolic driver gene-mediated proinflammatory activity and a pharmaceutically acceptable carrier to a subject having one or more somatic mutations in one or more HSC cardiometabolic driver genes in a sub-population of hematopoietic cells.

A “somatic mutation,” as used herein, refers to a change in the genetic structure of a subject that is not inherited from a parent, and also not passed to offspring. Hence, a somatic mutation is a genetic change that occurs in any cell after the first cell division, wherein the mutation is replicated in all cells that descend from the mutated cell. The somatic cells that descend from the original mutated cell comprise a clonal variant within the body of the subject. Where these mutations are present in cells of somatic origin and not present in the germline, they are often called a somatic cell mutation or an acquired mutation. Somatic mutations will be present in only a subset of the cells contributing DNA to an analysis, since the mutant sequence will be present in fewer than 50% of the sequence reads arising from that genomic site. In other words, somatic mutations are identified as when a specific sequence is measured as occurring at a fraction of total sequences that deviates significantly from the frequency expected for the far-larger number of inherited variants—namely around 0%, around 50% or around 100%.

Somatic mutations can occur in a sub-population of cells for example, such as a sub-population of hematopoietic cells. Somatic mutations in HSC cardiometabolic driver genes, such as TP53, JAK2, DNMT3A, ASXL1, TET-2 and/or PPM1D/WIP12, relevant to the compositions and methods described herein include any nucleic acid or consequent amino acid somatic mutations in HSC cardiometabolic driver genes, such as TP53, JAK2, DNMT3A, ASXL1, and/or PPM1D/WIP1, found in a subset of hematopoietic cells, that lead to increased pro-inflammatory IL-1β signaling, increased pro-inflammatory IL-6 signaling, and/or increased pro-inflammatory TNFα signaling. Such increased pro-inflammatory IL-1β signaling, increased pro-inflammatory IL-6 signaling, and/or increased pro-inflammatory TNFα signaling includes, but is not limited to, increased IL-1β, IL-6, and/or TNFα transcription, increased NLRP3 inflammasome-mediated IL-1β secretion, increased IL-1-Receptor 1-mediated IL-1β signaling, increased IL-6-Receptor a-mediated IL-6 signaling, increased gp130-mediated IL-6 signaling, increased JAK1/JAK2-mediated IL-6 signaling, increased STAT3/STAT1-mediated IL-6 signaling, increased TNFR1-mediated TNFα signaling, increased TNFR2-mediated TNFα signaling, and/or increased TRAF2/TRAF3-mediated TNFα signaling. Such somatic mutations in HSC cardiometabolic driver genes, such as TP53, JAK2, DNMT3A, ASXL1, and/or PPM1D/WIP1, can be disruptive, in that they have an observed or predicted effect on protein function, or non-disruptive. As used herein, a “non-disruptive mutation” is typically a missense mutation, in which a codon is altered such that it codes for a different amino acid, but the encoded protein, i.e., TP53, JAK2, DNMT3A, ASXL1, and/or PPM1D/WIP1, is still expressed. Somatic mutations in HSC cardiometabolic driver r genes, such as TP53, JAK2, DNMT3A, ASXL1, and/or PPM1D/WIP1 include, for example, frameshift mutations, nonsense mutations, missense mutations or splice-site variant mutations, as those terms are known to those of ordinary skill in the art.

In some embodiments, one or more somatic mutations in an HSC cardiometabolic driver gene, such as TP53, JAK2, DNMT3A, ASXL1, and/or PPM1D/WIP1, in addition to leading to increased pro-inflammatory IL-1β, IL-6, and/or TNFα signaling, also results in clonal hematopoiesis. As used herein, “clonal hematopoiesis” refers to clonal outgrowth of a sub-population of hematopoietic cells having one or more somatic mutations in any of the HSC cardiometabolic driver genes described herein, including TP53, JAK2, DNMT3A, ASXL1, and/or PPM1D/WIP1. Somatic mutations in these genes relevant to the compositions and methods described herein can be found in, for example, WO 2016/085876, the contents of which are herein incorporated in their entireties by reference.

TP53 mutations relevant to the compositions and methods described herein include, but are not limited to, any nucleic acid mutations selected from a G743A mutation in SEQ ID NO:2 (also known as dbSNP 138 ID rs11540652 or R248Q) and a A659G mutation in SEQ ID NO: 2.

JAK2 mutations relevant to the compositions and methods described herein include, but are not limited to, a nucleic acid mutation G1849T in SEQ ID NO: 56 (also known as dbSNP 138 ID rs386626619 or a V617F mutation).

DNMT3A mutations relevant to the compositions and methods described herein include, but are not limited to, any nucleic acid mutations selected from: an A2723G mutation in SEQ ID NO: 37; a G2719A mutation in SEQ ID NO: 37; a T2714G mutation in SEQ ID NO: 37; a T2714A mutation in SEQ ID NO: 37; a C2711T mutation in SEQ ID NO: 37 (also known as dbSNP 138 ID rs3149095705); a C2695T mutation in SEQ ID NO: 37; a C2683A mutation in SEQ ID NO: 37; a C2678A mutation in SEQ ID NO: 37; a CC2671_2672G mutation in SEQ ID NO: 37; a G2669A mutation in SEQ ID NO: 37; a G2645C mutation in SEQ ID NO: 37 (also known as dbSNP 138 ID rs147001633); a G2645A mutation in SEQ ID NO: 37 (also known as dbSNP 138 ID rs147001633); a G2645C mutation in SEQ ID NO: 37 (also known as dbSNP 138 ID rs147001633); a C2644Tmutation in SEQ ID NO: 37 (also known as dbSNP 138 ID rs377577594); a A2638G mutation in SEQ ID NO: 37; a T2578C mutation in SEQ ID NO: 37 (also known as dbSNP 138 ID rs37301470); a A2554G mutation in SEQ ID NO: 37; a G2527A mutation in SEQ ID NO: 37; a 2479-2A>G mutation in SEQ ID NO: 37; a 2478+1G>T mutation in SEQ ID NO: 37; a 2479-2A>G mutation in SEQ ID NO: 37; a C2446T mutation in SEQ ID NO: 37; a 2408+1G>A mutation in SEQ ID NO: 37; a G2387T mutation in SEQ ID NO: 37; a T2383C mutation in SEQ ID NO: 37; a G2375A mutation in SEQ ID NO: 37; a T2339C mutation in SEQ ID NO: 37 (also known as dbSNP 138 ID rs370751539); a T2339C mutation in SEQ ID NO: 37; a C2330G mutation in SEQ ID NO: 37; a 2323-1G>A mutation in SEQ ID NO: 37; a 2305_2319C deletion mutation in SEQ ID NO: 37; a C2331T mutation in SEQ ID NO: 37; a C2309A mutation in SEQ ID NO: 37; a T2306A mutation in SEQ ID NO: 37; a 2296_2298C deletion mutation in SEQ ID NO: 37; A2281G mutation in SEQ ID NO: 37; a G2268T mutation in SEQ ID NO: 37; a T2264C mutation in SEQ ID NO: 37; a G2259C mutation in SEQ ID NO: 37; a T2252G mutation in SEQ ID NO: 37; a C2245T mutation in SEQ ID NO: 37; a G2207A mutation in SEQ ID NO: 37 (also known as dbSNP 138 ID rs139293773); a C2206T mutation in SEQ ID NO: 37; a A2204G mutation in SEQ ID NO: 37 (also known as dbSNP 138 ID rs147828672); a A2198G mutation in SEQ ID NO: 37; a 2195_2197G deletion mutation in SEQ ID NO: 37; a 2197_2197delinsTG mutation in SEQ ID NO: 37; a 2193_2196T deletion mutation in SEQ ID NO: 37; a C2195G deletion mutation in SEQ ID NO: 37; a C2185T mutation in SEQ ID NO: 37 (also known as dbSNP 138 ID rs20018028); a C2141G mutation in SEQ ID NO: 37 (also known as dbSNP 138 ID rs367909007); a T2128A mutation in SEQ ID NO: 37; a G2117A mutation in SEQ ID NO: 37; a 2115_2116G deletion mutation in SEQ ID NO: 37; a 2107_2108T deletion mutation in SEQ ID NO: 37; a G2104A mutation in SEQ ID NO: 37; a G2089T mutation in SEQ ID NO: 37; a 2086_2087A mutation in SEQ ID NO: 37; a 2082+1G>A mutation in SEQ ID NO: 37; a G2063A mutation in SEQ ID NO: 37 (also known as dbSNP 138 ID rs369713081); a G2054A mutation in SEQ ID NO: 37; a 2042_2043C mutation in SEQ ID NO: 37; a C2043G mutation in SEQ ID NO: 37; a T2042A mutation in SEQ ID NO: 37; a G2027T mutation in SEQ ID NO: 37; a 2000_2006C deletion mutation in SEQ ID NO: 37; a G1993T mutation in SEQ ID NO: 37; a G1984A mutation in SEQ ID NO: 37; a G1969A mutation in SEQ ID NO: 37 (also known as dbSNP 138 ID rs368961181); a T1964A mutation in SEQ ID NO: 37; a 1851+1G>T mutation in SEQ ID NO: 37; a G1846Tmutation in SEQ ID NO: 37; a 1840_1841A mutation in SEQ ID NO: 37; a C1837G mutation in SEQ ID NO: 37; a T1814C mutation in SEQ ID NO: 37; a G1811A mutation in SEQ ID NO: 37; a G1797T mutation in SEQ ID NO: 37; a C1792T mutation in SEQ ID NO: 37; a C1789T mutation in SEQ ID NO: 37; a 1736_1742G deletion mutation in SEQ ID NO: 37; a C1739G mutation in SEQ ID NO: 37; a C1706T mutation in SEQ ID NO: 37; a A1666G mutation in SEQ ID NO: 37; a G1648A mutation in SEQ ID NO: 37; a G1627T mutation in SEQ ID NO: 37; a 1592_1595G mutation in SEQ ID NO: 37; a A1586T mutation in SEQ ID NO: 37; a C1560A mutation in SEQ ID NO: 37; a 1554+2T>G mutation in SEQ ID NO: 37; a 1554+1G>A mutation in SEQ ID NO: 37; a 1538_1538delinsAT mutation in SEQ ID NO: 37; a A1502G mutation in SEQ ID NO: 37 (also known as dbSNP 138 ID rs149738328); a G1490A mutation in SEQ ID NO: 37; a 1474+1G>A mutation in SEQ ID NO: 37; a G1451A mutation in SEQ ID NO: 37; a T1441G mutation in SEQ ID NO: 37; a G1405Tmutation in SEQ ID NO: 37; a 1397_1398G mutation in SEQ ID NO: 37; a A1378G mutation in SEQ ID NO: 37; a C1358T mutation in SEQ ID NO: 37; a G1319A mutation in SEQ ID NO: 37; a T1316C mutation in SEQ ID NO: 37; a 1283_1284G mutation in SEQ ID NO: 37; a G1267C mutation in SEQ ID NO: 37; a C1154T mutation in SEQ ID NO: 37; a C1135T mutation in SEQ ID NO: 37; a 1123-2A>C mutation in SEQ ID NO: 37; a T1115C mutation in SEQ ID NO: 37; a G1114A mutation in SEQ ID NO: 37 (also known as dbSNP 138 ID rs371677904); a T1091C mutation in SEQ ID NO: 37; a G1055A mutation in SEQ ID NO: 37 (also known as dbSNP 138 ID rs139053291); a 1052_1054AA mutation in SEQ ID NO: 37; a T1031C mutation in SEQ ID NO: 37; a 1023_1023delins GTT mutation in SEQ ID NO: 37; a C1015_splice mutation in SEQ ID NO: 37; a G995A mutation in SEQ ID NO: 37; a G994A mutation in SEQ ID NO: 37; a G990A mutation in SEQ ID NO: 37; a G976T mutation in SEQ ID NO: 37; a C958T mutation in SEQ ID NO: 37; a G942A mutation in SEQ ID NO: 37; a C920T mutation in SEQ ID NO: 37; a C920G mutation in SEQ ID NO: 37; a G918A mutation in SEQ ID NO: 37; a G886A mutation in SEQ ID NO: 37; a C883G mutation in SEQ ID NO: 37; a T875C mutation in SEQ ID NO: 37; a 737_737 delinsGC mutation in SEQ ID NO: 37; and a 696_697C mutation in SEQ ID NO: 37;

In some embodiments of the compositions and methods described herein, the one or more DNMT3A somatic mutations are selected from a T1115C mutation in SEQ ID NO: 37; a C2711T mutation in SEQ ID NO: 37 (also known as dbSNP 138 ID rs3149095705); a C1837G mutation in SEQ ID NO: 37; a A1666G mutation in SEQ ID NO: 37; a C1789T mutation in SEQ ID NO: 37; a G2719A mutation in SEQ ID NO: 37; a G1627T mutation in SEQ ID NO: 37; an A2723G mutation in SEQ ID NO: 37; a G1797T mutation in SEQ ID NO: 37; a T2252G mutation in SEQ ID NO: 37; a C1560A mutation in SEQ ID NO: 37; a T1031C mutation in SEQ ID NO: 37; a G2645A mutation in SEQ ID NO: 37 (also known as dbSNP 138 ID rs147001633); a C2043G mutation in SEQ ID NO: 37; a C2446T mutation in SEQ ID NO: 37; a A2198G mutation in SEQ ID NO: 37; A2281G mutation in SEQ ID NO: 37; a C920G mutation in SEQ ID NO: 37; a A2204G mutation in SEQ ID NO: 37 (also known as dbSNP 138 ID rs147828672); and a frameshift mutation in DNMT3A.

ASXL1 mutations relevant to the compositions and methods described herein include but are not limited to, any nucleic acid mutations selected from: a 920_921C mutation in SEQ ID NO: 61; a T1157A mutation in SEQ ID NO: 61; a C1294T mutation in SEQ ID NO: 61; a 1541_1543C mutation in SEQ ID NO: 61; a C1564T mutation in SEQ ID NO: 61; a 1621_1621delinsCGGCT mutation in SEQ ID NO: 61; a 1926_1926delinsAG mutation in SEQ ID NO: 61; a 1771_1771delinsTA mutation in SEQ ID NO: 61; a 1887_1910T deletion mutation in SEQ ID NO: 61; a 1899_1901T deletion mutation in SEQ ID NO: 61; a 1970_1970delinsAG mutation in SEQ ID NO: 61; a 2057_2059A mutation in SEQ ID NO: 61; a C2077T mutation in SEQ ID NO: 61; a 2109_2110T mutation in SEQ ID NO: 61; a 2109_2109delinsTC mutation in SEQ ID NO: 61; a A2173T mutation in SEQ ID NO: 61; a 2193_2194A mutation in SEQ ID NO: 61; a 2383_2384T mutation in SEQ ID NO: 61; a C2407T mutation in SEQ ID NO: 61; a 2466_2467A mutation in SEQ ID NO: 61; a G2476T mutation in SEQ ID NO: 61; a 2530_2530delinsAC mutation in SEQ ID NO: 61; a C2568A mutation in SEQ ID NO: 61; a G2694A mutation in SEQ ID NO: 61; a C2893T mutation in SEQ ID NO: 61; a 2958_2958delinsCGT mutation in SEQ ID NO: 61; a C3202T mutation in SEQ ID NO: 61; a 3758_3758delinsGC mutation in SEQ ID NO: 61; and a 4542_4544C mutation in SEQ ID NO: 61.

In some embodiments of the compositions and methods described herein, the one or more ASXL1 somatic mutations are selected from a C2407T mutation in SEQ ID NO: 61; a C2893T mutation in SEQ ID NO: 61; a 1926_1926delinsAG mutation in SEQ ID NO: 61; and a frameshift mutation in ASXL1.

PPM1D mutations relevant to the compositions and methods described herein include but are not limited to, any nucleic acid mutations selected from: a 346_346delinsGC mutation in SEQ ID NO: 64; a 883_885G mutation in SEQ ID NO: 64; a T1221A mutation in SEQ ID NO: 64; a 346_346delinsGC mutation in SEQ ID NO: 64; a 1279_1280T mutation in SEQ ID NO: 64; a C1372T mutation in SEQ ID NO: 64; a 1412_1413C mutation in SEQ ID NO: 64; a 1430_1431A mutation in SEQ ID NO: 64; a 1437_1437delinsTA mutation in SEQ ID NO: 64; a 1448_1448delinsCT mutation in SEQ ID NO: 64; a 1465_1466T mutation in SEQ ID NO: 64; a 1528_1528delinsCA mutation in SEQ ID NO: 64; a G1573T mutation in SEQ ID NO: 64; a G1618T mutation in SEQ ID NO: 64; and a C1714T mutation in SEQ ID NO: 64.

In some embodiments of the compositions and methods described herein, the one or more PPM1D somatic mutations are selected from a G1618T mutation in SEQ ID NO: 64; a C1372T mutation in SEQ ID NO: 64; and a frameshift mutation in PPM1D.

In some embodiments of the compositions and methods described herein, a subject also has a somatic mutation in TET2. Methylcytosine dioxygenase TET2” or “TET2” is a member of the family of TET proteins, which have been shown to be responsible for conversion of 5-methylcytosine (5mC) to 5-hydroxymethylcytosine (5hmC), as well as function in embryonic stem cell regulation, myelopoiesis, and zygote development (Dawlaty et al., 2011; Gu et al., 2011; Iqbal et al., 2011; Ito et al., 2010; Ko et al., 2010; Koh et al., 2011; Wossidlo et al., 2011). TET2 is a dioxygenase that catalyzes the conversion of the modified genomic base 5-methylcytosine (5mC) into 5-hydroxymethylcytosine (5hmC) and plays a key role in active DNA demethylation. TET2 has a preference for 5-hydroxymethylcytosine in CpG motifs, and has also been shown to mediate subsequent conversion of 5hmC into 5-formylcytosine (5fC), and conversion of 5fC to 5-carboxylcytosine (5caC). Methylation at the C5 position of cytosine bases is an epigenetic modification of the mammalian genome which plays an important role in transcriptional regulation. In addition to its role in DNA demethylation, TET2 has also been shown to be involved in the recruitment of the O-GlcNAc transferase OGT to CpG-rich transcription start sites of active genes, thereby promoting histone H2B GlcNAcylation by OGT. Similarly, TET2 has been reported to recruit histone deacetylases (HDACs) to specific gene promoters, contributing to histone deacetylation and gene repression (Zhang et al 2015).

Accordingly, the terms “TET2” or “TET-2,” as used herein, refers to the genomic sequence of NG_028191.1 (SEQ ID NO: 66) encoding: the mRNA sequence of NM_001127208.2 (isoform 1, SEQ ID NO: 67), which encodes the 2002 amino acid polypeptide having the amino acid sequence of NP_001120680.1 (isoform 1, SEQ ID NO: 68); the mRNA sequence of NM_017628.4 (isoform 2, SEQ ID NO: 69) encoding the 1165 amino acid polypeptide having the amino acid sequence of NP_060098.3 (isoform 2, SEQ ID NO: 70); together with any additional naturally occurring allelic, splice variants, and processed forms thereof. Typically, TET2 refers to human TET2. Reference to specific sub-fragments or sub-sequences of TET2 can be identified in the application, e.g., by “nucleic acids 211-402 of TET2.” Specific nucleic acid or amino acid residues of TET2 can be referred to as, for example, “S282 of TET2” or “S282 of SEQ ID NO: 68.”

Accordingly, TET2 mutations relevant to the compositions and methods described herein include any nucleic acid mutations in the genomic sequence of TET2 of SEQ ID NO: 66 leading to: an S460F mutation in SEQ ID NO: 68; a D666G mutation in SEQ ID NO: 68; a P941S mutation in SEQ ID NO: 68; a C1135Y missense mutation in SEQ ID NO: 68; a R73 frameshift insertion mutation in SEQ ID NO: 68, a Y85 frameshift deletion mutation in SEQ ID NO: 68; a S123 frameshift deletion mutation in SEQ ID NO: 68, an E170 frameshift deletion mutation in SEQ ID NO: 68; a D162 frameshift deletion mutation in SEQ ID NO: 68; an 1181 frameshift deletion mutation in SEQ ID NO: 68; a T221 frameshift insertion mutation in SEQ ID NO: 68; an L260 frameshift deletion mutation in SEQ ID NO: 68; an 1274 frameshift deletion mutation in SEQ ID NO: 68, a L311 frameshift insertion mutation in SEQ ID NO: 68; a Q341 nonsense mutation in SEQ ID NO: 68; a Q383 nonsense mutation in SEQ ID NO: 68; a S423 nonsense mutation in SEQ ID NO: 68, a L427 frameshift insertion mutation in SEQ ID NO: 68, a S420 frameshift deletion mutation in SEQ ID NO: 68; a S424 frameshift deletion mutation in SEQ ID NO: 68; a S462 frameshift deletion mutation in SEQ ID NO: 68; an 1472 frameshift deletion mutation in SEQ ID NO: 68; a Q481 nonsense mutation in SEQ ID NO: 68; a T518 frameshift insertion mutation in SEQ ID NO: 68; a S530 nonsense mutation in SEQ ID NO: 68; a S543 frameshift deletion mutation in SEQ ID NO: 68; a Q530 nonsense mutation in SEQ ID NO: 68; a L532 frameshift deletion mutation in SEQ ID NO: 68; a L532 nonsense mutation in SEQ ID NO: 68; a R544 nonsense mutation in SEQ ID NO: 68; a W585 nonsense mutation in SEQ ID NO: 68; a Q595 frameshift deletion mutation in SEQ ID NO: 68; a L579 frameshift insertion mutation in SEQ ID NO: 68; a S588 nonsense mutation in SEQ ID NO: 68; a G634 frameshift deletion mutation in SEQ ID NO: 68; a Q656 frameshift deletion mutation in SEQ ID NO: 68; a P690 frameshift deletion mutation in SEQ ID NO: 68; a R686 frameshift deletion mutation in SEQ ID NO: 68, an E692 frameshift insertion mutation in SEQ ID NO: 68; a Q705 nonsense mutation in SEQ ID NO: 68, a F713frameshift deletion mutation in SEQ ID NO: 68; a Q734 nonsense mutation in SEQ ID NO: 68, a S757 nonsense mutation in SEQ ID NO: 68; a L759 frameshift deletion mutation in SEQ ID NO: 68, a Q764 frameshift deletion mutation in SEQ ID NO: 68; a 1771 frameshift deletion mutation in SEQ ID NO: 68; a Q779 nonsense mutation in SEQ ID NO: 68; an H783 frameshift insertion mutation in SEQ ID NO: 68, a Q770 nonsense mutation in SEQ ID NO: 68, a E819 frameshift deletion mutation in SEQ ID NO: 68, a H839 frameshift deletion mutation in SEQ ID NO: 68; a K858 frameshift deletion mutation in SEQ ID NO: 68; a Q886 nonsense mutation in SEQ ID NO: 68; a L878 frameshift deletion mutation in SEQ ID NO: 68; an M906 frameshift insertion mutation in SEQ ID NO: 68; a Q909 nonsense mutation in SEQ ID NO: 68; a Q910 nonsense mutation in SEQ ID NO: 68; a Q912 frameshift deletion mutation in SEQ ID NO: 68; a Q937 nonsense mutation in SEQ ID NO: 68; a Q916 nonsense mutation in SEQ ID NO: 68; a P989 frameshift insertion mutation in SEQ ID NO: 68; an A1014 frameshift insertion mutation in SEQ ID NO: 68, a Q1042 nonsense mutation in SEQ ID NO: 68, a Q1030 nonsense mutation in SEQ ID NO: 68, an H1064 frameshift insertion mutation in SEQ ID NO: 68; a T1078 frameshift deletion mutation in SEQ ID NO: 68; a T1107 frameshift deletion mutation in SEQ ID NO: 68; a N1103frameshift deletion mutation in SEQ ID NO: 68; a T1114 frameshift deletion mutation in SEQ ID NO: 68; a Q1127 frameshift insertion mutation in SEQ ID NO: 68; an S282F mutation in SEQ ID NO: 68; a N312S mutation in SEQ ID NO: 68; an L346P mutation in SEQ ID NO: 68; and an G to A splice site mutation at position 106158509 of SEQ ID NO: 1.

In some embodiments of the compositions and methods described herein, the one or more TET2 somatic mutations are selected from an S282F mutation in SEQ ID NO: 68, an N312S mutation in SEQ ID NO: 68, an L346P mutation in SEQ ID NO: 68, an S460F mutation in SEQ ID NO: 68, a D666G mutation in SEQ ID NO: 68, a P941S mutation in SEQ ID NO: 68, and a C1135Y mutation in SEQ ID NO: 68.

The compositions and methods described herein require, in some embodiments, sequencing of at least part of the genome in a sample comprising hematopoietic cells, including, for example, an enriched for population of myeloid cells, obtained from a subject. Sequencing can be carried out according to any suitable technique, many of which are generally known in the art. Many proprietary sequencing systems are available commercially and can be used in the context of the methods described herein, such as for example from Illumina, USA. Single-cell sequencing methods are known in the art, as noted for example by Eberwine et al., Nature Methods 11, 25-27 (2014) doi:10.1038/nmeth.2769 Published online 30 Dec. 2013; and single-cell sequencing in microfluidic droplets (Nature 510, 363-369 (2014) doi: 10.1038/nature13437).

Sequencing of DNA can be performed on tissues or cells. Sequencing of specific cell types (for example, hematopoietic cells obtained by flow sorting or myeloid lineage hematopoietic cells) can identify mutations in specific cell types that provide specific predictive value for use with the compositions and methods described herein. Sequencing can also be conducted in single cells, using appropriate single-cell sequencing strategies. Single-cell analyses can be used to identify high-risk combinations of mutations co-occurring in the same cells. Co-occurrence signifies that the mutations are occurring in the same cell clone and carry a greater risk, and therefore have a greater predictive value, than occurrence of the same mutations in different individual cells, for example. Certain sequences, such as those with high GC content, repetitive elements and/or low sequence complexity are prone to sequencing errors and false positive creation due to artifacts caused by enzyme slippage and other reading errors. Hence, care must be taken to ensure that any sequence changes observed in these regions are real and not artifact.

Sequencing can be of specific genes only, specific parts of the genome, or the whole genome. Where specific genes are sequenced, the gene(s) sequences are preferably selected from the group consisting of TP53, JAK2, DNMT3A, ASXL1, and/or PPM1D/WIP1. In some embodiments, TET2 sequencing is also performed. In some embodiments, specific parts of genes can be sequenced. For example, for DNMT3A, exons 7 to 23 can be sequenced. In some embodiments, specific mutations can be interrogated, such as the JAK2 mutation V617F. Additionally, or alternatively, specific mutations can be avoided.

Where a part of a genome is sequenced, that part can be the exome. The exome is the part of the genome formed by exons, and thus an exon sequencing method sequences the expressed sequences in the genome. There are 180,000 exons in the human genome, which constitute about 1% of the genome, or approximately 30 million base pairs. Exome sequencing requires enrichment of sequencing targets for exome sequences; several techniques can be used, including PCR, molecular inversion probes, hybrid capture of targets, and solution capture of targets. Sequencing of targets can be conducted by any suitable technique.

Due to enrichment bias in exome libraries, allelic fractions for inherited heterozygous mutations are not expected to be centered around 50%. The average expected allelic fraction for the alternate allele of a heterozygous single nucleotide polymorphisms (SNPs) is approximately 470%-4%. For indels, this value is even lower, likely due to a mix of enrichment bias, sequence misalignment, and improper reporting of allelic counts. Therefore, depending on the exome library used, different thresholds are applied for SNPs and indels for the purpose of identifying putative somatic mutations.

In some embodiments of the aspects described herein, the analysis of the genomes of single cells by single cell sequencing can be used to provide information about the relationship between mutations and cell types. For example, the presence of a mutation in multiple cells of a defined cell type can further strengthen the conclusion that the mutation is clonal. Moreover, the presence of more than one mutation in a single cell can be evidence of clonal expansion, if the mutations are repeatedly found together.

The inhibitors of IL-1β, IL-6, and/or TNFα-mediated proinflammatory activity are particularly useful for subjects having one or more somatic mutations in an HSC cardiometabolic driver gene in a population of hematopoietic cells. As used herein, an “inhibitor of an HSC cardiometabolic driver gene mutation-mediated proinflammatory activity” refers to any agent or molecule that significantly blocks, inhibits, reduces, or interferes with the downstream effects of somatic mutations in an HSC cardiovascular driver gene that leads to increased IL-1β, IL-6, and/or TNFα proinflammatory activity or signaling in vitro, in situ, and/or in vivo. Such increased pro-inflammatory IL-1β signaling, increased pro-inflammatory IL-6 signaling, and/or increased pro-inflammatory TNFα signaling includes, but is not limited to, increased IL-1j, IL-6, and/or TNFα transcription, increased NLRP3 inflammasome-mediated IL-1β secretion, increased IL-1-Receptor 1-mediated IL-1β signaling, increased IL-6-Receptor a-mediated IL-6 signaling, increased gp130-mediated IL-6 signaling, increased JAK1/JAK2-mediated IL-6 signaling, increased STAT3/STAT1-mediated IL-6 signaling, increased TNFR1-mediated TNFα signaling, increased TNFR2-mediated TNFα signaling, and/or increased TRAF2/TRAF3-mediated TNFα signaling. Exemplary inhibitors of HSC cardiometabolic driver gene-mediated proinflammatory activity contemplated for use in the various aspects and embodiments described herein include, but are not limited to, antibodies or antigen-binding fragments thereof that specifically bind to IL-1β, IL-6, and/or TNFα, and/or antibodies or antigen-binding fragments thereof that specifically bind to their receptors, such as IL1R1, IL-6-Receptor a, gp130, TNFR1, or TNFR2, thereby inhibiting/reducing/blocking IL-1β, IL-6, and/or TNFα, interaction(s) with their receptors; small molecule agents that target or specifically bind IL-1β, IL-6, and/or TNFα, and/or IL-1β, IL-6, and/or TNFα signaling components, such as caspase-1, STAT3/STAT1, JAK1/JAK2, and TRAF2/TRAF3, and inhibit/reduce/block IL-1β, IL-6, and/or TNFα-mediated proinflammatory activity; RNA or DNA aptamers that bind to IL-1β, IL-6, and/or TNFα or any of their receptors and inhibit/reduce/block IL-1β, IL-6, and/or TNFα-mediated proinflammatory activity; and/or receptor fragments or fusion polypeptides thereof that block endogenous IL-1f, IL-6, and/or TNFα interactions with their endogenous receptors.

In regard to NLRP3 inflammasome-mediated IL-1β secretion, and inhibitors thereof for use as inhibitors of HSC cardiovascular driver gene mutation-mediated IL-1β (interleukin-1β) proinflammatory activity, as described herein, as known to those of skill in the art, the NLRP3 inflammasome is present primarily in immune and inflammatory cells following activation by inflammatory stimuli; these cells include macrophages, monocytes, DCs, and splenic neutrophils. Activation of the NLRP3 inflammasome occurs in two steps. The first step involves a priming or initiating signal, in which many PAMPs or DAMPs are recognized by TLRs, leading to activation of nuclear factor kappa B (NF-κB)-mediated signaling, which in turn up-regulates transcription of inflammasome-related components, including inactive NLRP3, proIL-1β, and prolL-18 (Bauernfeind et al., 2009; Franchi et al., 2012, 2014). The second step of inflammasome activation is the oligomerization of NLRP3 and subsequent assembly of NLRP3, ASC, and procaspase-1 into a complex. This triggers the transformation of procaspase-1 to caspase-1, as well as the production and secretion of mature IL-1β and IL-18 (Kim et al., 2015; Ozaki et al., 2015; Rabeony et al., 2015). An inhibitor of HSC cardiovascular driver gene mutation-mediated IL-1β (interleukin-1β) proinflammatory activity useful in the methods and compositions described herein can thus target any of the steps and/or components leading to NLRP3 inflammasome activation (see, for example, non-limiting examples in B-Z Shao et al., NLRP3 inflammasome and its inhibitors: a review; Front Pharmacol. 2015; 6: 262).

A non-limiting example of a NLRP3 inflammasome inhibitor useful in the methods and compositions described herein, includes: MCC950 (Selleckchem). MCC950 is a small molecule inhibitor of canonical and noncanonical activation of the NLRP3 inflammasome. MCC950 inhibits accelerated atherosclerosis and inhibits the accelerated pathological cardiac remodeling mediated by hematopoietic TET2 mutations (Fuster J J et al., Science. 2017 Feb. 24; 355(6327):842-847, Sano, S et al., J Am Coll Cardiol. 2018 Feb. 27; 71(8):875-886). In some embodiments of any of the aspects, the NLRP3 inflammasome inhibitor or inflammasome inhibitor is MCC950.

The inhibitors of HSC cardiometabolic driver gene mutation-mediated proinflammatory activity described herein result in a significant inhibition or reduction or decrease in any of the pathways leading to IL-1β, IL-6, and/or TNFα mediated proinflammatory activity or signaling, such as IL-1β, IL-6, and/or TNFα transcription, IL-1β, IL-6, and/or TNFα translation, NLRP3 inflammasome-mediated IL-1β secretion, and/or IL-6, and/or TNFα binding to their respective receptors and consequent signaling. As used herein, the terms reduce(s)/reduced/reducing/reduction, inhibit(s)/inhibiting/inhibited or decrease(s)/decreasing/decreased generally means either a reduction or inhibition of at least 5%, at least 10%, at least 25%, at least 50%, at least 60%, at least 70%, at least 80%, at least 90%, at least 95%, at least 98%, or more, compared to the level of IL-1β, IL-6, and/or TNFα transcription, IL-1β, IL-6, and/or TNFα translation, NLRP3 inflammasome-mediated IL-1β secretion, and/or IL-1β, IL-6, and/or TNFα binding to their respective receptors, and consequent receptor I (IL-1R1)-mediated IL-1β, IL-6, and/or TNFα signaling, under the same conditions but without the presence of inhibitors of HSC cardiometabolic driver gene mutation-mediated proinflammatory activity described herein. Assays for measuring such inhibition or reduced interactions are known in the art and are described herein in the Examples.

A disease or medical condition is considered to be mediated by “IL-1β (interleukin-1β proinflammatory activity” if the spontaneous or experimental disease or medical condition is associated with, or mediated by, for example, elevated levels of IL-1β in bodily fluids or tissue, or if cells or tissues taken from the body produce elevated levels of IL-1 in culture. In many cases, such diseases mediated by IL-1β proinflammatory activity are also recognized by the following additional two conditions: (1) pathological findings associated with the disease or medical condition can be mimicked experimentally in animals by the administration of IL-1β; and (2) the pathology induced in experimental animal models of the disease or medical condition can be inhibited or abolished by treatment with agents which inhibit the action of IL-1β. A non-limiting list of disorders and diseases known to be mediated by or exacerbated by aberrant, elevated IL-1β activity include hereditary syndromes with mutations in inflammasome-associated genes, such as cryopyrin-associated periodic syndromes (CAPS), Familial Mediterranean fever, Pyogenic arthritis, pyoderma gangrenosum and acne (PAPA) syndrome, Deficiency of IL-1Ra (DIRA); Crystal-induced arthropathies, such as gout; systemic-onset juvenile arthritis or Still disease; adult-onset Still disease; rheumatoid arthritis; osteoarthritis; Schnitzler syndrome; Behçet disease; Crohn's disease; periodontal diseases; COPD (Chronic Obstructive Pulmonary Disease); and neutrophil-triggered skin diseases, such as pyoderma gangrenosum, psoriasis pustulosa, Sweet syndrome; and chronic kidney disorders.

A disease or medical condition is considered to be mediated by “IL-6 (interleukin-6) proinflammatory activity” if the spontaneous or experimental disease or medical condition is associated with, or mediated by, for example, elevated levels of IL-6 in bodily fluids or tissue, or if cells or tissues taken from the body produce elevated levels of IL-6 in culture. In many cases, such diseases mediated by IL-6 proinflammatory activity are also recognized by the following additional two conditions: (1) pathological findings associated with the disease or medical condition can be mimicked experimentally in animals by the administration of IL-6; and (2) the pathology induced in experimental animal models of the disease or medical condition can be inhibited or abolished by treatment with agents which inhibit the action of IL-6. A non-limiting list of disorders and diseases known to be mediated by or exacerbated by aberrant, elevated IL-6 activity include cardiac myxoma, rheumatoid arthritis, Castleman's disease, systemic lupus erythematosus, systemic sclerosis, inflammatory myopathies, Diabetes mellitus (type 2), obesity, Graves' ophthalmopathy, Polymyalgia rheumatic, Giant-cell arteritis, Steroid refractory acute GVHD, Non-ST elevation myocardial infarction, Noninfectious uveitis, JIA-associated uveitis, Recurrent ovarian cancer, Behcet's syndrome, Schizophrenia, Erdheim-Chester disease, Primary Sjogren's syndrome, and fibrous dysplasia of bone.

A disease or medical condition is considered to be mediated by “TNFα (Tumor Necrosis Factor α) proinflammatory activity” if the spontaneous or experimental disease or medical condition is associated with, or mediated by, for example, elevated levels of TNFα in bodily fluids or tissue, or if cells or tissues taken from the body produce elevated levels of TNFα in culture. In many cases, such diseases mediated by TNFα proinflammatory activity are also recognized by the following additional two conditions: (1) pathological findings associated with the disease or medical condition can be mimicked experimentally in animals by the administration of TNFα; and (2) the pathology induced in experimental animal models of the disease or medical condition can be inhibited or abolished by treatment with agents which inhibit the action of TNFα. A non-limiting list of disorders and diseases known to be mediated by or exacerbated by aberrant, elevated TNF (activity include rheumatoid arthritis, inflammatory bowel disease, psoriasis, psoriatic arthritis, ankylosing spondylitis, axial spondyloarthropathies, Crohn's disease, ulcerative colitis, juvenile idiopathic arthritis (JIA), acute cardav

In some embodiments of the compositions and methods described herein, a disease mediated by IL-1β proinflammatory activity, IL-6 proinflammatory activity, and/or TNFα proinflammatory activity is a cardiometabolic disease. Cardiometabolic diseases include cardiovascular diseases, as well as those disorders that complicate the risk and clinical management of cardiovascular conditions by potentiating and/or exacerbating hypertension, hyperlipidemia, atherosclerosis and cardiomyopathy, and include insulin resistance, hyperglycemia, obesity, type 2 diabetes mellitus, metabolic syndrome, hyperlipidemia and oxidative stress.

As used herein, the phrase “cardiovascular condition, disease or disorder” is intended to include all disorders characterized by insufficient, undesired or abnormal blood vessel or cardiac function, e.g. hypertension, ischemic heart disease, hypertensive heart disease and pulmonary hypertensive heart disease, valvular disease, cardiac arrhythmia, vascular disease, myocardial infarction, congestive heart failure, myocarditis, atherosclerosis, restenosis, and any condition which leads to congestive heart failure in a subject, particularly a human subject. Insufficient or abnormal cardiac function can be the result of disease, injury and/or aging. The term “myocardial ischemia” refers to circulatory disturbances caused by coronary atherosclerosis and/or inadequate oxygen supply to the myocardium. For example, an acute myocardial infarction represents an irreversible ischemic insult to myocardial tissue. This insult results in an occlusive (e.g., thrombotic or embolic) event in the coronary circulation and produces an environment in which the myocardial metabolic demands exceed the supply of oxygen to the myocardial tissue.

In some embodiments of the compositions and methods described herein, a disease mediated by IL-1β proinflammatory activity, IL-6 proinflammatory activity, and/or TNFα proinflammatory activity is a chronic kidney disease.

In regard to the methods of treating chronic kidney disease mediated by IL-1β proinflammatory activity, IL-6 proinflammatory activity, and/or TNFα proinflammatory activity, the term “chronic kidney disease” or CKD refers to renal diseases that slowly and progressively worsen over time due to the progressive loss of nephrons and consequent loss of renal function. In the early stages, there may be no symptoms. The loss of function usually takes months or years to occur. It may be so slow that symptoms do not appear until kidney function is less than one-tenth of normal. The final stage of chronic kidney disease is called end-stage renal disease (ESRD). At this stage, the kidneys are no longer able to remove enough wastes and excess fluids from the body. The patient needs dialysis or a kidney transplant. Diabetes, which leads to diabetic nephropathy, and high blood pressure are the two most common causes of chronic kidney disease and account for most cases. Other diseases and conditions that can damage the kidneys and lead to chronic kidney disease, include, but are not limited to: autoimmune disorders (such as systemic lupus erythematosus and scleroderma); birth defects of the kidneys (such as polycystic kidney disease); certain toxic chemicals; glomerulonephritis; injury or trauma; kidney stones and infection; problems with the arteries leading to or inside the kidneys; some pain medications and other drugs (such as cancer drugs); reflux nephropathy (in which the kidneys are damaged by the backward flow of urine into the kidneys); etc. As used herein, “proteinuria” refers to the presence of an excess of serum proteins in the urine. Proteinuria can, in some embodiments, be indicative of kidney disease, but, by itself, is not conclusive. In some embodiments of these aspects and all such aspects described herein, a subject having or at risk for a chronic kidney disease has diabetic nephropathy.

In some embodiments of the aspects descried herein, an inhibitor of HSC cardiometabolic driver gene mutation-mediated proinflammatory activity is an IL-1β inhibitory compound. As used herein, an “IL-1β inhibitory compound” or “IL-1β inhibitor” or “inhibitor of IL-1β” refers to a compound or agent capable of specifically inhibiting or specifically preventing activation of cellular receptors to IL-1β and consequent downstream effects of IL-1β signaling. Classes of interleukin-1β inhibitors include: interleukin-1 receptor antagonists such as IL-1ra; anti-IL-1 receptor antibodies (e.g., EP 623674), the contents of which is hereby incorporated by reference in its entirety; IL-1 binding proteins such as soluble IL-1 receptors (e.g., U.S. Pat. Nos. 5,492,888, 5,488,032, and 5,464,937, 5,319,071, and 5,180,812, the contents of which are hereby incorporated by reference in their entireties); anti-IL-1β monoclonal antibodies (e.g., WO 9501997, WO 9402627, WO 9006371, U.S. Pat. No. 4,935,343, EP 364778, EP 267611 and EP 220063, the contents of which are hereby incorporated by reference in their entireties); IL-1 receptor I accessory proteins (e.g., WO 96/23067, the disclosure of which is hereby incorporated by reference), and other compounds and proteins which block in vivo synthesis, including in vivo transcription, in vivo translation, and/or extracellular release of IL-1β.

In some embodiments of the aspects described herein, the inhibitor of HSC cardiometabolic driver gene mutation-mediated proinflammatory activity is selected from any of the IL-1β or inflammasome inhibitors listed in Table 1.

TABLE 1
Exemplary List of IL-1β inhibitors for use as HSC cardiometabolic driver
gene mutation-mediated IL-1β (interleukin-1β) proinflammatory activity.

| | Active | Molecule | | Mode of | Route of | Product |
|---|---|---|---|---|---|---|
| Product Name | Ingredient | Type | Target | Action | Administration | Description |
| ABT981 | — | Large | Interleukin 1A | Interleukin- | Subcutaneous | ABT981 is a dual |
| | | molecule- | (IL1A), | 1alpha | | variable |
| | | Antibody | Interleukin 1B | (IL-1alpha) | | immunoglobulin |
| | | | (IL1B) | Inhibitor, | | (DVD-Ig) |
| | | | | Interleukin- | | consisting of |
| | | | | 1beta | | interleukin 1, beta |
| | | | | (IL-1beta) | | antibody and |
| | | | | Inhibitor | | interleukin 1, alpha |
| | | | | | | antibody. It binds |
| | | | | | | and inhibits the |
| | | | | | | interleukin-1 alpha, |
| | | | | | | beta (IL-1 a/β). |
| AC201 | diacerein | Small | Caspase, | Caspase-1 | Oral, Topical | AC201 contains |
| Also known as AC | | molecule | Apoptosis- | Inhibitor, | | diacerein as an |
| 201, AC 203, | | | Related | Interleukin- | | active ingredient. |
| AC203 | | | Cysteine | 1beta | | Diacerein is a small |
| | | | Peptidase | (IL-1beta) | | molecule which |
| | | | 1 (CASP1), | Inhibitor | | inhibits the |
| | | | Interleukin 1B | | | production and |
| | | | (IL1B) | | | activity of caspase- |
| | | | | | | 1 and the cytokine |
| | | | | | | interleukin-1beta |
| | | | | | | (IL-1Beta), and |
| | | | | | | down-regulate IL- |
| | | | | | | 1Beta receptors. |
| | | | | | | AC201 reduces the |
| | | | | | | HbA1c/blood sugar |
| | | | | | | levels. |
| Anti-interleukin-1 | | Large | Interleukin 1B | Interleukin- | | Anti-interleukin-1 |
| Beta antibody by | | molecule- | (IL1B) | 1beta | | Beta antibody is a |
| ABZYME | | Antibody | | (IL-1beta) | | targeted human |
| | | | | Inhibitor | | monoclonal |
| | | | | | | antibody. |
| APX002 | interleukin 1, | Large | Interleukin 1B | Interleukin- | | APX002 is a |
| Also known as | beta | molecule- | (IL1B) | 1beta | | humanized |
| APX 002, TK 002, | monoclonal | Antibody | | (IL-1beta) | | monoclonal |
| TK002 | antibody | | | Inhibitor | | antibody which |
| | (humanized) | | | | | inhibits |
| | | | | | | interleukin- |
| | | | | | | 1-beta. |
| Canakinumab/Ilaris | canakinumab | Large | Interleukin 1B | Interleukin- | Subcutaneous | Canakinumab is an |
| Also known as IL 1 | | molecule- | (IL1B) | 1beta | | interleukin 1 beta |
| Beta Mab | | Antibody | | (IL-1beta) | | monoclonal |
| NOVARTIS, IL 1 | | | | Inhibitor | | antibody derived |
| Beta Monoclonal | | | | | | from a mouse |
| Antibody | | | | | | monoclonal |
| NOVARTIS, | | | | | | antibody (mAb) |
| Interleukin 1 Beta | | | | | | acting against IL-1 |
| Monoclonal | | | | | | beta. |
| Antibody | | | | | | |
| NOVARTIS | | | | | | |
| CDP48 | interleukin 1, | Large | Interleukin 1 | Interleukin- | Subcutaneous | CDP484 is a |
| Also known as | beta antibody | molecule- | Receptor, | 1 Beta | | PEGylated antibody |
| CDP 484 | (pegylated) | Antibody | Type II | (IL-1 Beta) | | fragment targeting |
| | | | (IL1R2) | Receptor | | pro-inflammatory |
| | | | | Antagonist | | cytokine interleukin |
| | | | | | | 1-beta. |
| CP412245 | | Small | Interleukin 1 | Interleukin- | | CP412245 is a |
| Also known as CP | | molecule | Receptor, | 1 Beta | | potent inhibitor of |
| 412, 245, CP | | | Type II | (IL-1 Beta) | | stimulus-coupled |
| 412245, | | | (IL1R2) | Receptor | | IL-1beta post- |
| CP412, 245 | | | | Antagonist | | translational |
| | | | | | | processing. It is a |
| | | | | | | diarylsulfonylurea |
| | | | | | | compound that |
| | | | | | | blocks formation of |
| | | | | | | mature IL-1 without |
| | | | | | | increasing the |
| | | | | | | amount of |
| | | | | | | procytokine that is |
| | | | | | | released |
| | | | | | | extracellularly. |
| CYT013 IL1bQb, | | | Interleukin 1 | Interleukin- | Subcutaneous | CYT013IL1bQb is |
| interleukin 1 beta | | | Receptor, | 1 Beta | | a therapeutic |
| Also known as | | | Type II | (IL-1 Beta) | | vaccine consisting |
| interleukin 1 | | | (IL1R2) | Receptor | | of modified |
| receptor antagonist | | | | Antagonist | | interleukin-1 beta |
| protein | | | | | | molecules coupled |
| | | | | | | to the virus-like |
| | | | | | | particle Qb. The |
| | | | | | | vaccine induces |
| | | | | | | antibodies |
| | | | | | | production against |
| | | | | | | IL-1 beta to |
| | | | | | | decrease |
| | | | | | | inflammation and |
| | | | | | | reduce disease |
| | | | | | | progression. |
| MCC 950 or | | Small | | | | NLRP3 |
| MCC950/CRID3/ | | molecule | | | | inflammasome |
| CP-456773 | | | | | | inhibitor. Blocking |
| | | | | | | apoptosis- |
| | | | | | | associated speck- |
| | | | | | | like protein (ASC) |
| | | | | | | oligomerization, |
| | | | | | | Inhibiting of |
| | | | | | | canonical and non- |
| | | | | | | canonical NLRP3 |
| | | | | | | inflammasome. |
| immunereszumab | interleukin 1, | Large | Interleukin 1B | | | Targeted against |
| | beta antibody | molecule- | (IL1B) | | | interleukin-1 beta. |
| | | Antibody | | | | |
| Inflabion | diacerein | Small | Interleukin 1 | Interleukin-1 | Oral | Inflabion contains |
| | | molecule | (IL1) | (IL-1) | | diacerein as an |
| | | | | Inhibitor | | active ingredient. |
| | | | | | | Diacerin (diacerein) |
| | | | | | | is an anthraquinone |
| | | | | | | derivative that acts |
| | | | | | | via inhibition of |
| | | | | | | interleukin-1beta. |
| Inflammasome | | Small | Interleukin 1B | Interleukin- | | Inflammasome |
| modulator | | molecule | (IL1B) | 1beta | | modulator interferes |
| OPSONA | | | | (IL-1beta) | | with inflammasome |
| | | | | Inhibitor | | mediated release of |
| | | | | | | interleukin (IL)- |
| | | | | | | 1beta. It is a |
| | | | | | | specific IL1-β |
| | | | | | | inhibitor. |
| LY2189102 | interleukin 1, | Large | Interleukin 1 | Interleukin-1 | Intravenous, | LY2189102 |
| Also known as LY | beta | molecule- | Receptor, | Beta | Subcutaneous | contains interleukin |
| 2189102 | monoclonal | Antibody | Type II | (IL-1 Beta) | | 1, beta monoclonal |
| | antibody | | (IL1R2) | Receptor | | antibody as an |
| | (humanized) | | | Antagonist | | active ingredient. It |
| | | | | | | is targeted against |
| | | | | | | interleukin-1 beta. |
| MEDI8968 | interleukin 1 | Large | Interleukin 1A | Interleukin-1 | Subcutaneous | MEDI8968 is a |
| Also known as | receptor | molecule- | (IL1A), | alpha | | fully human IgG2 |
| MEDI 8968 | monoclonal | Antibody | Interleukin 1B | (IL-1 alpha) | | monoclonal |
| | antibody | | (IL1B) | Inhibitor, | | antibody (mAb) |
| | (human) | | | Interleukin- | | that binds |
| | | | | 1beta | | selectively to |
| | | | | (IL-1beta) | | Interleukin-1 |
| | | | | Inhibitor | | Receptor I (IL-1R1) |
| | | | | | | to inhibit the |
| | | | | | | binding of IL-1 |
| | | | | | | alpha and IL-1 beta. |
| PGE3935199 | | | Caspase, | Caspase-1 | Oral | PGE3935199 is a |
| Also known as | | | Apoptosis- | Inhibitor | | caspase-1 inhibitor. |
| PGE 3935199 | | | Related | | | Interleukin-1β |
| | | | Cysteine | | | converting enzyme |
| | | | Peptidase 1 | | | (Caspase-1, ICE) is |
| | | | (CASP1) | | | involved in the |
| | | | | | | processing of Pro- |
| | | | | | | IL-1β to the active |
| | | | | | | cytokine IL-1β. |
| PGE527667 | | | Caspase, | Caspase-1 | Oral | PGE527667 is a |
| Also known as | | | Apoptosis- | Inhibitor | | Caspase-1 inhibitor. |
| PGE 527667 | | | Related | | | Interleukin-1β |
| | | | Cysteine | | | converting enzyme |
| | | | Peptidase 1 | | | (Caspase-1, ICE) is |
| | | | (CASP1) | | | involved in the |
| | | | | | | processing of Pro- |
| | | | | | | IL-1β to the active |
| | | | | | | cytokine IL-1β. |
| TRK530 | | | Interleukin 1B | Interleukin- | Oral | TRK530 is an |
| Also known as | | | (IL1B) | 1beta | | immunomodulatory |
| TRK530 | | | | (IL-1beta) | | bisphosphonate |
| | | | | Inhibitor | | derivative that is |
| | | | | | | directed against |
| | | | | | | interleukin 1b. |
| XL 130 | | Large | Interleukin 1 | Interleukin-1 | | XL130 contains |
| Also known as | | molecule | Receptor | (IL-1) | | PASylated |
| interleukin 1 | | | (IL1R) | Receptor | | interleukin 1 |
| receptor antagonist | | | | Antagonist | | receptor antagonist |
| protein (pasylated) | | | | | | protein as an active |
| | | | | | | ingredient. |
| | | | | | | Interleukin 1 |
| | | | | | | receptor antagonist |
| | | | | | | protein acts by |
| | | | | | | preventing the |
| | | | | | | interaction of IL-1 |
| | | | | | | with the receptor. |
| XOMA052 | gevokizumab | Large | Interleukin 1B | Interleukin- | Intravenous, | XOMA052 contains |
| Also known as S | | molecule- | (IL1B) | 1beta | Subcutaneous | gevokizumab as an |
| 78989, S78989, | | Antibody | | (IL-1beta) | | active ingredient. |
| XMA005.2, | | | | Inhibitor | | Gevokizumab is a |
| XOMA 052 | | | | | | humanized |
| | | | | | | monoclonal |
| | | | | | | antibody directed |
| | | | | | | against interleukin |
| | | | | | | 1b. |
| AMG108 | interleukin 1 | Large | Interleukin 1 | Interleukin-1 | Subcutaneous | AMG108 is a fully |
| Also known as | receptor | molecule- | (IL1) | (IL-1) | | human interleukin 1 |
| AMG 108 | monoclonal | Antibody | | Inhibitor | | receptor |
| | antibody | | | | | monoclonal |
| | (human) | | | | | antibody that binds |
| | | | | | | to and inhibits the |
| | | | | | | action of |
| | | | | | | interleukin-1 (IL-1). |
| HL 2351, IL1Ra | | | Interleukin 1 | Interleukin-1 | Subcutaneous | HL2351 is a long |
| hyFc | | | (IL1) | (IL-1) | | acting fusion |
| | | | | Inhibitor | | protein of IL-1Ra |
| | | | | | | and hybrid fc |
| | | | | | | fragment (hyFc) |
| | | | | | | which inhibits |
| | | | | | | interleukin-1. |
| IL1Hy1 | | | Interleukin 1 | Interleukin-1 | | IL1Hy1 is an |
| Also known as IL | | | Receptor | (IL-1) | | interleukin-1 |
| 1F5, IL 1Hy1, | | | (IL1R) | Receptor | | receptor antagonist |
| interleukin 1 | | | | Antagonist | | that acts by |
| family, member 5, | | | | | | blocking the |
| interleukin 1 HY1 | | | | | | binding of |
| | | | | | | interleukin-1 (IL-1) |
| | | | | | | to cell receptors. |
| Interleukin 1 ra | | Large | Interleukin 1 | Interleukin-1 | | Recombinant |
| AXXO | | molecule | Receptor | (IL-1) | | human interleukin 1 |
| Also known as | | | (IL1R) | Receptor | | receptor antagonist |
| interleukin 1 | | | | Antagonist | | protein acts by |
| receptor antagonist | | | | | | preventing the |
| protein | | | | | | interaction of IL-1 |
| (recombinant, | | | | | | with the receptor. |
| human) | | | | | | |
| Orthokine | | | Interleukin 1 | Interleukin-1 | Intra-articular | Orthokine is an |
| Also known as | | | Receptor | (IL-1) | | autologous serum |
| interleukin 1 | | | (IL1R) | Receptor | | solution derived |
| receptor antagonist | | | | Antagonist | | from the patient's |
| protein | | | | | | blood. It contains |
| | | | | | | the interleukin-1 |
| | | | | | | receptor antagonist |
| | | | | | | (IL-1Ra) protein |
| | | | | | | that prevents the |
| | | | | | | interaction of IL-1 |
| | | | | | | with the receptor. |
| PRT 1000 | | | Interleukin 1 | Interleukin-1 | | PRT1000 contains |
| Also known as | | | Receptor | (IL-1) | | MB-IL1RA which |
| interleukin 1 | | | (IL1R) | Receptor | | is an interleukin-1 |
| receptor antagonist | | | | Antagonist | | receptor antagonist |
| protein | | | | | | protein (IL1RA), |
| | | | | | | fused with a matrix |
| | | | | | | binding domain. It |
| | | | | | | is a potent cytokine |
| | | | | | | inhibitor and |
| | | | | | | prevents the |
| | | | | | | interaction of IL1 |
| | | | | | | with the receptor. |
| Anakinra/Kineret | | Large | Interleukin 1 | Interleukin-1 | | interleukin 1 (IL1) |
| | | molecule | Receptor | (IL-1) | | receptor antagonist. |
| | | | (IL1R) | Receptor | | |
| | | | | Antagonist | | |
| Rilonacept | | Large | | | | Rilonacept has one |
| | | molecule | | | | extracellular |
| | | | | | | domain of IL-1 |
| | | | | | | receptor type 1 (IL- |
| | | | | | | 1R1) and one of IL- |
| | | | | | | 1 receptor accessory |
| | | | | | | protein (IL-1RAcP) |
| | | | | | | bound to the Fc |
| | | | | | | portion of IgG |
| β-hydroxybutyrate | | Small | | | | Blocking ASC |
| (BHB) | | molecule | | | | oligomerization, |
| | | NLRP3 | | | | Inhibiting |
| | | inhibitor | | | | K+/potassium |
| | | | | | | efflux; |
| MicroRNA-223 | | Micro | | | | Suppressing |
| | | RNA | | | | NLRP3 protein |
| | | | | | | expression by |
| | | | | | | binding to a |
| | | | | | | conserved site in |
| | | | | | | the3′ UTR of the |
| | | | | | | NLRP3 transcript,. |

In some embodiments of the aspects described herein, the IL-1β inhibitor is an IL-1β inhibitor antibody or antigen-binding fragment thereof that binds to IL-1β and reduces/inhibits/prevents IL-1β binding to its receptor(s), thereby inhibiting IL-1β-mediated pro-inflammatory activity. As used herein, “antibodies” or “antigen-binding fragments” thereof include monoclonal, human, humanized or chimeric antibodies, single chain antibodies, Fab fragments, F(ab′) fragments, fragments produced by a Fab expression library, and/or binding fragments of any of the above. Antibodies also refer to immunoglobulin molecules and immunologically active portions of immunoglobulin molecules, i.e., molecules that contain antigen or target binding sites or “antigen-binding fragments.” The immunoglobulin molecules described herein can be of any type (e.g., IgG, IgE, IgM, IgD, IgA and IgY), class (e.g., IgG1, IgG2, IgG3, IgG4, IgA1 and IgA2) or subclass of immunoglobulin molecule, as is understood by one of skill in the art.

Accordingly, in some embodiments of the aspects described herein, the IL-1 inhibitor antibody or antigen-binding fragment thereof is selected from ABT981, an anti-interleukin-10 inhibitor antibody by ABZYME, APX002, Canakinumab/Ilaris, CDP48, immunereszumab, LY2189102, MEDI8968, and XOMA052.

In some embodiments of the aspects described herein, the IL-1β inhibitor is an IL-1 receptor antagonist. As used herein, an “interleukin-1 receptor antagonist” (“IL-1ra”) is any agent or molecule, including small molecules and antibody or antigen-binding fragments thereof, that binds to an interleukin-1 receptor thereby preventing binding of IL-1β to the receptor and thereby inhibiting IL-1β-mediated pro-inflammatory activity. Interleukin 1 receptor antagonists, as well as methods of making and using thereof, are described in, for example, U.S. Pat. No. 5,075,222; WO 91/08285; WO 91/17184; AU 9173636; WO 92/16221; WO93/21946; WO 94/06457; WO 94/21275; FR 2706772; WO 94/21235; DE 4219626, WO 94/20517; WO 96/22793 and WO 97/28828, the contents of which are incorporated herein by reference in their entireties. The proteins include glycosylated as well as non-glycosylated IL-1 receptor antagonists.

Accordingly, in some embodiments of the aspects described herein, the IL-1 receptor antagonist is selected from CDP484, CP412245, CYT013 IL1bQb, XL 130, AMG108, HL 2351, IL1Hyl, AXXO, orthokine, PRT 1000, anakinra, and rilonacept.

In some embodiments of the aspects described herein, the IL-1β inhibitor is a small molecule or microRNA inhibitor that inhibits IL-1β-mediated pro-inflammatory activity. Such small molecule inhibitors can target or specifically bind IL-1β, IL-1-receptors, and/or IL-1 signaling components, such as caspase-1, and/or the NLRP3 inflammasome, or components thereof, thereby inhibiting/reducing/blocking IL-1β-mediated proinflammatory activity. As used herein, “small molecule inhibitors” include, but are not limited to, small peptides or peptide-like molecules, soluble peptides, and synthetic non-peptidyl organic or inorganic compounds. A small molecule inhibitor or antagonist can have a molecular weight of any of about 100 to about 20,000 daltons (Da), about 500 to about 15,000 Da, about 1000 to about 10,000 Da.

Accordingly, in some embodiments of the aspects described herein, the small molecule or microRNA IL-1β inhibitor is selected from, AC201, MCC950 or CRID3, inflabion, inflammasome modulator OPSONA, PGE3935199, PGE527667, TRK530, β-hydroxybutyrate (BHB), and microRNA-223. In some embodiments of the aspects described herein, the small molecule IL-1β inhibitor is MCC950.

In some embodiments of the aspects described herein, an inhibitor of HSC cardiometabolic driver gene mutation-mediated proinflammatory activity is an IL-6 inhibitor. As used herein, “IL-6 inhibitor” refers to a therapeutic agent that inhibits IL-6 directly or indirectly (for example, via the IL-6 receptor(s), or via inhibiting JAK-STAT signaling).

In some embodiments of the aspects described herein, the IL-6 inhibitor is an IL-6 inhibitor antibody or antigen-binding fragment thereof that binds to IL-6 and reduces/inhibits/prevents IL-6 binding to its receptor(s), thereby inhibiting IL-6-mediated pro-inflammatory activity. As used herein, “antibodies” or “antigen-binding fragments” thereof include monoclonal, human, humanized or chimeric antibodies, single chain antibodies, Fab fragments, F(ab′) fragments, fragments produced by a Fab expression library, and/or binding fragments of any of the above. Antibodies also refer to immunoglobulin molecules and immunologically active portions of immunoglobulin molecules, i.e., molecules that contain antigen or target binding sites or “antigen-binding fragments.” The immunoglobulin molecules described herein can be of any type (e.g., IgG, IgE, IgM, IgD, IgA and IgY), class (e.g., IgG1, IgG2, IgG3, IgG4, IgA1 and IgA2) or subclass of immunoglobulin molecule, as is understood by one of skill in the art.

Accordingly, in some embodiments of the aspects described herein, the IL-6 inhibitor antibody or antigen-binding fragment thereof is selected from Siltuximab, Olokizumab, Elsilimomab, mAb 1339, Sirukumab, Clazakizumab (formerly ALD518 and BMS-945429), ARGX-109, FM101, and C326.

In some embodiments of the aspects described herein, the IL-6 inhibitor is an IL-6 receptor antagonist. As used herein, an “IL-6 receptor antagonist” (“IL-6ra”) is any agent or molecule, including small molecules and antibody or antigen-binding fragments thereof, that binds to an IL-6 receptor thereby preventing binding of IL-6 to the receptor and thereby inhibiting IL-6-mediated pro-inflammatory activity.

Accordingly, in some embodiments of the aspects described herein, the IL-6 receptor antagonist is selected from tocilizumab (also known as atlizumab), sarilumab, REGN88, FE301, and LMT-28.

In some embodiments of the aspects described herein, the IL-6 inhibitor is a small molecule or microRNA inhibitor that inhibits IL-6-mediated pro-inflammatory activity. Such small molecule inhibitors can target or specifically bind IL-6, IL-6-receptors, and/or IL-6 signaling components, such as JAK or STAT molecules, or components thereof, thereby inhibiting/reducing/blocking IL-6-mediated proinflammatory activity. As used herein, “small molecule inhibitors” include, but are not limited to, small peptides or peptide-like molecules, soluble peptides, and synthetic non-peptidyl organic or inorganic compounds. A small molecule inhibitor or antagonist can have a molecular weight of any of about 100 to about 20,000 daltons (Da), about 500 to about 15,000 Da, about 1000 to about 10,000 Da.

In some embodiments of the aspects described herein, a small molecule IL-6 inhibitor is ALX-0061 or LMT-28.

In some embodiments of the aspects described herein, an IL-6 inhibitor is a JAK-STAT inhibitor. As used herein, “JAK-STAT” inhibitors refer to agents that inhibit the activity of one or more of the Janus kinase family of enzymes (JAK1, JAK2, JAK3, TYK2), thereby interfering with the JAK-STAT signaling pathway. Examples of JAK-STAT inhibitors include, but are not limited to baricitinib, decernotinid, filgotinib, INCB-039110, ruxolitinib, tofacitinib, Oclacitinib, Gandotinib (LY-2784544), Lestaurtinib (CEP-701), Momelotinib (GS-0387, CYT-387), Pacritinib (SB1518), PF-04965842, Upadacitinib (ABT-494), and Peficitinib (ASP015K, JNJ-54781532).

In some embodiments of the aspects described herein, an inhibitor of HSC cardiometabolic driver gene mutation-mediated proinflammatory activity is a TNFα or TNF-α inhibitor. As used herein, “TNFα inhibitor” refers to a therapeutic agent that inhibits TNFα directly or indirectly (for example, via the TNFα receptor(s), or via inhibiting TRAF2/TRAF3 signaling).

In some embodiments of the aspects described herein, the TNFα inhibitor is an TNFα inhibitor antibody or antigen-binding fragment thereof that binds to TNFα and reduces/inhibits/prevents TNFα binding to its receptor(s), thereby inhibiting TNFα-mediated pro-inflammatory activity. As used herein, “antibodies” or “antigen-binding fragments” thereof include monoclonal, human, humanized or chimeric antibodies, single chain antibodies, Fab fragments, F(ab′) fragments, fragments produced by a Fab expression library, and/or binding fragments of any of the above. Antibodies also refer to immunoglobulin molecules and immunologically active portions of immunoglobulin molecules, i.e., molecules that contain antigen or target binding sites or “antigen-binding fragments.” The immunoglobulin molecules described herein can be of any type (e.g., IgG, IgE, IgM, IgD, IgA and IgY), class (e.g., IgG1, IgG2, IgG3, IgG4, IgA1 and IgA2) or subclass of immunoglobulin molecule, as is understood by one of skill in the art.

Accordingly, in some embodiments of the aspects described herein, the TNFα inhibitor antibody or antigen-binding fragment thereof is selected from adalimumab (HUMIRA), Adalimumab-atto (Amjevita), certolizumab pegol, golimumab, infliximab,

In some embodiments of the aspects described herein, the TNFα inhibitor is a TNFα receptor antagonist. As used herein, an “TNFα receptor antagonist” (“TNFαra”) is any agent or molecule, including small molecules and antibody or antigen-binding fragments thereof, that binds to a TNFα receptor thereby preventing binding of TNFα to the receptor and thereby inhibiting TNFα-mediated pro-inflammatory activity.

Accordingly, in some embodiments of the aspects described herein, the TNFα receptor antagonist is etanercept.

In some embodiments of the aspects described herein, the TNFα inhibitor is a small molecule or microRNA inhibitor that inhibits TNFα-mediated pro-inflammatory activity. Such small molecule inhibitors can target or specifically bind TNFα, TNFα-receptors, and/or TNFα signaling components, such as TRAF2/TRAF3 molecules, or components thereof, thereby inhibiting/reducing/blocking TNFα-mediated proinflammatory activity. As used herein, “small molecule inhibitors” include, but are not limited to, small peptides or peptide-like molecules, soluble peptides, and synthetic non-peptidyl organic or inorganic compounds. A small molecule inhibitor or antagonist can have a molecular weight of any of about 100 to about 20,000 daltons (Da), about 500 to about 15,000 Da, about 1000 to about 10,000 Da.

In some embodiments of the aspects described herein, a TNFα inhibitor is a TRAF2/TRAF3 inhibitor. As used herein, “TRAF2/TRAF3” inhibitors refer to agents that inhibit the activity of one or more of the TNF receptor associated family (TRAF) of enzymes (JAK1, JAK2, JAK3, TYK2), thereby interfering with the JAK-STAT signaling pathway.

In some embodiments of the aspects descried herein, an inhibitor of inhibitor of HSC cardiometabolic driver gene mutation-mediated proinflammatory activity is a HSC cardiometabolic driver gene activating compound or HSC cardiometabolic driver gene potentiatior. As used herein, the terms “HSC cardiometabolic driver gene activating compound” or “HSC cardiometabolic driver gene potentiatior” or “HSC cardiometabolic driver gene activator” or “HSC cardiometabolic driver gene agonist” refer to a molecule or agent that mimics or up-regulates (e.g., increases, potentiates or supplements) the biological activity of a HSC cardiometabolic driver gene, such as TP53, JAK2, DNMT3A, ASXL1, and/or PPM1D/WIP1, thereby decreasing or inhibiting proinflammatory activity caused by deficient HSC cardiometabolic driver gene activity. A HSC cardiometabolic driver gene potentiator or agonist can be, in some embodiments, a protein fragment or derivative encoded by a HSC cardiometabolic driver gene thereof having at least one bioactivity of the wild-type protein encoded by the HSC cardiometabolic driver gene. Exemplary HSC cardiometabolic driver gene activating compounds or agonists contemplated for use in the various aspects and embodiments described herein include, but are not limited to, RNA or DNA aptamers; structural analogs or fragments, derivatives, or fusion polypeptides thereof of the protein encoded by the HSC cardiometabolic driver gene; and small molecule agents that target or bind to HSC cardiometabolic driver gene products and act as functional mimics.

A subject in need of the pharmaceutical compositions and methods comprising compositions comprising inhibitors of HSC cardiometabolic driver gene mutation-mediated proinflammatory activity and a pharmaceutically acceptable carrier described herein has or is identified as having one or more somatic mutations in an HSC cardiometabolic driver genes in a sub-population of their hematopoietic cells. As used herein, a “sub-population” of hematopoietic cells comprising the one or more mutations in HSC cardiometabolic driver genes in the subject refers to at least 1% of hematopoietic cells, at least 2% of hematopoietic cells, at least 3% of hematopoietic cells, at least 4% of hematopoietic cells, at least 5% of hematopoietic cells, at least 6% of hematopoietic cells, at least 7% of hematopoietic cells, at least 8% of hematopoietic cells, at least 9% of hematopoietic cells, at least 10% of hematopoietic cells, at least 11% of hematopoietic cells, at least 12% of hematopoietic cells, at least 13% of hematopoietic cells, at least 15% of hematopoietic cells, at least 15% of hematopoietic cells, at least 20% of hematopoietic cells, or more, or between 1-5% of hematopoietic cells, between 1-10% of hematopoietic cells, between 1-15% of hematopoietic cells, between 1-20% of hematopoietic cells, between 5-10% of hematopoietic cells, between 5-15% of hematopoietic cells, between 5-20% of hematopoietic cells, between 10-15% of hematopoietic cells, between 10-20% of hematopoietic cells, between 15-20% of hematopoietic cells, present in a sample obtained from the subject. In some embodiments, a sub-population of cells in a subject can refer to a specific cell type or lineage within the hematopoietic cells in the subject, such as myeloid lineage cells or macrophages. In such embodiments, the “sub-population” of cells comprising the one or more mutations in an HSC cardiometabolic driver gene in the subject refers to at least 1% of myeloid cells, at least 2% of myeloid cells, at least 3% of myeloid cells, at least 4% of myeloid cells, at least 5% of myeloid cells, at least 6% of myeloid cells, at least 7% of myeloid cells, at least 8% of myeloid cells, at least 9% of myeloid cells, at least 10% of myeloid cells, at least 11% of myeloid cells, at least 12% of myeloid cells, at least 13% of myeloid cells, at least 15% of myeloid cells, at least 15% of myeloidcells, at least 20% of myeloidcells, or more, or between 1-5% of myeloid cells, between 1-10% of myeloid cells, between 1-15% of myeloid cells, between 1-20% of myeloid cells, between 5-10% of myeloid cells, between 5-15% of myeloid cells, between 5-20% of myeloid cells, between 10-15% of myeloid cells, between 10-20% of myeloid cells, between 15-20% of myeloid cells, or greater than 20% of myeloid cells present in a sample obtained from the subject.

The terms “biological sample” or “sample” as used herein refers to a cell or population of cells or a quantity of tissue or fluid from a subject comprising one or more hematopoietic cells. Most often, the biological sample has been removed from a subject, but the term “biological sample” can also refer to cells or tissue analyzed in vivo, i.e., without removal from the subject. Thus, a “sample” of hematopoietic cells can be obtained from any tissue or organ in the subject comprising cells of hematopoietic origin, including blood, spleen, lymph nodes, cord blood, placenta, and bone marrow. Hematopoietic cells (HSCs) include myeloid cells (monocytes and macrophages, neutrophils, basophils, eosinophils, erythrocytes, megakaryocytes/platelets, dendritic cells), and lymphoid lineages (T-cells, B-cells, NKT-cells, NK-cells), as well as progenitor cell populations, including multipotent cells, such as hematopoietic stem cells. As used herein, the term “population of hematopoietic cells” encompasses a heterogeneous or homogeneous population of hematopoietic cells and/or hematopoietic progenitor cells. In other words, a population of hematopoietic cells comprising at least two different cell types is referred to herein as a “heterogeneous population.”

In some aspects, provided herein are sensitive and specific companion diagnostic and treatment methods, also referred to herein as “theranostic methods,” to detect and closely monitor mutations in HSC cardiometabolic driver genes associated with disease, particularly IL-1β, IL-6, and/or TNFα mediated disorders, including cardiometabolic diseases. As used herein, a “companion diagnostic” refers to a diagnostic method and or reagent that are used to identify subjects susceptible to treatment with a particular treatment or to monitor treatment and/or to identify an effective dosage for a subject or sub-group or other group of subjects.

Accordingly, in some aspects, provided herein are methods for detecting a subject having, or at risk for, a cardiometabolic driver gene mutation-mediated proinflammatory disease comprising: (a) obtaining a hematopoietic cell sample from a subject, and (b) sequencing the hematopoietic cell sample from the subject to detect one or more somatic mutations in one or more HSC cardiometabolic driver genes in the hematopoietic cell sample.

In some aspects, provided herein are theranostic methods for treating a subject having, or at risk for, a HSC cardiometabolic driver gene mutation-mediated proinflammatory disease comprising: (a) sequencing a hematopoietic cell sample from the subject to identify one or more somatic mutations in one or more HSC cardiometabolic driver genes, and (b) administering a therapeutically effective amount of a pharmaceutical composition comprising an inhibitor of HSC cardiometabolic driver gene mutation-mediated proinflammatory activity and a pharmaceutically acceptable carrier if one or more somatic mutations in an HSC cardiometabolic driver gene are identified in the hematopoietic cell sample.

A sample of hematopoietic cells for use in the methods and uses described herein can, in some embodiments, undergo further processing, such as via flow cytometric sorting and/or magnetic bead based sorting methods, to become an enriched population of hematopoietic cells for analysis of HSC cardiometabolic driver gene mutations, using any method known to one of skill in the art.

In some embodiments of the aspects described herein, a sample comprising hematopoietic cells isolated from a subject, such as a sample obtained from peripheral blood, is then further processed, for example, by cell sorting (e.g., magnetic sorting or FACS), to obtain a population of enriched or isolated hematopoietic cells or a sub-population thereof, for example, myeloid-derived cells.

The terms “isolate” and “methods of isolation,” as used herein, refer to any process whereby a cell or population of cells, such as a population of hematopoietic cells, is removed from a subject or sample in which it was originally found, or a descendant of such a cell or cells. The term “isolated population,” as used herein, refers to a population of cells that has been removed and separated from a biological sample, or a mixed or heterogeneous population of cells found in such a sample. Such a mixed population includes, for example, a population of hematopoietic cells obtained from peripheral blood. In some embodiments, an isolated population is a substantially pure population of cells as compared to the heterogeneous population from which the cells were isolated or enriched from. In some embodiments of this aspect and all such aspects described herein, the isolated population is an isolated population of myeloid cells. In other embodiments of this aspect and all aspects described herein, the isolated population comprises a substantially pure population of myeloid cells as compared to a heterogeneous population of hematopoietic cells comprising various other cells types.

The term “substantially pure,” with respect to a particular cell population, refers to a population of cells that is at least about 75%, at least about 80%, at least about 85%, at least about 90%, at least about 95%, at least about 98%, or at least about 99% pure, with respect to the cells making up a total cell population.

The terms “enriching” or “enriched” are used interchangeably herein and mean that the yield (fraction) of cells of one type, such as hematopoietic cells for use in the methods and uses described herein, is increased by at least 15%, by at least 20%, by at least 25%, by at least 30%, by at least 35%, by at least 40%, by at least 45%, by at least 50%, by at least 55%, by at least 60%, by at least 65%, by at least 70%, or by at least 75%, over the fraction of cells of that type in the starting biological sample, culture, or preparation.

In some embodiments of the aspects described herein, markers specific for different hematopoietic cell types are used to isolate or enrich for these cells. A “marker,” as used herein, describes the characteristics and/or phenotype of a cell. Markers can be used for selection of cells comprising characteristics of interest. Markers will vary with specific cells. Markers are characteristics, whether morphological, functional or biochemical (enzymatic), particular to a cell type, or molecules expressed by the cell type. Preferably, such markers are proteins, and more preferably, possess an epitope for antibodies or other binding molecules available in the art. However, a marker may consist of any molecule found in a cell including, but not limited to, proteins (peptides and polypeptides), lipids, polysaccharides, nucleic acids and steroids. Examples of morphological characteristics or traits include, but are not limited to, shape, size, appearance (e.g., smooth, translucent), and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers may be detected by any method available to one of skill in the art.

Accordingly, as used herein, a “cell-surface marker” refers to any molecule that is expressed on the surface of a cell. Cell-surface expression usually requires that a molecule possesses a transmembrane domain. Some molecules that are normally not found on the cell-surface can be engineered by recombinant techniques to be expressed on the surface of a cell. Many naturally occurring cell-surface markers are termed “CD” or “cluster of differentiation” molecules. Cell-surface markers often provide antigenic determinants to which antibodies can bind to.

A cell can be designated “positive” or “negative” for any cell-surface marker, and both such designations are useful for the practice of the methods described herein. A cell is considered “positive” for a cell-surface marker if it expresses the marker on its cell-surface in amounts sufficient to be detected using methods known to those of skill in the art, such as contacting a cell with an antibody that binds specifically to that marker, and subsequently performing flow cytometric analysis of such a contacted cell to determine whether the antibody is bound the cell. It is to be understood that while a cell may express messenger RNA for a cell-surface marker, in order to be considered positive for the methods described herein, the cell must express it on its surface. Similarly, a cell is considered “negative” for a cell-surface marker if it does not express the marker on its cell-surface in amounts sufficient to be detected using methods known to those of skill in the art, such as contacting a cell with an antibody that binds specifically to that marker and subsequently performing flow cytometric analysis of such a contacted cell to determine whether the antibody is bound the cell. In some embodiments, where agents specific for cell-surface lineage markers used, the agents can all comprise the same label or tag, such as fluorescent tag, and thus all cells positive for that label or tag can be excluded or removed, to leave uncontacted hematopoietic stem or progenitor cells for use in the methods described herein. In some embodiments of the aspects described herein, an agent specific for a cell-surface molecule, such as an antibody or antigen-binding fragment, is labeled with a tag to facilitate the isolation of the hematopoietic stem cells. The terms “label” or “tag”, as used herein, refer to a composition capable of producing a detectable signal indicative of the presence of a target, such as, the presence of a specific cell-surface marker in a biological sample. Suitable labels include fluorescent molecules, radioisotopes, nucleotide chromophores, enzymes, substrates, chemiluminescent moieties, magnetic particles, bioluminescent moieties, and the like. As such, a label is any composition detectable by spectroscopic, photochemical, biochemical, immunochemical, electrical, optical or chemical means needed for the methods to isolate and enrich endothelial cell progenitor cells.

In some embodiments of the aspects described herein, a variety of methods to isolate a substantially pure or enriched population of cells, such as myeloid cells, are available to a skilled artisan, including immunoselection techniques, such as high-throughput cell sorting using flow cytometric methods, affinity methods with antibodies labeled to magnetic beads, biodegradable beads, non-biodegradable beads, and antibodies panned to surfaces including dishes, and any combination of such methods.

As defined herein, “positive selection” refers to techniques that result in the isolation or enrichment of cells expressing specific cell-surface markers, while “negative selection” refers techniques that result in the isolation or enrichment of cells not expressing specific cell-surface markers. In some embodiments, beads can be coated with antibodies by a skilled artisan using standard techniques known in the art, such as commercial bead conjugation kits. In some embodiments, a negative selection step is performed to remove cells expressing one or more lineage markers, followed by fluorescence activated cell sorting to positively select cells expressing one or more specific cell-surface markers.

As defined herein, “flow cytometry” refers to a technique for counting and examining microscopic particles, such as cells and chromosomes, by suspending them in a stream of fluid and passing them through an electronic detection apparatus. Flow cytometry allows simultaneous multiparametric analysis of the physical and/or chemical parameters of up to thousands of particles per second, such as fluorescent parameters. Modern flow cytometric instruments usually have multiple lasers and fluorescence detectors. Increasing the number of lasers and detectors allows for labeling by multiple antibodies, and can more precisely identify a target population by their phenotypic markers. Certain flow cytometric instruments can take digital images of individual cells, allowing for the analysis of fluorescent signal location within or on the surface of cells.

A common variation of flow cytometric techniques is to physically sort particles based on their properties, so as to purify populations of interest, using “fluorescence-activated cell sorting” As defined herein, “fluorescence-activated cell sorting” or “flow cytometric based sorting” methods refer to flow cytometric methods for sorting a heterogeneous mixture of cells from a single biological sample into one or more containers, one cell at a time, based upon the specific light scattering and fluorescent characteristics of each cell and provides fast, objective and quantitative recording of fluorescent signals from individual cells as well as physical separation of cells of particular interest. Accordingly, in some embodiments, fluorescence-activated cell sorting (FACS) can be used with the methods described herein to isolate and enrich for populations of cells, such as myeloid cells, from a sample of hematopoietic cells.

In some embodiments of the methods described herein, the methods further comprise monitoring clonality of a HSC cardiometabolic driver gene somatic mutations in a subject. In other words, following the treatment, the size or percentage of the clone harboring a somatic mutation in a HSC cardiometabolic driver gene is determined to monitor the effectiveness of the treatment.

In some embodiments of the methods described herein, the methods further comprise decreasing the number or percentage of hematopoietic clones comprising the one or more a HSC cardiometabolic driver gene mutation(s) in the subject by transfusing the subject with hematopoietic stem cells in which the mutations are absent or reduced, for example, by administering a bone marrow transplant.

In some such embodiments, the subject is transfused with autologous bone marrow. Alternatively, or additionally, in some embodiments, the subject is transfused with allogeneic bone marrow.

A bone marrow transplant is a procedure where healthy bone marrow stem cells, are infused into a subject to replace damaged or diseased bone marrow, or to replace damaged peripheral blood cells generated from bone marrow stem cells. Prior to the transplant, chemotherapy, radiation, or both can be given. In what is known as ablative (myeloablative) treatment, typically used for cancer treatments, high-dose chemotherapy, radiation, or both are given to kill peripheral cells, as well as all healthy bone marrow that remains, and allows new stem cells to grow in the bone marrow. Reduced intensity treatments, also called a mini transplant, can also be performed where lower doses of chemotherapy and radiation are received before a transplant. For the methods described herein, where the issues arise from somatic mutations in the periphery, total ablation of the bone marrow may not be required.

If an autologous stem cell transplant is used, apheresis can be used to collect blood stem cells. Briefly, blood is withdrawn from the subject's body and one or more blood components are removed, such as all leukocytes or all myeloid cells, and transfusion of the remaining cells are performed. Before apheresis, daily injections of growth factor can be administered to increase stem cell production and move stem cells into circulating blood so they can be collected. During apheresis, blood is drawn from a vein and circulated through a machine. The machine separates blood into different parts, including hematopoietic stem cells. These stem cells can be collected and frozen for future use in the bone marrow transplant.

In some embodiments of the methods described herein, the methods further comprise decreasing the number or percentage of hematopoietic cells or clones comprising the one or more a HSC cardiometabolic driver gene mutations in the subject by performing therapeutic cytapheresis on the subject.

Therapeutic cytapheresis removes cellular components from blood, returning plasma. It is most often used to remove defective RBCs and substitute normal ones in patients with sickle cell anemia who have the following conditions: acute chest syndrome, stroke, pregnancy, or frequent, severe sickle cell crises. Other known uses of cytapheresis include collection of peripheral blood stem cells for autologous or allogeneic bone marrow reconstitution (an alternative to bone marrow transplantation) and collection of lymphocytes for use in immune modulation cancer therapy (adoptive immunotherapy).

In the methods described herein, a subject undergoing therapeutic cytapheresis can also be administered one or more agents to stimulate hematopoietic stem cell migration from the bone marrow to the blood, following removal of all cellular components from the blood using therapeutic cytapheresis.

In some embodiments, a subject undergoing therapeutic cytapheresis can further be transfused with autologous blood. Alternatively, or additionally, in some embodiments, the subject is further transfused with allogeneic blood.

In those embodiments where the subject is transfused with autologous blood, the blood can undergo processing steps prior to transfusion to remove and/or decrease the number of hematopoietic cells having the one or more a HSC cardiometabolic driver gene mutations. Such processing steps can include flow cytometric or magnetic bead-based sorting and enrichment methods to remove hematopoietic cells having the one or more a HSC cardiometabolic driver gene mutations

In some embodiments of the methods described herein, the subject is administered or transfused with hematopoietic cells that have been modified to correct any somatic mutations in a HSC cardiometabolic driver gene, using any method known in the art to modify or incorporate target genes into the genome of a cell so as to facilitate the expression of such genes, also referred to herein as “gene targeting” or “gene therapy” methods.

One system for the integration or modification of target genes into the genome of a hematopoietic cell is the clustered regularly interspaced short palindromic repeats (CRISPR)/Cas system, a system that originally evolved as an adaptive defense mechanism in bacteria and archaea against viral infection. The CRISPR/Cas system includes palindromic repeat sequences within plasmid DNA and an associated Cas9 nuclease. This ensemble of DNA and protein directs site specific DNA cleavage of a target sequence by first incorporating foreign DNA into CRISPR loci. Polynucleotides containing these foreign sequences and the repeat-spacer elements of the CRISPR locus are in turn transcribed in a host cell to create a guide RNA, which can subsequently anneal to a target sequence and localize the Cas9 nuclease to this site. In this manner, highly site-specific cas9-mediated DNA cleavage can be engendered in a foreign polynucleotide because the interaction that brings cas9 within close proximity of the target DNA molecule is governed by RNA:DNA hybridization. As a result, one can theoretically design a CRISPR/Cas system to cleave any target DNA molecule of interest. This technique has been exploited in order to edit eukaryotic genomes (Hwang et al. Nature Biotechnology 31:227 (2013) and can be used as an efficient means of site-specifically editing hematopoietic stem cell genomes in order to cleave DNA prior to the incorporation of a gene encoding a target gene, such as an HSC cardiometabolic driver gene gene lacking the somatic mutations described herein. The use of CRISPR/Cas to modulate gene expression has been described in, e.g., U.S. Pat. No. 8,697,359, the disclosure of which is incorporated herein by reference. Alternative methods for site-specifically cleaving genomic DNA prior to the incorporation of a gene of interest in a hematopoietic cell include the use of zinc finger nucleases (ZFNs) and transcription activator-like effector nucleases (TALENs). Unlike the CRISPR/Cas system, these enzymes do not contain a guiding polynucleotide to localize to a specific target sequence. Target specificity is instead controlled by DNA binding domains within these enzymes. The use of ZFNs and TALENs in genome editing applications is described, e.g., in Urnov et al. Nature Reviews Genetics 11:636 (2010); and in Joung et al. Nature Reviews Molecular Cell Biology 14:49 (2013), the disclosure of both of which are incorporated herein by reference.

Another method that can be used for incorporating polynucleotides encoding target genes into hematopoietic stem cells involves the use of transposons. Transposons are polynucleotides that encode transposase enzymes and contain a polynucleotide sequence or gene of interest flanked by 5′ and 3′ excision sites. Once a transposon has been delivered into a cell, expression of the transposase gene commences and results in active enzymes that cleave the gene of interest from the transposon. This activity is mediated by the site-specific recognition of transposon excision sites by the transposase. In certain cases, these excision sites may be terminal repeats or inverted terminal repeats. Once excised from the transposon, the gene of interest can be integrated into the genome of a mammalian cell by transposase-catalyzed cleavage of similar excision sites that exist within the nuclear genome of the cell. This allows the gene of interest to be inserted into the cleaved nuclear DNA at the complementary excision sites, and subsequent covalent ligation of the phosphodiester bonds that join the gene of interest to the DNA of the mammalian cell genome completes the incorporation process. In certain cases, the transposon may be a retrotransposon, such that the gene encoding the target gene is first transcribed to an RNA product and then reverse-transcribed to DNA before incorporation in the mammalian cell genome. Exemplary transposon systems include the piggybac transposon (described in detail in, e.g., WO 2010/085699) and the sleeping beauty transposon (described in detail in, e.g., US2005/0112764), the disclosures of each of which are incorporated herein by reference.

Additional genome editing techniques that can be used to incorporate polynucleotides encoding target genes into the genome of a hematopoietic cell include the use of ARCUS™ meganucleases that can be rationally designed so as to site-specifically cleave genomic DNA. The use of these enzymes for the incorporation of genes encoding target genes into the genome of a mammalian cell is advantageous in view of the defined structure-activity relationships that have been established for such enzymes. Single chain meganucleases can be modified at certain amino acid positions in order to create nucleases that selectively cleave DNA at desired locations, enabling the site-specific incorporation of a target gene into the nuclear DNA of a hematopoietic stem cell. These single-chain nucleases have been described extensively in, e.g., U.S. Pat. Nos. 8,021,867 and 8,445,251, the disclosures of each of which are incorporated herein by reference.

Another example of a platform that can be used to facilitate the expression of a target gene in a hematopoietic cell is by the integration of the polynucleotide encoding a target gene into the nuclear genome of the cell. A variety of techniques have been developed for the introduction of exogenous genes into a eukaryotic genome. One such technique involves the insertion of a target gene into a vector, such as a viral vector. Vectors for use with the compositions and methods of the invention can be introduced into a cell by a variety of methods, including transformation, transfection, direct uptake, projectile bombardment, and by encapsulation of the vector in a liposome, and are well known in the art. Examples of suitable methods of transfecting or transforming cells include calcium phosphate precipitation, electroporation, microinjection, infection, lipofection and direct uptake. Such methods are described in more detail, for example, in Green, et al., Molecular Cloning: A Laboratory Manual, Fourth Edition, Cold Spring Harbor University Press, New York (2014); and Ausubel, et al., Current Protocols in Molecular Biology, John Wiley & Sons, New York (2015), the disclosures of each of which are incorporated herein by reference.

Examples of viral vectors useful in the methods described herein include a retrovirus, adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including herpes virus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D-type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996, the disclosure of which is incorporated herein by reference). Other examples of viral vectors include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described in, e.g., U.S. Pat. No. 5,801,030, the disclosure of which is incorporated herein by reference.

In some embodiments, the methods further comprise initiating a monitoring regimen following the administration of one or more treatments to the subject. For example, monitoring includes repeating the diagnostic steps of the method on the subject on a monthly, bi-monthly or quarterly basis to determine whether there is, for example, reduced IL-1β, IL-6, and/or TNFα proinflammatory activity or decreased percentages of hematopoietic cells in the blood having one or more a HSC cardiometabolic driver gene mutations described herein.

In some embodiments of the methods described herein, the theranostic methods comprise further administering one or more additional therapeutic agents to the subject, in addition to the inhibitor of a HSC cardiometabolic driver gene mutation-mediated proinflammatory activity. Such an additional therapeutic agent can be co-administered with the inhibitor of an HSC cardiometabolic driver gene mutation-mediated proinflammatory activity. As used herein, the phrase “co-administering” or to “co-administer” means the administration of an inhibitor described herein and another compound, e.g., a therapeutic agent, separately, simultaneously, and/or sequentially over a period of time as determined by a qualified care giver.

Non-limiting examples of additional therapeutic agents that can be administered to a subject having one or more somatic mutations in one or more HSC cardiometabolic driver genes include quinidine, procainamide, disopyramide, lidocaine, phenytoin, mexiletine, flecainide, propafenone, moricizine, propranolol, esmolol, timolol, metoprolol, atenolol, bisoprolol, amiodarone, sotalol, ibutilide, dofetilide, dronedarone, E-4031, verapamil, diltiazem, adenosine, digoxin, magnesium sulfate, warfarin, heparins, anti-platelet drugs (e.g., aspirin and clopidogrel), beta blockers (e.g., metoprolol and carvedilol), angiotensin-converting enzyme (ACE) inhibitors (e.g., captopril, zofenopril, enalapril, ramipril, quinapril, perindopril, lisinopril, benazepril, fosinopril, casokinins and lactokinins), statins (e.g., atorvastatin, cerivastatin, fluvastatin, lovastatin, pitavastatin, mevastatin, pravastatin, rosuvastatin, and simvastatin), aldosterone antagonist agents (e.g., eplerenone and spironolactone), digitalis, diuretics, digoxin, inotropes (e.g., Milrinone), vasodilators and omega-3 fatty acids and combinations thereof.

In some aspects and embodiments of the methods directed to treatment of chronic kidney diseases, the additional therapeutic agent is an angiotensin-converting enzyme (ACE) inhibitor, an angiotensin II receptor blocker (ARB), or a mineralocorticoid receptor (MR) antagonist.

ACE inhibitors for use with the compositions and methods described herein include, but are not limited to, benazepril (marketed in the U.S. as LOTENSIN™), captopril (marketed in the U.S. as CAPOTEN™), enalapril/enalaprilat (marketed in the U.S. as VASOTEC™ oral and injectable), fosinopril (marketed in the U.S. as MONOPRIL™), lisinopril (marketed in the U.S. as ZESTRIL™ and PRINIVIL™), moexipril (marketed in the U.S. as UNIVASC™), perindopril (marketed in the U.S. as ACEON™), quinapril (marketed in the U.S. as ACCUPRIL™), ramipril (marketed in the U.S. as ALTACE™), and trandolapril (marketed in the U.S. as MAVIK™). ARBs for use with the inhibitors described herein include candesartan (marketed in the U.S. as ATACAND™), irbesartan (marketed in the U.S. as AVAPRO™), olmesartan (marketed in the U.S. as BENICARM), losartan (marketed in the U.S. as COZAAR™), valsartan (marketed in the U.S. as DIOVAN™), telmisartan (marketed in the U.S. as MICARDIS™), and eprosartan (marketed in the U.S. as TEVETEN™).

In some embodiments of these methods and all such methods described herein, the method further comprises administering to the subject an effective amount of a diuretic. Diuretics include, but are not limited to, torsemide (marketed in the U.S. as DEMADEX™), furosemide (marketed in the U.S. as LASIX™), bumetanide (marketed in the U.S. as BUMEX™), ethacrynic acid (marketed in the U.S. as EDECRIN™), torsemide (marketed in the U.S. as DEMADEX™), amiloride, (marketed in the U.S. as MIDAMOR™), acetazolamide (marketed in the U.S. as DIAMOXm), pamabrom (marketed in the U.S. as AQUA-BAN™), mannitol (marketed in the U.S. as ARIDOL™ or OSMITROL™), traimterene (marketed in the U.S. as DYRENIUM™), spironolactone (marketed in the U.S. as ALDACTONE), amiloride (marketed in the U.S. as MIDAMOR™), indapamide (marketed in the U.S. as LOZOLh), hydrochlorothiazide (marketed in the U.S. as HYDRODIURIL™), metolazone (marketed in the U.S. as ZAROXOLYN™ or MYKROX™), methylclothiazide (marketed in the U.S. as AQUATENSEN™ or ENDURON™), hydrocholorthiazide (marketed in the U.S. as AQUAZIDE H™ or ESIDRIX™ or MICROZIDE™), chlorothiazide (marketed in the U.S. as DIURIL™), bendroflumethiazide (marketed in the U.S. as NATURETIN™), polythiazide (marketed in the U.S. as RENESE™), hydroflumethiazide (marketed in the U.S. as SALURON™), and chlorthalidone (marketed in the U.S. as THALITONE™). For a complete listing also see, e.g., Physician's Desk Reference, 2017 Edition, PDR Network (2016).

As used herein, the terms “treat” or “treatment” or “treating” as used herein in reference to use of inhibitors of a HSC cardiometabolic driver gene mutation-mediated IL-1β, IL-6, and/or TNFα proinflammatory activity for the treatment of a cardiovascular disease or disorder refers to therapeutic treatment, wherein the object is to prevent or slow the development of the disease, such as slow down the development of a cardiac disorder, or reducing at least one adverse effect or symptom of a cardiovascular condition, disease or disorder, i.e., any disorder characterized by insufficient or undesired cardiac function. Adverse effects or symptoms of cardiac disorders are well-known in the art and include, but are not limited to, dyspnea, chest pain, palpitations, dizziness, syncope, edema, cyanosis, pallor, fatigue and death. Treatment is generally “effective” if one or more symptoms or clinical markers are reduced as that term is defined herein. Alternatively, a treatment is “effective” if the progression of a disease is reduced or halted. That is, “treatment” includes not just the improvement of symptoms or decrease of markers of the disease, but also a cessation or slowing of progress or worsening of a symptom that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment.

In some embodiments, the term “treating” when used in reference to a treatment of a cardiovascular disease or disorder is used to refer to the reduction of a symptom and/or a biochemical marker of a cardiovascular disease or disorder, for example a reduction in at least one biochemical marker of a cardiovascular disease by at least about 10% would be considered an effective treatment. Examples of such biochemical markers of cardiovascular disease include a reduction of, for example, creatine phosphokinase (CPK), aspartate aminotransferase (AST), lactate dehydrogenase (LDH) in the blood, and/or a decrease in a symptom of cardiovascular disease, such as atherosclerosis, and/or an improvement in blood flow and cardiac function as determined by someone of ordinary skill in the art as measured by electrocardiogram (ECG or EKG), or echocardiogram (heart ultrasound), Doppler ultrasound and nuclear medicine imaging. A reduction in a symptom of a cardiovascular disease by at least about 10% would also be considered effective treatment by the methods as disclosed herein. As alternative examples, a reduction in a symptom of cardiovascular disease, for example a reduction of at least one of the following; dyspnea, chest pain, palpitations, dizziness, syncope, edema, cyanosis etc. by at least about 10% or a cessation of such systems, or a reduction in the size one such symptom of a cardiovascular disease by at least about 10% would also be considered as affective treatments by the methods as disclosed herein. In some embodiments, it is preferred, but not required that the therapeutic agent actually eliminate the cardiovascular disease or disorder, rather just reduce a symptom to a manageable extent.

The term “effective amount” as used herein refers to the amount of therapeutic agent of pharmaceutical composition to alleviate at least one or more symptom of the disease or disorder, and relates to a sufficient amount of pharmacological composition to provide the desired effect. With reference to the treatment of, for example, a cardiovascular condition or disease in a subject, the term “effective amount” refers to the amount that is safe and sufficient to prevent or delay the development or a cardiovascular disease or disorder. The amount can thus cure or cause the cardiovascular disease or disorder to go into remission, slow the course of cardiovascular disease progression, slow or inhibit a symptom of a cardiovascular disease or disorder, slow or inhibit the establishment of secondary symptoms of a cardiovascular disease or disorder or inhibit the development of a secondary symptom of a cardiovascular disease or disorder. The effective amount for the treatment of the cardiovascular disease or disorder depends on the type of cardiovascular disease to be treated, the severity of the symptoms, the subject being treated, the age and general condition of the subject, the mode of administration and so forth. Thus, it is not possible to specify the exact “effective amount”. However, for any given case, an appropriate “effective amount” can be determined by one of ordinary skill in the art using only routine experimentation. The efficacy of treatment can be judged by an ordinarily skilled practitioner, for example, efficacy can be assessed in animal models of a cardiovascular disease or disorder as discussed herein, and any treatment or administration of the compositions or formulations that leads to a decrease of at least one symptom of the cardiovascular disease or disorder as disclosed herein, for example, decreased levels of atherosclerosis in the blood vessels, increased heart ejection fraction, decreased rate of heart failure, decreased infarct size, decreased associated morbidity (pulmonary edema, renal failure, arrhythmias) improved exercise tolerance or other quality of life measures, and decreased mortality indicates effective treatment.

By “reduce” or “inhibit” in terms of the methods of treatment of chronic kidney disease and proteinuria described herein is meant the ability to cause an overall decrease preferably of 20% or greater, 30% or greater, 40% or greater, 45% or greater, more preferably of 50% or greater, of 55% or greater, of 60% or greater, of 65% or greater, of 70% or greater, and most preferably of 75% or greater, 80% or greater, 85% or greater, 90% or greater, or 95% or greater, for a given parameter or symptom of a chronic kidney disease. Reduce or inhibit can refer to, for example, symptoms of the disorder being treated, for example, high blood pressure, protein in the urine, etc.

High blood pressure is almost always present during all stages of chronic kidney disease. A nervous system exam may show signs of nerve damage. The health care provider may hear abnormal heart or lung sounds when listening with a stethoscope. The early symptoms of chronic kidney disease are also symptoms of other illnesses. These symptoms can be the only signs of kidney disease until the condition is more advanced. Symptoms of chronic kidney disease can include: appetite loss; general ill feeling and fatigue; headaches; itching (pruritus) and dry skin; nausea; weight loss without trying to lose weight; etc. Other symptoms that can develop, especially when kidney function has gotten worse, include: abnormally dark or light skin; bone pain; brain and nervous system symptoms; drowsiness and confusion; problems concentrating or thinking; numbness in the hands, feet, or other areas; muscle twitching or cramps; breath odor; easy bruising, bleeding, or blood in the stool; excessive thirst; frequent hiccups; low level of sexual interest and impotence; stopping of menstrual periods (amenorrhea); shortness of breath; sleep problems, such as insomnia, restless leg syndrome, and obstructive sleep apnea; swelling of the feet and hands (edema); vomiting, typically in the morning.

Accordingly, in some embodiments of the methods described herein, an effective amount of a composition comprising an inhibitor of an HSC cardiometabolic driver gene mutation-mediated proinflammatory activity described herein is administered to a subject in order to alleviate one or more symptoms of chronic kidney disease. As used herein, “alleviating a symptom chronic kidney disease” is ameliorating any condition or symptom associated with the chronic kidney disease. Alternatively, alleviating a symptom of a chronic kidney disease can involve reducing one or more symptoms of the chronic kidney disease in the subject relative to an untreated control suffering from chronic kidney disease or relative to the subject prior to the treatment. As compared with an equivalent untreated control, or the subject prior to the treatment with the inhibitor, such reduction or degree of prevention is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more, as measured by any standard technique. Desirably, the chronic kidney disease is significantly reduced or undetectable, as detected by any standard method known in the art, in which case the chronic kidney disease is considered to have been treated. A patient who is being treated for a chronic kidney disease is one who a medical practitioner has diagnosed as having such a condition. Diagnosis can be by any suitable means known to one of ordinary skill in the art. Diagnosis and monitoring can involve, for example, detecting the level of specific proteins or molecules in a urine, blood, or serum sample, such as, for example, albumin, calcium, cholesterol, complete blood count (CBC), electrolytes, magnesium, phosphorous, potassium, sodium, or any combination thereof, assays to detect, for example, creatinine clearance; creatinine levels; BUN (blood urea nitrogen); through the use of specific techniques or procedures, such as an abdominal CT scan, abdominal MRI, abdominal ultrasound, kidney biopsy, kidney scan, kidney ultrasound; via detection of changes in results of assays or tests for erythropoietin, PTH; bone density test, or Vitamin D; or any combination of such detection methods and assays.

The compositions and methods described herein ideally result in a therapeutic significant reduction in one or more symptoms. A therapeutically significant reduction in a symptom is, e.g. at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject. Measured or measurable parameters include clinically detectable markers of disease, for example, elevated or depressed levels of a biological marker, as well as parameters related to a clinically accepted scale of symptoms or markers for a disease or disorder. It will be understood, that the total daily usage of the compositions and formulations as disclosed herein will be decided by the attending physician within the scope of sound medical judgment. The exact amount required will vary depending on factors such as the type of disease being treated.

The inhibitors of a HSC cardiometabolic driver gene mutation-mediated proinflammatory activity described herein can be administered using any means or route known to those of ordinary skill in the art and known to provide desired effects. As used herein, the terms “administering,” and “introducing” are used interchangeably herein and refer to the placement of the therapeutic agents as disclosed herein into a subject by a method or route which results in delivering of such agent(s) at a desired site. The compounds can be administered by any appropriate route which results in an effective treatment in the subject, including topical administration.

Routes of administration include, but are not limited to aerosol, direct injection, intradermal, transdermal (e.g., in slow release polymers), intravitreal, intramuscular, intraperitoneal, intravenous, subcutaneous, intranasal, epidural, topical, oral, transmucosal, buccal, rectal, vaginal, transdermal, intranasal and parenteral routes.

The phrases “parenteral administration” and “administered parenterally” as used herein mean modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “systemic administration,” “administered systemically”, “peripheral administration” and “administered peripherally” as used herein mean the administration therapeutic compositions other than directly into a tumor such that it enters the animal's system and, thus, is subject to metabolism and other like processes.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or solid filler, diluent, excipient, solvent or encapsulating material, involved in maintaining the activity of or carrying or transporting the subject agents from one organ, or portion of the body, to another organ, or portion of the body. In addition to being “pharmaceutically acceptable” as that term is defined herein, each carrier must also be “acceptable” in the sense of being compatible with the other ingredients of the formulation. The pharmaceutical formulation contains a compound of the invention in combination with one or more pharmaceutically acceptable ingredients. The carrier can be in the form of a solid, semi-solid or liquid diluent, cream or a capsule. These pharmaceutical preparations are a further object of the invention. Usually the amount of active compounds is between 0.1-95% by weight of the preparation, preferably between 0.2-20% by weight in preparations for parenteral use and preferably between 1 and 50% by weight in preparations for oral administration. For the clinical use of the methods of the present invention, targeted delivery composition of the invention is formulated into pharmaceutical compositions or pharmaceutical formulations for parenteral administration, e.g., intravenous; mucosal, e.g., intranasal; enteral, e.g., oral; topical, e.g., transdermal; ocular, e.g., via corneal scarification or other mode of administration. The pharmaceutical composition contains a compound of the invention in combination with one or more pharmaceutically acceptable ingredients. The carrier can be in the form of a solid, semi-solid or liquid diluent, cream or a capsule.

It is understood that the foregoing description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

All patents and other publications identified are expressly incorporated herein by reference for the purpose of describing and disclosing, for example, the methodologies described in such publications that could be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Some embodiments of the technology described herein can be defined according to any of the following numbered paragraphs:

1. A method for treating a subject having, or at risk for, a HSC (hematopoietic stem cell) cardiometabolic driver gene mutation-mediated proinflammatory disease comprising: administering a therapeutically effective amount of a pharmaceutical composition comprising an inhibitor of HSC cardiometabolic driver gene mutation-mediated proinflammatory activity and a pharmaceutically acceptable carrier to a subject having one or more somatic mutations in one or more HSC cardiometabolic driver gene in a sub-population of peripheral blood hematopoietic cells.
2. The method of paragraph 1, wherein the one or more somatic mutations in the one or more HSC cardiometabolic driver genes in the sub-population of peripheral blood hematopoietic cells cause clonal hematopoiesis in the subject.
3. The method of any one of paragraphs 1 or 2, wherein at least 2% of the peripheral blood hematopoietic cells have the one or more somatic mutations in the one or more HSC cardiometabolic driver genes.
4. The method of any one of paragraphs 1-3, wherein the one or more HSC cardiometabolic driver genes are selected from TP53, JAK2, DNMT3A, ASXL1, and PPM1D/WIP1.
5. The method of paragraph 4, wherein the one or more somatic mutations are in TP53 and are selected from a G743A mutation in SEQ ID NO:2 and a A659G mutation in SEQ ID NO: 2.
6. The method of any one of paragraphs 4 or 5, wherein the one or more somatic mutations are in JAK2 and is a G1849T in SEQ ID NO: 56.
7. The method of any one of paragraphs 4-6, wherein the one or more somatic mutations are in DNMT3A and are selected from a T1115C mutation in SEQ ID NO: 37; a C2711T mutation in SEQ ID NO: 37; a C1837G mutation in SEQ ID NO: 37; a A1666G mutation in SEQ ID NO: 37; a C1789T mutation in SEQ ID NO: 37; a G2719A mutation in SEQ ID NO: 37; a G1627T mutation in SEQ ID NO: 37; an A2723G mutation in SEQ ID NO: 37; a G1797T mutation in SEQ ID NO: 37; a T2252G mutation in SEQ ID NO: 37; a C1560A mutation in SEQ ID NO: 37; a T1031C mutation in SEQ ID NO: 37; a G2645A mutation in SEQ ID NO: 37; a C2043G mutation in SEQ ID NO: 37; a C2446T mutation in SEQ ID NO: 37; a A2198G mutation in SEQ ID NO: 37; A2281G mutation in SEQ ID NO: 37; a C920G mutation in SEQ ID NO: 37; a A2204G mutation in SEQ ID NO: 37; and a frameshift mutation in DNMT3A.
8. The method of any one of paragraphs 4-7, wherein the one or more somatic mutations are in ASXL1 and are selected from a C2407T mutation in SEQ ID NO: 61; a C2893T mutation in SEQ ID NO: 61; a 1926_1926delinsAG mutation in SEQ ID NO: 61; and a frameshift mutation in ASXL1.
9. The method of any one of paragraphs 4-8, wherein the one or more somatic mutations are in PPM1D and are selected from a G1618T mutation in SEQ ID NO: 64; a C1372T mutation in SEQ ID NO: 64; and a frameshift mutation in PPM1D.
10. The method of any one of paragraphs 1-9, wherein the subject further has one or more somatic mutations in TET2 in a sub-population of peripheral blood hematopoietic cells selected from an S282F mutation in SEQ ID NO: 68, an N312S mutation in SEQ ID NO: 68, an L346P mutation in SEQ ID NO: 68, an S460F mutation in SEQ ID NO: 68, a D666G mutation in SEQ ID NO: 68, a P941S mutation in SEQ ID NO: 68, and a C1135Y mutation in SEQ ID NO: 68.
11. The method of any one of paragraphs 1-10, wherein the inhibitor of HSC cardiometabolic driver gene mutation-mediated proinflammatory activity is an IL-1β inhibitor.
12. The method of paragraph 11, wherein the IL-1β inhibitor is an IL-1β inhibitor antibody or antigen-binding fragment thereof that binds to IL-1β and reduces IL-1β binding to its receptor(s).
13. The method of paragraph 12, wherein the IL-1β inhibitor antibody or antigen-binding fragment thereof is selected from ABT981, an anti-interleukin-11 inhibitor antibody by ABZYME, APX002, Canakinumab/Ilaris, CDP48, immunereszumab, LY2189102, MEDI8968, and XOMA052.
14. The method of paragraph 11, wherein the IL-1β inhibitor is an IL-1 receptor antagonist.
15. The method of paragraph 14, wherein the IL-1 receptor antagonist is selected from CDP484, CP412245, CYT013 IL1bQb, XL 130, AMG108, HL 2351, IL1Hyl, AXXO, orthokine, PRT 1000, anakinra, and rilonacept.
16. The method of paragraph 11, wherein the IL-1β inhibitor is a small molecule or microRNA inhibitor that inhibits IL-1β-mediated pro-inflammatory activity.
17. The method of paragraph 16, wherein the small molecule or microRNA inhibitor is selected from AC201, CP412245, MCC950 or CRID3, inflabion, inflammasome modulator OPSONA, PGE3935199, PGE527667, TRK530, β-hydroxybutyrate (BHB), and microRNA-223.
18. The method of paragraph 17, wherein the small molecule inhibitor is MCC950.
19. The method of any one of paragraphs 1-18, wherein the inhibitor of HSC cardiometabolic driver gene mutation-mediated proinflammatory activity is an IL-6 inhibitor.
20. The method of paragraph 19, wherein the IL-6 inhibitor is an IL-6 inhibitor antibody or antigen-binding fragment thereof that binds to IL-6 and reduces IL-6 binding to its receptor(s).
21. The method of paragraph 20, wherein the IL-6 inhibitor antibody or antigen-binding fragment thereof is selected from Siltuximab, Olokizumab, Elsilimomab, mAb 1339, Sirukumab, Clazakizumab, ARGX-109, FM101, and C326.
22. The method of paragraph 19, wherein the IL-6 inhibitor is an IL-6 receptor antagonist.
23. The method of paragraph 22, wherein the IL-6 receptor antagonist is selected from tocilizumab, sarilumab, REGN88, FE301, and LMT-28.
24. The method of paragraph 19, wherein the IL-6 inhibitor is a small molecule or microRNA inhibitor.
25. The method of paragraph 24, wherein the small molecule IL-6 inhibitor is ALX-0061 or LMT-28.
26. The method of paragraph 19, wherein the IL-6 inhibitor is a JAK-STAT inhibitor.
27. The method of paragraph 26, wherein the JAK-STAT inhibitor is selected from baricitinib, decernotinid, filgotinib, INCB-039110, ruxolitinib, tofacitinib, Oclacitinib, Gandotinib, Lestaurtinib, Momelotinib, Pacritinib, PF-04965842, Upadacitinib, and Peficitinib.
28. The method of any one of paragraphs 1-27, wherein the inhibitor of HSC cardiometabolic driver gene mutation-mediated proinflammatory activity is a TNFα inhibitor.
29. The method of paragraph 28, wherein the TNFα inhibitor is a TNFα inhibitor antibody or antigen-binding fragment thereof that binds to TNFα and reduces TNFα binding to its receptor(s).
30. The method of paragraph 29, wherein the TNFα inhibitor antibody or antigen-binding fragment thereof is selected from adalimumab, Adalimumab-atto, certolizumab pegol, golimumab, infliximab,
31. The method of paragraph 28, wherein the TNFα inhibitor is a TNFα receptor antagonist.
32. The method of paragraph 31, wherein the TNFα receptor antagonist is etanercept.
33. The method of paragraph 28, wherein the TNFα inhibitor is a small molecule or microRNA inhibitor.
34. The method of any one of paragraphs 1-33, further comprising monitoring hematopoietic cell clonality, IL-1β proinflammatory activity, IL-6 proinflammatory activity, TNFα proinflammatory activity or any combination thereof following the administration of the inhibitor of HSC cardiometabolic driver gene mutation-mediated proinflammatory activity.
35. The method of any one of paragraphs 1-34, further comprising decreasing the number or percentage of hematopoietic cells comprising the one or more somatic mutations in the one or more HSC cardiometabolic driver genes in the subject by performing therapeutic cytapheresis on the subject.
36. The method of any one of paragraphs 1-35, further comprising administering one or more additional therapeutic agents to the subject.
37. A method for treating a subject having, or at risk for, a HSC cardiometabolic driver gene mutation-mediated proinflammatory disease comprising:

(a) sequencing a hematopoietic cell sample from a subject to identify one or more somatic mutations in one or more HSC cardiometabolic driver genes in the hematopoietic cell sample; and

(b) administering a therapeutically effective amount of a pharmaceutical composition comprising an inhibitor of HSC cardiometabolic driver gene mutation-mediated proinflammatory activity and a pharmaceutically acceptable carrier if one or more somatic mutations in one or more HSC cardiometabolic driver genes are identified in the hematopoietic cell sample.

38. The method of paragraph 37, wherein the hematopoietic cell sample is a peripheral blood hematopoietic cell sample.
39. The method of any one of paragraphs 37 or 38, wherein the hematopoietic cell sample is enriched for myeloid-derived cells.
40. The method of any one of paragraphs 37-39, wherein the one or more somatic mutations in the one or more HSC cardiometabolic driver genes identified in the hematopoietic cell sample cause clonal hematopoiesis in the subject.
41. The method of any one of paragraphs 37-40, wherein at least 2% of the hematopoietic cells are identified as having one or more HSC cardiometabolic driver gene mutations.
42. The method of any one of paragraphs 37-41, wherein the one or more HSC cardiometabolic driver genes are selected from TP53, JAK2, DNMT3A, ASXL1, and PPM1D/WIP1.
43. The method of paragraph 42, wherein the one or more somatic mutations are in TP53 and are selected from a G743A mutation in SEQ ID NO:2 and a A659G mutation in SEQ ID NO: 2.
44. The method of any one of paragraphs 42 or 43, wherein the one or more somatic mutations are in JAK2 and is a G1849T in SEQ ID NO: 56.
45. The method of any one of paragraphs 42-44, wherein the one or more somatic mutations are in DNMT3A and are selected from a T1115C mutation in SEQ ID NO: 37; a C2711T mutation in SEQ ID NO: 37; a C1837G mutation in SEQ ID NO: 37; a A1666G mutation in SEQ ID NO: 37; a C1789T mutation in SEQ ID NO: 37; a G2719A mutation in SEQ ID NO: 37; a G1627T mutation in SEQ ID NO: 37; an A2723G mutation in SEQ ID NO: 37; a G1797T mutation in SEQ ID NO: 37; a T2252G mutation in SEQ ID NO: 37; a C1560A mutation in SEQ ID NO: 37; a T1031C mutation in SEQ ID NO: 37; a G2645A mutation in SEQ ID NO: 37; a C2043G mutation in SEQ ID NO: 37; a C2446T mutation in SEQ ID NO: 37; a A2198G mutation in SEQ ID NO: 37; A2281G mutation in SEQ ID NO: 37; a C920G mutation in SEQ ID NO: 37; a A2204G mutation in SEQ ID NO: 37; and a frameshift mutation in DNMT3A.
46. The method of any one of paragraphs 42-45, wherein the one or more somatic mutations are in ASXL1 and are selected from a C2407T mutation in SEQ ID NO: 61; a C2893T mutation in SEQ ID NO: 61; a 1926_1926delinsAG mutation in SEQ ID NO: 61; and a frameshift mutation in ASXL1.
47. The method of any one of paragraphs 42-46, wherein the one or more somatic mutations are in PPM1D and are selected from a G1618T mutation in SEQ ID NO: 64; a C1372T mutation in SEQ ID NO: 64; and a frameshift mutation in PPM1D.
48. The method of any one of paragraphs 37-47, wherein the subject further has one or more somatic mutations in TET2 in a sub-population of peripheral blood hematopoietic cells selected from an S282F mutation in SEQ ID NO: 68, an N312S mutation in SEQ ID NO: 68, an L346P mutation in SEQ ID NO: 68, an S460F mutation in SEQ ID NO: 68, a D666G mutation in SEQ ID NO: 68, a P941S mutation in SEQ ID NO: 68, and a C1135Y mutation in SEQ ID NO: 68.
49. The method of any one of paragraphs 37-48, wherein the inhibitor of HSC cardiometabolic driver gene mutation-mediated proinflammatory activity is an IL-1β inhibitor.
50. The method of paragraph 49, wherein the IL-1β inhibitor is an IL-1β inhibitor antibody or antigen-binding fragment thereof that binds to IL-1β and reduces IL-1β binding to its receptor(s).
51. The method of paragraph 50, wherein the IL-1β inhibitor antibody or antigen-binding fragment thereof is selected from ABT981, an anti-interleukin-11 inhibitor antibody by ABZYME, APX002, Canakinumab/Ilaris, CDP48, immunereszumab, LY2189102, MEDI8968, and XOMA052.
52. The method of paragraph 49, wherein the IL-1β inhibitor is an IL-1 receptor antagonist.
53. The method of paragraph 52, wherein the IL-1 receptor antagonist is selected from CDP484, CP412245, CYT013 IL1bQb, XL 130, AMG108, HL 2351, IL1Hyl, AXXO, orthokine, PRT 1000, anakinra, and rilonacept.
54. The method of paragraph 49, wherein the IL-1β inhibitor is a small molecule or microRNA inhibitor that inhibits IL-1β-mediated pro-inflammatory activity.
55. The method of paragraph 54, wherein the small molecule or microRNA inhibitor is selected from AC201, CP412245, MCC950 or CRID3, inflabion, inflammasome modulator OPSONA, PGE3935199, PGE527667, TRK530, β-hydroxybutyrate (BHB), and microRNA-223.
56. The method of paragraph 55, wherein the small molecule inhibitor is MCC950.
57. The method of any one of paragraphs 37-56, wherein the inhibitor of HSC cardiometabolic driver gene mutation-mediated proinflammatory activity is an IL-6 inhibitor.
58. The method of paragraph 57, wherein the IL-6 inhibitor is an IL-6 inhibitor antibody or antigen-binding fragment thereof that binds to IL-6 and reduces IL-6 binding to its receptor(s).
59. The method of paragraph 58, wherein the IL-6 inhibitor antibody or antigen-binding fragment thereof is selected from Siltuximab, Olokizumab, Elsilimomab, mAb 1339, Sirukumab, Clazakizumab, ARGX-109, FM101, and C326.
60. The method of paragraph 57, wherein the IL-6 inhibitor is an IL-6 receptor antagonist.
61. The method of paragraph 60, wherein the IL-6 receptor antagonist is selected from tocilizumab, sarilumab, REGN88, FE301, and LMT-28.
62. The method of paragraph 57, wherein the IL-6 inhibitor is a small molecule or microRNA inhibitor.
63. The method of paragraph 62, wherein the small molecule IL-6 inhibitor is ALX-0061 or LMT-28.
64. The method of paragraph 63, wherein the IL-6 inhibitor is a JAK-STAT inhibitor.
65. The method of paragraph 64, wherein the JAK-STAT inhibitor is selected from baricitinib, decernotinid, filgotinib, INCB-039110, ruxolitinib, tofacitinib, Oclacitinib, Gandotinib, Lestaurtinib, Momelotinib, Pacritinib, PF-04965842, Upadacitinib, and Peficitinib.
66. The method of any one of paragraphs 37-65, wherein the inhibitor of HSC cardiometabolic driver gene mutation-mediated proinflammatory activity is a TNFα inhibitor.
67. The method of paragraph 66, wherein the TNFα inhibitor is a TNFα inhibitor antibody or antigen-binding fragment thereof that binds to TNFα and reduces TNFα binding to its receptor(s).
68. The method of paragraph 67, wherein the TNFα inhibitor antibody or antigen-binding fragment thereof is selected from adalimumab, Adalimumab-atto, certolizumab pegol, golimumab, infliximab,
69. The method of paragraph 66, wherein the TNFα inhibitor is a TNFα receptor antagonist.
70. The method of paragraph 69, wherein the TNFα receptor antagonist is etanercept.
71. The method of paragraph 66, wherein the TNFα inhibitor is a small molecule or microRNA inhibitor.
72. The method of any one of paragraphs 37-71, further comprising monitoring hematopoietic cell clonality, IL-1β proinflammatory activity, IL-6 proinflammatory activity, TNFα proinflammatory activity or any combination thereof following the administration of the inhibitor of HSC cardiometabolic driver gene mutation-mediated proinflammatory activity.
73. The method of any one of paragraphs 37-72, further comprising decreasing the number or percentage of hematopoietic cells comprising the one or more somatic mutations in the one or more HSC cardiometabolic driver genes in the subject by performing therapeutic cytapheresis on the subject.
74. The method of any one of paragraphs 37-73, further comprising administering one or more additional therapeutic agents to the subject.
75. The method of any one of paragraphs 37-74, wherein the HSC cardiometabolic driver gene mutation-mediated proinflammatory disease is a cardiometabolic disease or disorder.
76. The method of any one of paragraphs 37-74, wherein the HSC cardiometabolic driver gene mutation-mediated proinflammatory disease is a chronic kidney disease or disorder.
77. A pharmaceutical composition comprising an inhibitor of HSC cardiometabolic driver gene mutation-mediated proinflammatory activity and a pharmaceutically acceptable carrier for use in a subject having one or more somatic mutations in one or more HSC cardiometabolic driver genes in a sub-population of hematopoietic cells.
78. The pharmaceutical composition of paragraph 77, wherein the one or more somatic mutations in the one or more HSC cardiometabolic driver genes in the sub-population of hematopoietic cells cause clonal hematopoiesis in the subject.
79. The pharmaceutical composition of any one of paragraphs 77 or 78, wherein at least 2% of the hematopoietic cells in the subject have the one or more mutations in one or more HSC cardiometabolic driver genes.
80. The pharmaceutical composition of any one of paragraphs 77-79, wherein the one or more HSC cardiometabolic driver genes are selected from TP53, JAK2, DNMT3A, ASXL1, and PPM1D/WIP1.
81. The pharmaceutical composition of paragraph 80, wherein the one or more somatic mutations are in TP53 and are selected from a G743A mutation in SEQ ID NO:2 and a A659G mutation in SEQ ID NO: 2.
82. The pharmaceutical composition of any one of paragraphs 80 or 81, wherein the one or more somatic mutations are in JAK2 and is a G1849T in SEQ ID NO: 56.
83. The pharmaceutical composition of any one of paragraphs 80-82, wherein the one or more somatic mutations are in DNMT3A and are selected from a T1115C mutation in SEQ ID NO: 37; a C2711T mutation in SEQ ID NO: 37; a C1837G mutation in SEQ ID NO: 37; a A1666G mutation in SEQ ID NO: 37; a C1789T mutation in SEQ ID NO: 37; a G2719A mutation in SEQ ID NO: 37; a G1627T mutation in SEQ ID NO: 37; an A2723G mutation in SEQ ID NO: 37; a G1797T mutation in SEQ ID NO: 37; a T2252G mutation in SEQ ID NO: 37; a C1560A mutation in SEQ ID NO: 37; a T1031C mutation in SEQ ID NO: 37; a G2645A mutation in SEQ ID NO: 37; a C2043G mutation in SEQ ID NO: 37; a C2446T mutation in SEQ ID NO: 37; a A2198G mutation in SEQ ID NO: 37; A2281G mutation in SEQ ID NO: 37; a C920G mutation in SEQ ID NO: 37; a A2204G mutation in SEQ ID NO: 37; and a frameshift mutation in DNMT3A.
84. The pharmaceutical composition of any one of paragraphs 80-83, wherein the one or more somatic mutations are in ASXL1 and are selected from a C2407T mutation in SEQ ID NO: 61; a C2893T mutation in SEQ ID NO: 61; a 1926_1926delinsAG mutation in SEQ ID NO: 61; and a frameshift mutation in ASXL1.
85. The pharmaceutical composition of any one of paragraphs 80-84, wherein the one or more somatic mutations are in PPM1D and are selected from a G1618T mutation in SEQ ID NO: 64; a C1372T mutation in SEQ ID NO: 64; and a frameshift mutation in PPM1D.
86. The pharmaceutical composition of any one of paragraphs 77-85, wherein the subject further has one or more somatic mutations in TET2 in a sub-population of peripheral blood hematopoietic cells selected from an S282F mutation in SEQ ID NO: 68, an N312S mutation in SEQ ID NO: 68, an L346P mutation in SEQ ID NO: 68, an S460F mutation in SEQ ID NO: 68, a D666G mutation in SEQ ID NO: 68, a P941S mutation in SEQ ID NO: 68, and a C1135Y mutation in SEQ ID NO: 68.
87. The pharmaceutical composition of any one of paragraphs 77-86, wherein the inhibitor of HSC cardiometabolic driver gene mutation-mediated proinflammatory activity is an IL-1β inhibitor.
88. The pharmaceutical composition of paragraph 87, wherein the IL-1β inhibitor is an IL-1β inhibitor antibody or antigen-binding fragment thereof that binds to IL-1β and reduces IL-1β binding to its receptor(s).
89. The pharmaceutical composition of paragraph 88, wherein the IL-1β inhibitor antibody or antigen-binding fragment thereof is selected from ABT981, an anti-interleukin-11 inhibitor antibody by ABZYME, APX002, Canakinumab/Ilaris, CDP48, immunereszumab, LY2189102, MEDI8968, and XOMA052.
90. The pharmaceutical composition of paragraph 87, wherein the IL-1β inhibitor is an IL-1 receptor antagonist.
91. The pharmaceutical composition of paragraph 90, wherein the IL-1 receptor antagonist is selected from CDP484, CP412245, CYT013 IL1bQb, XL 130, AMG108, HL 2351, IL1Hyl, AXXO, orthokine, PRT 1000, anakinra, and rilonacept.
92. The pharmaceutical composition of paragraph 87, wherein the IL-1β inhibitor is a small molecule or microRNA inhibitor that inhibits IL-1β-mediated pro-inflammatory activity.
93. The pharmaceutical composition of paragraph 92, wherein the small molecule or microRNA inhibitor is selected from AC201, CP412245, MCC950 or CRID3, inflabion, inflammasome modulator OPSONA, PGE3935199, PGE527667, TRK530, β-hydroxybutyrate (BHB), and microRNA-223.
94. The pharmaceutical composition of paragraph 93, wherein the small molecule inhibitor is MCC950.
95. The pharmaceutical composition of any one of paragraphs 77-94, wherein the inhibitor of HSC cardiometabolic driver gene mutation-mediated proinflammatory activity is an IL-6 inhibitor.
96. The pharmaceutical composition of paragraph 95, wherein the IL-6 inhibitor is an IL-6 inhibitor antibody or antigen-binding fragment thereof that binds to IL-6 and reduces IL-6 binding to its receptor(s).
97. The pharmaceutical composition of paragraph 96, wherein the IL-6 inhibitor antibody or antigen-binding fragment thereof is selected from Siltuximab, Olokizumab, Elsilimomab, mAb 1339, Sirukumab, Clazakizumab, ARGX-109, FM101, and C326.
98. The pharmaceutical composition of paragraph 95, wherein the IL-6 inhibitor is an IL-6 receptor antagonist.
99. The pharmaceutical composition of paragraph 98, wherein the IL-6 receptor antagonist is selected from tocilizumab, sarilumab, REGN88, FE301, and LMT-28.
100. The pharmaceutical composition of paragraph 95, wherein the IL-6 inhibitor is a small molecule or microRNA inhibitor.
101. The pharmaceutical composition of paragraph 100, wherein the small molecule IL-6 inhibitor is ALX-0061 or LMT-28.
102. The pharmaceutical composition of paragraph 101, wherein the IL-6 inhibitor is a JAK-STAT inhibitor.
103. The pharmaceutical composition of paragraph 102, wherein the JAK-STAT inhibitor is selected from baricitinib, decernotinid, filgotinib, INCB-039110, ruxolitinib, tofacitinib, Oclacitinib, Gandotinib, Lestaurtinib, Momelotinib, Pacritinib, PF-04965842, Upadacitinib, and Peficitinib.
104. The pharmaceutical composition of any one of paragraphs 77-103, wherein the inhibitor of HSC cardiometabolic driver gene mutation-mediated proinflammatory activity is a TNFα inhibitor.
105. The pharmaceutical composition of paragraph 104, wherein the TNFα inhibitor is a TNFα inhibitor antibody or antigen-binding fragment thereof that binds to TNFα and reduces TNFα binding to its receptor(s).
106. The pharmaceutical composition of paragraph 105, wherein the TNFα inhibitor antibody or antigen-binding fragment thereof is selected from adalimumab, Adalimumab-atto, certolizumab pegol, golimumab, infliximab,
107. The pharmaceutical composition of paragraph 104, wherein the TNFα inhibitor is a TNFα receptor antagonist.
108. The pharmaceutical composition of paragraph 107, wherein the TNFα receptor antagonist is etanercept.
109. The pharmaceutical composition of paragraph 104, wherein the TNFα inhibitor is a small molecule or microRNA inhibitor.
110. The pharmaceutical composition of any one of paragraphs 77-109, wherein the HSC cardiometabolic driver gene mutation-mediated proinflammatory disease is a cardiometabolic disease or disorder.
111. The pharmaceutical composition of any one of paragraphs 77-109, wherein the HSC cardiometabolic driver gene mutation-mediated proinflammatory disease is a chronic kidney disease or disorder.
112. A method for detecting a subject having, or at risk for, a cardiometabolic driver gene mutation-mediated proinflammatory disease comprising:
(i) obtaining a hematopoietic cell sample from a subject, and
(ii) sequencing the hematopoietic cell sample from the subject to detect one or more somatic mutations in one or more HSC cardiometabolic driver genes in the hematopoietic cell sample.
   113. The method of paragraph 112, wherein the hematopoietic cell sample is a peripheral blood hematopoietic cell sample.
   114. The method of any one of paragraphs 112 or 113, wherein the hematopoietic cell sample is enriched for myeloid-derived cells.
   115. The method of any one of paragraphs 112-114, wherein the one or more somatic mutations in the one or more HSC cardiometabolic driver genes identified in the hematopoietic cell sample cause clonal hematopoiesis in the subject.
   116. The method of any one of paragraphs 112-115, wherein at least 2% of the hematopoietic cells are identified as having one or more HSC cardiometabolic driver gene mutations.
   117. The method of any one of paragraphs 112-116, wherein the one or more HSC cardiometabolic driver genes are selected from TP53, JAK2, DNMT3A, ASXL1, and PPM1D/WIP1.
   118. The method of paragraph 117, wherein the one or more somatic mutations are in TP53 and are selected from a G743A mutation in SEQ ID NO:2 and a A659G mutation in SEQ ID NO: 2.
   119. The method of any one of paragraphs 117 or 118, wherein the one or more somatic mutations are in JAK2 and is a G1849T in SEQ ID NO: 56.
   120. The method of any one of paragraphs 117-119, wherein the one or more somatic mutations are in DNMT3A and are selected from a T1115C mutation in SEQ ID NO: 37; a C2711T mutation in SEQ ID NO: 37; a C1837G mutation in SEQ ID NO: 37; a A1666G mutation in SEQ ID NO: 37; a C1789T mutation in SEQ ID NO: 37; a G2719A mutation in SEQ ID NO: 37; a G1627T mutation in SEQ ID NO: 37; an A2723G mutation in SEQ ID NO: 37; a G1797T mutation in SEQ ID NO: 37; a T2252G mutation in SEQ ID NO: 37; a C1560A mutation in SEQ ID NO: 37; a T1031C mutation in SEQ ID NO: 37; a G2645A mutation in SEQ ID NO: 37; a C2043G mutation in SEQ ID NO: 37; a C2446T mutation in SEQ ID NO: 37; a A2198G mutation in SEQ ID NO: 37; A2281G mutation in SEQ ID NO: 37; a C920G mutation in SEQ ID NO: 37; a A2204G mutation in SEQ ID NO: 37; and a frameshift mutation in DNMT3A.
   121. The method of any one of paragraphs 117-120, wherein the one or more somatic mutations are in ASXL1 and are selected from a C2407T mutation in SEQ ID NO: 61; a C2893T mutation in SEQ ID NO: 61; a 1926_1926delinsAG mutation in SEQ ID NO: 61; and a frameshift mutation in ASXL1.
   122. The method of any one of paragraphs 117-121, wherein the one or more somatic mutations are in PPM1D and are selected from a G1618T mutation in SEQ ID NO: 64; a C1372T mutation in SEQ ID NO: 64; and a frameshift mutation in PPM1D.
   123. The method of any one of paragraphs 117-122, wherein the subject further has one or more somatic mutations in TET2 in a sub-population of peripheral blood hematopoietic cells selected from an S282F mutation in SEQ ID NO: 68, an N312S mutation in SEQ ID NO: 68, an L346P mutation in SEQ ID NO: 68, an S460F mutation in SEQ ID NO: 68, a D666G mutation in SEQ ID NO: 68, a P941S mutation in SEQ ID NO: 68, and a C1135Y mutation in SEQ ID NO: 68.
   124. The method of any one of paragraphs 117-123, further comprising administering a therapeutically effective amount of a pharmaceutical composition comprising an inhibitor of HSC cardiometabolic driver gene mutation-mediated proinflammatory activity and a pharmaceutically acceptable carrier if one or more somatic mutations in one or more HSC cardiometabolic driver genes are identified in the hematopoietic cell sample.
   125. The method of paragraph 124, wherein the inhibitor of HSC cardiometabolic driver gene mutation-mediated proinflammatory activity is an IL-1β inhibitor.
   126. The method of paragraph 125, wherein the IL-1β inhibitor is an IL-1β inhibitor antibody or antigen-binding fragment thereof that binds to IL-1β and reduces IL-1β binding to its receptor(s).
   127. The method of paragraph 126, wherein the IL-1β inhibitor antibody or antigen-binding fragment thereof is selected from ABT981, an anti-interleukin-1β inhibitor antibody by ABZYME, APX002, Canakinumab/Ilaris, CDP48, immunereszumab, LY2189102, MEDI8968, and XOMA052.
   128. The method of paragraph 125, wherein the IL-1β inhibitor is an IL-1 receptor antagonist.
   129. The method of paragraph 128, wherein the IL-1 receptor antagonist is selected from CDP484, CP412245, CYT013 IL1bQb, XL 130, AMG108, HL 2351, IL1Hyl, AXXO, orthokine, PRT 1000, anakinra, and rilonacept.
   130. The method of paragraph 125, wherein the IL-1β inhibitor is a small molecule or microRNA inhibitor that inhibits IL-1β-mediated pro-inflammatory activity.
   131. The method of paragraph 130, wherein the small molecule or microRNA inhibitor is selected from AC201, CP412245, MCC950 or CRID3, inflabion, inflammasome modulator OPSONA, PGE3935199, PGE527667, TRK530, β-hydroxybutyrate (BHB), and microRNA-223.
   132. The method of paragraph 131, wherein the small molecule inhibitor is MCC950.
   133. The method of any one of paragraphs 124-132, wherein the inhibitor of HSC cardiometabolic driver gene mutation-mediated proinflammatory activity is an IL-6 inhibitor.
   134. The method of paragraph 133, wherein the IL-6 inhibitor is an IL-6 inhibitor antibody or antigen-binding fragment thereof that binds to IL-6 and reduces IL-6 binding to its receptor(s).
   135. The method of paragraph 134, wherein the IL-6 inhibitor antibody or antigen-binding fragment thereof is selected from Siltuximab, Olokizumab, Elsilimomab, mAb 1339, Sirukumab, Clazakizumab, ARGX-109, FM101, and C326.
   136. The method of paragraph 133, wherein the IL-6 inhibitor is an IL-6 receptor antagonist.
   137. The method of paragraph 136, wherein the IL-6 receptor antagonist is selected from tocilizumab, sarilumab, REGN88, FE301, and LMT-28.
   138. The method of paragraph 133, wherein the IL-6 inhibitor is a small molecule or microRNA inhibitor.
   139. The method of paragraph 138, wherein the small molecule IL-6 inhibitor is ALX-0061 or LMT-28.
   140. The method of paragraph 133, wherein the IL-6 inhibitor is a JAK-STAT inhibitor.
   141. The method of paragraph 140, wherein the JAK-STAT inhibitor is selected from baricitinib, decernotinid, filgotinib, INCB-039110, ruxolitinib, tofacitinib, Oclacitinib, Gandotinib, Lestaurtinib, Momelotinib, Pacritinib, PF-04965842, Upadacitinib, and Peficitinib.
   142. The method of any one of paragraphs 124-141, wherein the inhibitor of HSC cardiometabolic driver gene mutation-mediated proinflammatory activity is a TNFα inhibitor.
   143. The method of paragraph 142, wherein the TNFα inhibitor is a TNFα inhibitor antibody or antigen-binding fragment thereof that binds to TNFα and reduces TNFα binding to its receptor(s).
   144. The method of paragraph 143, wherein the TNFα inhibitor antibody or antigen-binding fragment thereof is selected from adalimumab, Adalimumab-atto, certolizumab pegol, golimumab, infliximab,
   145. The method of paragraph 142, wherein the TNFα inhibitor is a TNFα receptor antagonist.
   146. The method of paragraph 145, wherein the TNFα receptor antagonist is etanercept.
   147. The method of paragraph 142, wherein the TNFα inhibitor is a small molecule or microRNA inhibitor.
   148. The method of any one of paragraphs 124-147, further comprising monitoring hematopoietic cell clonality, IL-1β proinflammatory activity, IL-6 proinflammatory activity, TNFα proinflammatory activity or any combination thereof following the administration of the inhibitor of HSC cardiometabolic driver gene mutation-mediated proinflammatory activity.
   149. The method of any one of paragraphs 124-148, further comprising decreasing the number or percentage of hematopoietic cells comprising the one or more somatic mutations in the one or more HSC cardiometabolic driver genes in the subject by performing therapeutic cytapheresis on the subject.
   150. The method of any one of paragraphs 124-149, further comprising administering one or more additional therapeutic agents to the subject.
   151. The method of any one of paragraphs 112-150, wherein the HSC cardiometabolic driver gene mutation-mediated proinflammatory disease is a cardiometabolic disease or disorder.
   152. The method of any one of paragraphs 112-150, wherein the HSC cardiometabolic driver gene mutation-mediated proinflammatory disease is a chronic kidney disease or disorder.

Some embodiments of the technology described herein can be defined according to any of the following numbered paragraphs:

1. A method for treating a subject having, or at risk for, a HSC (hematopoietic stem cell) cardiometabolic driver gene mutation-mediated proinflammatory disease comprising: administering a therapeutically effective amount of a pharmaceutical composition comprising an inhibitor of HSC cardiometabolic driver gene mutation-mediated proinflammatory activity and a pharmaceutically acceptable carrier to a subject having one or more somatic mutations in one or more HSC cardiometabolic driver gene in a sub-population of peripheral blood hematopoietic cells.
2. The method of paragraph 1, wherein the one or more somatic mutations in the one or more HSC cardiometabolic driver genes in the sub-population of peripheral blood hematopoietic cells cause clonal hematopoiesis in the subject.
3. The method of any one of paragraphs 1 or 2, wherein at least 2% of the peripheral blood hematopoietic cells have the one or more somatic mutations in the one or more HSC cardiometabolic driver genes.
4. The method of any one of paragraphs 1-3, wherein the one or more HSC cardiometabolic driver genes are selected from TP53, JAK2, DNMT3A, ASXL1, TET2, and PPM1D/WIP1.
5. The method of paragraph 4, wherein the one or more somatic mutations are in TP53 and are selected from a G743A mutation in SEQ ID NO:2 and a A659G mutation in SEQ ID NO: 2.
6. The method of any one of paragraphs 4 or 5, wherein the one or more somatic mutations are in JAK2 and is a G1849T in SEQ ID NO: 56.
7. The method of any one of paragraphs 4-6, wherein the one or more somatic mutations are in DNMT3A and are selected from a T1115C mutation in SEQ ID NO: 37; a C2711T mutation in SEQ ID NO: 37; a C1837G mutation in SEQ ID NO: 37; a A1666G mutation in SEQ ID NO: 37; a C1789T mutation in SEQ ID NO: 37; a G2719A mutation in SEQ ID NO: 37; a G1627T mutation in SEQ ID NO: 37; an A2723G mutation in SEQ ID NO: 37; a G1797T mutation in SEQ ID NO: 37; a T2252G mutation in SEQ ID NO: 37; a C1560A mutation in SEQ ID NO: 37; a T1031C mutation in SEQ ID NO: 37; a G2645A mutation in SEQ ID NO: 37; a C2043G mutation in SEQ ID NO: 37; a C2446T mutation in SEQ ID NO: 37; a A2198G mutation in SEQ ID NO: 37; A2281G mutation in SEQ ID NO: 37; a C920G mutation in SEQ ID NO: 37; a A2204G mutation in SEQ ID NO: 37; and a frameshift mutation in DNMT3A.
8. The method of any one of paragraphs 4-7, wherein the one or more somatic mutations are in ASXL1 and are selected from a C2407T mutation in SEQ ID NO: 61; a C2893T mutation in SEQ ID NO: 61; a 1926_1926delinsAG mutation in SEQ ID NO: 61; and a frameshift mutation in ASXL1.
9. The method of any one of paragraphs 4-8, wherein the one or more somatic mutations are in PPM1D and are selected from a G1618T mutation in SEQ ID NO: 64; a C1372T mutation in SEQ ID NO: 64; and a frameshift mutation in PPM1D.
10. The method of any one of paragraphs 4-8, wherein one or more somatic mutations are in TET2 and are selected from an S282F mutation in SEQ ID NO: 68, an N312S mutation in SEQ ID NO: 68, an L346P mutation in SEQ ID NO: 68, an S460F mutation in SEQ ID NO: 68, a D666G mutation in SEQ ID NO: 68, a P941S mutation in SEQ ID NO: 68, and a C1135Y mutation in SEQ ID NO: 68.
11. The method of any one of paragraphs 1-9, wherein the subject further has one or more somatic mutations in TET2 in a sub-population of peripheral blood hematopoietic cells selected from an S282F mutation in SEQ ID NO: 68, an N312S mutation in SEQ ID NO: 68, an L346P mutation in SEQ ID NO: 68, an S460F mutation in SEQ ID NO: 68, a D666G mutation in SEQ ID NO: 68, a P941S mutation in SEQ ID NO: 68, and a C1135Y mutation in SEQ ID NO: 68.
12. The method of any one of paragraphs 1-11, wherein the inhibitor of HSC cardiometabolic driver gene mutation-mediated proinflammatory activity is an IL-1β inhibitor.
13. The method of paragraph 12, wherein the IL-1β inhibitor is an IL-1β inhibitor antibody or antigen-binding fragment thereof that binds to IL-1β and reduces IL-1β binding to its receptor(s).
14. The method of paragraph 13, wherein the IL-1β inhibitor antibody or antigen-binding fragment thereof is selected from ABT981, an anti-interleukin-1J inhibitor antibody by ABZYME, APX002, Canakinumab/Ilaris, CDP48, immunereszumab, LY2189102, MEDI8968, and XOMA052.
15. The method of paragraph 12, wherein the IL-1β inhibitor is an IL-1 receptor antagonist.
16. The method of paragraph 15, wherein the IL-1 receptor antagonist is selected from CDP484, CP412245, CYT013 IL1bQb, XL 130, AMG108, HL 2351, IL1Hyl, AXXO, orthokine, PRT 1000, anakinra, and rilonacept.
17. The method of paragraph 12, wherein the IL-1β inhibitor is a small molecule or microRNA inhibitor that inhibits IL-1β-mediated pro-inflammatory activity.
18. The method of paragraph 17, wherein the small molecule or microRNA inhibitor is selected from AC201, CP412245, MCC950 or CRID3, inflabion, inflammasome modulator OPSONA, PGE3935199, PGE527667, TRK530, β-hydroxybutyrate (BHB), and microRNA-223.
19. The method of paragraph 18, wherein the small molecule inhibitor is MCC950.
20. The method of any one of paragraphs 1-19, wherein the inhibitor of HSC cardiometabolic driver gene mutation-mediated proinflammatory activity is an IL-6 inhibitor.
21. The method of paragraph 20, wherein the IL-6 inhibitor is an IL-6 inhibitor antibody or antigen-binding fragment thereof that binds to IL-6 and reduces IL-6 binding to its receptor(s).
22. The method of paragraph 21, wherein the IL-6 inhibitor antibody or antigen-binding fragment thereof is selected from Siltuximab, Olokizumab, Elsilimomab, mAb 1339, Sirukumab, Clazakizumab, ARGX-109, FM101, and C326.
23. The method of paragraph 20, wherein the IL-6 inhibitor is an IL-6 receptor antagonist.
24. The method of paragraph 23, wherein the IL-6 receptor antagonist is selected from tocilizumab, sarilumab, REGN88, FE301, and LMT-28.
25. The method of paragraph 20, wherein the IL-6 inhibitor is a small molecule or microRNA inhibitor.
26. The method of paragraph 25, wherein the small molecule IL-6 inhibitor is ALX-0061 or LMT-28.
27. The method of paragraph 20, wherein the IL-6 inhibitor is a JAK-STAT inhibitor.
28. The method of paragraph 27, wherein the JAK-STAT inhibitor is selected from baricitinib, decernotinid, filgotinib, INCB-039110, ruxolitinib, tofacitinib, Oclacitinib, Gandotinib, Lestaurtinib, Momelotinib, Pacritinib, PF-04965842, Upadacitinib, and Peficitinib.
29. The method of any one of paragraphs 1-28, wherein the inhibitor of HSC cardiometabolic driver gene mutation-mediated proinflammatory activity is a TNFα inhibitor.
30. The method of paragraph 29, wherein the TNFα inhibitor is a TNFα inhibitor antibody or antigen-binding fragment thereof that binds to TNFα and reduces TNFα binding to its receptor(s).
31. The method of paragraph 30, wherein the TNFα inhibitor antibody or antigen-binding fragment thereof is selected from adalimumab, Adalimumab-atto, certolizumab pegol, golimumab, infliximab,
32. The method of paragraph 29, wherein the TNFα inhibitor is a TNFα receptor antagonist.
33. The method of paragraph 32, wherein the TNFα receptor antagonist is etanercept.
34. The method of paragraph 29, wherein the TNFα inhibitor is a small molecule or microRNA inhibitor.
35. The method of any one of paragraphs 1-34, further comprising monitoring hematopoietic cell clonality, IL-1β proinflammatory activity, IL-6 proinflammatory activity, TNFα proinflammatory activity or any combination thereof following the administration of the inhibitor of HSC cardiometabolic driver gene mutation-mediated proinflammatory activity.
36. The method of any one of paragraphs 1-35, further comprising decreasing the number or percentage of hematopoietic cells comprising the one or more somatic mutations in the one or more HSC cardiometabolic driver genes in the subject by performing therapeutic cytapheresis on the subject.
37. The method of any one of paragraphs 1-36, further comprising administering one or more additional therapeutic agents to the subject.
38. A method for treating a subject having, or at risk for, a HSC cardiometabolic driver gene mutation-mediated proinflammatory disease comprising:

(a) sequencing a hematopoietic cell sample from a subject to identify one or more somatic mutations in one or more HSC cardiometabolic driver genes in the hematopoietic cell sample; and

(b) administering a therapeutically effective amount of a pharmaceutical composition comprising an inhibitor of HSC cardiometabolic driver gene mutation-mediated proinflammatory activity and a pharmaceutically acceptable carrier if one or more somatic mutations in one or more HSC cardiometabolic driver genes are identified in the hematopoietic cell sample.

39. The method of paragraph 38, wherein the hematopoietic cell sample is a peripheral blood hematopoietic cell sample.
40. The method of any one of paragraphs 38 or 39, wherein the hematopoietic cell sample is enriched for myeloid-derived cells.
41. The method of any one of paragraphs 38-40, wherein the one or more somatic mutations in the one or more HSC cardiometabolic driver genes identified in the hematopoietic cell sample cause clonal hematopoiesis in the subject.
42. The method of any one of paragraphs 38-41, wherein at least 2% of the hematopoietic cells are identified as having one or more HSC cardiometabolic driver gene mutations.
43. The method of any one of paragraphs 38-42, wherein the one or more HSC cardiometabolic driver genes are selected from TP53, JAK2, DNMT3A, ASXL1, TET2 and PPM1D/WIP1.
44. The method of paragraph 43, wherein the one or more somatic mutations are in TP53 and are selected from a G743A mutation in SEQ ID NO:2 and a A659G mutation in SEQ ID NO: 2.
45. The method of any one of paragraphs 43 or 44, wherein the one or more somatic mutations are in JAK2 and is a G1849T in SEQ ID NO: 56.
46. The method of any one of paragraphs 43-45, wherein the one or more somatic mutations are in DNMT3A and are selected from a T1115C mutation in SEQ ID NO: 37; a C2711T mutation in SEQ ID NO: 37; a C1837G mutation in SEQ ID NO: 37; a A1666G mutation in SEQ ID NO: 37; a C1789T mutation in SEQ ID NO: 37; a G2719A mutation in SEQ ID NO: 37; a G1627T mutation in SEQ ID NO: 37; an A2723G mutation in SEQ ID NO: 37; a G1797T mutation in SEQ ID NO: 37; a T2252G mutation in SEQ ID NO: 37; a C1560A mutation in SEQ ID NO: 37; a T1031C mutation in SEQ ID NO: 37; a G2645A mutation in SEQ ID NO: 37; a C2043G mutation in SEQ ID NO: 37; a C2446T mutation in SEQ ID NO: 37; a A2198G mutation in SEQ ID NO: 37; A2281G mutation in SEQ ID NO: 37; a C920G mutation in SEQ ID NO: 37; a A2204G mutation in SEQ ID NO: 37; and a frameshift mutation in DNMT3A.
47. The method of any one of paragraphs 43-46, wherein the one or more somatic mutations are in ASXL1 and are selected from a C2407T mutation in SEQ ID NO: 61; a C2893T mutation in SEQ ID NO: 61; a 1926_1926delinsAG mutation in SEQ ID NO: 61; and a frameshift mutation in ASXL1.
48. The method of any one of paragraphs 43-47, wherein the one or more somatic mutations are in PPM1D and are selected from a G1618T mutation in SEQ ID NO: 64; a C1372T mutation in SEQ ID NO: 64; and a frameshift mutation in PPM1D.
49. The method of any one of paragraphs 43-48, wherein one or more somatic mutations are in TET2 and are selected from an S282F mutation in SEQ ID NO: 68, an N312S mutation in SEQ ID NO: 68, an L346P mutation in SEQ ID NO: 68, an S460F mutation in SEQ ID NO: 68, a D666G mutation in SEQ ID NO: 68, a P941S mutation in SEQ ID NO: 68, and a C1135Y mutation in SEQ ID NO: 68.
50. The method of any one of paragraphs 38-49, wherein the subject further has one or more somatic mutations in TET2 in a sub-population of peripheral blood hematopoietic cells selected from an S282F mutation in SEQ ID NO: 68, an N312S mutation in SEQ ID NO: 68, an L346P mutation in SEQ ID NO: 68, an S460F mutation in SEQ ID NO: 68, a D666G mutation in SEQ ID NO: 68, a P941S mutation in SEQ ID NO: 68, and a C1135Y mutation in SEQ ID NO: 68.
51. The method of any one of paragraphs 38-49, wherein the inhibitor of HSC cardiometabolic driver gene mutation-mediated proinflammatory activity is an IL-1β inhibitor.
52. The method of paragraph 51, wherein the IL-1β inhibitor is an IL-1β inhibitor antibody or antigen-binding fragment thereof that binds to IL-1β and reduces IL-1β binding to its receptor(s).
53. The method of paragraph 52, wherein the IL-1β inhibitor antibody or antigen-binding fragment thereof is selected from ABT981, an anti-interleukin-11 inhibitor antibody by ABZYME, APX002, Canakinumab/Ilaris, CDP48, immunereszumab, LY2189102, MEDI8968, and XOMA052.
54. The method of paragraph 51, wherein the IL-1β inhibitor is an IL-1 receptor antagonist.
55. The method of paragraph 54, wherein the IL-1 receptor antagonist is selected from CDP484, CP412245, CYT013 IL1bQb, XL 130, AMG108, HL 2351, IL1Hyl, AXXO, orthokine, PRT 1000, anakinra, and rilonacept.
56. The method of paragraph 51, wherein the IL-1β inhibitor is a small molecule or microRNA inhibitor that inhibits IL-1β-mediated pro-inflammatory activity.
57. The method of paragraph 56, wherein the small molecule or microRNA inhibitor is selected from AC201, CP412245, MCC950 or CRID3, inflabion, inflammasome modulator OPSONA, PGE3935199, PGE527667, TRK530, J-hydroxybutyrate (BHB), and microRNA-223.
58. The method of paragraph 57, wherein the small molecule inhibitor is MCC950.
59. The method of any one of paragraphs 38-58, wherein the inhibitor of HSC cardiometabolic driver gene mutation-mediated proinflammatory activity is an IL-6 inhibitor.
60. The method of paragraph 59, wherein the IL-6 inhibitor is an IL-6 inhibitor antibody or antigen-binding fragment thereof that binds to IL-6 and reduces IL-6 binding to its receptor(s).
61. The method of paragraph 60, wherein the IL-6 inhibitor antibody or antigen-binding fragment thereof is selected from Siltuximab, Olokizumab, Elsilimomab, mAb 1339, Sirukumab, Clazakizumab, ARGX-109, FM101, and C326.
62. The method of paragraph 59, wherein the IL-6 inhibitor is an IL-6 receptor antagonist.
63. The method of paragraph 62, wherein the IL-6 receptor antagonist is selected from tocilizumab, sarilumab, REGN88, FE301, and LMT-28.
64. The method of paragraph 59, wherein the IL-6 inhibitor is a small molecule or microRNA inhibitor.
65. The method of paragraph 64, wherein the small molecule IL-6 inhibitor is ALX-0061 or LMT-28.
66. The method of paragraph 65, wherein the IL-6 inhibitor is a JAK-STAT inhibitor.
67. The method of paragraph 66, wherein the JAK-STAT inhibitor is selected from baricitinib, decernotinid, filgotinib, INCB-039110, ruxolitinib, tofacitinib, Oclacitinib, Gandotinib, Lestaurtinib, Momelotinib, Pacritinib, PF-04965842, Upadacitinib, and Peficitinib.
68. The method of any one of paragraphs 38-67, wherein the inhibitor of HSC cardiometabolic driver gene mutation-mediated proinflammatory activity is a TNFα inhibitor.
69. The method of paragraph 68, wherein the TNFα inhibitor is a TNFα inhibitor antibody or antigen-binding fragment thereof that binds to TNFα and reduces TNFα binding to its receptor(s).
70. The method of paragraph 69, wherein the TNFα inhibitor antibody or antigen-binding fragment thereof is selected from adalimumab, Adalimumab-atto, certolizumab pegol, golimumab, infliximab,
71. The method of paragraph 68, wherein the TNFα inhibitor is a TNFα receptor antagonist.
72. The method of paragraph 71, wherein the TNFα receptor antagonist is etanercept.
73. The method of paragraph 68, wherein the TNFα inhibitor is a small molecule or microRNA inhibitor.
74. The method of any one of paragraphs 38-73, further comprising monitoring hematopoietic cell clonality, IL-1β proinflammatory activity, IL-6 proinflammatory activity, TNFα proinflammatory activity or any combination thereof following the administration of the inhibitor of HSC cardiometabolic driver gene mutation-mediated proinflammatory activity.
75. The method of any one of paragraphs 38-74, further comprising decreasing the number or percentage of hematopoietic cells comprising the one or more somatic mutations in the one or more HSC cardiometabolic driver genes in the subject by performing therapeutic cytapheresis on the subject.
76. The method of any one of paragraphs 38-75, further comprising administering one or more additional therapeutic agents to the subject.
77. The method of any one of paragraphs 1-76, wherein the HSC cardiometabolic driver gene mutation-mediated proinflammatory disease is a cardiometabolic disease or disorder.
78. The method of any one of paragraphs 1-76, wherein the HSC cardiometabolic driver gene mutation-mediated proinflammatory disease is a chronic kidney disease or disorder.
79. A pharmaceutical composition comprising an inhibitor of HSC cardiometabolic driver gene mutation-mediated proinflammatory activity and a pharmaceutically acceptable carrier for use in a subject having one or more somatic mutations in one or more HSC cardiometabolic driver genes in a sub-population of hematopoietic cells.
80. The pharmaceutical composition of paragraph 79, wherein the one or more somatic mutations in the one or more HSC cardiometabolic driver genes in the sub-population of hematopoietic cells cause clonal hematopoiesis in the subject.
81. The pharmaceutical composition of any one of paragraphs 79 or 80, wherein at least 2% of the hematopoietic cells in the subject have the one or more mutations in one or more HSC cardiometabolic driver genes.
82. The pharmaceutical composition of any one of paragraphs 79-81, wherein the one or more HSC cardiometabolic driver genes are selected from TP53, JAK2, DNMT3A, ASXL1, TET2, and PPM1D/WIP1.
83. The pharmaceutical composition of paragraph 82, wherein the one or more somatic mutations are in TP53 and are selected from a G743A mutation in SEQ ID NO:2 and a A659G mutation in SEQ ID NO: 2.
84. The pharmaceutical composition of any one of paragraphs 82 or 83, wherein the one or more somatic mutations are in JAK2 and is a G1849T in SEQ ID NO: 56.
85. The pharmaceutical composition of any one of paragraphs 82-84, wherein the one or more somatic mutations are in DNMT3A and are selected from a T1115C mutation in SEQ ID NO: 37; a C2711T mutation in SEQ ID NO: 37; a C1837G mutation in SEQ ID NO: 37; a A1666G mutation in SEQ ID NO: 37; a C1789T mutation in SEQ ID NO: 37; a G2719A mutation in SEQ ID NO: 37; a G1627T mutation in SEQ ID NO: 37; an A2723G mutation in SEQ ID NO: 37; a G1797T mutation in SEQ ID NO: 37; a T2252G mutation in SEQ ID NO: 37; a C1560A mutation in SEQ ID NO: 37; a T1031C mutation in SEQ ID NO: 37; a G2645A mutation in SEQ ID NO: 37; a C2043G mutation in SEQ ID NO: 37; a C2446T mutation in SEQ ID NO: 37; a A2198G mutation in SEQ ID NO: 37; A2281G mutation in SEQ ID NO: 37; a C920G mutation in SEQ ID NO: 37; a A2204G mutation in SEQ ID NO: 37; and a frameshift mutation in DNMT3A.
86. The pharmaceutical composition of any one of paragraphs 82-85, wherein the one or more somatic mutations are in ASXL1 and are selected from a C2407T mutation in SEQ ID NO: 61; a C2893T mutation in SEQ ID NO: 61; a 1926_1926delinsAG mutation in SEQ ID NO: 61; and a frameshift mutation in ASXL1.
87. The pharmaceutical composition of any one of paragraphs 82-86, wherein the one or more somatic mutations are in PPM1D and are selected from a G1618T mutation in SEQ ID NO: 64; a C1372T mutation in SEQ ID NO: 64; and a frameshift mutation in PPM1D.
88. The pharmaceutical composition of any one of paragraphs 82-87, wherein the one or more somatic mutations are in TET2 and are selected from an S282F mutation in SEQ ID NO: 68, an N312S mutation in SEQ ID NO: 68, an L346P mutation in SEQ ID NO: 68, an S460F mutation in SEQ ID NO: 68, a D666G mutation in SEQ ID NO: 68, a P941S mutation in SEQ ID NO: 68, and a C1135Y mutation in SEQ ID NO: 68.
89. The pharmaceutical composition of any one of paragraphs 82-87, wherein the subject further has one or more somatic mutations in TET2 in a sub-population of peripheral blood hematopoietic cells selected from an S282F mutation in SEQ ID NO: 68, an N312S mutation in SEQ ID NO: 68, an L346P mutation in SEQ ID NO: 68, an S460F mutation in SEQ ID NO: 68, a D666G mutation in SEQ ID NO: 68, a P941S mutation in SEQ ID NO: 68, and a C1135Y mutation in SEQ ID NO: 68.
90. The pharmaceutical composition of any one of paragraphs 79-89, wherein the inhibitor of HSC cardiometabolic driver gene mutation-mediated proinflammatory activity is an IL-1β inhibitor.
91. The pharmaceutical composition of paragraph 90, wherein the IL-1β inhibitor is an IL-1β inhibitor antibody or antigen-binding fragment thereof that binds to IL-1β and reduces IL-1β binding to its receptor(s).
92. The pharmaceutical composition of paragraph 91, wherein the IL-1β inhibitor antibody or antigen-binding fragment thereof is selected from ABT981, an anti-interleukin-1β inhibitor antibody by ABZYME, APX002, Canakinumab/Ilaris, CDP48, immunereszumab, LY2189102, MEDI8968, and XOMA052.
93. The pharmaceutical composition of paragraph 90, wherein the IL-1β inhibitor is an IL-1 receptor antagonist.
94. The pharmaceutical composition of paragraph 93, wherein the IL-1 receptor antagonist is selected from CDP484, CP412245, CYT013 IL1bQb, XL 130, AMG108, HL 2351, IL1Hyl, AXXO, orthokine, PRT 1000, anakinra, and rilonacept.
95. The pharmaceutical composition of paragraph 90, wherein the IL-1β inhibitor is a small molecule or microRNA inhibitor that inhibits IL-1β-mediated pro-inflammatory activity.
96. The pharmaceutical composition of paragraph 95, wherein the small molecule or microRNA inhibitor is selected from AC201, CP412245, MCC950 or CRID3, inflabion, inflammasome modulator OPSONA, PGE3935199, PGE527667, TRK530, β-hydroxybutyrate (BHB), and microRNA-223.
97. The pharmaceutical composition of paragraph 96, wherein the small molecule inhibitor is MCC950.
98. The pharmaceutical composition of any one of paragraphs 79-97, wherein the inhibitor of HSC cardiometabolic driver gene mutation-mediated proinflammatory activity is an IL-6 inhibitor.
99. The pharmaceutical composition of paragraph 98, wherein the IL-6 inhibitor is an IL-6 inhibitor antibody or antigen-binding fragment thereof that binds to IL-6 and reduces IL-6 binding to its receptor(s).
100. The pharmaceutical composition of paragraph 99, wherein the IL-6 inhibitor antibody or antigen-binding fragment thereof is selected from Siltuximab, Olokizumab, Elsilimomab, mAb 1339, Sirukumab, Clazakizumab, ARGX-109, FM101, and C326.
101. The pharmaceutical composition of paragraph 98, wherein the IL-6 inhibitor is an IL-6 receptor antagonist.
102. The pharmaceutical composition of paragraph 101, wherein the IL-6 receptor antagonist is selected from tocilizumab, sarilumab, REGN88, FE301, and LMT-28.
103. The pharmaceutical composition of paragraph 98, wherein the IL-6 inhibitor is a small molecule or microRNA inhibitor.
104. The pharmaceutical composition of paragraph 103, wherein the small molecule IL-6 inhibitor is ALX-0061 or LMT-28.
105. The pharmaceutical composition of paragraph 104, wherein the IL-6 inhibitor is a JAK-STAT inhibitor.
106. The pharmaceutical composition of paragraph 105, wherein the JAK-STAT inhibitor is selected from baricitinib, decernotinid, filgotinib, INCB-039110, ruxolitinib, tofacitinib, Oclacitinib, Gandotinib, Lestaurtinib, Momelotinib, Pacritinib, PF-04965842, Upadacitinib, and Peficitinib.
107. The pharmaceutical composition of any one of paragraphs 79-106, wherein the inhibitor of HSC cardiometabolic driver gene mutation-mediated proinflammatory activity is a TNFα inhibitor.
108. The pharmaceutical composition of paragraph 107, wherein the TNFα inhibitor is a TNFα inhibitor antibody or antigen-binding fragment thereof that binds to TNFα and reduces TNFα binding to its receptor(s).
109. The pharmaceutical composition of paragraph 108, wherein the TNFα inhibitor antibody or antigen-binding fragment thereof is selected from adalimumab, Adalimumab-atto, certolizumab pegol, golimumab, infliximab,
110. The pharmaceutical composition of paragraph 107, wherein the TNFα inhibitor is a TNFα receptor antagonist.
111. The pharmaceutical composition of paragraph 1110, wherein the TNFα receptor antagonist is etanercept.
112. The pharmaceutical composition of paragraph 107, wherein the TNFα inhibitor is a small molecule or microRNA inhibitor.
113. The pharmaceutical composition of any one of paragraphs 79-112, wherein the HSC cardiometabolic driver gene mutation-mediated proinflammatory disease is a cardiometabolic disease or disorder.
114. The pharmaceutical composition of any one of paragraphs 79-112, wherein the HSC cardiometabolic driver gene mutation-mediated proinflammatory disease is a chronic kidney disease or disorder.
115. A method for detecting a subject having, or at risk for, a cardiometabolic driver gene mutation-mediated proinflammatory disease comprising:
(iii) obtaining a hematopoietic cell sample from a subject, and
(iv) sequencing the hematopoietic cell sample from the subject to detect one or more somatic mutations in one or more HSC cardiometabolic driver genes in the hematopoietic cell sample.
   116. The method of paragraph 115, wherein the hematopoietic cell sample is a peripheral blood hematopoietic cell sample.
   117. The method of any one of paragraphs 115 or 116, wherein the hematopoietic cell sample is enriched for myeloid-derived cells.
   118. The method of any one of paragraphs 115-117, wherein the one or more somatic mutations in the one or more HSC cardiometabolic driver genes identified in the hematopoietic cell sample cause clonal hematopoiesis in the subject.
   119. The method of any one of paragraphs 115-118, wherein at least 2% of the hematopoietic cells are identified as having one or more HSC cardiometabolic driver gene mutations.
   120. The method of any one of paragraphs 115-119, wherein the one or more HSC cardiometabolic driver genes are selected from TP53, JAK2, DNMT3A, ASXL1, TET2, and PPM1D/WIP1.
   121. The method of paragraph 120, wherein the one or more somatic mutations are in TP53 and are selected from a G743A mutation in SEQ ID NO:2 and a A659G mutation in SEQ ID NO: 2.
   122. The method of any one of paragraphs 120 or 121, wherein the one or more somatic mutations are in JAK2 and is a G1849T in SEQ ID NO: 56.
   123. The method of any one of paragraphs 120-121, wherein the one or more somatic mutations are in DNMT3A and are selected from a T1115C mutation in SEQ ID NO: 37; a C2711T mutation in SEQ ID NO: 37; a C1837G mutation in SEQ ID NO: 37; a A1666G mutation in SEQ ID NO: 37; a C1789T mutation in SEQ ID NO: 37; a G2719A mutation in SEQ ID NO: 37; a G1627T mutation in SEQ ID NO: 37; an A2723G mutation in SEQ ID NO: 37; a G1797T mutation in SEQ ID NO: 37; a T2252G mutation in SEQ ID NO: 37; a C1560A mutation in SEQ ID NO: 37; a T1031C mutation in SEQ ID NO: 37; a G2645A mutation in SEQ ID NO: 37; a C2043G mutation in SEQ ID NO: 37; a C2446T mutation in SEQ ID NO: 37; a A2198G mutation in SEQ ID NO: 37; A2281G mutation in SEQ ID NO: 37; a C920G mutation in SEQ ID NO: 37; a A2204G mutation in SEQ ID NO: 37; and a frameshift mutation in DNMT3A.
   124. The method of any one of paragraphs 120-123, wherein the one or more somatic mutations are in ASXL1 and are selected from a C2407T mutation in SEQ ID NO: 61; a C2893T mutation in SEQ ID NO: 61; a 1926_1926delinsAG mutation in SEQ ID NO: 61; and a frameshift mutation in ASXL1.
   125. The method of any one of paragraphs 120-124, wherein the one or more somatic mutations are in PPM1D and are selected from a G1618T mutation in SEQ ID NO: 64; a C1372T mutation in SEQ ID NO: 64; and a frameshift mutation in PPM1D.
   126. The method of any one of paragraphs 120-125, wherein the one or more somatic mutations are in TET2 and are selected from an S282F mutation in SEQ ID NO: 68, an N312S mutation in SEQ ID NO: 68, an L346P mutation in SEQ ID NO: 68, an S460F mutation in SEQ ID NO: 68, a D666G mutation in SEQ ID NO: 68, a P941S mutation in SEQ ID NO: 68, and a C1135Y mutation in SEQ ID NO: 68.
   127. The method of any one of paragraphs 120-125, wherein the subject further has one or more somatic mutations in TET2 in a sub-population of peripheral blood hematopoietic cells selected from an S282F mutation in SEQ ID NO: 68, an N312S mutation in SEQ ID NO: 68, an L346P mutation in SEQ ID NO: 68, an S460F mutation in SEQ ID NO: 68, a D666G mutation in SEQ ID NO: 68, a P941S mutation in SEQ ID NO: 68, and a C1135Y mutation in SEQ ID NO: 68.
   128. The method of any one of paragraphs 120-127, further comprising administering a therapeutically effective amount of a pharmaceutical composition comprising an inhibitor of HSC cardiometabolic driver gene mutation-mediated proinflammatory activity and a pharmaceutically acceptable carrier if one or more somatic mutations in one or more HSC cardiometabolic driver genes are identified in the hematopoietic cell sample.
   129. The method of paragraph 128, wherein the inhibitor of HSC cardiometabolic driver gene mutation-mediated proinflammatory activity is an IL-1β inhibitor.
   130. The method of paragraph 129, wherein the IL-1β inhibitor is an IL-1β inhibitor antibody or antigen-binding fragment thereof that binds to IL-1β and reduces IL-1β binding to its receptor(s).
   131. The method of paragraph 130, wherein the IL-1β inhibitor antibody or antigen-binding fragment thereof is selected from ABT981, an anti-interleukin-1β inhibitor antibody by ABZYME, APX002, Canakinumab/Ilaris, CDP48, immunereszumab, LY2189102, MEDI8968, and XOMA052.
   132. The method of paragraph 129, wherein the IL-1β inhibitor is an IL-1 receptor antagonist.
   133. The method of paragraph 132, wherein the IL-1 receptor antagonist is selected from CDP484, CP412245, CYT013 IL1bQb, XL 130, AMG108, HL 2351, IL1Hyl, AXXO, orthokine, PRT 1000, anakinra, and rilonacept.
   134. The method of paragraph 129, wherein the IL-1β inhibitor is a small molecule or microRNA inhibitor that inhibits IL-1β-mediated pro-inflammatory activity.
   135. The method of paragraph 134, wherein the small molecule or microRNA inhibitor is selected from AC201, CP412245, MCC950 or CRID3, inflabion, inflammasome modulator OPSONA, PGE3935199, PGE527667, TRK530, β-hydroxybutyrate (BHB), and microRNA-223.
   136. The method of paragraph 135, wherein the small molecule inhibitor is MCC950.
   137. The method of any one of paragraphs 128-136, wherein the inhibitor of HSC cardiometabolic driver gene mutation-mediated proinflammatory activity is an IL-6 inhibitor.
   138. The method of paragraph 137, wherein the IL-6 inhibitor is an IL-6 inhibitor antibody or antigen-binding fragment thereof that binds to IL-6 and reduces IL-6 binding to its receptor(s).
   139. The method of paragraph 138, wherein the IL-6 inhibitor antibody or antigen-binding fragment thereof is selected from Siltuximab, Olokizumab, Elsilimomab, mAb 1339, Sirukumab, Clazakizumab, ARGX-109, FM101, and C326.
   140. The method of paragraph 137, wherein the IL-6 inhibitor is an IL-6 receptor antagonist.
   141. The method of paragraph 140, wherein the IL-6 receptor antagonist is selected from tocilizumab, sarilumab, REGN88, FE301, and LMT-28.
   142. The method of paragraph 137, wherein the IL-6 inhibitor is a small molecule or microRNA inhibitor.
   143. The method of paragraph 141, wherein the small molecule IL-6 inhibitor is ALX-0061 or LMT-28.
   144. The method of paragraph 137, wherein the IL-6 inhibitor is a JAK-STAT inhibitor.
   145. The method of paragraph 144, wherein the JAK-STAT inhibitor is selected from baricitinib, decernotinid, filgotinib, INCB-039110, ruxolitinib, tofacitinib, Oclacitinib, Gandotinib, Lestaurtinib, Momelotinib, Pacritinib, PF-04965842, Upadacitinib, and Peficitinib.
   146. The method of any one of paragraphs 127-145, wherein the inhibitor of HSC cardiometabolic driver gene mutation-mediated proinflammatory activity is a TNFα inhibitor.
   147. The method of paragraph 146, wherein the TNFα inhibitor is a TNFα inhibitor antibody or antigen-binding fragment thereof that binds to TNFα and reduces TNFα binding to its receptor(s).
   148. The method of paragraph 147, wherein the TNFα inhibitor antibody or antigen-binding fragment thereof is selected from adalimumab, Adalimumab-atto, certolizumab pegol, golimumab, infliximab,
   149. The method of paragraph 146, wherein the TNFα inhibitor is a TNFα receptor antagonist.
   150. The method of paragraph 149, wherein the TNFα receptor antagonist is etanercept.
   151. The method of paragraph 146, wherein the TNFα inhibitor is a small molecule or microRNA inhibitor.
   152. The method of any one of paragraphs 127-151, further comprising monitoring hematopoietic cell clonality, IL-1β proinflammatory activity, IL-6 proinflammatory activity, TNFα proinflammatory activity or any combination thereof following the administration of the inhibitor of HSC cardiometabolic driver gene mutation-mediated proinflammatory activity.
   153. The method of any one of paragraphs 127-152, further comprising decreasing the number or percentage of hematopoietic cells comprising the one or more somatic mutations in the one or more HSC cardiometabolic driver genes in the subject by performing therapeutic cytapheresis on the subject.
   154. The method of any one of paragraphs 127-153, further comprising administering one or more additional therapeutic agents to the subject.
   155. The method of any one of paragraphs 115-154, wherein the HSC cardiometabolic driver gene mutation-mediated proinflammatory disease is a cardiometabolic disease or disorder.
   156. The method of any one of paragraphs 1-155, wherein the HSC cardiometabolic driver gene mutation-mediated proinflammatory disease is a chronic kidney disease or disorder.

### EXAMPLES

### Example 1: TP53 Mediated Hematopoietic Cell Expansion Contributes to Pathological Remodeling in Experimental Heart Failure

Somatic inactivating mutations in TP53 are associated with the phenomenon of clonal hematopoiesis, a common condition found in the elderly population that generally has little or no impact on the abundance of hematocrit or levels of leukocytes and platelets, but has been associated with increased mortality due to heart disease and stroke.

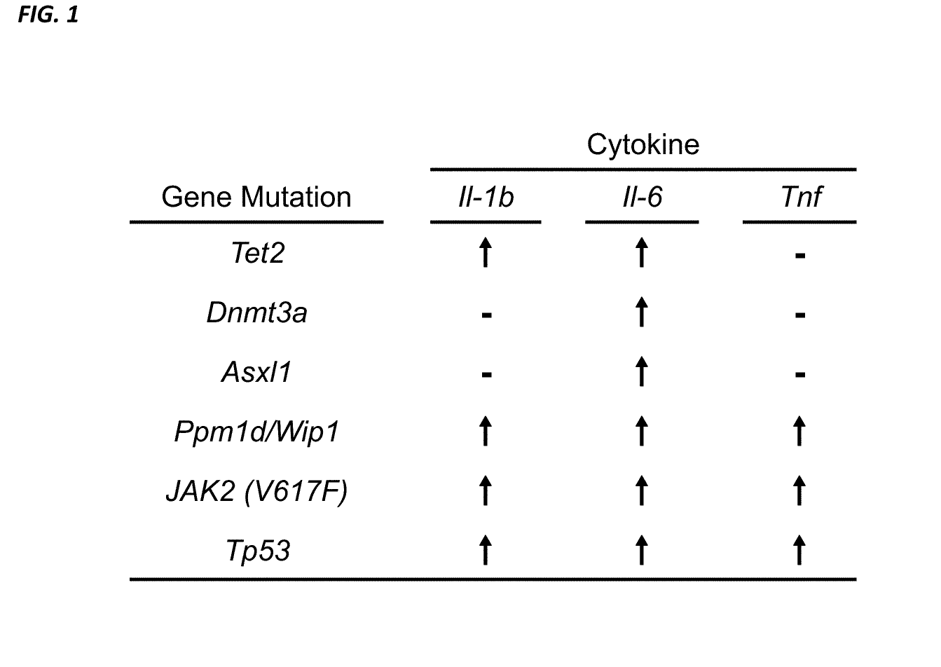

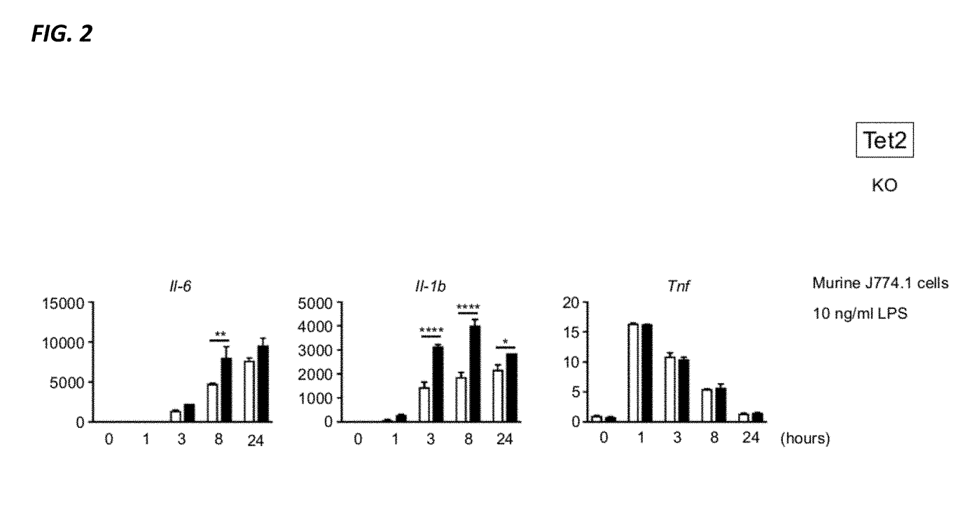

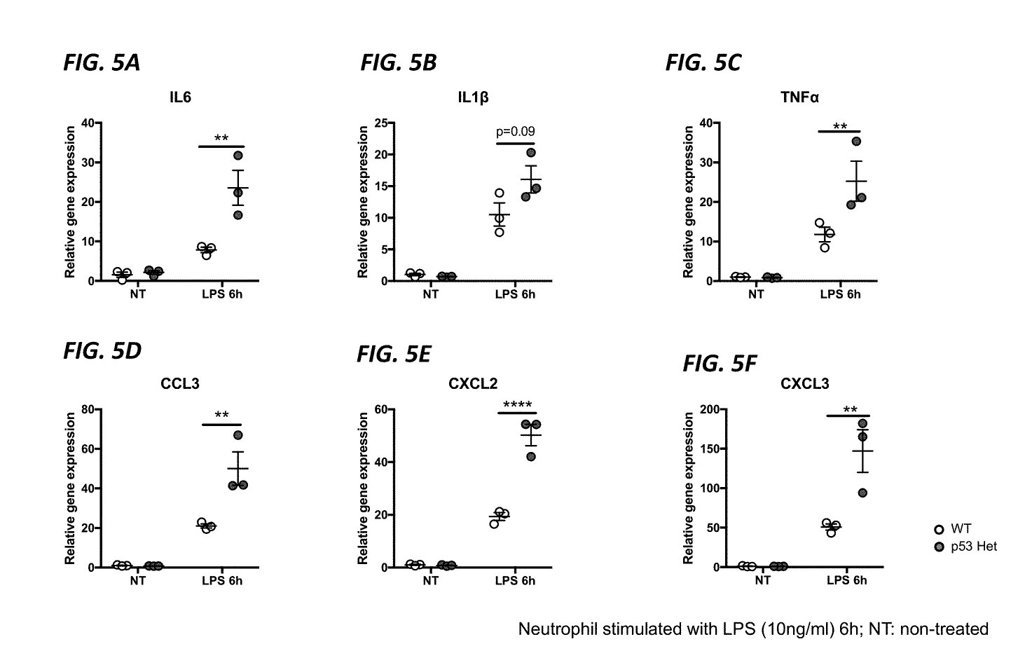

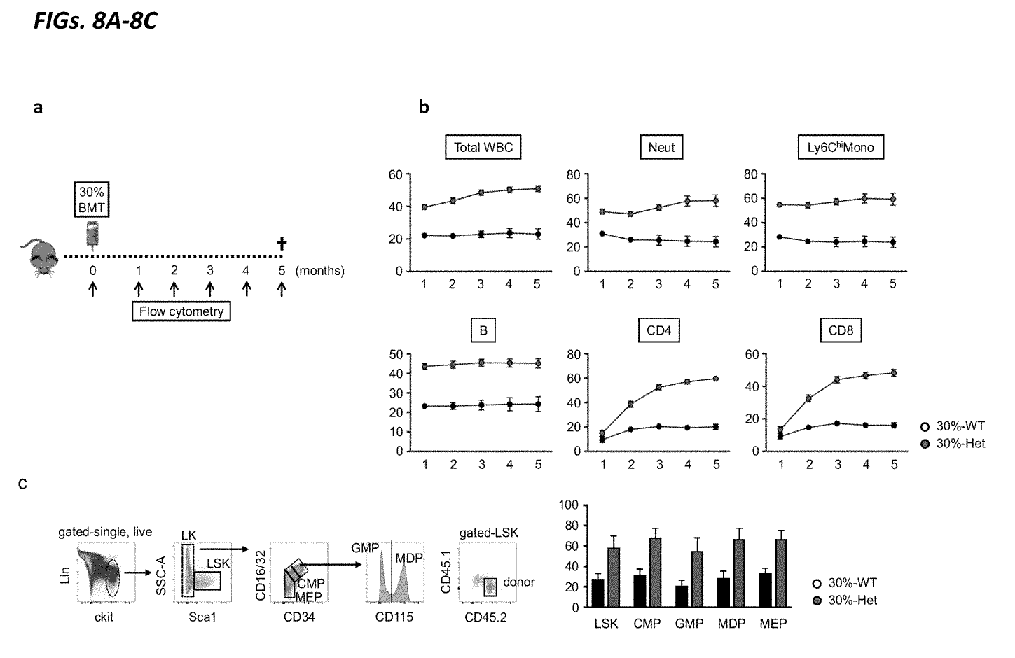

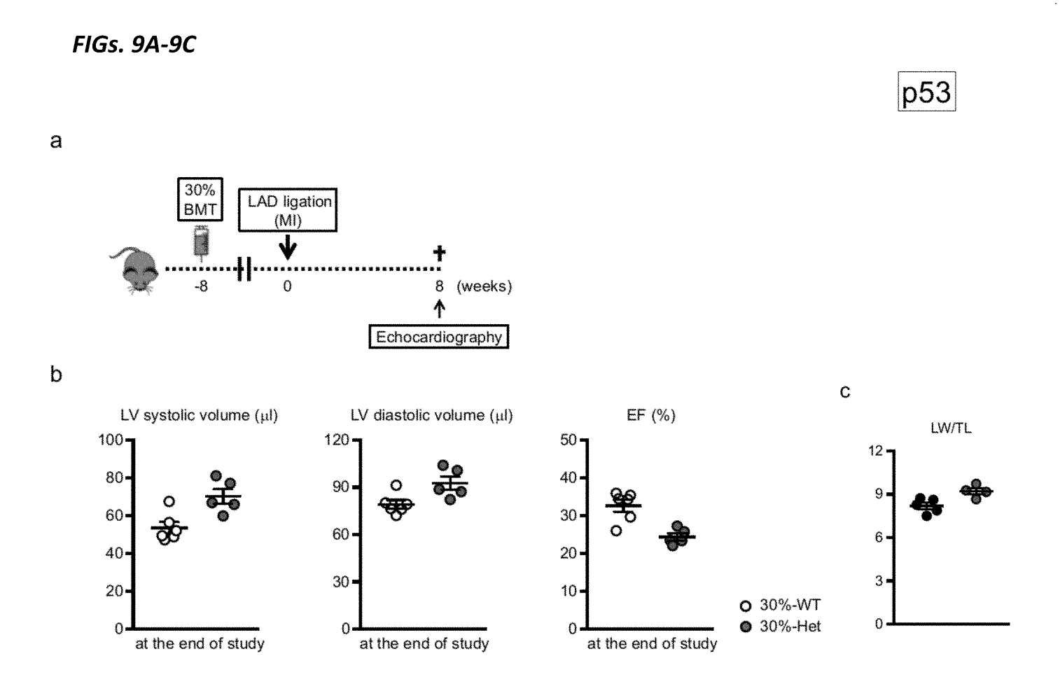

As demonstrated herein, competitive bone marrow transplantation studies in mice (FIG. 8A) revealed the selective expansion TP53-deficient cells into multiple blood cell lineages (FIG. 8B). Selective expansion of TP53 was also observed in the lineage marker−, c-Kit+, Sca1+(LSK) and hematopoietic progenitor cells of the irradiated mice (FIG. 8C). The effect of TP53 loss of function on LPS-induced cytokine and chemokine production was assessed. Murine neutrophils were isolated from bone marrow of TP53-heterozygous mice or WT mice. Cytokine and chemokine transcript levels were measured at indicated time points after stimulation with 10 ng/ml LPS (FIGS. 46A-46F). Analysis of transcripts revealed that IL6 (FIG. 2 and FIG. 5A), IL13 (FIG. 2 and FIG. 5B), TNFα (FIG. 2 and FIG. 5C), CCL3 (FIG. 5D), CXCL2 (FIG. 5E), and CXCL3 (FIG. 5F) were upregulated. A summary of the effects of the clonal hematopoiesis genes Tet2, Dnmt3a, Asxl1, Ppm1d/Wip1, JAK2 (V617F) and TP53 on cytokine expression is shown in FIG. 1 and FIG. 45. When mice underwent a competitive BMT with TP53-deficient (30%) and wild type (70%) cells followed by permanent LAD ligation (FIG. 9A), hearts displayed greater remodeling and diminished function (FIG. 9B) and lungs displayed greater congestion (FIG. 9C), consistent with an exacerbated heart failure phenotype. These data demonstrate that TP53-mediated clonal hematopoiesis can contribute to pathological cardiac remodeling following myocardial injury and facilitate heart failure.

### Example 2: Myeloid JAK2V617F Expression Contributes to Pathological Remodeling in Experimental Heart Failure in the Absence of Changes in Hematopoietic Cell Number

The somatic activating mutation V617F in JAK2 in hematopoietic cells is associated with polycythemia vera, a neoplasm involving the overproduction of red blood cells and platelets. Recently, it has been found that the JAK2V617F mutation is also associated with the phenomenon of clonal hematopoiesis, a common condition found in the elderly population that generally has little or no impact on the abundance of red or white blood cells but has been associated with increased mortality due to heart disease and stroke.

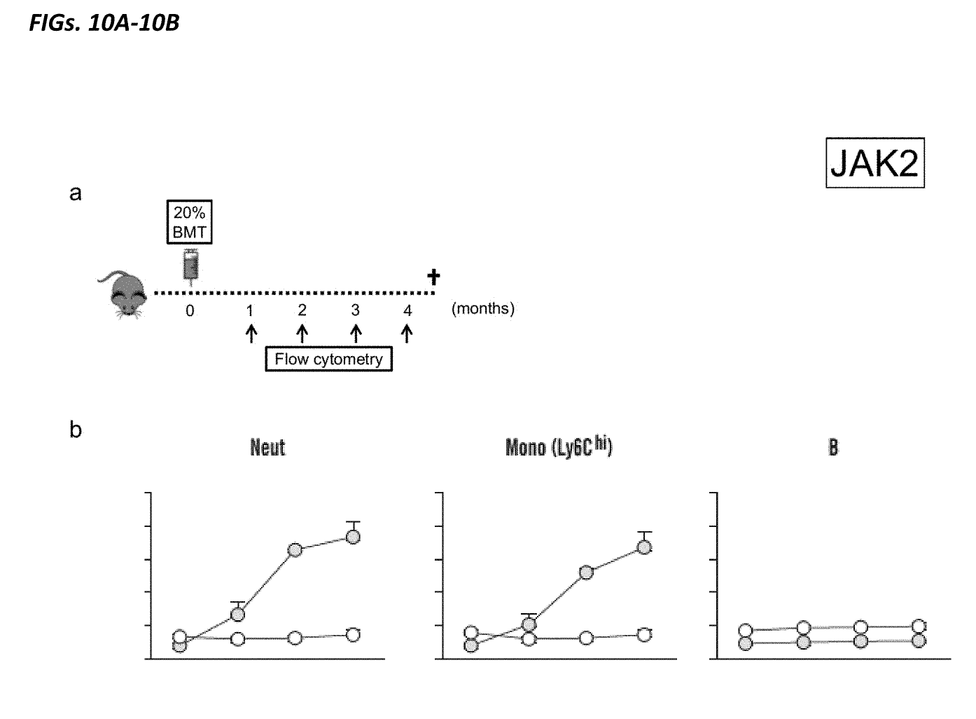

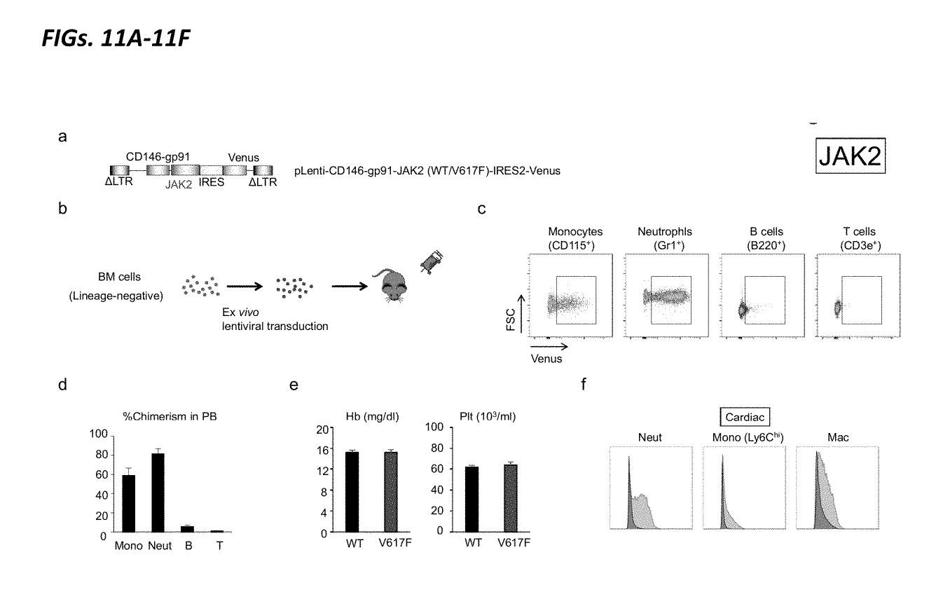

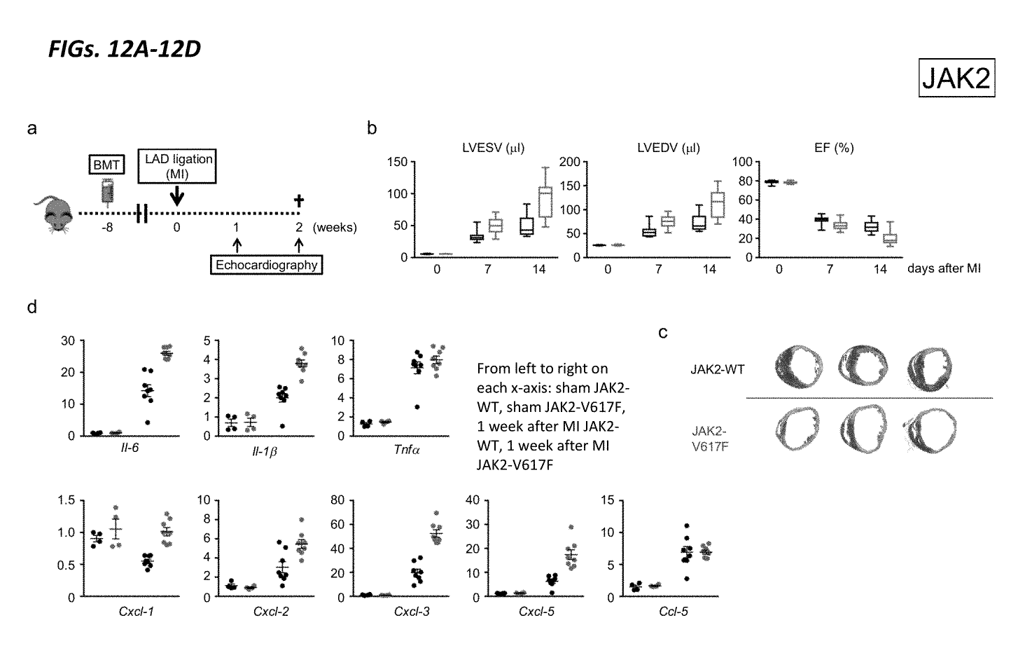

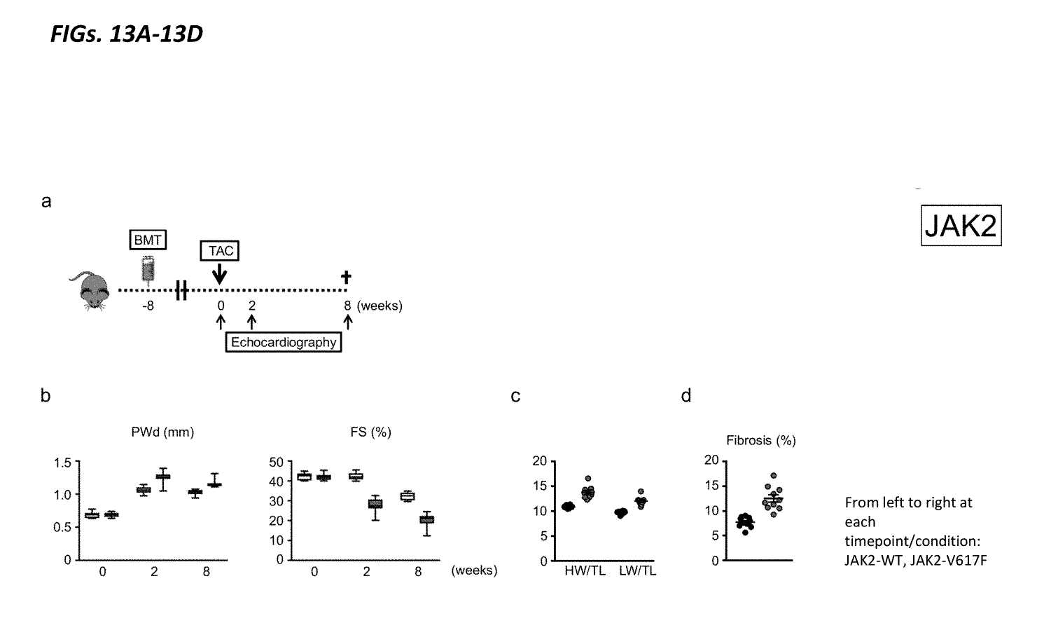

As shown herein, competitive bone marrow transplantation studies in mice revealed that the expansion of human JAK2V617F in leukocytes occurred almost exclusively in the myeloid lineage (FIGS. 10A-10B). To examine the consequences of myeloid JAK2V617F expression that are independent of changes in hematopoietic cell number, a myeloid-specific lentivirus expression vector was constructed whereby the human JAK2V617F cDNA was expressed from the CD146-gp91 promoter (FIG. 11A). Bone marrow was transduced with the JAK2V617F and control lentivirus vectors and implanted into lethally-irradiated recipient mice (FIG. 11B). A high level of JAK2V617F chimerism was observed in monocytes and neutrophils, but not in B or T cells (FIGS. 11C, 11D). Levels of red blood cells and platelets were unaffected by the transduction of bone marrow with the JAK2V617F vector. Subjecting JAK2V617F-transduced mice to permanent LAD ligation or transverse aortic constriction (TAC) led to greater pathological remodeling of the heart following injury (FIGS. 12A-12D, 13A-13D respectively). The greater pathological remodeling was accompanied by the broad over-activation of cytokines, including I1-6, IL-1β and TNFα, in the heart (FIG. 12D). These data indicate that JAK2V617F-mediated clonal hematopoiesis can contribute to pathological cardiac remodeling following myocardial injury and facilitate heart failure independent of changes in hematocrit and platelet levels.

### Example 3: Dnmt3a-Deficiency in Hematopoietic Cells Contributes to Pathological Cardiac Remodeling in Experimental Heart Failure

Somatic inactivating mutations in DNMT3A are associated with the phenomenon of clonal hematopoiesis, a common condition found in the elderly population that generally has little or no impact on the abundance of hematocrit or levels of leukocytes and platelets, but has been associated with increased mortality due to heart disease and stroke.

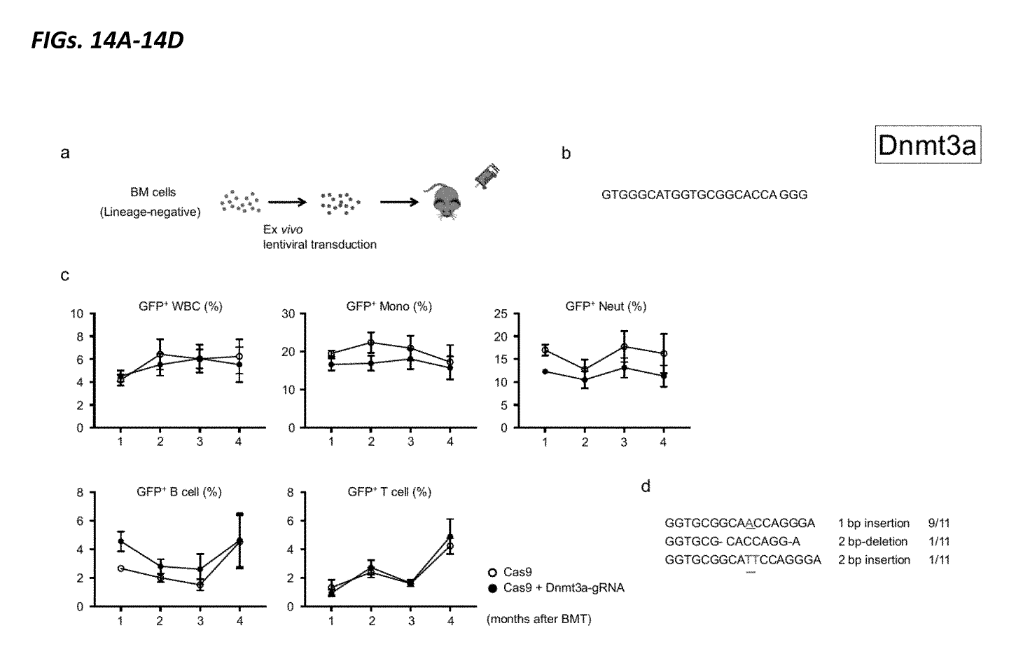

As demonstrated herein, lethally-irradiated mice underwent bone marrow transplantation (FIG. 14A) using a lentivirus vector expressing GFP and a CRISPR guide sequence that targets the Dnmt3a gene (FIG. 14B). Flow cytometry revealed that the stable expression of GFP in various hematopoietic cell lineages (FIG. 14C). Expansion of mutant cells was not observed, consistent with prior studies of Dnmt3a in the murine hematopoietic system (G A Challen et al., 2012 Nature Genetics 44:23-31). Genomic DNA was isolated from the lentivirus-transduced Lin-negative cells, and segments of the targeted site DNA were amplified by PCR and subcloned into a TA plasmid vector for sequencing. Sequence analysis of the mutations introduced into the Dnmt3a gene are shown in FIG. 14D.

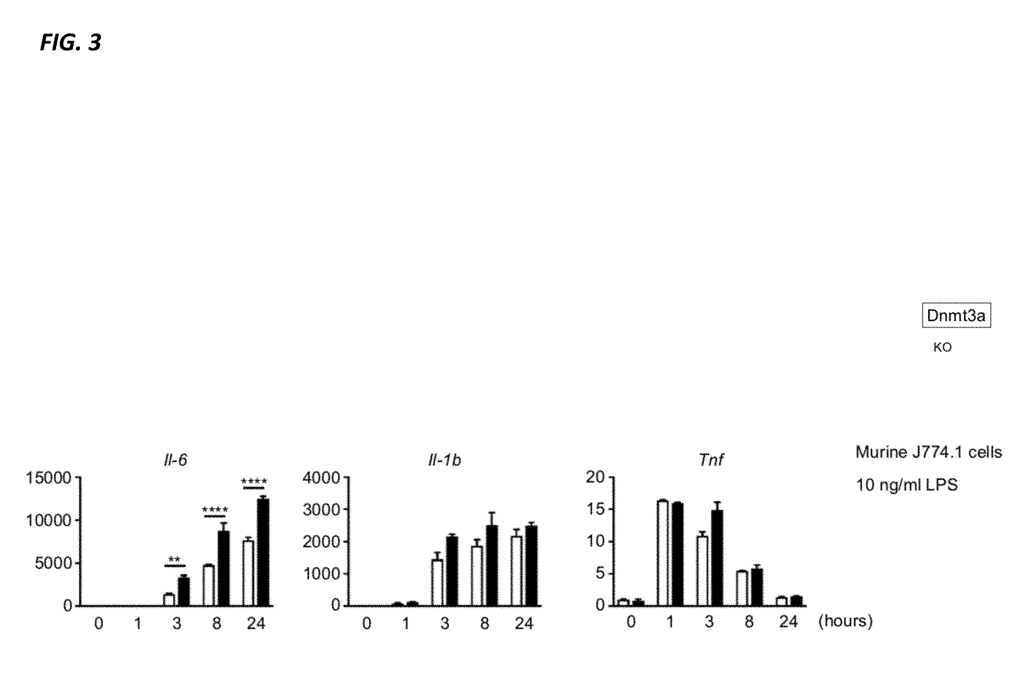

As demonstrated herein, the effects of Dnmt3a inactivation on LPS-induced cytokine production was assessed. Murine J774.1 cells (macrophage cell line) were stably transformed with a lentivirus vector that inactivates the expression of Dnmt3a (closed bars) or a control vector (open bars). Cytokine transcript levels were measured at the indicated time points after stimulation with 10 ng/ml LPS. Analysis of transcripts revealed that IL6 (FIG. 3), IL13 (FIG. 3) and TNFα (FIG. 3) were upregulated. A summary of the effects of the clonal hematopoiesis genes Tet2, Dnmt3a, Asxl1, Ppm1d/Wip1, JAK2 (V617F) and TP53 on cytokine expression is shown in FIG. 1 and FIG. 45.

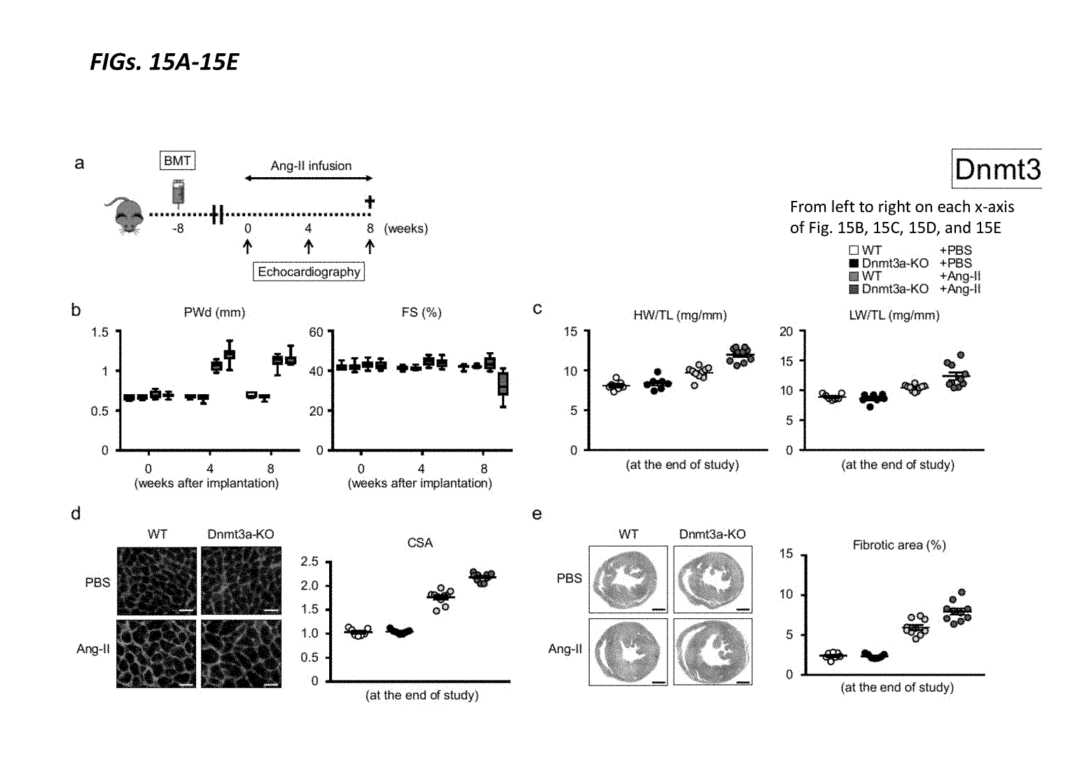

Mice underwent BMT with bone marrow treated with the lentivuirus Dnmt3a-CRISPR vector and were treated with Angiotensin II to induce cardiac stress (FIG. 15A). Hematopoietic cell mutation of Dnmt3a led to diminished cardiac function (FIG. 15B), increased cardiac hypertrophy (FIG. 15C), increased myocyte hypertrophy (FIG. 15D) and increased cardiac fibrosis (FIG. 15E). These data indicate that DNMT3A-mediated clonal hematopoiesis can contribute to pathological cardiac remodeling following injury and facilitate heart failure.

### Example 4: Tet2-Mediated Clonal Hematopoiesis Accelerates Heart Failure Through a Mechanism Involving the IL-1f3/NLRP3 Inflammasome

Recent studies have shown that hematopoietic stem cells can undergo clonal expansion due to somatic mutations in leukemia-related genes, leading to an age-dependent accumulation of mutant leukocytes in the blood. This somatic mutation-related clonal hematopoiesis is common in the healthy elderly, but it has been associated with an increased incidence of future cardiovascular disease.

Clonal hematopoiesis resulting from somatic mutations in hematopoietic stem cells is common in the elderly population and has been associated with increased incidence of cardiovascular disease. Here, it is shown that the inactivation of Tet2 in hematopoietic cells promotes their expansion and exacerbates cardiac remodeling in experimental models of heart failure via an IL-1β/NLRP3 inflammasome mechanism. These data demonstrate that post-Myocardial Infarction (MI) patients with somatic mutations in TET2 may exhibit a greater benefit from therapies that target IL-1β or the NLRP3 inflammasome.

The epigenetic regulator TET2 is frequently mutated in blood cells of individuals exhibiting clonal hematopoiesis. Tet2 deficiency in hematopoietic cells is associated with greater cardiac dysfunction in murine models of heart failure due to elevated IL-1β signaling. These data demonstrate that individuals with TET2-mediated clonal hematopoiesis may be at greater risk of developing heart failure and respond better to IL-1β/NLRP3 inflammasome inhibition.

As demonstrated herein, Tet2 mutations within hematopoietic cells can contribute to heart failure in two models of cardiac injury. Heart failure was induced in mice by pressure overload, achieved by transverse aortic constriction or chronic ischemia induced by the permanent ligation of the left anterior descending artery. Competitive bone marrow transplantation strategies using Tet2-deficient cells were used to mimic TET2 mutation-driven clonal hematopoiesis. Tet2 was also specifically ablated in myeloid cells using Cre recombinase expressed from the LysM promoter.

As demonstrated herein, in both experimental heart failure models, hematopoietic or myeloid Tet2 deficiency worsened cardiac remodeling and function, in parallel with increased IL-1β expression. Treatment with a selective NLRP3 inflammasome inhibitor (e.g. MCC950) protected against the development of heart failure and eliminated the differences in cardiac parameters between Tet2-deficient and wild-type mice.

TABLE 2
List of abbreviations.

| Full name | Abbreviation |
|---|---|
| Bone Marrow transplantation | BMT |
| Canakinumab Anti-inflammatory | CANTOS |
| Thrombosis Outcomes Study | |
| Cluster of differentiation 45 antigen | CD45 |
| C-reactive protein | CRP |
| cross-sectional area | CSA |
| Cardiovascular disease | CVD |
| DNA (cytosine-5)- | DNMT3A |
| methyltransferase 3A | |
| Ejection fraction | EF |
| Fractional shortening | FS |
| Hematopoietic stem/progenitor cells | HSPC |
| Heart weight | HW |
| Interleukin 1 beta | IL-1β |
| Knockout | KO |
| Left anterior descending | LAD |
| Left ventricle | LV |
| Left ventricle posterior wall | LVPWTd |
| thickness at diastole | |
| Lung weight | LW |
| Mycocardial infarction | MI |
| NACHT, LRR and PYD | NLRP3 |
| domains-containing protein 3 | |
| Not significant | NS |
| Phosphate buffered saline | PBS |
| quantitative polymerase chain reaction | qPCR |
| ST-Elevation Myocardial Infarction | STEMI |
| Transverse aortic constriction | TAC |
| Ten-eleven translocation 2 | TET2 |
| Tibia length | TL |
| White blood cells | WBC |
| Wild-type | WT |

Introduction

The prevalence of heart failure approximately doubles with each decade of life (1,2), and due to dramatic increases in the aging population, the heart failure epidemic is anticipated to grow significantly in the coming decades (3). While aging is the major risk factor for heart failure and overall cardiovascular disease, it is not understood how this condition promotes disease progression.

The accumulation of somatic DNA mutations in proliferative tissues is an inevitable consequence of the aging process that leads to the generation of tissues that are a mosaic of slightly different genotypes (4). In this context, the highly proliferative hematopoietic system is known to accumulate a substantial number of somatic mutations, which leads to an intense Darwinian selection due to the competition among the different mutant clones (5). Thus, the accumulation of mutations that provide a selective proliferative advantage to hematopoietic stem/progenitor cells (HSPC) will over time lead to the clonal expansion of specific mutations that become selectively overrepresented in the blood cells genomes. This phenomenon of somatic mutation-induced clonal hematopoiesis has been found to be relatively common in non-symptomatic, cancer-free, elderly individuals (6-8). Recent longitudinal studies have reported that detectable clonal hematopoiesis in non-symptomatic individuals is associated with all-cause mortality (6,7,9). While clonal hematopoiesis increases the risk of developing a hematological malignancy, this condition is relatively rare and does not significantly contribute to overall mortality under these conditions. Instead mortality associated with clonal hematopoiesis is attributed to larger increases in the incidence of coronary heart disease and stroke (7).

The protein encoded by TET2 (ten-eleven translocation 2) is an epigenetic regulatory enzyme that modulates HSPC self-renewal (10-13). TET2 gene was the first gene reported to exhibit somatic mutations in blood cells in individuals with clonal hematopoiesis in the absence of hematological malignancies (14). More than 130 distinct mutations have been reported in the TET2 gene in cancer-free individuals (6-9,14-18), the majority being small insertions/deletions, nonsense mutations, etc., that are predicted to inactivate the enzyme. The inventors have previously demonstrated that somatic TET2 mutations in blood cells play a causal role in atherosclerosis (19).

As demonstrated herein, partial bone marrow reconstitution with Tet2-deficient hematopoietic cells led to their clonal expansion and markedly increased atherosclerotic plaque size in hyperlipidemic mice. Mechanistically, Tet2-deficient macrophages exhibited greater interleukin-1β secretion, and the administration of an NLRP3 inhibitor showed greater athero-protective activity in chimeric mice reconstituted with Tet2-deficient cells than in non-chimeric mice and suppressed differences in plaque size between genotypes.

There have been no experimental studies in model systems to test whether somatic TET2 mutations in hematopoietic cells are causally linked to myocardial infarction or heart failure. As demonstrated herein, the effects of genetic inactivation of hematopoietic Tet2 in murine models of heart failure involving chronic ischemia and pressure overload hypertrophy were investigated.

Methods

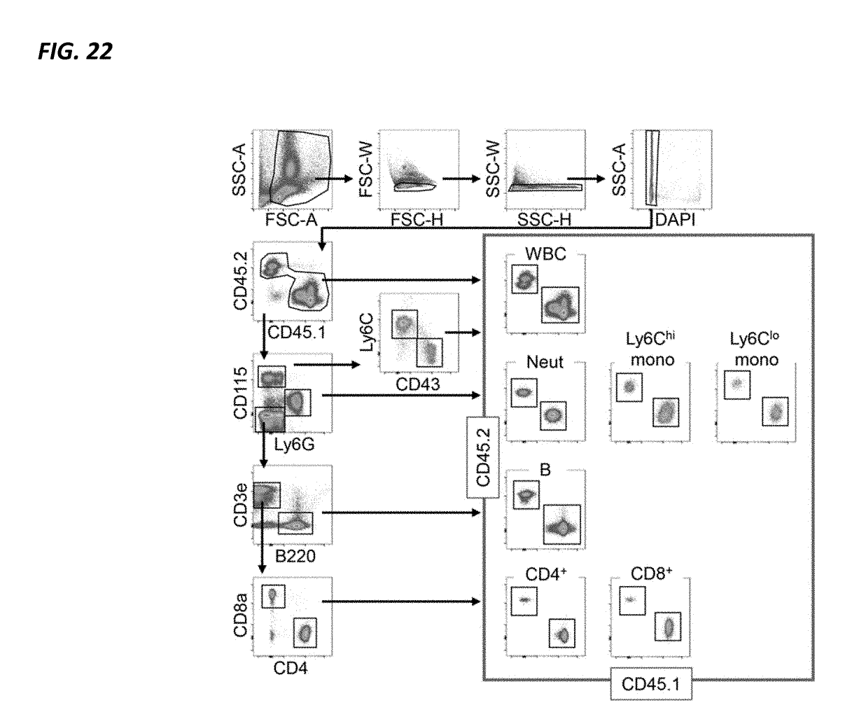

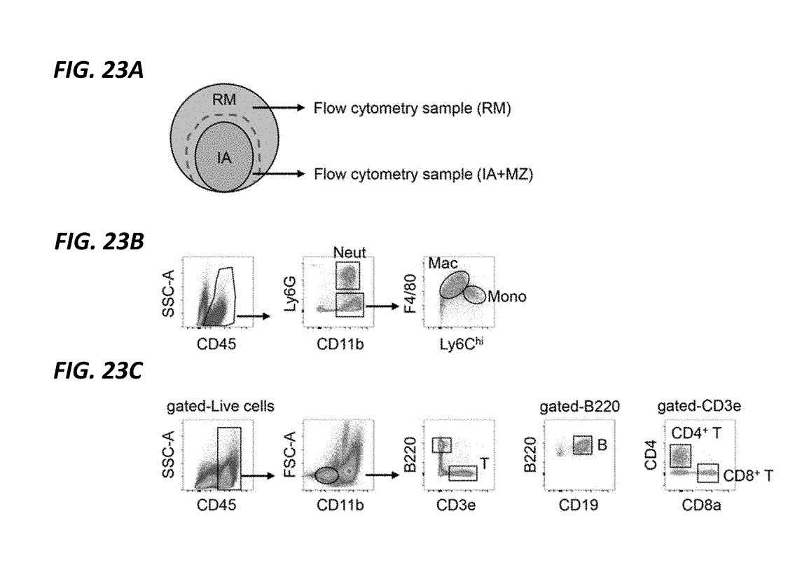

C57Bl/6J Tet2-deficient mice (Tet2^{-/-} and ^{+/-}), C57Bl/6J LysM-Cre mice, C57Bl/6J Tet2-floxed mice and C57Bl/6 Cd45.1⁺ PepBoy mice were used for these studies. These mice were used to examine the consequences of hematopoietic Tet2-deficiency using competitive bone marrow transplantations (BMT, 10% CD45.2 and 90% CD45.1 bone marrow cells) or conditional myeloid-restricted inactivation of Tet2. Both of these models of genetic Tet2 deficiency were employed in two surgical models of murine heart failure: the permanent ligation of the left anterior descending artery (LAD) and transverse aortic constriction (TAC) as described previously (20-26). Surgically-treated and sham mice were characterized by echocardiographic, histological and qPCR analyses. In some experiments, mice were continuously infused with the NLRP3 inflammasome inhibitor MCC950 (27) at a dose of 5 mg per kg per day or with phosphate-buffered saline (PBS) vehicle via subcutaneous osmotic pumps. In the BMT approach, the expansion of Tet2-deficient HSPC into the different blood lineages was assessed by flow cytometry using the gating strategy shown in FIG. 22. Leukocyte levels in the heart were determined by flow cytometry using the gating strategy outlined in FIG. 23 (28,29). For some assays, bone marrow-derived macrophages from conditional or whole-body Tet2-deficient mice were utilized for qPCR analyses. Primers for gene expression experiments are provided herein.

Statistics

Data were expressed as mean+SEM, or median (minimum to maximum). Data distribution was evaluated by Shapiro-Wilk normality test. An F-test was used to evaluate homogeneity of variance. Normally distributed data with only one variable were analyzed by parametric analysis: unpaired (two-tailed) Student's t test (with welch correction when variance was unequal) for two groups and one-way ANOVA with Tukey's multiple comparison test for three or more groups. Non-normally distributed data with only one variable were analyzed by non-parametric analysis: Mann-Whitney U test (two-tailed) for two groups. Data with more than one variable were evaluated by two-way ANOVA, with post-hoc Tukey's multiple comparison tests. Results of echography were evaluated by two-way repeated measure ANOVA with post-hoc Sidak's or Tukey's multiple comparison tests. In survival analysis, Kaplan Meier survival curves were compared by log-rank test. All the statistical analysis was performed by GraphPad Prism 7 software (GraphPad Software).

Results

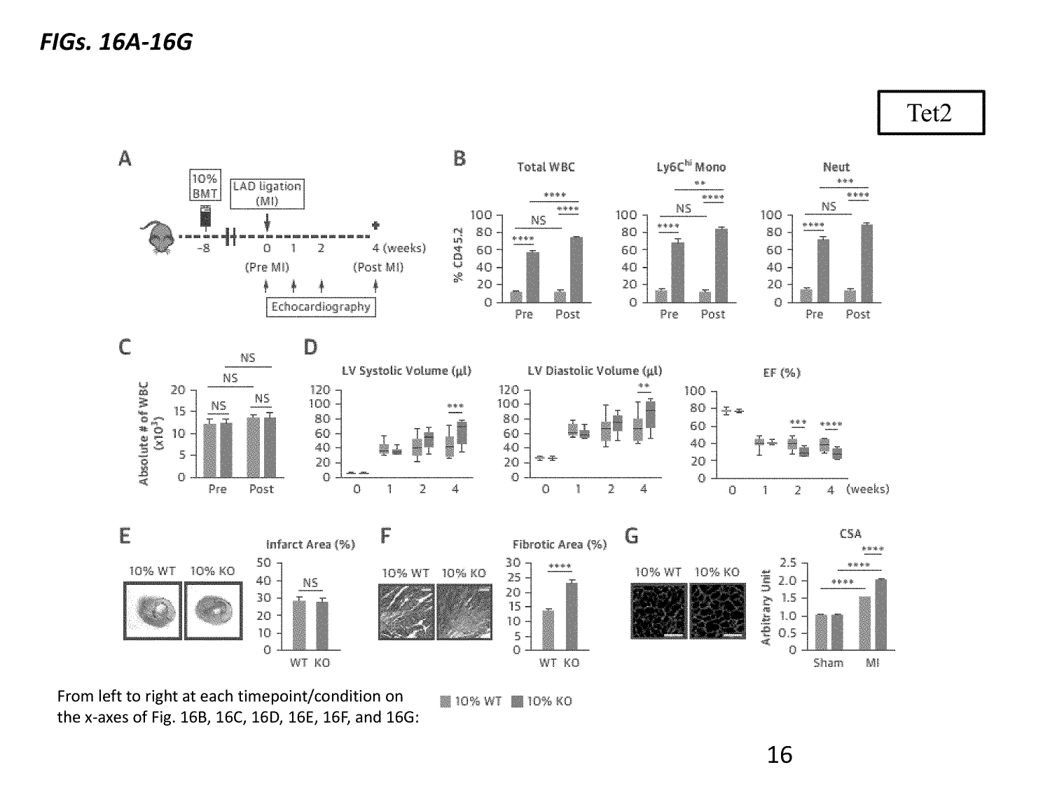

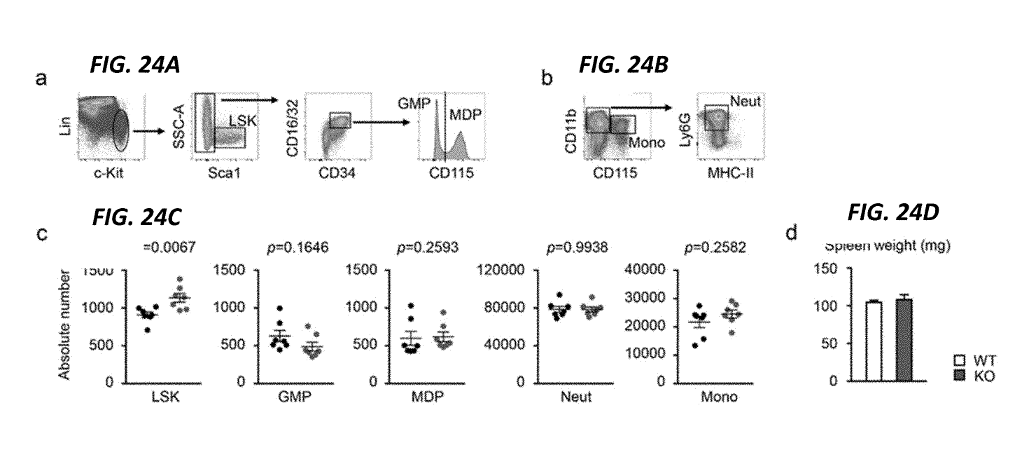

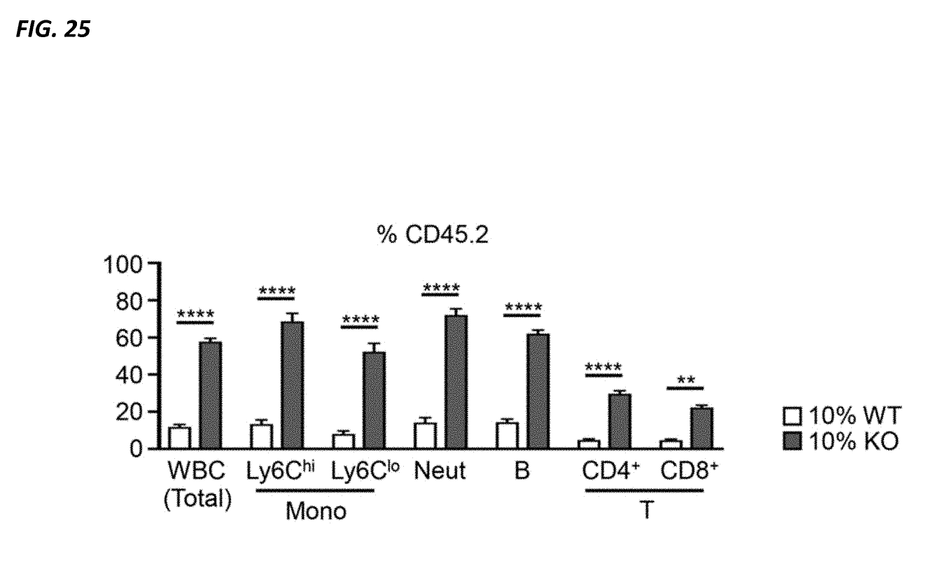

A competitive bone marrow transplantation (BMT) strategy was employed to mimic the clonal hematopoiesis that results from Tet2 inactivation and study its effects on experimental MI (FIG. 16A). Lethally irradiated mice were transplanted with bone marrow cells containing 10% Tet2^{-/-} cells and 90% Tet2^{+/+} cells (i.e. the 10% knock out (KO) test condition). To distinguish between these different genotypes, wild-type Tet2^{+/+} cells were harvested from mice carrying the CD45.1 variant of the CD45 antigen, and Tet2^{-/-} cells were harvested from mice harboring the CD45.2 variant. Control wild-type (WT) mice received 10% CD45.2⁺Tet2^{+/+} and 90% CD45.⁺Tet2^{+/+} cells. At 8 weeks after BMT and at 4 weeks after LAD ligation in BMT-treated animals (pre and post, respectively) flow cytometry was performed on blood leukocytes to assess the expansion of Tet2^{-/-} cells (FIG. 16B). Consistent with our prior report (19), Tet2^{-/-} CD45.2⁺ cells displayed a time dependent expansion into the white blood cells (WBC), Ly6C^{hi} monocytes and neutrophils (FIG. 16B). Although Tet2-deficient donor mice displayed a small elevation in lineage marker, c-Kit⁺, Sca1⁺ (LSK), this difference did not appear to account for the large increase in Tet2^{-/-} cell expansion into the difference lineages (FIGS. 24A-24D). Tet2-deficient donor mice also did not display differences in levels of GMP or MDP progenitor cells, neutrophils, or monocytes, or spleen weights at this age. These Tet2^{-/-} cells expanded into multiple blood lineages, but a reduced expansion into the T cell lineage was observed (FIG. 25). Total WBC levels were unaffected by genotype (FIG. 16C).

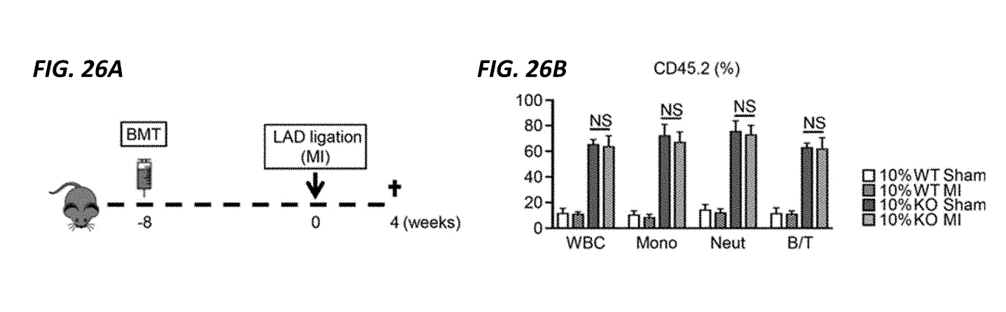

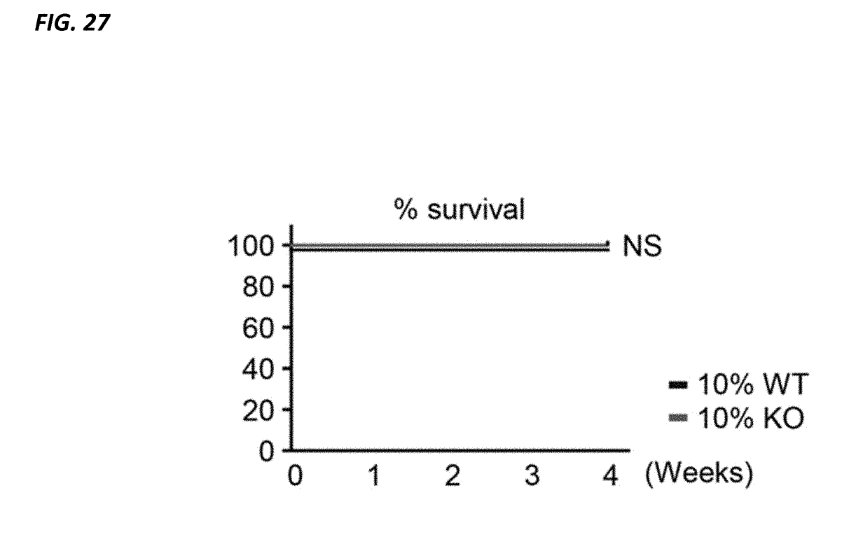

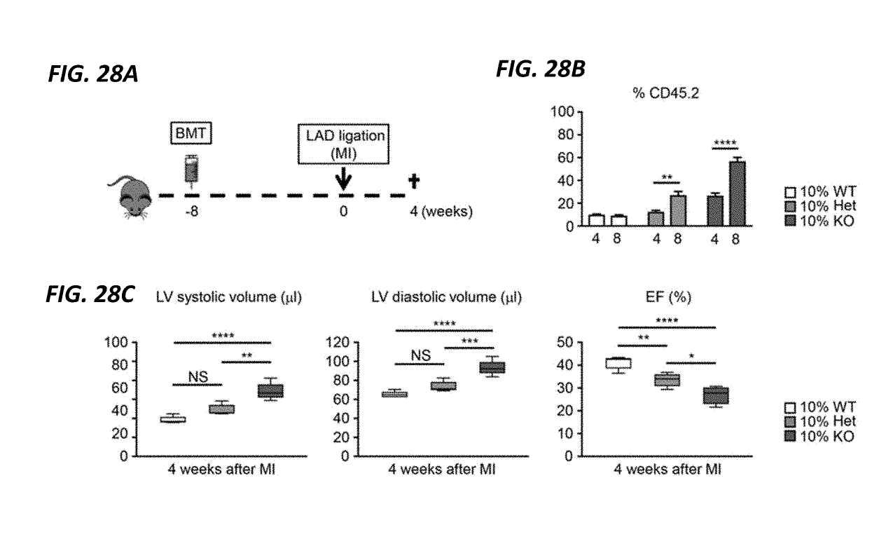

At the 8 week time point, mice from both experimental groups underwent permanent LAD ligation surgery as described previously (20,25,26). Permanent LAD ligation surgery did not affect the expansion of Tet2^{-/-} cells (FIGS. 26A-26B). Consistent with prior studies (30), all transplanted mice survived surgery following irradiation (FIG. 27). At 4 weeks after myocardial infarction (MI) surgery, the 10% KO-BMT mice displayed statistically significant increases in left ventricle (LV) systolic volume and LV diastolic volume, indicative of greater systolic dysfunction, compared to control mice that also underwent BMT and MI surgery (FIG. 16D). Trends in these echocardiographic parameters were observed at the 2 week time point but did not reach statistical significance. Ejection fraction was significantly reduced in the 10% KO-BMT group at 2 and 4 weeks post-MI. Consistent with a lack of difference in echocardiographic parameters at the 1 week time point, there was no detectable difference in infarct size between 10% KO-BMT and 10% WT-BMT mice (FIG. 16E). There was a statistically significant increase in fibrosis in the marginal zone of the 10% KO-BMT mice (FIG. 16F). Wheat germ agglutinin staining was employed on tissue sections, to determine cardiac myocyte cross-sectional area, revealed that myocytes from 10% KO-BMT mice displayed greater hypertrophy at the marginal zone (FIG. 16G), consistent with greater post-MI cardiac remodeling. Analysis of the infarct marginal zone for F4-80-positive cells revealed greater macrophage infiltration at the 4 week post-MI time point (FIG. 25). In separate studies, competitive BMT experiments with Tet2^{+/-} cells revealed that Tet2 heterozygosity is sufficient to promote cardiac dysfunction in this model, albeit at a reduced level compared to homozygous Tet2-deficient cells (FIGS. 28A-28C). These findings are consistent with the slower kinetics of Tet2-heterozygous cell expansion.

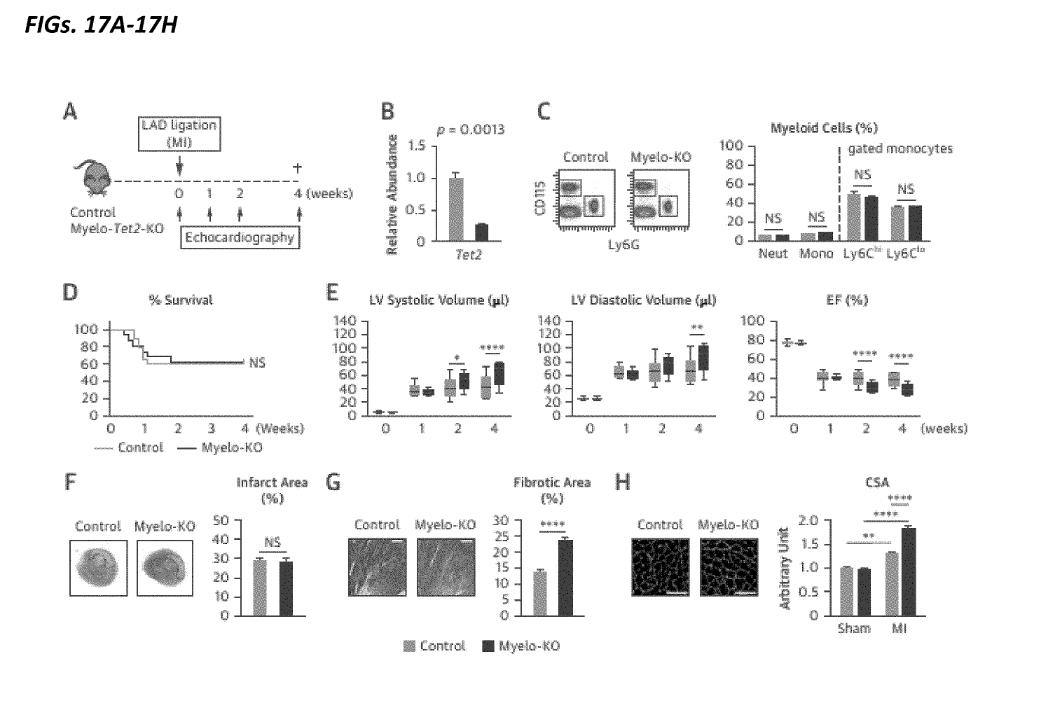

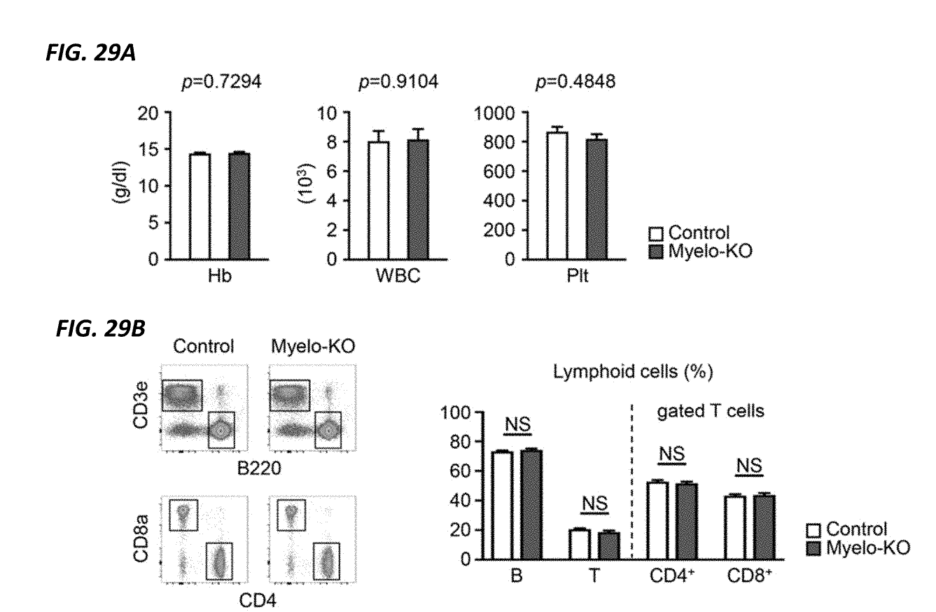

Due to the central role of macrophage activation in post-MI remodeling (31) and the preferential expansion of human TET2-mutant cells into the myeloid lineage in mice (32), it was investigated whether the targeted ablation of Tet2 would be sufficient to promote post-MI remodeling. Myeloid-deficient Tet2 mice (Myelo-Tet2-KO mice) were generated using LysM-Cre/LoxP strategies and subjected to permanent LAD ligation (FIG. 17A). Using this system, the ablation of Tet2 in bone marrow-derived macrophages occurred at an efficiency of 74% (FIG. 17B). This condition did not lead to detectable changes in the levels of hematopoietic cells (FIG. 17C and FIGS. 29A-29B). No differences in mouse survival following LAD ligation was detected between Tet2-deficient and control strains of mice over the time course of this experiment (FIG. 17D). However, Myelo-Tet2-KO mice displayed a statistically significant increase in LV systolic and LV diastolic volumes at the 4 week post-MI time point, and trends toward increases in these parameters at the 2 week time point, compared with control mice (FIG. 17E). Calculated ejection fraction showed statistically significant reductions at both 2 and 4 week time points in the Myelo-Tet2-KO mice. Infarct size was unaffected by the different myeloid Tet2 genotypes (FIG. 17F). At the terminal 4 week time point hearts from Myelo-Tet2-KO mice displayed an increase in fibrosis in cardiac tissue sections from the marginal zone (FIG. 17G), and an increase in cardiac myocyte cross sectional area (FIG. 17H), consistent with greater cardiac remodeling.

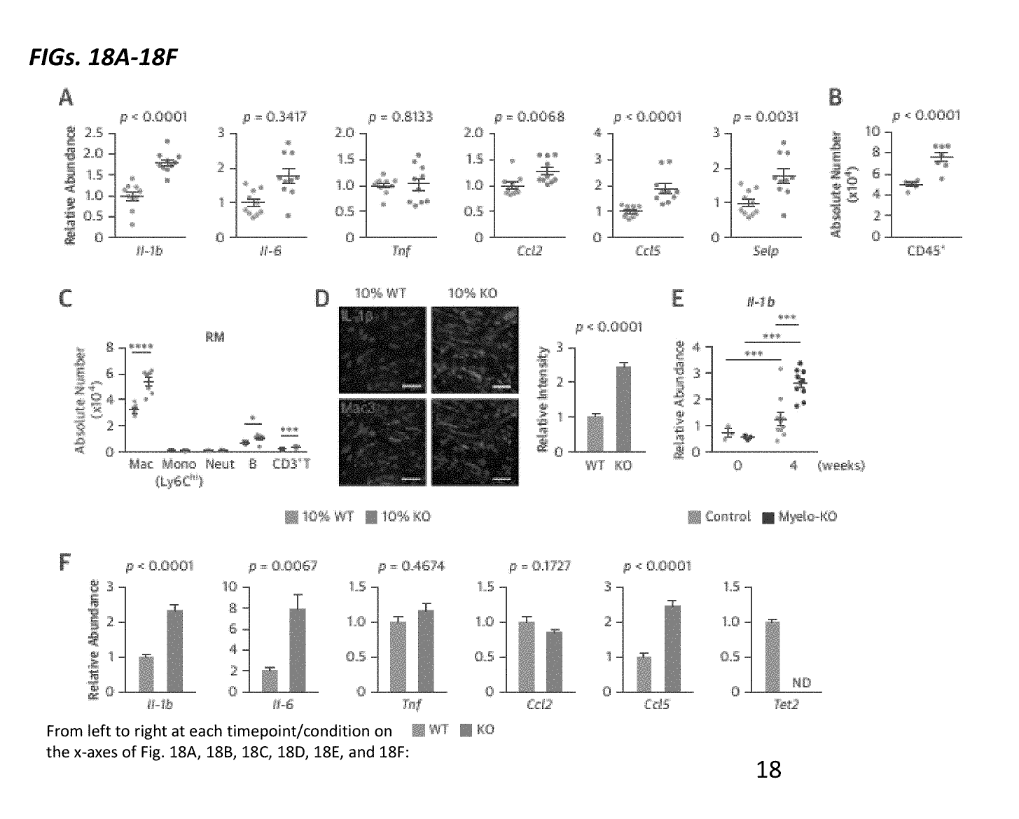

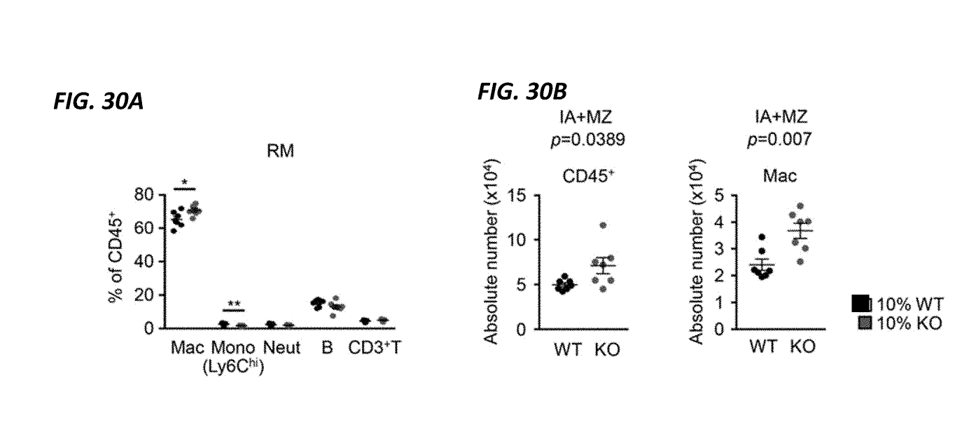

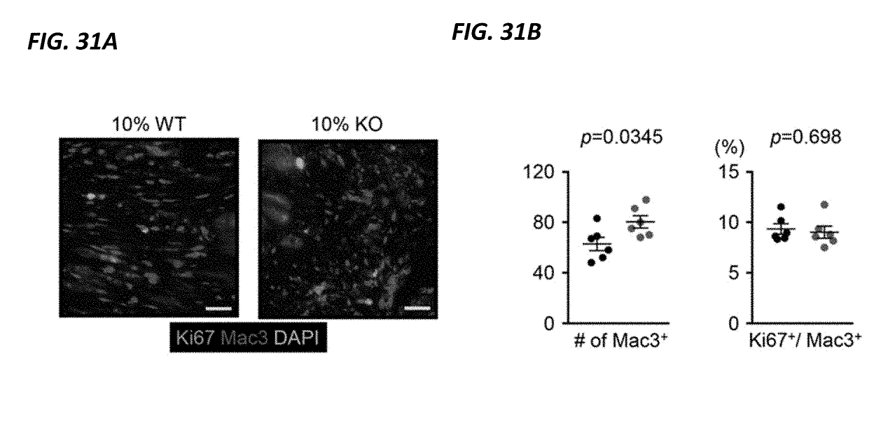

As demonstrated herein, analysis of transcripts revealed that interleukin 1 beta (IL-1β), IL-18, Cxcl2, Ccl2, Ccl5 and P-selectin were upregulated in the post-MI hearts of 10% KO-BMT mice compared to 10% WT-BMT mice (FIG. 18A). In contrast, little or no statistical differences were detected in the transcript levels of IL-6, TNFα. The 10% KO-BMT, post-MI hearts also displayed an increase in CD45⁺ leukocytes that could primarily by attributed to the abundance of macrophages (FIG. 18B, 18C and FIGS. 30A-30B). The increase in macrophage number was not accompanied by an increase in proliferation, as assessed by Ki67 (FIGS. 31A-31B). To document changes in IL-1β protein, confocal microscopy immunofluorescence staining of IL-1β was performed on marginal zone sections of hearts at the 4 week post-MI time point. Cardiac sections from 10% KO-BMT mice displayed significantly greater IL-1β expression than sections of 10% WT-BMT mice (FIG. 18D). Consistent with these results, IL-1β transcript levels were upregulated in the myocardium of the Tet2-KO mice compared to control mice at the 4 week post-MI time point (FIG. 18E). Bone marrow-derived macrophages isolated from whole body Tet2-deficient mice also displayed elevated IL-1β, IL-6 and Ccl5 transcript expression (FIG. 18F).

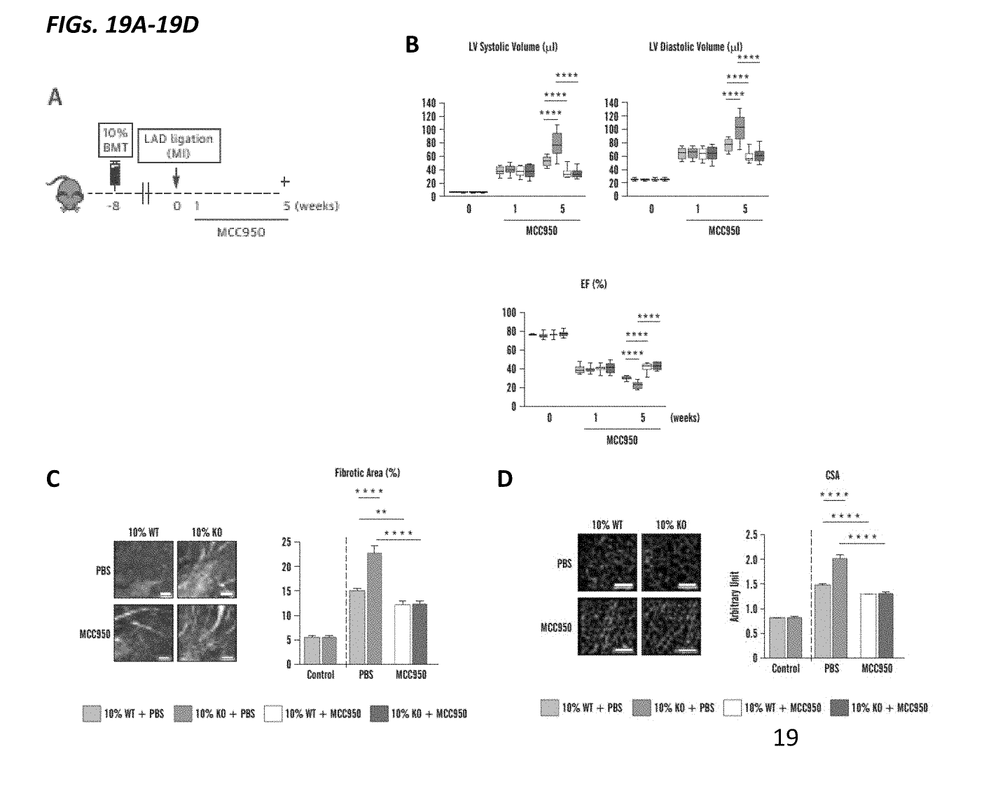

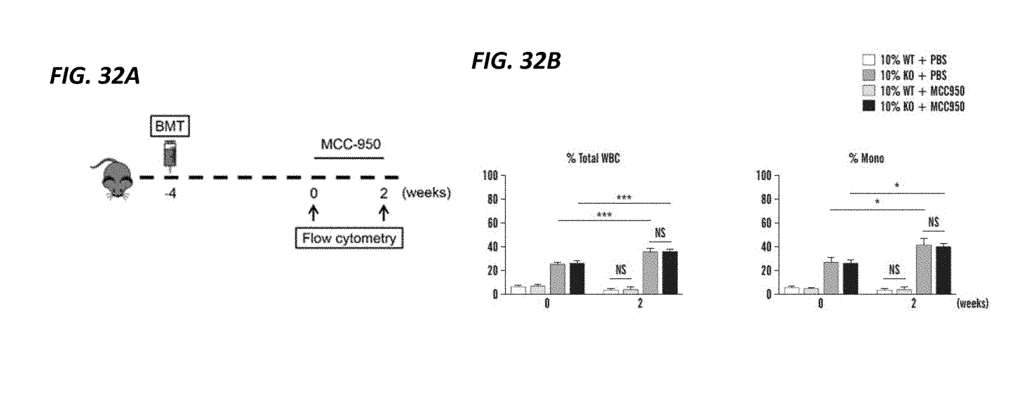

As demonstrated herein, the functional significance of NLRP3-mediated IL-1β production in cardiac remodeling was assessed. 10% KO-BMT and 10% WT-BMT mice were infused with the specific NLRP3 inhibitor MCC950 starting at one week post-MI and continued until sacrifice at 5 weeks (FIG. 19A). At the one week time point (initiation of MCC950 infusion), there were no detectable differences in echocardiographic parameters in the different experimental groups of mice (FIG. 19B). However, by week 5 post-MI, MCC950 treatment led to significant protection against cardiac remodeling in both 10% KO-BMT and 10% WT-BMT mice. Notably, the MCC950 treatment completely eliminated echocardiographic differences in LV systolic volume, LV diastolic volume or ejection fraction, between the Tet2-deficient and WT experimental conditions (FIG. 19B). Treatment with MCC950 infusion also diminished marginal zone fibrosis at the 5 week time point in both 10% KO-BMT and 10% WT-BMT mice and eliminated the difference in this parameter between these experimental groups (FIG. 19C). Similarly, cardiac myocyte hypertrophy was markedly reduced by MCC950 treatment in both Tet2-deficient and WT mice and the drug eliminated the difference in this parameter between experimental groups (FIG. 19D). In a separate experiment, treatment with MCC950 had no effect on the expansion of Tet2^{-/-} HSPC into the different blood lineages (FIGS. 32A-32B), consistent with our previous study (19).

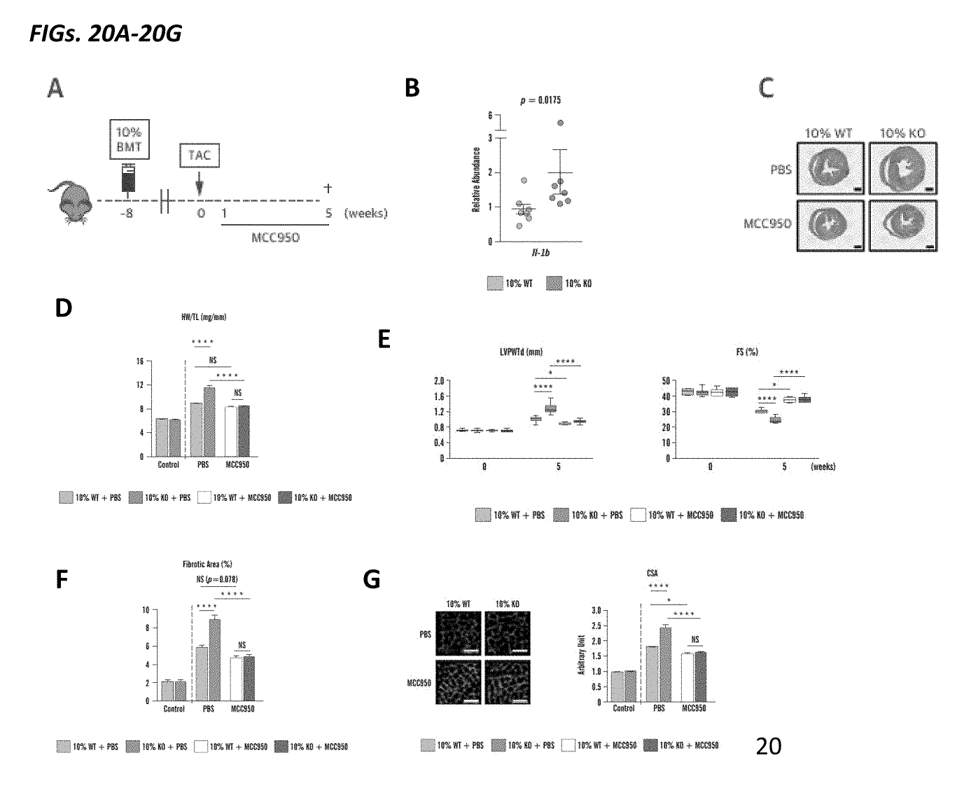

To corroborate these findings in a different model of heart failure, experimental groups of mice were also subjected to pressure overload hypertrophy by performing transverse aortic constriction (TAC) (FIG. 20A). In this model, the heart initially undergoes compensatory hypertrophy, but transitions to decompensated hypertrophy due to capillary rarefaction and other chronic pathological alterations (33,34). Because 10% KO-BMT mice exhibited increased IL-1β expression in this model (FIG. 20B), similar to our observation in the LAD model, TAC experiments were performed in mice treated with the NLRP3 inflammasome inhibitor MCC950 or vehicle control as described above to assess the effects of blockade of NLRP3 inflammasome-mediated IL-1β secretion. In this setting, MCC950 treatment was started at the time of surgery and maintained until termination of the experiment. Vehicle-infused 10% KO-BMT mice exhibited marked cardiac hypertrophy after TAC, as reflected by a greater increase in relative heart size and heart weight to tibia length ratio compared to 10% WT-BMT mice (FIGS. 20C-20D). 10% KO-BMT mice also displayed greater LV posterior wall thickness and a greater impairment in fractional shortening in the hearts of the 10% KO-BMT mice (FIG. 20E). Correspondingly, 10% KO-BMT mice displayed more fibrosis (FIG. 20F) and cardiac myocyte hypertrophy (FIG. 20G) following LAD ligation. As with the partial BMT, myeloid cell-specific ablation of Tet2 led to greater cardiac hypertrophy (FIGS. 28A-28C) and diminished cardiac function (FIGS. 29A-29B) compared to control mice. Mye-Tet2-KO mice also displayed greater expression of IL-1β after TAC than the control strain (FIGS. 30A-30B). Treatment with MCC950 had a marked inhibitory effect on the development of pressure overload-induced cardiac hypertrophy and it suppressed the differences in heart size, cardiac function, myocyte hypertrophy and interstitial fibrosis observed between the 10% KO-BMT and 10% WT-BMT mice in this model (FIGS. 20C-20G).

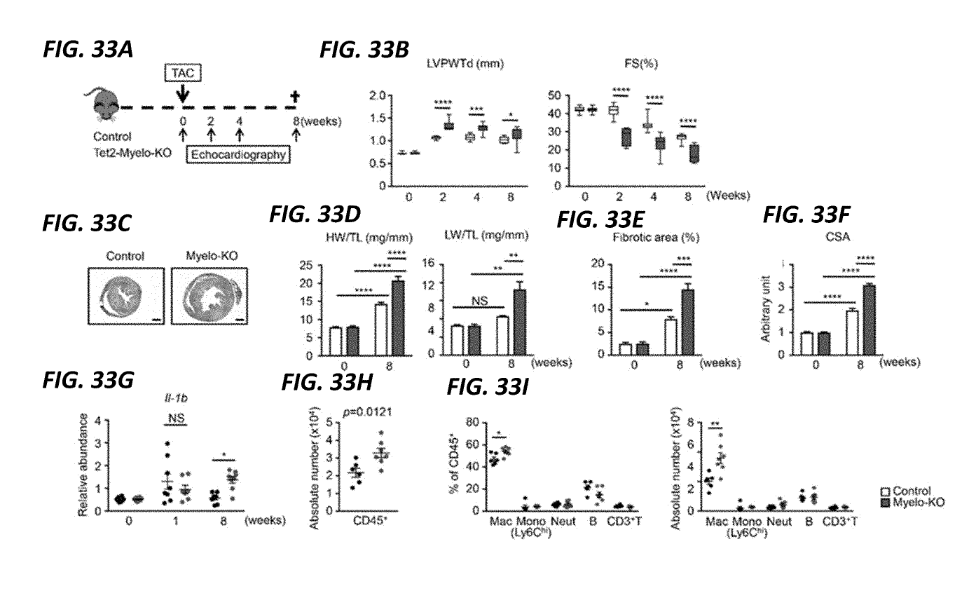

In separate experiments employing the TAC model of heart failure, the targeted ablation of Tet2 in myeloid cells also led to greater cardiac hypertrophy and diminished function compared to control mice (FIGS. 33A-33I). Myeloid Tet2-deficiency also led to greater lung congestion, myocardial fibrosis and myocyte hypertrophy. Hearts from these mice displayed greater expression of IL-1β transcript and more macrophages in the myocardium.

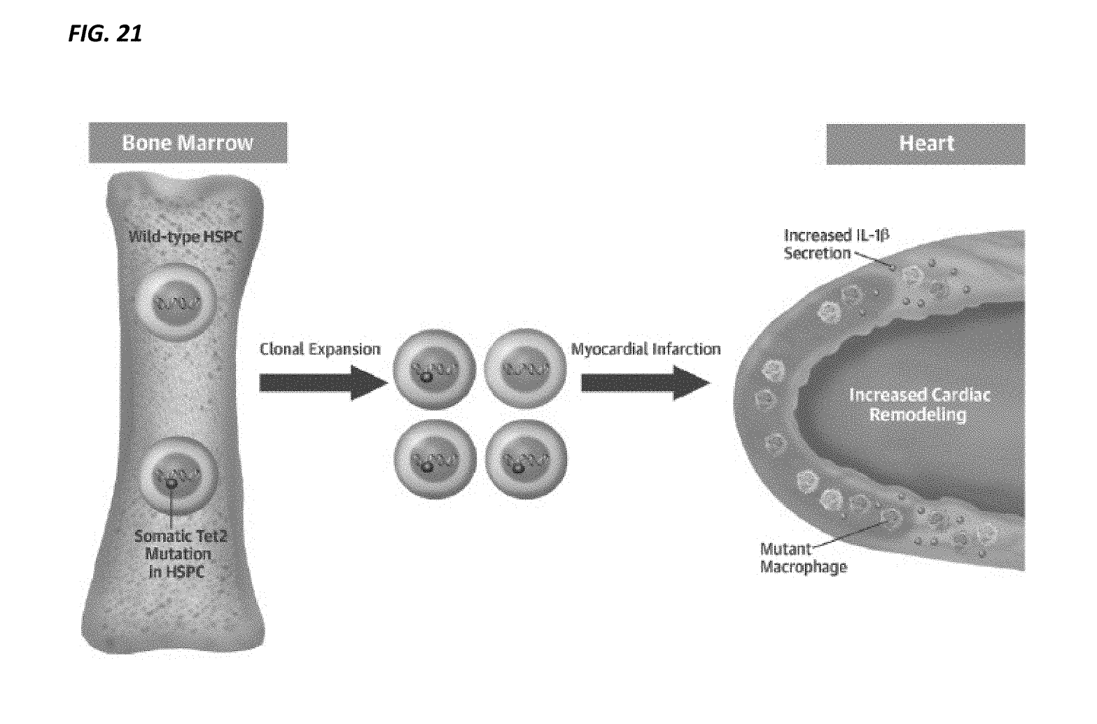

A number of recent studies have shown that it is common to find non-symptomatic individuals that exhibit clonal hematopoiesis associated with the expansion of hematopoietic cells that contain a mutation in one of a number of cancer-related genes (FIG. 21). The most common mutations occur in epigenetic regulatory genes, such as TET2, that are frequently mutated in hematological cancers (6-8). Deep sequencing of mutational hotspots within candidate genes or whole exome sequencing of a well characterized cohort revealed that clonal hematopoiesis is common in the elderly (6-9,16-18,35). Remarkably, a study employing highly sensitive targeted error-corrected sequencing revealed that 95% of individuals in their 50's possesses low levels of these mutations (predominantly DNMT3A and TET2), demonstrating that the seeding of bone marrow with mutated hematopoietic cells is essentially ubiquitous by middle age (36). These findings have led to the compelling notion that age-associated chronic diseases, both cardiovascular disease (CVD) and non-CVD, may be influenced by the occurrence of somatic mutations that lead to clonal hematopoiesis. While previous studies in our laboratory demonstrated that clonal hematopoiesis associated with Tet2 deficiency accelerates atherosclerosis in mice (19), our experimental findings presented herein demonstrate that this phenomenon may have broader implications in the context of CVD, as it may also promote heart failure in post-MI patients.

The results of the experiments show for the first time that the partial inactivation of Tet2 in hematopoietic cells, a condition that contributes to clonal hematopoiesis in humans, will promote cardiac dysfunction in two murine models of heart failure. The permanent ligation of the LAD artery is a model of severe ischemia that leads to myocardial necrosis and the remodeling of the heart in response to scar formation. The TAC model initially induces a cardiac hypertrophic response to pressure overload, and this is followed by systolic dysfunction in response to capillary rarefaction (33,34). In both of these models, Tet2-deficiency in hematopoietic cells, either by specific ablation in myeloid cells or by partial reconstitution of the bone marrow with Tet2-deficient HSPC, was associated with worse late-stage cardiac remodeling and reduced cardiac function.

As demonstrated herein, Tet2-mediated clonal hematopoiesis leads to the up-regulation of IL-1β in two models of heart failure. IL-103 is processed to an active form and secreted following cleavage in the cytosol of innate immune cells by the NLRP3 inflammasome. Treatment with the NLRP3 inflammasome inhibitor MCC950 protected against the development of heart failure in both the LAD ligation and TAC models and eliminated the differences in cardiac parameters between Tet2-deficient and wild-type (WT) mice. These findings strongly demonstrate a central role for exacerbated IL-1β production in the maladaptive cardiac remodeling observed in conditions of hematopoietic Tet2 loss of function. Consistent with this possibility, previous studies have demonstrated an important role of IL-1β in CVD. A number of compelling studies in animal models have shown that the neutralization of IL-1β or inhibition of the inflammasome will protect the ischemic heart (37-41). IL-1β levels are elevated after ST-Elevation Myocardial Infarction (STEMI) and associated with maladaptive remodeling (42), and an IL-1β neutralizing antibody has recently shown efficacy in high risk post-MI patients (43). IL-1β has been shown to have local effects on the heart (44) as well as early systemic actions that includes the enhancement of HSPC proliferation within 1 day of the infarct (45). However, neither inflammasome inhibition nor LAD ligation had a detectable impact on Tet2-deficient leukocyte expansion in the current study.

Tet2 is a multifaceted epigenetic regulator that is able to facilitate both transcription activation and repression depending on context. Previous studies have shown that Tet2 acts as a negative regulator of pro-inflammatory macrophage activation and that it can function to repress LPS-induced IL-6 expression (46,47). A mechanistic link between Tet2 inactivation and NLRP3-mediated IL-1β production has also been documented (19). Tet2-deficient macrophages express higher levels of IL-1β transcript and pro-IL-1β protein, its inactive precursor. Tet2-deficiency also upregulates components of the NLRP3 inflammasome, and there are increases in the levels of cleaved and secreted IL-1β that exceed the level of its transcriptional activation demonstrating that Tet2 modulates multiple steps in the IL-1β pathway. Tet2-mediated IL-1β regulation is independent of its catalytic activity, i.e. the oxidation of 5-methylcytosine, but involves changes in the recruitment of histone deacetylase to the IL-1β promoter (19,46).

The Canakinumab Anti-inflammatory Thrombosis Outcomes Study (CANTOS) has shown that the IL-1β neutralizing antibody canakinumab can reduce major adverse cardiovascular events in patients with stable coronary artery disease and elevated levels of C-reactive protein, predominantly due to reductions in the incidence of repeat myocardial infarction (43). Based upon the findings of the current study and our previous atherosclerosis studies (19), it is contemplated herein that subjects with somatic mutations in TET2 can exhibit a superior response to this treatment. In view of the infection side effect of canakinumab therapy, the identification of patients who would most benefit from this drug is warranted.

As demonstrated herein, mice with Tet2-deficiency in hematopoietic cells display greater maladaptive cardiac remodeling and dysfunction in models of pressure overload hypertrophy and permanent LAD ligation. NLRP3 inflammasome inhibition had a disproportionately greater protective effect in these models when mice were engineered to have inactivating mutations in the Tet2 gene of hematopoietic cells. Given that somatic mutation in TET2 are relatively common in the elderly population, these data demonstrate that IL-1β blockade or NLRP3 inflammasome inhibition will be particularly effective for the treatment of CVD in individuals who carry these mutations.

Clinical Perspectives

Clinical Competencies:

Elderly individuals frequently exhibit a condition referred to as clonal hematopoiesis that results from somatic, pre-leukemic gene mutations in hematopoietic stem cells. One of these commonly mutated genes is the epigenetic regulator TET2. Recent epidemiological studies have associated somatic TET2 mutations with an increased incidence of cardiovascular disease. Individuals exhibiting TET2-mediated clonal hematopoiesis may be at greater risk of developing heart failure. These data further support the concept that a precancerous state can contribute to cardiovascular diseases.

### REFERENCES

1. de Freitas E V, Batlouni M, Gamarsky R. Heart failure in the elderly. J Geriatr Cardiol 2012; 9:101-7.
2. Belsky D W, Caspi A, Houts R et al. Quantification of biological aging in young adults. Proc Natl Acad Sci USA 2015; 112:E4104-10.
3. Vigen R, Maddox T M, Allen L A. Aging of the United States population: impact on heart failure. Curr Heart Fail Rep 2012; 9:369-74.
4. Vijg J. Somatic mutations, genome mosaicism, cancer and aging. Curr Opin Genet Dev 2014; 26:141-9.
5. Shlush L I, Zandi S, Itzkovitz S, Schuh A C. Aging, clonal hematopoiesis and preleukemia: not just bad luck? Int J Hematol 2015; 102:513-22.
6. Genovese G, Kahler A K, Handsaker R E et al. Clonal hematopoiesis and blood-cancer risk inferred from blood DNA sequence. N Engl J Med 2014; 371:2477-87.
7. Jaiswal S, Fontanillas P, Flannick J et al. Age-related clonal hematopoiesis associated with adverse outcomes. N Engl J Med 2014; 371:2488-98.
8. Xie M, Lu C, Wang J et al. Age-related mutations associated with clonal hematopoietic expansion and malignancies. Nat Med 2014; 20:1472-8.
9. Zink F, Stacey S N, Norddahl G L et al. Clonal hematopoiesis, with and without candidate driver mutations, is common in the elderly. Blood 2017.
10. Ko M, Bandukwala H S, An J et al. Ten-Eleven-Translocation 2 (TET2) negatively regulates homeostasis and differentiation of hematopoietic stem cells in mice. Proc Natl Acad Sci USA 2011; 108:14566-71.
11. Moran-Crusio K, Reavie L, Shih A et al. Tet2 loss leads to increased hematopoietic stem cell self-renewal and myeloid transformation. Cancer Cell 2011; 20:11-24.
12. Quivoron C, Couronne L, Della Valle V et al. TET2 inactivation results in pleiotropic hematopoietic abnormalities in mouse and is a recurrent event during human lymphomagenesis. Cancer Cell 2011; 20:25-38.
13. Li Z, Cai X, Cai C L et al. Deletion of Tet2 in mice leads to dysregulated hematopoietic stem cells and subsequent development of myeloid malignancies. Blood 2011; 118:4509-18.
14. Busque L, Patel J P, Figueroa M E et al. Recurrent somatic TET2 mutations in normal elderly individuals with clonal hematopoiesis. Nat Genet 2012; 44:1179-81.
15. Ko M, Huang Y, Jankowska A M et al. Impaired hydroxylation of 5-methylcytosine in myeloid cancers with mutant TET2. Nature 2010; 468:839-43.
16. Jaiswal S, Natarajan P, Silver A J et al. Clonal Hematopoiesis and Risk of Atherosclerotic Cardiovascular Disease. N Engl J Med 2017.
17. Buscarlet M, Provost S, Zada Y F et al. DNMT3A and TET2 dominate clonal hematopoiesis and demonstrate benign phenotypes and different genetic predispositions. Blood 2017; 130:753-762.
18. Acuna-Hidalgo R, Sengul H, Steehouwer M et al. Ultra-sensitive Sequencing Identifies High Prevalence of Clonal Hematopoiesis-Associated Mutations throughout Adult Life. Am J Hum Genet 2017; 101:50-64.
19. Fuster J J, MacLauchlan S, Zuriaga M A et al. Clonal hematopoiesis associated with TET2 deficiency accelerates atherosclerosis development in mice. Science 2017; 355:842-847.
20. Maruyama S, Nakamura K, Papanicolaou K N et al. Follistatin-like 1 promotes cardiac fibroblast activation and protects the heart from rupture. EMBO Mol Med 2016; 8:949-66.
21. Shibata R, Izumiya Y, Sato K et al. Adiponectin protects against the development of systolic dysfunction following myocardial infarction. J Mol Cell Cardiol 2007; 42:1065-74.
22. Shimano M, Ouchi N, Nakamura K et al. Cardiac myocyte-specific ablation of follistatin-like 3 attenuates stress-induced myocardial hypertrophy. J Biol Chem 2011; 286:9840-8.
23. Shimano M, Ouchi N, Nakamura K et al. Cardiac myocyte follistatin-like 1 functions to attenuate hypertrophy following pressure overload. Proc Natl Acad Sci USA 2011; 108:E899-906.
24. Shibata R, Ouchi N, Ito M et al. Adiponectin-mediated modulation of hypertrophic signals in the heart. Nat Med 2004; 10:1384-9.
25. Shibata R, Sato K, Kumada M et al. Adiponectin accumulates in myocardial tissue that has been damaged by ischemia-reperfusion injury via leakage from the vascular compartment. Cardiovasc Res 2007; 74:471-9.
26. wei K, Serpooshan V, Hurtado C et al. Epicardial FSTL1 reconstitution regenerates the adult mammalian heart. Nature 2015; 525:479-85.
27. Coll R C, Robertson A A, Chae J J et al. A small-molecule inhibitor of the NLRP3 inflammasome for the treatment of inflammatory diseases. Nat Med 2015; 21:248-55.
28. Anzai A, Choi J L, He S et al. The infarcted myocardium solicits GM-CSF for the detrimental oversupply of inflammatory leukocytes. J Exp Med 2017; 214:3293-3310.
29. Hilgendorf I, Gerhardt L M, Tan T C et al. Ly-6Chigh monocytes depend on Nr4a1 to balance both inflammatory and reparative phases in the infarcted myocardium. Circ Res 2014; 114:1611-22.
30. Protti A, Mongue-Din H, Mylonas K J et al. Bone marrow transplantation modulates tissue macrophage phenotype and enhances cardiac recovery after subsequent acute myocardial infarction. J Mol Cell Cardiol 2016; 90:120-8.
31. Swirski F K, Nahrendorf M. Leukocyte behavior in atherosclerosis, myocardial infarction, and heart failure. Science 2013; 339:161-6.
32. Delhommeau F, Dupont S, Della Valle V et al. Mutation in TET2 in myeloid cancers. N Engl J Med 2009; 360:2289-301.
33. Shiojima I, Sato K, Izumiya Y et al. Disruption of coordinated cardiac hypertrophy and angiogenesis contributes to the transition to heart failure. J Clin Invest 2005; 115:2108-18.
34. Izumiya Y, Shiojima I, Sato K, Sawyer D B, Colucci W S, Walsh K. Vascular endothelial growth factor blockade promotes the transition from compensatory cardiac hypertrophy to failure in response to pressure overload. Hypertension 2006; 47:887-93.
35. McKerrell T, Park N, Moreno T et al. Leukemia-associated somatic mutations drive distinct patterns of age-related clonal hemopoiesis. Cell Rep 2015; 10:1239-45.
36. Young A L, Challen G A, Birmann B M, Druley T E. Clonal haematopoiesis harbouring AML-associated mutations is ubiquitous in healthy adults. Nat Commun 2016; 7:12484.
37. Toldo S, Schatz A M, Mezzaroma E et al. Recombinant human interleukin-1 receptor antagonist provides cardioprotection during myocardial ischemia reperfusion in the mouse. Cardiovasc Drugs Ther 2012; 26:273-6.
38. Toldo S, Mezzaroma E, Bressi E et al. Interleukin-1beta blockade improves left ventricular systolic/diastolic function and restores contractility reserve in severe ischemic cardiomyopathy in the mouse. J Cardiovasc Pharmacol 2014; 64:1-6.
39. Abbate A, Salloum F N, Van Tassell B W et al. Alterations in the interleukin-1/interleukin-1 receptor antagonist balance modulate cardiac remodeling following myocardial infarction in the mouse. PLoS One 2011; 6:e27923.
40. Van Tassell B W, Arena R, Biondi-Zoccai G et al. Effects of interleukin-1 blockade with anakinra on aerobic exercise capacity in patients with heart failure and preserved ejection fraction (from the D-HART pilot study). Am J Cardiol 2014; 113:321-7.
41. van Hout G P, Bosch L, Ellenbroek G H et al. The selective NLRP3-inflammasome inhibitor MCC950 reduces infarct size and preserves cardiac function in a pig model of myocardial infarction. Eur Heart J 2016.
42. Orn S, Ueland T, Manhenke C et al. Increased interleukin-1beta levels are associated with left ventricular hypertrophy and remodelling following acute ST segment elevation myocardial infarction treated by primary percutaneous coronary intervention. J Intern Med 2012; 272:267-76.
43. Ridker P M, Everett B M, Thuren T et al. Antiinflammatory Therapy with Canakinumab for Atherosclerotic Disease. N Engl J Med 2017.
44. Saxena A, Chen W, Su Y et al. IL-1 induces proinflammatory leukocyte infiltration and regulates fibroblast phenotype in the infarcted myocardium. J Immunol 2013; 191:4838-48.
45. Sager H B, Heidt T, Hulsmans M et al. Targeting Interleukin-1beta Reduces Leukocyte Production After Acute Myocardial Infarction. Circulation 2015; 132:1880-90.
46. Zhang Q, Zhao K, Shen Q et al. Tet2 is required to resolve inflammation by recruiting Hdac2 to specifically repress IL-6. Nature 2015; 525:389-93.
47. Cull A H, Snetsinger B, Buckstein R, wells R A, Rauh M J. Tet2 restrains inflammatory gene expression in macrophages. Exp Hematol 2017.

Material and Methods

Mice

C57Bl/6J Tet2-deficient mice (Tet2^{-/-} and ^{+/-}) (1), C57Bl/6J LysM-Cre mice, C57Bl/6J Tet2 floxed mice (2) and C57Bl/6 Cd45.1 Pep Boy mice were obtained from Jackson Laboratories. Mice with myeloid-restricted Tet2 ablation were generated by crossing Tet2-floxed mice (Tet2 flox/flox) with LysM-Cre mice. Male mice were used for the in vivo experiments unless otherwise noted. Mice were maintained on a 12-h light/dark schedule in a specific pathogen-free animal facility and given food and water ad libitum. The number of mice included in each study is indicated in the figures or the associated legends. The Institutional Animal Care and Use Committee (IACUC) of Boston University approved all study procedures.

Cell Culture

Bone marrow-derived macrophages (BMDM) were isolated and cultured in alpha-MEM supplemented with 10% FBS and penicillin/streptomycin/L-glutamine (Complete Media). Bone marrow was flushed from the tibia and femurs of female Tet2 KO (n=3) and littermate controls (n=4) at 10 weeks of age. Following lysis of red blood cells (BioLegend), cells were washed and cultured overnight in Complete Media in the presence of M-CSF (100 ng/mL). Differential plating was used to purify monocytes, that were identified as those cells unattached to tissue culture plastic following 16 hrs of culture. Macrophage proliferation and differentiation was induced by 2 days of culture in Complete Media in the presence of M-CSF. Cells were differentiated for the indicated periods of time and were detached by trypsin and scraping. RNA was isolated from pelleted cells using Qiazol and the miRNeasy Micro Kit (Qiagen) according to the manufacturer's instructions. Equal amounts of RNA were transcribed using a High Capacity Reverse transcription kit (Applied Biosystems) according to the manufacturer's instructions. SYBR-based detection was used to detect transcripts via qPCR through analysis on a ViiA7 (Applied Biosystems). Fold changes were calculated using the ΔΔCT method and normalized to 36b4.

In Vivo Inhibition of the NLRP3 Inflammasome

MCC950, a small molecule inhibitor of the NLRP3 inflammasome, was synthesized as previously described (22) or purchased from Selleckchem.com (FIGS. 26A-26B). It was diluted in PBS and delivered in vivo at a dose of 5 mg/kg/day via subcutaneous mini-osmotic pumps (Alzet 2004), starting 1 weeks after LAD ligation or TAC surgeries. Control mice were infused with PBS. The period of infusion is indicated in the figures or associated legends.

Competitive Bone Marrow Transplantation

6-8 week old lethally irradiated C57Bl/6 Cd45.1 Pep Boy recipients were transplanted with suspensions of BM cells containing 10% Cd45.2⁺Tet2^{-/-} cells and 90% Cd45.1⁺ Tet2^{+/+} cells (10% KO-BMT mice), or 10% Cd45.2⁺Tet2^{+/+} cells and 90% Cd45.1⁺Tet2^{+/+} cells (10% WT-BMT mice). BM cells were isolated from femurs and tibias of donor 6-8 week old mice after euthanasia. Donor Cd45.2⁺ cells were obtained from Tet2^{+/+} or ^{-/-} littermates; donor Cd45.1⁺ cells were obtained from Pep Boy mice. Recipient mice were irradiated in a pie cage (Braintree Scientific) to limit mobility and ensure an equal dose of irradiation, and were exposed to two radiation doses of 450 rad four hours apart using an X-RAD 320 Biological Irradiator. After the second irradiation, each recipient mouse was injected with 5×10⁶ BM cells via the retro-orbital vein plexus. Sterilized caging, food and water were provided during the first 14 days post-transplantation, and water was supplemented with antibiotics (Sulfatrim). 8 weeks after BMT, mice underwent cardiac surgery.

Hematopoietic Parameter Measurements

Peripheral blood cells were obtained from retro-orbital vein and collected in EDTA-coated tubes (BD). Hematopoietic parameters were analyzed by Hemavet 950FS (Drew Scientific).

Flow Cytometry Analyses of Blood and Tissue Samples

Peripheral Blood:

Antibodies: The following antibodies were used for flow cytometric analyses: anti-CD45.2-eFluor,104 (eBioscience), anti-CD45.1-PE-Cy7, A20 (eBioscience), anti-CD115-PE, AFS98 (eBioscience) anti-CD4-FITC, RM4-5 (eBioscience), anti-CD3e-PE-eFluor610, 145-2C 11 (eBioscience), anti-CD8a-BV510, 53-6.7 (BioLegend), anti-Ly6C-APC, AL-21 (BD Pharmingen™), anti-CD45R-APC-Cy7, RA-6B2 (BD Pharmingen™), anti-Ly6G-PerCP-Cy5.5, 1A8 (BD Pharmingen™), anti-CD43-BUV737, S7 (BD Horizon™). Staining strategies: Peripheral blood cells were obtained from retro-orbital vein. Red blood cells were lysed with 1×RBC Lysis Buffer (eBioscience™) for 5 minutes on ice followed by the 10 minutes incubation with Fc blocker (1:50) on ice. Incubation with antibodies were done for 20 minutes at room temperature in the dark. Dead cells were excluded from analysis by DAPI staining.

Cardiac Immune Cells:

Antibodies: For myeloid cell staining, cell suspensions were labeled with anti-CD45-Pacific Blue, 30-F11 (BioLegend), anti-CD11b-APC-Cy7, M1/70 (BioLegend), anti-Ly6G-PE, 1A8 (BioLegend), anti-Ly6C-FITC, HK1.4 (BioLegend), and anti-F4/80-PE-Cy7, BM (BioLegend). For lymphoid cell staining, cells were labeled with anti-CD45-Pacific Blue, 30-F11 (BioLegend), anti-CD3e-PE-eFluor610, 145-2C11 (eBioscience), anti-CD4-FITC, RM4-5 (eBioscience), anti-CD8a-BV510, 53-6.7 (BioLegend), anti-CD11b-AF700, M1/70 (BioLegend), anti-CD45R/B220-PE-Cy7, RA3-6B2 (BioLegend), anti-CD 19-APC-Cy7, 6D5 (BioLegend).

Staining Strategies:

Heart was extensively flushed with PBS to avoid blood contamination and then excised. Remote myocardium was separated from infarct and marginal zone as indicated in FIG. 22, minced with scissors and digested in collagenase I (450 U/ml), collagenase XI (125 U/ml), DNase I (60 U/ml), and hyaluronidase (450 U/ml) (catalog #C0130, C7657, D4513, and H3506, respectively, Sigma-Aldrich) at 225 rpm at 37° C. for 50 minutes (3). TAC-induced hypertrophied myocardium was digested in the same method. Hearts were subsequently homogenized through a 70-μm nylon mesh (Fisher Scientific). Incubation with antibodies were done for 20 minutes at room temperature in the dark. Dead cells were excluded from analysis by DAPI staining. 123count eBeads™ Counting Beads (BD Bioscience) was used for data acquisition.

Bone Marrow Cells:

Antibodies and strategies: Bone marrow cells were flushed out from bones (femur and tibia). Red blood cells were lysed with 1×RBC Lysis Buffer (eBioscience™) for 5 minutes on ice. For hematopoietic stem/progenitor cell staining, cells were labeled with biotin-conjugated anti-mouse antibodies directed against CD11b, M1/70 (BioLegend), Gr-1, RB6-8C5 (BioLegend), Ter-119, TER-119 (BioLegend), CD45R/B220, RA3-6B2 (BioLegend), CD3e, 145-2C11 (BioLegend), and CD127, A7R34 (BioLegend) followed by a labeling with an anti-biotin PE-conjugated streptavidin antibody (BioLegend), anti-c-kit-APC, 2B8 (BioLegend), anti-Sca-1-PE-Cy7, D7 (BioLegend), anti-CD16/32-APC-Cy7, 93 (BioLegend), anti-CD34-FITC, RAM34 (eBioscience), and anti-CD115-BV421, AFS98 (BioLegend). For neutrophil and monocyte staining, anti-CD11b-APC-Cy7, M1/70 (BioLegend), anti-CD115-APC, AFS98 (BioLegend), anti-Ly6G-PE, 1A8 (BioLegend), anti-I-A/I-E (MHC-class II)-AF700, M5/114.15.2 (BioLegend) were used. Dead cells were excluded from analysis by DAPI staining. BD LSR II Flow Cytometer (BD Bioscience) was used for data acquisition. Cells were defined as described in FIG. 22 (peripheral blood cells), FIG. 23 (cardiac immune cells), and FIGS. 24A-24D (bone marrow hematopoietic stem/progenitor cells). Data were analyzed with FlowJo Software.

Experimental LAD ligation

Mice initially underwent partial (10%) bone marrow reconstitution with Tet2-deficient cells or wild type cells following lethal irradiation, followed by permanent LAD ligation after 8 weeks of recovery. For myeloid-restricted Tet2 ablation, LAD ligation was performed when mice were 8-12 weeks old. Surgery was performed as previously described (4) with some modifications. Briefly, following anesthetization (isoflurane inhalation) and tracheal intubation, the chest cavity was open from the 4^{th} intercostal space. Left ascending coronary artery was ligated tightly with a tapered 8-0 vicryl suture (J401G, Ethicon) under microscopy. Myocardial ischemia was confirmed by ST-T segment elevation in electrocardiogram and color changes in the segment of left ventricle subjected to coronary flow occlusion. To close the wound, braided 6-0 vicryl suture (J212H, Ethicon) was used. Post-surgery, mice were monitored every day for 3 days, and treated with an intraperitoneal injection of buprenorphine (0.1 mg/kg) twice a day. Mice that died within 12 hours of surgery were excluded from analysis, and perioperative survival rate in this study was more than 95%. Surgery to each groups of mice was performed by an individual who was blinded to the identity of the mouse genotype.

Transverse Aortic Constriction (TAC)

Surgery was performed as previously described (5). Briefly, mice were anaesthetized with isoflurane inhalation. After the chest cavity was exposed by cutting open the proximal portion of the sternum, aortic constriction was produced by ligation of the transverse thoracic aorta between the innominate artery and left common carotid artery using a 27-gauge blunt needle and 7-0 silk suture. All mice were treated twice daily with buprenorphine (0.1 mg/kg) for 2 days, starting on the day of the surgery. Sham-operated mice without constriction served as controls. Surgery to each groups of mice was performed by an individual who was blinded to the identity of the mouse genotype.

Echocardiography

Cardiac function was assessed several times before and after the surgery (MI and TAC), at indicated in time points, using Vevo2100 ultrasound system equipped with MS550D probe (VisualSonics, Fujifilm). Mice were anesthetized with isoflurane at a concentration of 5% (induction) and 1-1.5% (maintenance). Each animal was placed on the heating table in a supine position with the extremities tied to the table through four electrocardiography leads. The chest was shaved using chemical hair remover, and ultrasound gel was applied to the thorax surface to optimize the visibility of the cardiac chambers. For MI study, left ventricular ejection fraction (LVEF), LV systolic and diastolic volume were measured from long-axis view by tracing end-diastolic and end-systolic endocardium. For TAC study, LV posterior wall thickness diameter (LVPWTd) and fractional shortening (FS, %) were measured from M-mode images obtained by short-axis view visualizing both papillary muscles. Measurements were performed by an individual who was blinded to the identity of the experimental groups of mice.

Measurement of the Initial Infarct Size

Mice were sacrificed 2 days after LAD ligation. Hearts were perfused with 20 ml of PBS from the apex of the heart as well as right ventricle to remove the peripheral blood. The hearts were excised and frozen in −20° C. domestic freezer for 2 hours, followed by sectioning into 2 mm slices from the ligation site. Hearts were incubated with 1% 2, 3, 5-triphenyltetrazolium chloride (TTC) in PBS for 15 minutes at 37° C. The size of infarct area was determined by computerized planimetry using Image J software.

Histology

The heart tissues were obtained at indicated time course after LAD ligation or TAC surgery. Heart tissues were perfused with cold PBS from apex and fixed in 10% formalin overnight and embedded in paraffin. About 7-μm-thick sections were de-paraffinized and rehydrated.

Mason's trichrome staining was performed according to the manufacturer's instructions (Sigma-Aldrich). For Picrosirius red staining, sections were incubated with freshly prepared staining buffer (1.2%/w picric acid in water, 0.1% o/w Fast Green FCF and 0.1%/w Direct Red 80 solved in PBS) for 1 h at room temperature (all products from Sigma-Aldrich). Sections were washed briefly in dH₂O and dehydrated. The slides were mounted by coverslip using Permount mounting medium (Fisher Scientific). The images were analyzed by ImageJ software (NIH) for quantification of fibrosis. Myocardial fibrosis size was expressed as a percentage of total LV area.

To measure cardiomyocyte cross-sectional area (CSA), heart sections were stained using Alexa Fluor 488 conjugated-WGA (Life Technologies). An operator who was blinded to genotype quantified cardiomyocyte CSA by computer-assisted morphometric analysis of microscopy images acquired on a Keyence BZ-9000 microscope. The average CSA of randomly selected 50-80 round-shaped cardiomyocytes per each sample was used for analysis.

Cell Proliferation was assessed by double immunofluorescent staining with monoclonal antibodies against the proliferating cell antigen Ki-67 (rabbit IgG, clone SP6, Vector Laboratories, 1:100 dilution) and the macrophage maker Mac3 (rat IgG, clone M3/84, Santa Cruz Biotechnology, 1:400 dilution). After deparaffinization, antigen unmasking with citric acid buffer was performed. Avidin/biotin blocking was performed by using avidin/biotin blocking kit following manufacturer's protocol (Vector Laboratories). Sections were blocked with 2.5% goat serum for 1 h and then incubated with primary antibodies at 4° C. overnight. Ki-67 was visualized with biotinylated anti-rabbit IgG following fluorescein labelled streptavidin (Vector Laboratories) and Mac3 with Alexa Flour 594-conjugated anti-rat IgG (Life Technologies). DAPI was used to detect nuclei.

For IL-1β staining, deparaffinization, antigen unmasking with citric acid buffer was performed, followed by blocking with 2.5% goat serum for 1 h. Sections were incubated with primary antibody specific for IL-1β (polyclonal rabbit IgG, Bioss, 1:100 dilution), and Mac3 (rat IgG, clone M3/84, Santa Cruz Biotechnology, 1:400 dilution) for overnight at 4° C. IL-1β was visualized with biotinylated anti-rabbit IgG following fluorescein labelled streptavidin (Vector Laboratories) and Mac3 with Alexa Flour 594-conjugated anti-rat IgG (Life Technologies). Nuclei were stained with DAPI and slides were incubated with Sudan Black B (Sigma-Aldrich) to reduce autofluorescence.

Fluorescent images were taken by confocal microscope LSM710 (Zeiss) or a BZ-9000 Keyence microscope. Fluorescent signal was quantified as relative integrated fluorescence intensity by using ImageJ software (NIH).

Gene Expression Analysis by qRT-PCR.

Total RNA from tissues and cultured cells was isolated using QIAzol reagent and RNeasy kits (Qiagen). RNA (0.5-1.2 μg) was reverse transcribed with QuantiTect Reverse Transcription Kit (Qiagen). qRT-PCR was performed with Power SYBR® Green reagent (ThermoFisher Scientific) in a ViiA7 PCR system. Primers for mouse gene expression studies are shown table 3. Results were analyzed with the ΔΔCt method. 36b4 or 18S rRNA were used as reference genes for normalization.

TABLE 3
Forward and reverse primer sequences used for quantitative PCR analysis.
Table 3 discloses SEQ ID NOS 71-88, respectively, in order of appearance.

| Gene name | Species | Forward | Reverse |
|---|---|---|---|
| Il-1b | Mus Musculus | 5′-TGACAGTGATGAGAATGACCTGTTC-3′ | 5′-TTGGAAGCAGCCCTTCATCT-3′ |
| Il-6 | Mus Musculus | 5′-GCTACCAAACTGGATATAATCAGGA-3′ | 5′-CCAGGTAGCTATGGTACTCCAGAA-3′ |
| Tnf | Mus Musculus | 5′-CGGAGTCCGGGCAGG-3′ | 5′-GCTGGGTAGAGAATGGATGAA-3′ |
| Ccl2 | Mus Musculus | 5′-CAGCCAGATGCAGTTAACGC-3′ | 5′-GCCTACTCATTGGGATCATCTTG-3′ |
| Ccl5 | Mus Musculus | 5′-CAGCAGCAAGTGCTCCAATC-3′ | 5′-CACACACTTGGCGGTTCCTT-3′ |
| Selp | Mus Musculus | 5′-CATCTGGTTCAGTGCTTTGATCT-3′ | 5′-ACCCGTGAGTTATTCCATGAGT-3′ |
| Tet2 | Mus Musculus | 5′-ACATCCCTGAGAGCTCTTGC-3′ | 5′-AGAGCCTCAAGCAACCAAAA-3′ |
| 36b4 | Mus Musculus | 5′-GCTCCAAGCAGATGCAGCA-3′ | 5′-CCGGATGTGAGGCAGCAG-3′ |
| 18S rRNA | Mus Musculus | 5′-AATCAAGAACGAAAGTCGGAGG-3′ | 5′-GCGGGTCATGGGAATAACG-3′ |

### REFERENCES FOR MATERIALS AND METHODS SECTION

1. Ko M, Bandukwala H S, An J et al. Ten-Eleven-Translocation 2 (TET2) negatively regulates homeostasis and differentiation of hematopoietic stem cells in mice. Proc Natl Acad Sci USA 2011; 108:14566-71.
2. Moran-Crusio K, Reavie L, Shih A et al. Tet2 loss leads to increased hematopoietic stem cell self-renewal and myeloid transformation. Cancer Cell 2011; 20:11-24.
3. Anzai A, Choi J L, He S et al. The infarcted myocardium solicits G M-CSF for the detrimental oversupply of inflammatory leukocytes. J Exp Med 2017; 214:3293-3310.
4. Maruyama S, Nakamura K, Papanicolaou K N et al. Follistatin-like 1 promotes cardiac fibroblast activation and protects the heart from rupture. EMBO Mol Med 2016; 8:949-66.
5. Shibata R, Ouchi N, Ito M et al. Adiponectin-mediated modulation of hypertrophic signals in the heart. Nat Med 2004; 10:1384-9.

### Example 5: TET2-Driven Clonal Hematopoiesis Predicts Enhanced Response to Anti-IL-1βAntibody Therapy

Clonal Hematopoiesis of Indeterminate Potential (CHIP) is associated with increased risk of atherosclerotic cardiovascular disease and preclinical work in mice has demonstrated that CHIP due to somatic mutations in the TET2 gene leads to accelerated atherosclerosis secondary to activation of the IL-1β signaling pathway. In the Canakinumab Anti-inflammatory Thrombosis Outcome Study (CANTOS), canakinumab, an anti-IL-1βantibody, reduced the relative risk of major adverse cardiovascular events (MACE) by 15% in patients with a history of myocardial infarction and increased inflammation as indicated by an hsCRP >2 mg/L.

A targeted deep sequencing of genes associated with CHIP in a subset (n=3925) of CANTOS trial participants was performed using genomic DNA prepared from baseline peripheral blood samples. 344 patients (8.8%) in this subset were identified with evidence for clonal hematopoiesis. As expected, the incidence of CHIP increased with age, with the mean age of CHIP(+) patients in CANTOS being 66 years old, while that of the non-CHIP patients was 61.5 years old. In contrast to populations not selected for elevated hsCRP, in the CANTOS population TET2 mutations were the most commonly detected mutations leading to CHIP (120 mutations in 104 subjects), while mutations in DNMT3A were less prevalent (86 mutations in 83 patients). Placebo-treated CHIP(+) patients showed a trend toward an increased rate of MACE compared to non-CHIP patients using a Cox proportional hazard model (HR=1.36, p=0.16). In exploratory analyses of placebo-treated patients with a somatic mutation in either TET2 or DNMT3A (n=58), a greater magnitude of risk for MACE was observed (HR=1.76, p=0.037).

CHIP(+) patients had a trend toward an improved response to canakinumab, with TET2 mutant patients having an improved response to canakinumab (HR=0.36, p=0.034). The data show that CHIP(+) patients are at increased risk for cardiovascular events and also demonstrate that those with TET2 mutations may respond better to canakinumab than those patients without CHIP.

### Example 6: Inactivation of Tet2 and Dnmt3 in Hematopoietic Cells Promotes Cardiac Dysfunction and Renal Fibrosis in Mice

Clonal hematopoiesis has been associated with increased mortality and cardiovascular disease. This condition can arise from somatic mutations in preleukemic driver genes within hematopoietic stem/progenitor cells. Approximately 40 candidate driver genes have been identified, but mutations in only 1 of these genes, TET2 (ten-eleven translocation-2), has been shown herein to casually contribute to cardiovascular disease in murine models.

To develop a facile system to evaluate the disease characteristics of different clonal hematopoiesis driver genes using lentivirus vector and CRISPR/Cas9 (clustered regularly interspaced short palindromic repeats/clustered regularly interspaced short palindromic repeat-associated 9) methodology. As demonstrated herein, it was shown that Dnmt3a (DNA [cytosine-5]-methyltransferase 3a)—a commonly occurring clonal hematopoiesis driver gene—causally contributes to cardiovascular disease.

Lentivirus vectors were used to deliver Cas9 and guide RNA to introduce inactivating mutations in Tet2 and Dnmt3a in lineage-negative bone marrow cells. After implantation into lethally irradiated mice, these cells were engrafted and gave rise to labeled blood cell progeny. When challenged with an infusion of Ang II (angiotensin II), mice with inactivating mutations in Tet2 or Dnmt3a displayed greater cardiac hypertrophy, diminished cardiac function, and greater cardiac and renal fibrosis. In comparison with Tet2, inactivation of Dnmt3a did not lead to detectable expansion of the mutant hematopoietic cells during the time course of these experiments. Tet2 inactivation promoted the expression of IL (interleukin) 1β, IL-6, and Ccl5, whereas Dnmt3a inactivation promoted the expression of Cxcl1 (CXC chemokine ligand), Cxcl2, IL-6, and Ccl5 in a lipopolysaccharide-stimulated macrophage cell line.

As demonstrated herein, inactivating DNMT3A mutations in hematopoietic cells using lentivirus vector/CRISPR methodology contributes to cardiovascular disease. As demonstrated herein by comparative analysis, inactivation of Tet2 and Dnmt3 was similar in their ability to promote Ang II-induced cardiac dysfunction and renal fibrosis in mice. Gene-specific actions were demonstrated by differences in kinetics of hematopoietic stem/progenitor cell expansion and different patterns of inflammatory gene expression.

TABLE 4
List of abbreviations.

| Full name | Abbreviation |
|---|---|
| Age-related clonal hematopoiesis | ARCH |
| Angiotensin II | AngII |
| Clonal hematopoiesis of | CHIP |
| indeterminate potential | |
| Guide RNA | gRNA |
| Insertion and deletion | Indel |

Somatic DNA mutations accumulate over time in many tissues, and this is a hallmark of the aging process (1). In particular, somatic mutations in preleukemic driver genes within hematopoietic stem/progenitor cell (HSPC) can confer fitness advantages leading to their clonal amplification. This process is referred to as clonal hematopoiesis (2). This condition is prevalent in the elderly population, and it has been termed clonal hematopoiesis of indeterminate potential 3 or age-related clonal hematopoiesis (4) Several recent studies have associated advanced clonal hematopoiesis with increased mortality (4-6) and with increased frequencies of cardiovascular disease (CVD) and stroke (4,7) Recently, we provided causal evidence for a link between somatic mutations in TET2 (ten-eleven translocation-2) and CVD in models of murine atherosclerosis and heart failure and provided details about its mechanism (8, 9) TET2 is 1 of ≈40 candidate driver genes that have been associated with clonal hematopoiesis (10). To date, there is an absence of information about the roles of driver genes, other than Tet2, in chronic disease. As demonstrated herein, the development of procedures to facilitate the analysis of additional candidate driver genes is shown.

CRISPR/Cas9-Mediated Tet2 Gene Disruption Promotes Hematopoietic Cell Expansion

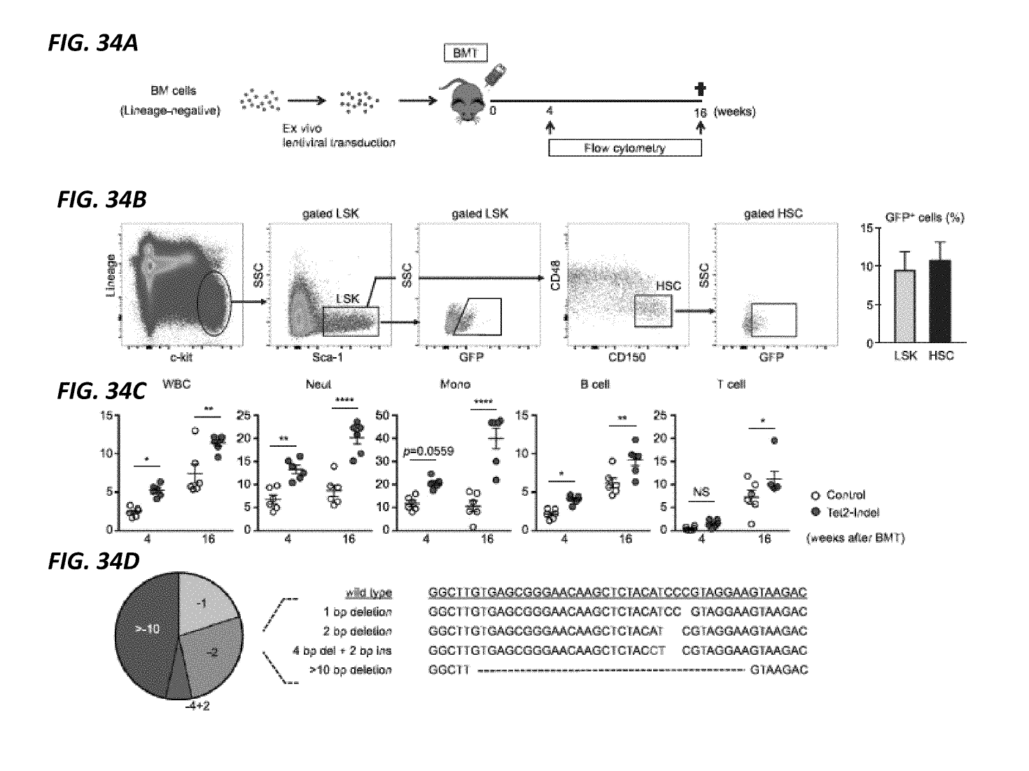

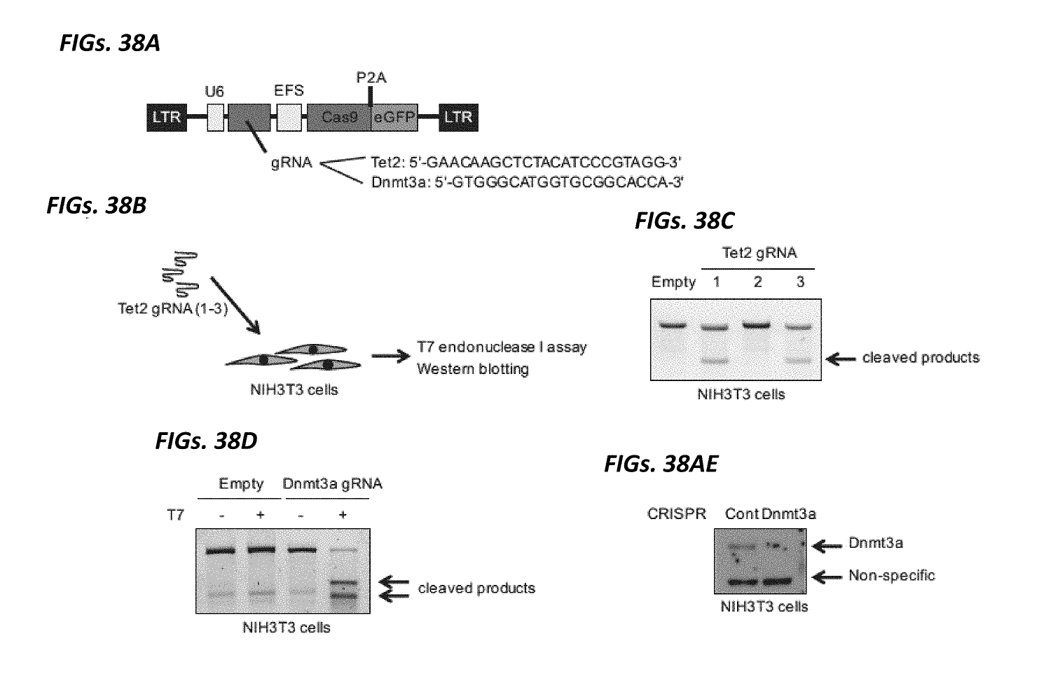

As demonstrated herein, the efficiency of the lentivirus system to mutate driver genes in HSPCs was evaluated, lineage-negative cells were isolated from wild-type mice and transduced ex vivo with a lentivirus vector encoding Cas9 (clustered regularly interspaced short palindromic repeat-associated 9)-GFP (green fluorescent protein) and transplanted into lethally irradiated wild-type mice (FIG. 34A). At 8 weeks post-transplantation, the detection of GFP-positive cells in LSK− (lineage−, c-kit+, Sca-1+) or LT-HSC (hematopoietic stem cell)-gated populations revealed successful HSPC transduction and engraftment with the lentivirus system (FIG. 34B). Guide RNA candidates targeting Tet2 were selected after evaluation in NIH/3T3 by the T7 endonuclease I mismatch cleavage assay (FIGS. 38A-38E). To assess whether ex vivo Tet2 gene-edited HSPCs replicate the expansion characteristics of bone marrow cells from Tet2-knockout (KO) mice, the fractions of GFP-positive cells in the different peripheral blood cell lineages were determined at the 4- and 16-week time points (FIG. 34C). This analysis revealed expansion characteristics that were similar to what was observed in our prior studies with Tet2-KO mice (8,9). To determine the degree of Tet2 editing by the lentivirus vector, DNA sequencing of the Tet2 locus was performed on DNA-isolated GFP-positive white blood cells. All the sequence reads revealed out-of-frame indel (insertion and deletion) mutations (3n+1, 3n+2) within exon 3 (FIG. 34D), which are predicted to lead to a loss of gene function.

Phenotypes of Tet2 Gene Disruption in the Ang II Infusion Model

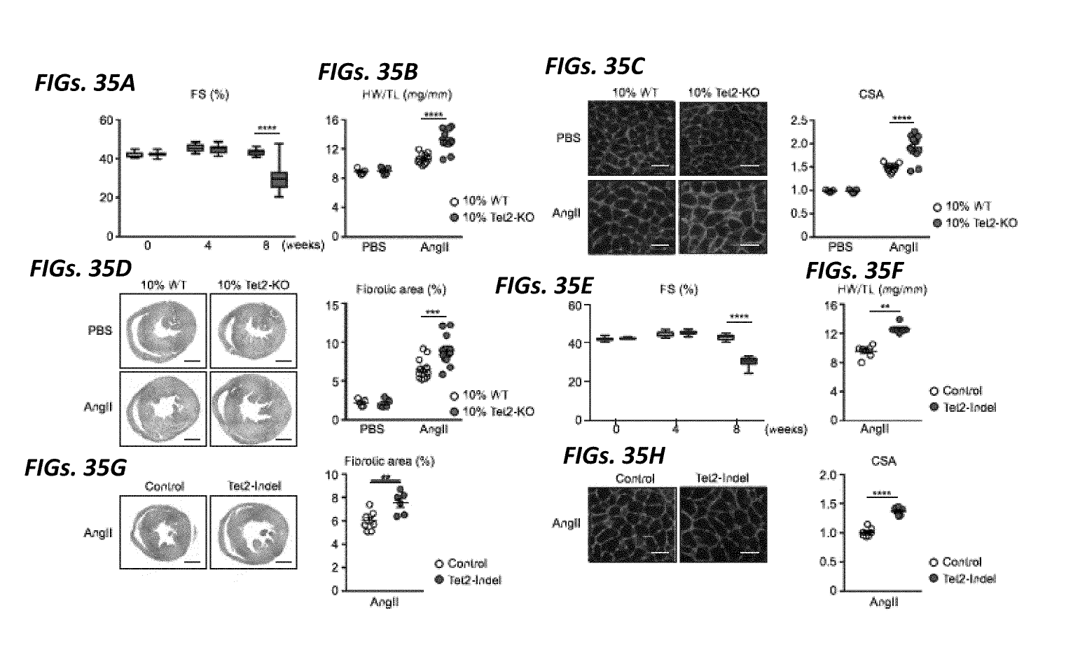

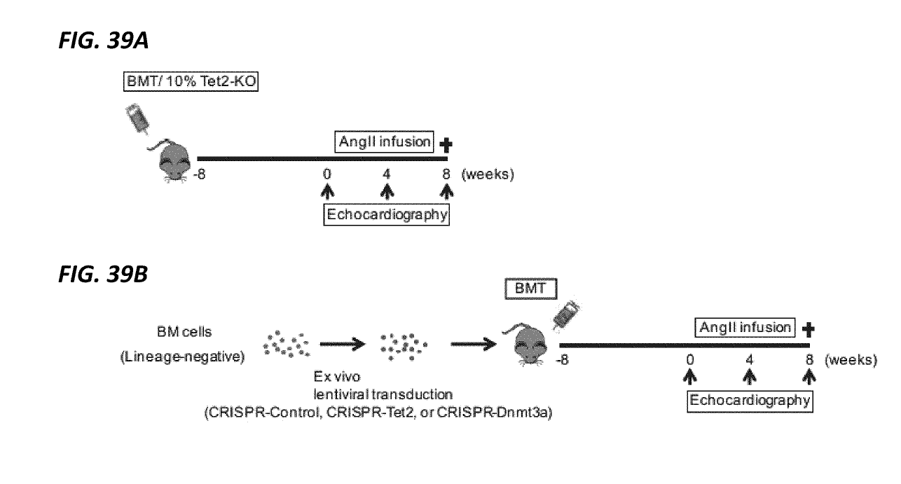

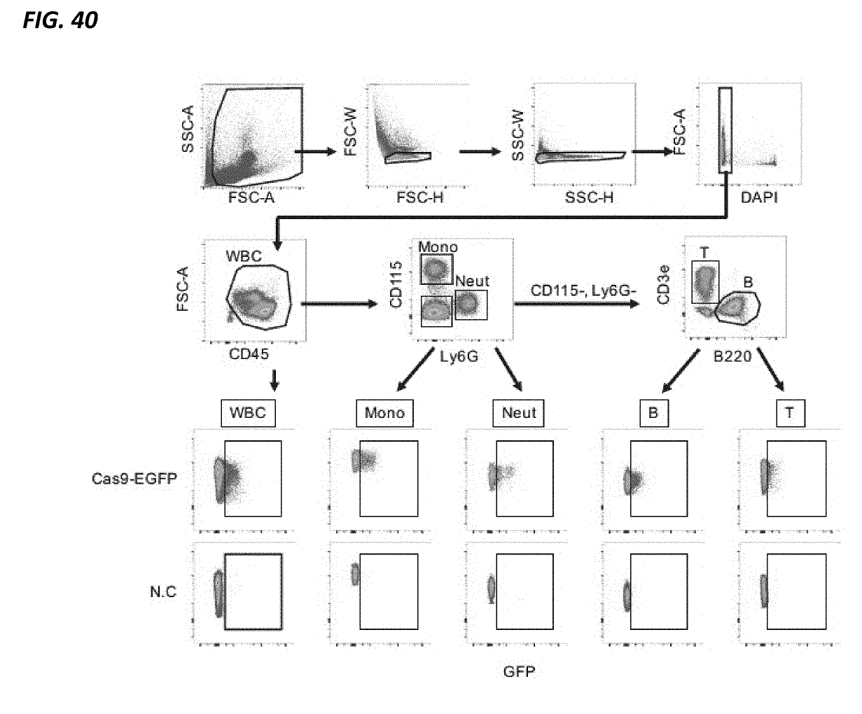

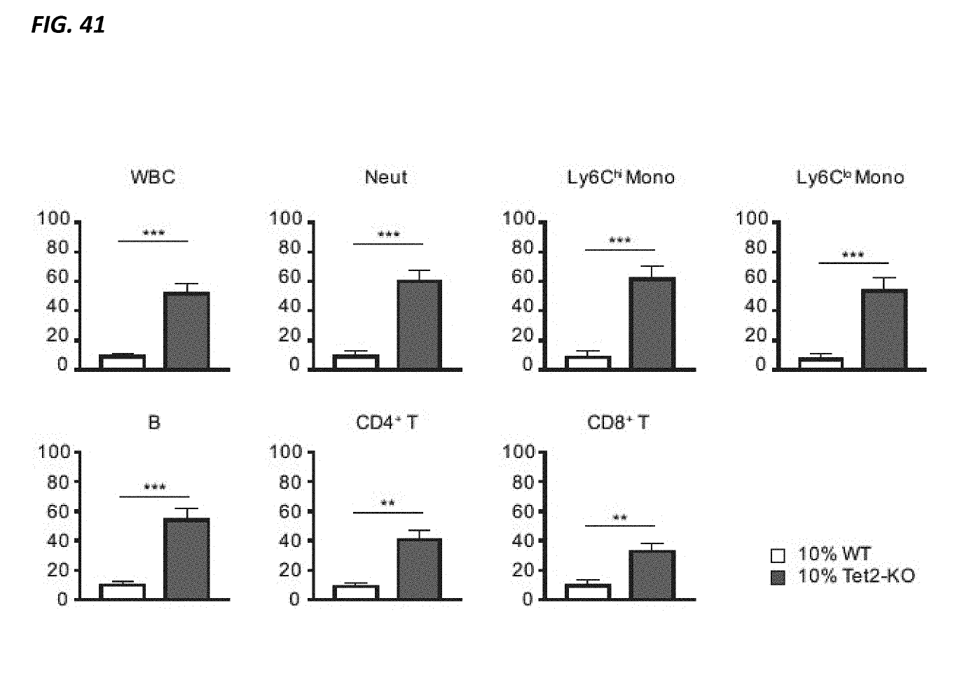

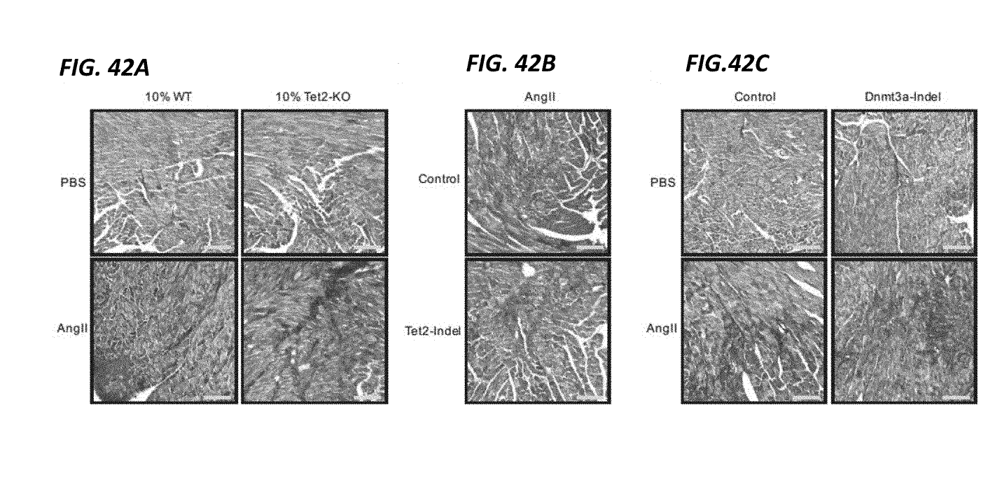

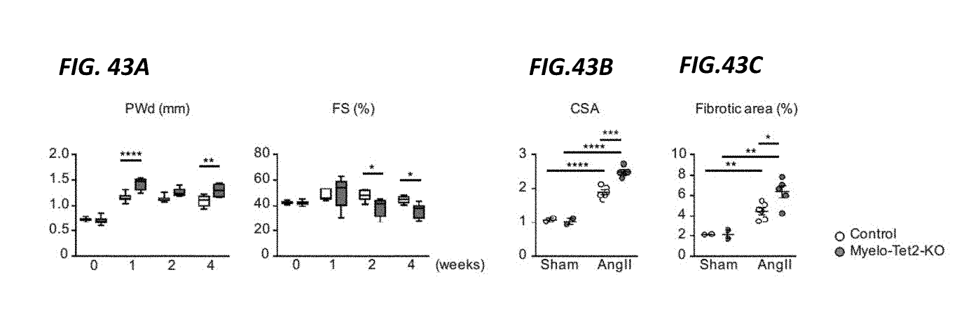

Having demonstrated the efficiency of the lentivirus delivery system on HSPC expansion, it was examined whether Tet2 gene editing in HSPCs promotes pathological cardiac remodeling. The Ang II infusion model was chosen because it does not involve a major surgical manipulation. To validate this new model for studies of clonal hematopoiesis, Ang II was infused to mice that had undergone a competitive bone marrow transplantation using 10% conventional CD45.2+ Tet2− KO mice and 90% CD45.1+ wild-type mice (FIGS. 39A-39B) (8,9). Flow cytometry analysis of the different blood cell lineages, according to the gating strategy shown in FIG. 40, revealed the selective expansion of the Tet2-KO hematopoietic cells (FIG. 41). In this model, mice transplanted with 10% Tet2-deficient bone marrow cells displayed greater deterioration of cardiac function by echocardiography after 8 weeks of systemic Ang II infusion compared with mice transplanted with 100% wild-type cells that were comprised of 10% CD45.2+ cells (FIG. 35A). At the termination of the experiment, hearts in mice receiving 10% Tet2-KO bone marrow displayed an increase in cardiac weight (FIG. 35B), an increase in cardiac myocyte cross sectional area (FIG. 35C), and an increase in interstitial fibrosis (FIG. 35D and FIGS. 42A-42C). Finally, a similar cardiac phenotype could also be detected in myeloid-specific, Tet2-deficient mice that were infused with Ang II (FIGS. 43A-43C), consistent with observations in other models of myocardial dysfunction (9).

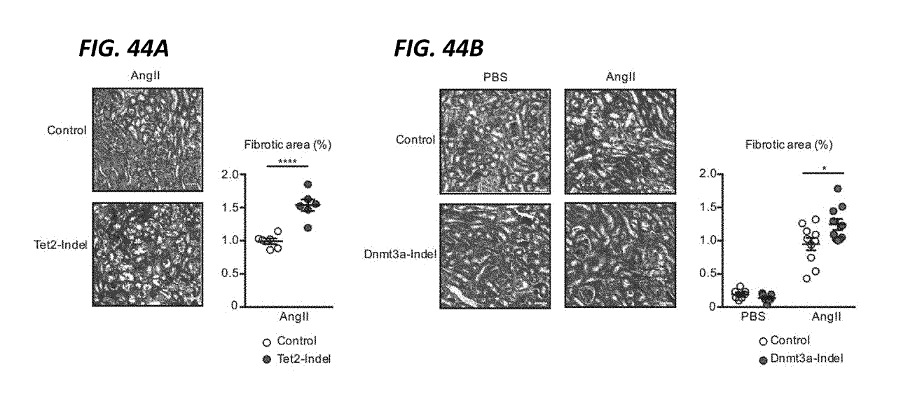

The next series of Ang II-infusion experiments involved mice transplanted with HSPC that underwent Tet2 editing using the lentivirus/CRISPR (clustered regularly interspaced short palindromic repeats) system (FIGS. 39A-39B). Similar to mice subjected to the conventional, competitive bone marrow transplantation with Tet2-KO cells, mice transplanted with CRISPR-edited HSPC displayed greater reductions in cardiac function as determined by echocardiography (FIG. 35E). These mice also displayed an increase in cardiac mass, cardiac fibrosis, and cardiac myocyte cross-sectional area (FIGS. 35F-35H and FIG. 42B). Histological analyses also revealed greater kidney fibrosis in mice that were implanted with the CRISPR-edited HSPC (FIGS. 44A-44B).

CRISPR/Cas9-Mediated Hematopoietic Dnmt3a Gene Disruption Promotes Cardiac Dysfunction

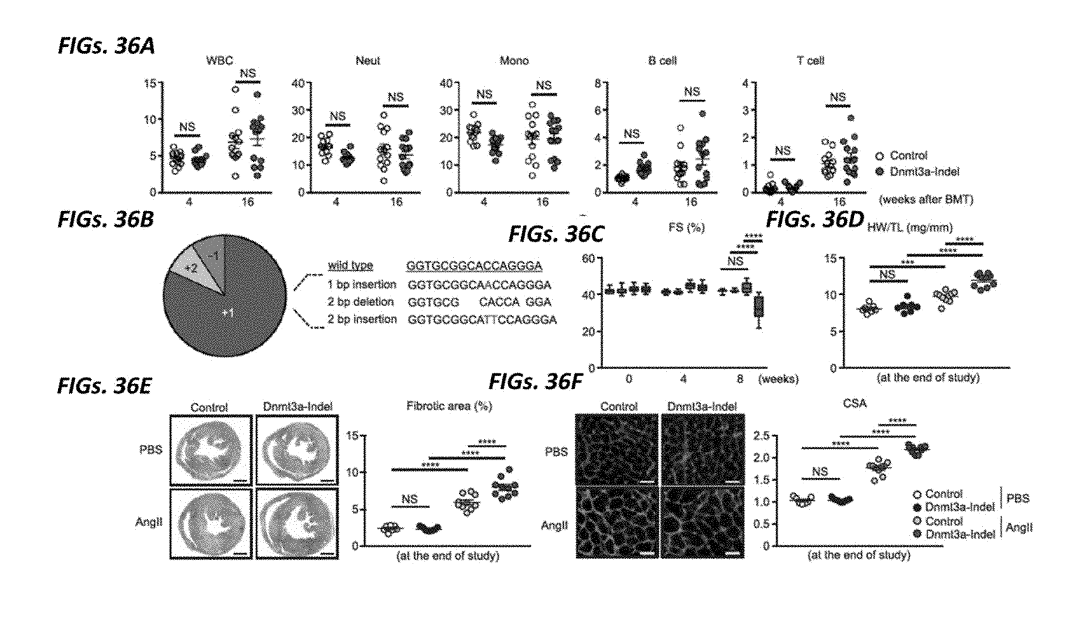

Having demonstrated that the lentivirus/CRISPR/Ang II system can be used to analyze the role of clonal hematopoiesis in CVD processes, mice with Dnmt3a (DNA [cytosine-5]-methyltransferase 3a)-edited HSPC were evaluated in the Ang II model. The validation of guide RNA targeting Dnmt3a was performed using the T7 endonuclease I mismatch cleavage assay and immunoblotting (FIG. 38D-38E). Consistent with previous reports (11-13), Dnmt3a-disrupted HSPCs did not undergo selective expansion during the 4-month time course of these experiments (FIG. 36A). Thus, a relatively low level of chimerism, similar to that of nonedited cells, was observed in the peripheral blood populations. To determine the status of Dnmt3a editing by the lentivirus vector, sequencing of the Dnmt3a locus was performed on DNA isolated from GFP-positive white blood cells. All sequence reads revealed out-of-frame mutations (3n+1, 3n+2), which lead to premature stop codons in exon 17 of Dnmt3a gene (FIG. 36B) and a loss of enzymatic activity (14). Mice transplanted with Dnmt3a-edited HSPC showed a reduction in cardiac function by echocardiographic analysis and an increase in cardiac mass 8 weeks after systemic Ang II administration (FIGS. 36C-36D). Mice receiving Dnmt3a-edited HSPC also displayed greater cardiac fibrosis and greater cardiac myocyte cross-sectional area in tissue sections (FIGS. 36E-36F; FIG. 42C). The Dnmt3a-edited HSPC condition led to greater renal fibrosis (FIGS. 44A-44B).

Dnmt3a Disruption Enhances Inflammation

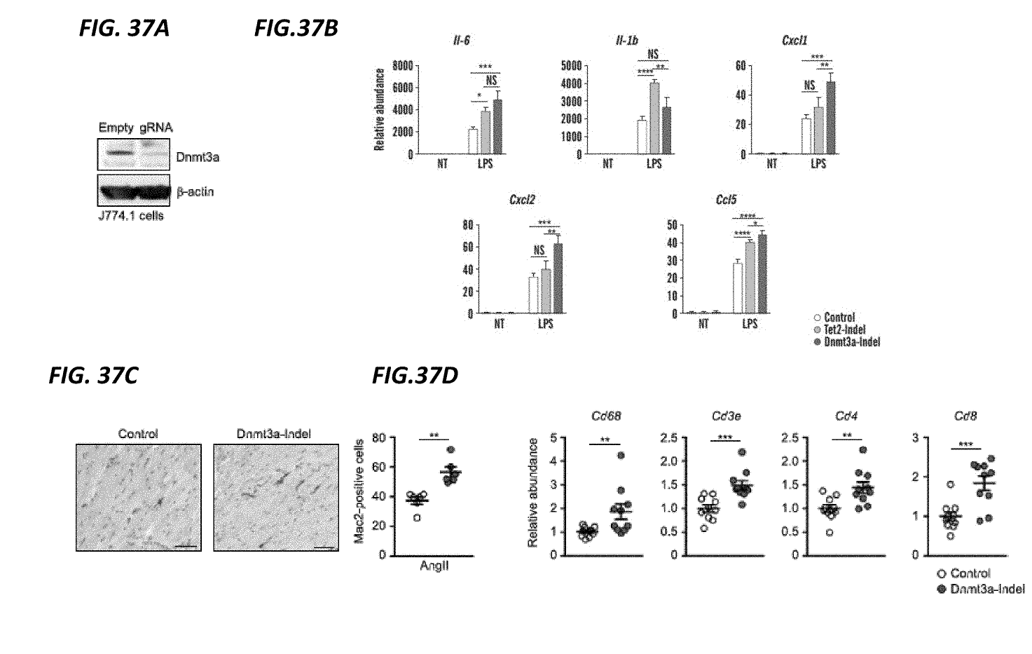

The recruitment and activation of peripheral myeloid cells contributes to cardiac dysfunction in the Ang II infusion model (15,16). To address the role of Dnmt3a gene disruption in myeloid cells, Dnmt3a-deficient J774.1 myeloid cell line using the lentivirus/CRISPR system was established. After sorting and amplification, GFP-positive cells displayed a marked reduction in Dnmt3a protein expression by immunoblotting (FIG. 37A). Using the same system, Tet2 deficiency was also established in J774.1 cells for comparative analyses. Consistent with our previous results, Tet2 disruption led to increased inflammatory chemokine/cytokine transcript expression after lipopolysaccharide stimulation. 8,9 Dnmt3a deficiency in J774.1 cells also displayed greater inflammatory responses after lipopolysaccharide stimulation (FIG. 37B). However, the patterns of inflammatory mediator induction in the Dnmt3a− and Tet2− deficient states differed. Whereas IL (interleukin)-6 and Ccl5 were similarly induced in both conditions, IL-1β was significantly upregulated in Tet2-edited cells, but there was only a trend in Dnmt3a-edited cells. Conversely, Cxcll (CXC chemokine ligand) and Cxcl2 were upregulated only in Dnmt3a-deficient cells compared with the control condition.

Consistent with observations of greater inflammatory responses in the Dnmt3a-deficient state, greater macrophage accumulation was observed in the myocardium after Ang II in-fusion (FIG. 37C). This was accompanied by elevated levels of transcripts of immune cell markers, including CD68, CD3e, CD4, and CD8, demonstrating that hematopoietic Dnmt3a deficiency leads to an increase in myocardial inflammation (FIG. 37D).

TABLE 5
Forward primers used for quantitative PCR analysis.

| Gene name | Species | Sequence |
|---|---|---|
| 36b4 | Mus Musculus | 5′-GCTCCAAGCAGATGCAGCA-3′ (SEQ ID No: 85) |
| 1l-1 b | Mus Musculus | 5′-TGACAGTGATGAGAATGACCTGTTC-3′ (SEQ ID No: 71) |
| 1l-6 | Mus Musculus | 5′-GCTACCAAACTGGATATAATCAGGA-3′ (SEQ ID No: 73) |
| 1l-1 8 | Mus Musculus | 5′-CAAACCTTCCAAATCACTTCCT-3′ (SEQ ID No: 89) |
| Tnf | Mus Musculus | 5′-CGGAGTCCGGGCAGG-3′ (SEQ ID No: 75) |
| Cxcll | Mus Musculus | 5′-CCGAAGTCATAGCCACACTCAA-3′ (SEQ ID No: 90) |
| Cxcl2 | Mus Musculus | 5′-TGACTTCAAGAACATCCAGAGCTT-3′ (SEQ ID No: 91) |
| Ccl5 | Mus Musculus | 5′-CAGCAGCAAGTGCTCCAATC-3′ (SEQ ID No: 79) |
| Cd68 | Mus Musculus | 5′-GGACTACATGGCGGTGGAATAC-3′ (SEQ ID No: 92) |
| Cd3e | Mus Musculus | 5′-AACACGTACTTGTACCTGAAAGC-3′ (SEQ ID No: 93) |
| Cd4 | Mus Musculus | 5′-TCCTTCCCACTCAACTTTGC-3′ (SEQ ID No: 94) |
| Cd8 | Mus Musculus | 5′-GCTCAGTCATCAGCAACTCG-3′ (SEQ ID No: 95) |

TABLE 6
Reverse primers used for quantitative PCR analysis.

| Gene name | Species | Sequence |
|---|---|---|
| 36b4 | Mus Musculus | 5′-CCGGATGTGAGGCAGCAG-3′ (SEQ ID No: 86) |
| 1l-1 b | Mus Musculus | 5′-TTGGAAGCAGCCCTTCATCT-3′ (SEQ ID No: 72) |
| 1l-6 | Mus Musculus | 5′-CCAGGTAGCTATGGTACTCCAGAA-3′ (SEQ ID No: 74) |
| 1l-1 8 | Mus Musculus | 5′-TCCTTGAAGTTGACGCAAGA-3′ (SEQ ID No: 96) |
| Tnf | Mus Musculus | 5′-GCTGGGTAGAGAATGGATGAA-3′ (SEQ ID No: 76) |
| Cxcll | Mus Musculus | 5′-CAAGGGAGCTTCAGGGTCAA-3′ (SEQ ID No: 97) |
| Cxcl2 | Mus Musculus | 5′-CTTGAGAGTGGCTATGACTTCTGTCT-3′ (SEQ ID No: 98) |
| Ccl5 | Mus Musculus | 5′-CACACACTTGGCGGTTCCTT-3′ (SEQ ID No: 80) |
| Cd68 | Mus Musculus | 5′-GAGAGCAGGTCAAGGTGAACAG-3′ (SEQ ID No: 99) |
| Cd3e | Mus Musculus | 5′-GATGATTATGGCTACTGCTGTCA-3′ (SEQ ID No: 100) |
| Cd4 | Mus Musculus | 5′-AAGCGAGACCTGGGGTATCT-3′ (SEQ ID No: 101) |
| Cd8 | Mus Musculus | 5′-ATCACAGGCGAAGTCCAATC-3′ (SEQ ID No: 102) |

Studies have associated the clonal expansion of HSPC with mortality and increased CVD risk (4, 5, 7) Clonal hematopoiesis can be caused by somatic mutations in >40 candidate driver genes, and it is likely that these different drivers will confer gene-specific effects on pathological processes. To date, studies focused on Tet2, which functions to promote a DNA hydroxymethylation and to recruit histone deacetylases to gene regulatory sequences (2) have provided the only evidence that these somatic mutations can causally contribute to CVD. Given the challenge of evaluating many other driver genes, we have developed procedures to facilitate the analysis of clonal hematopoiesis in CVD. The described system uses lentiviral vectors to transduce HSPC and CRISPR methodology to create indel mutations in candidate driver gene. Although off-target mutations are a potential concern, this system allows the ex vivo manipulation of HSPC that can then be implanted into wild-type mice and thereby provide a more versatile and rapid approach to assess HSPC clonal expansion and its systemic consequences than the conventional murine transgenic/knockout technology. Similar techniques have been used to edit driver genes in a model of myeloid malignancy (17). To evaluate CVD, gene-edited mice were infused with Ang II-a peptide hormone involved in the pathologies of vascular, renal, and cardiac diseases. 18 Under these conditions, CRISPR-mediated indels in Tet2 of HSPC led to the expansion of mutant cells and worsened cardiac remodeling, serving to validate the system and extend our prior findings (8, 9).

This technology was applied to evaluate the consequences of inactivating mutations in Dnmt3a. This protein is a de novo DNA methyltransferase, and it is ex-pressed in long-term HSPC (19). Here, we show that lentivirus/CRISPR-mediated disruption of hematopoietic Dnmt3a results in greater cardiac hypertrophy, diminished ejection fraction, and increased fibrosis after Ang II administration. Cardiac dysfunction was associated with greater macrophage accumulation and the elevated expression of immune cell markers in the myocardium. Consistent with previous studies on Tet (2, 8, 9) these effects tended to be observed late in the course of the pathological process, demonstrating that clonal hematopoiesis impairs the resolution of inflammation. In cell culture studies, the inactivation of either Dnmt3a or Tet2 led to increased cytokine expression, but the effects differed qualitatively and quantitatively demonstrating that they confer gene-specific effects resulting from mechanistic differences between these 2 proteins.

As demonstrated herein, inactivating mutations in Dnmt3a and Tet2 lead to markedly different HSPC expansion characteristics. The robust expansion of Tet2-deficient cells into multiple blood cell lineages was observed after the implantation of CRISPR-edited HSPC, and these results are consistent with observations from experiments that using a competitive transplantation approach using a 1:9 ratio of Tet2-KO: wild-type bone marrow (8, 9) (FIGS. 34A-34D and FIGS. 39A-39B). In contrast, there was no detectable expansion of Dnmt3a-deficient peripheral blood cells over the time course of our study. This finding is consistent with previous reports showing that Dnmt3a-null HSPCs are observed to expand in aged mice or only after sequential bone marrow transplantations (12, 13, 20). Although Dnmt3a and Tet2 appeared to similarly affect cardiac pathology in the Ang II model, the lack of Dnmt3a-deficient hematopoietic cell expansion demonstrates that Dnmt3a may be more potent than Tet2 in promoting CVD.

In the current study, we show that Dnmt3a deficiency can alter the function of myeloid cells and promote the inflammation via the upregulation of specific cytokines and chemokines. These findings demonstrate that Dnmt3a-mediated clonal hematopoiesis are causally associated with CVDs by promoting prolonged inflammatory responses in myeloid cells.

### REFERENCES

1. Vijg J. Somatic mutations, genome mosaicism, cancer and aging. Curr Opin Genet Dev. 2014; 26:141-149.
2. Fuster J J Walsh K. Somatic mutations and clonal hematopoiesis: unexpected potential new drivers of age-related cardiovascular disease. Circ Res. 2018; 122:523-532.
3. Steensma D P, Bejar R, Jaiswal S, Lindsley R C, Sekeres M A, Hasserjian R P, Ebert B L. Clonal hematopoiesis of indeterminate potential and its distinction from myelodysplastic syndromes. Blood. 2015; 126:9-16.
4. Jaiswal S, Fontanillas P, Flannick J, et al. Age-related clonal hematopoiesis associated with adverse outcomes. N Engl J Med. 2014; 371:2488-2498.
5. Genovese G, Kaihler A K, Handsaker R E, et al. Clonal hematopoiesis and blood-cancer risk inferred from blood DNA sequence. N Engl J Med. 2014; 371:2477-2487.
6. Zink F, Stacey S N, Norddahl G L, et al. Clonal hematopoiesis, with and without candidate driver mutations, is common in the elderly. Blood. 2017; 130:742-752.
7. Jaiswal S, Natarajan P, Silver A J, et al. Clonal hematopoiesis and risk of atherosclerotic cardiovascular disease. N Engl J Med. 2017; 177:111-121.
8. Fuster J J, MacLauchlan S, Zuriaga M A, et al. Clonal hematopoiesis associated with TET2 deficiency accelerates atherosclerosis development in mice. Science. 2017; 355:842-847.
9. Sano S, Oshima K, Wang Y, MacLauchlan S, Katanasaka Y, Sano M, Zuriaga M A, Yoshiyama M, Goukassian D, Cooper M A, Fuster J J, Walsh K. Tet2-mediated clonal hematopoiesis accelerates heart failure through a mechanism involving the IL-1β/NLRP3 inflammasome. J Am Coll Cardiol. 2018; 71:875-886.
10. Xie M, Lu C, Wang J, et al. Age-related mutations associated with clonal hematopoietic expansion and malignancies. Nat Med. 2014; 20:1472-1478.
11. Jeong M, Park H J, Celik H, Ostrander E L, Reyes J M, Guzman A, Rodriguez B, Lei Y, Lee Y, Ding L, Guryanova O A, Li W, Goodell M A, Challen G A. Loss of Dnmt3a immortalizes hematopoietic stem cells in vivo. Cell Rep. 2018; 23:1-10. doi: 10.1016/j.celrep.2018.03.025.
12. Challen G A, Sun D, Jeong M, et al. Dnmt3a is essential for hematopoietic stem cell differentiation. Nat Genet. 2011; 44:23-31.
13. Cole C B, Russler-Germain D A, Ketkar S, et al. Haploinsufficiency for DNA methyltransferase 3A predisposes hematopoietic cells to myeloid malignancies. J Clin Invest. 2017; 127:3657-3674.
14. Yang L, Rau R, Goodell M A. DNMT3A in haematological malignancies. Nat Rev Cancer. 2015; 15:152-165.
15. Epelman S, Lavine K J, Beaudin A E, et al. Embryonic and adult-derived resident cardiac macrophages are maintained through distinct mechanisms at steady state and during inflammation. Immunity. 2014; 40:91-104.
16. Hulsmans M, Sam F, Nahrendorf M. Monocyte and macrophage contributions to cardiac remodeling. J Mol Cell Cardiol. 2016; 93:149-155.
17. Heckl D, Kowalczyk M S, Yudovich D, Belizaire R, Puram R V, McConkey M E, Thielke A, Aster J C, Regev A, Ebert B L. Generation of mouse models of myeloid malignancy with combinatorial genetic lesions using CRISPR-Cas9 genome editing. Nat Biotechnol. 2014; 32:941-946.
18. Danilczyk U, Penninger J M. Angiotensin-converting enzyme II in the heart and the kidney. Circ Res. 2006; 98:463-471.
19. Li X, Zhang Q, Ding Y, Liu Y, Zhao D, Zhao K, Shen Q, Liu X, Zhu X, Li N, Cheng Z, Fan G, Wang Q, Cao X. Methyltransferase Dnmt3a upregulates HDAC9 to deacetylate the kinase TBK1 for activation of antiviral innate immunity. Nat Immunol. 2016; 17:806-815.
20. Zhang X, Su J, Jeong M, Ko M, Huang Y, Park H J, Guzman A, Lei Y, Huang Y H, Rao A, Li W, Goodell M A. DNMT3A and TET2 compete and cooperate to repress lineage-specific transcription factors in hematopoietic stem cells. Nat Genet. 2016; 48:1014-1023.
21. Leoni C, Montagner S, Rinaldi A, Bertoni F, Polletti S, Balestrieri C, Monticelli S. Dnmt3a restrains mast cell inflammatory responses. Proc Natl Acad Sci USA. 2017; 114:E1490-E1499.
22. Yang F, Zhou S, Wang C, Huang Y, Li H, Wang Y, Zhu Z, Tang J, Yan M. Epigenetic modifications of interleukin-6 in synovial fibroblasts from osteoarthritis patients. Sci Rep. 2017; 7:43592. doi: 10.1038/srep43592.

Materials and Methods

Plasmids and lentivirus production: pSpCas9(BB)-2A-Puro (PX459) V2.0 was a gift from Feng Zhang (Addgene plasmid #62988). LentiCRISPRv2GFP was as gift from David Feidser (Addgene plasmid #82416). psPAX2 and pMD2.G was a gift from Didier Trono (Addgene plasmid #12260 and 12259). Small gRNA sequences targeting to mouse Dnmt3a (5′-gtgggcatggtgcggcacca-3′ SEQ ID NO: 103) and Tet2 (5′-gaacaagctctacatcccgt-3′ SEQ ID NO: 104) were individually subcloned into the vector with BsmBI restriction enzyme and lentivirus particles were generated as described 1, 2. Briefly, the plasmids (LentiCRISPRv2GFP-sgRNA, psPAX2, pMD2.G) were co-transfected to HEK293T cells with polyethylenimine and the supernatant was collected 48 hours after transfection. After filtration (0.45 μm), virus particles were concentrated by ultracentrifugation at the speed of 20,000 rpm for 3 hours. The virus pellet was suspended with StemSpan medium and kept at −80° C. Lentiviral particle titer was determined using Lenti-XTM qRT-PCR Titration Kit (Clontech).

Validation of gRNA. NI H-3T3, HEK293T and J774.1 cells were purchased from ATCC and Sigma Aldrich, respectively. The cells were cultured in DMEM supplemented with 10% fetal bovine serum with penicillin and streptomycin at 37° C. Three guide RNAs for each gene were screened for activity in NI H-3T3 cells. J774.1 cells were transduced with the lentivirus particles (with or without gRNAs). After culturing for at least 72 h, GFP positive cells were collected with FACSAria (BD Bioscience) and used in subsequent experiments.

Isolation of lineage-negative cells and lentivirus transduction. Lineage-negative cells were isolated from bone marrow of C57BL/6J wild type mice using the Lineage-depletion kit (Miltenyi Biotech) according to manufactures instructions. Cells were pre-incubated with the StemSpan medium for 1.5 hours at 37° C. Lentivirus transduction was performed in the presence of 20 ng/mL of TPO, 50 ng/mL of SCF-1, 4 μg/ml of polybrene, and 5 μg/ml of rapamycin for 16-20 hours. Cells were washed and re-suspended with RPMI medium before transplantation.

Mice. C57BL/6J Tet2-deficient mice, C57BL/6J Tet2 floxed mice, C57BL/6J LysM-Cre mice, C57BL/6 wild type mice, and C57BL/6 Cd45.1 Pep Boy mice were obtained from Jackson Laboratories as described previously 3. Male mice were used for all in vivo experiments. The Institutional Animal Care and Use Committee (IACUC) of Boston University and the Institutional ACAC at the University of Virginia approved all study protocols.

Bone marrow transplantation. For competitive transplantation, lethally irradiated CD45.1+ mice were transplanted with bone marrow cells containing 10% CD45.2+ Tet2−/− cells and 90% CD45.1+ Tet2+/+ cells (10% Tet2-KO mice) or 10% CD45.2+ Tet2−/− cells and 90% CD45.1+ Tet2+/+ cells (10% WT mice). For lineage-negative cell transplantation, lentivirus-transduced cells (5×105 cells/mouse) were retro-orbitally injected into lethally irradiated 8 week old C57BL/6 wild type mice. The 10% competitive bone marrow transplantation was performed as described previously 3. Briefly, 8 week old lethally irradiated C57BL/6 Cd45.1 Pep Boy recipient mice were transplanted with bone marrow cells containing 10% Cd45.2+ Tet2−/− cells and 90% Cd45.1+ Tet2+/+ cells or 10% Cd45.2+ Tet2+/+ cells and 90% Cd45.1+ Tet2+/+ cells (defined as 10% Tet2 KO-BMT mice and 10% WT-BMT mice, respectively). In these experiments, 5×106 of total cells were injected. In both models, recipient mice were exposed to two radiation doses of 450 rad 4 hours apart using an X-RAD 320 Biological Irradiator.

Flow cytometry Bone marrow cells were flushed out from femur and red blood cells were lysed with 1×RBC Lysis Buffer (eBioscience™) for 5 minutes on ice. For hematopoietic stem cell staining, cells were labeled with biotin-conjugated anti-mouse antibodies against CD11b, Gr-1, Ter-119, CD45R/B220, CD3e, and CD 127, followed by a labeling with an anti-biotin PE-conjugated streptavidin antibody and antibodies against c-kit, Sca-1, CD48, and CD150 (see also table 7).

TABLE 7
Flow cytometry antibodies used.

| Antigen | Clone | Conjugation | Supplier | Identifier |
|---|---|---|---|---|
| BM HSPC | | | | |
| CD11b | M1/70 | Biotin | BioLegend | #101203 |
| Gr-1 | RB6-8C5 | Biotin | BioLegend | #108403 |
| Ter119 | Ter-119 | Biotin | BioLegend | #116203 |
| CD45R/ | RA 3-6 B 2 | Biotin | BioLegend | #103203 |
| B220 | | | | |
| CD3e | 145-2C11 | Biotin | BioLegend | #100303 |
| CD127 | A7R34 | Biotin | BioLegend | #135005 |
| Streptavidin | — | BV650 | BioLegend | #405231 |
| c-Kit | 2B8 | PE | BioLegend | #105807 |
| Sca 1 | D7 | PE-Cy7 | BioLegend | #108114 |
| CD48 | HM48-1 | BV421 | BioLegend | #103427 |
| CD150 | TC15-12F12.2 | PerCP/Cy5.5 | BioLegend | #115921 |
| Peripheral | | | | |
| blood | | | | |
| CD45 | 30-F11 | PE-Cy7 | eBioscience | #25-0451- |
| | | | | 82 |
| CD115 | AFS98 | PE | eBioscience | #12-1152- |
| | | | | 82 |
| CD3e | 145-2C11 | APC-Cy7 | eBioscience | #17-0031- |
| | | | | 81 |
| Gr-1 | 1A8 | PerCP-Cy5.5 | BD Biosciences | #560602 |
| CD45R/ | RA 3-6 B 2 | APC-Cy7 | BD Biosciences | #552094 |
| B220 | | | | |

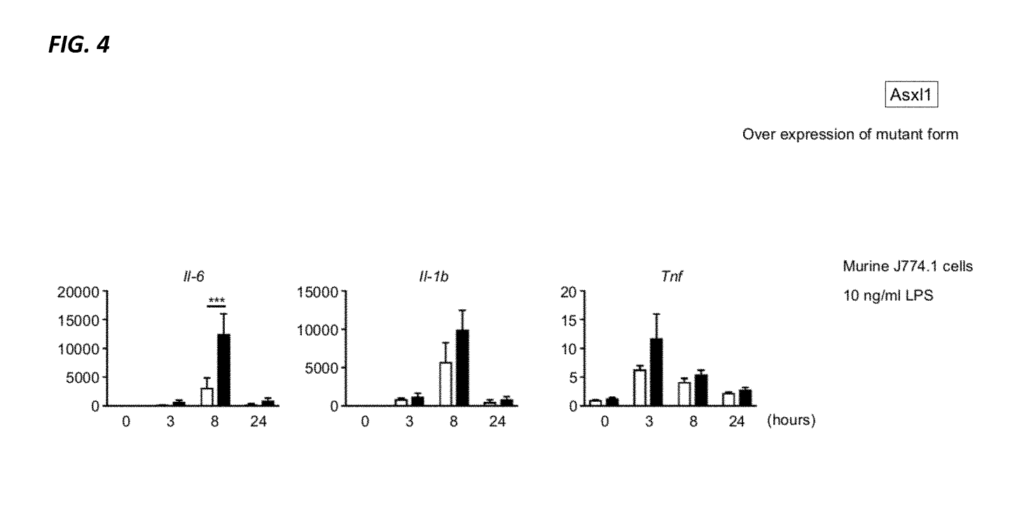

Peripheral blood cells were lysed with 1×RBC Lysis Buffer (eBioscience™) for 5 minutes on ice. After Fc blocking, cells were stained with following antibodies against CD45, CD115, Gr-1, CD45/B220, CD3e. Dead cells were excluded from analysis by DAPI staining. The detailed list of antibodies used in this study are shown in the Online Table 1. For data acquisition, BD LSR II Flow Cytometer (BD Bioscience) was used. Cells were defined as described in FIG. 1a (bone marrow hematopoietic stem cell) and Online FIG. 4 (peripheral blood cells). Data were analyzed with FlowJo Software.

Angiotensin-II infusion model. 1.5 mg/kg/min of human AngII (Sigma Aldrich) was infused subcutaneously by Alzet mini-osmotic pumps (Model 2004, Durect Corp.) to bone marrow-reconstituted mice for a period of 4 weeks. At this point, pumps were replaced to allow a continuous infusion for the entire duration of the study. Control mice were infused with PBS.

Echocardiography. Cardiac function was assessed before and after the AngII administration, at indicated in time points, using Vevo2100 ultrasound system equipped with MS550D probe (VisualSonics, Fujifilm). Mice were anesthetized with isoflurane at a concentration of 5% (induction) and 1% (maintenance). Each animal was placed on the heating table in a supine position. The chest was shaved using chemical hair remover, and ultrasound gel was applied to the thorax surface to optimize the visibility of the cardiac chambers. Fractional shortening (FS, %) was measured from M-mode images obtained by short-axis view visualizing both papillary muscles. Measurements were performed by an individual who was blinded to the identity of the experimental groups of mice.

Histology. Hearts were perfused with cold PBS from apex and fixed in 10% formalin overnight and processed for paraffin embedding. 7-pm-thick sections were de-paraffinized, rehydrated. For Picrosirius red staining, sections were incubated with freshly prepared staining buffer (1.2%/w picric acid in water, 0.1%/w Fast Green FCF and 0.1%/w Direct Red 80 solved in PBS) for 1 hour at room temperature (all products from Sigma-Aldrich). Sections were washed briefly in distilled H2O and dehydrated. The slides were mounted by coverslip using Permount mounting medium (Fisher Scientific). The images were analyzed by ImageJ software (NI H) for quantification of fibrosis. Myocardial fibrosis size was expressed as a percentage of total LV area. To measure cardiomyocyte cross-sectional area (CSA), heart sections were stained using Alexa Fluor 488 conjugated-WGA (Life Technologies). An operator who was blinded to mouse genotype quantified cardiomyocyte CSA by computer-assisted morphometric analysis of microscopy images acquired on a Keyence BZ-9000 microscope. The average CSA of randomly selected 50-80 round-shaped cardiomyocytes per each sample was used for analysis. For Mac2 immunohistochemistry staining, antigen unmasking was performed by boiling slides in citric acid buffer (Vector Laboratories, Inc.). Endogenous peroxidase was inactivated by using 0.3% H2O2for 30 min. Sections were blocked 5% horse serum for 1 hour and then incubated with anti-rabbit Mac2 antibody (clone H-160, catalog #sc-20157, Santa Cruz) at 1:500 dilution at 4° C. overnight. After washing slides, sections were incubated anti-rabbit IgG conjugated with peroxidase (Vector Laboratories, Inc.) for 30 minutes and developed using DAB substrate kit (Vector Laboratories, Inc.). Sections were counterstained with Harris hematoxylin. The number of Mac2 positive cell was counted in five high-power fields per section and per animal. For morphologic evaluation of the kidney, specimens were fixed in 10% formalin, paraffin embedded and sectioned at 7 μm. Masson's trichrome staining was performed according to the manufacturer's instructions (Sigma-Aldrich). Interstitial fibrosis was assessed at 200× original magnification with 10 randomly selected fields for each animal. The images were analyzed by Image J software (NI H) for quantification of fibrosis.

Quantitative PCR analysis. Total RNA from tissues and cultured cells was isolated using QIAzol reagent (Qiagen) and NucleoSpin RNA Kit (Clontech). RNA (0.7-1.0 Ng) was reverse transcribed with High-capacity RNA-to-cDNA Kit (Thermo Fisher Scientific). qRT-PCR was performed with Power SYBR® Green reagent (Thermo Fisher Scientific) in a ViiA7 PCR system. The list of primers for mouse gene expression studies are shown in the table 5 and 6. Results were analyzed with the AACt method. 36b4 was used as reference genes for normalization.

Statistics. Data were shown as mean±SEM, or median (minimum to maximum). Data distribution was evaluated by Shapiro-Wilk normality test. An F-test was used to evaluate homogeneity of variance. Statistical significance of differences in experiments with two groups and only one independent variable was assessed by two-tailed unpaired Student's t tests (normally distributed data with equal variance) or Mann-Whitney U Tests (non-normally distributed data). Differences in experiments with more than one independent variable were evaluated by two-way ANOVA, with post-hoc Sidak's or Tukey's multiple comparison tests. Results of echography and peripheral blood flow cytometry were assessed by two-way repeated measure ANOVA with post-hoc Sidak's or Tukey's multiple comparison tests. *p<0.0⁵, **p<0.01, ***p<0.001, ****p<0.0001. All statistical analyses were performed using GraphPad Prism 7 software (GraphPad Software Inc.).

### REFERENCES FOR MATERIALS AND METHODS

Heckl D, Kowalczyk M S, Yudovich D, Belizaire R, Puram R V, McConkey M E, Thielke A, Aster J C, Regev A and Ebert B L. Generation of mouse models of myeloid malignancy with combinatorial genetic lesions using CRISPR-Cas9 genome editing. Nat Biotechnol. 2014; 32:941-6.
Walter D M, Venancio O S, Buza E L, Tobias J W, Deshpande C, Gudiel A A, Kim-Kiselak C, Cicchini M, Yates T J and Feldser D M. Systematic In Vivo Inactivation of Chromatin-Regulating Enzymes Identifies Setd2 as a Potent Tumor Suppressor in Lung Adenocarcinoma. Cancer Res. 2017; 77:1719-1729.
Sano S, Oshima K, Wang Y, MacLauchlan S, Katanasaka Y, Sano M, Zuriaga M A, Yoshiyama M, Goukassian D, Cooper M A, Fuster J J and Walsh K. Tet2-Mediated Clonal Hematopoiesis Accelerates Heart Failure Through a Mechanism Involving the IL-1beta/NLRP3 Inflammasome. J Am Coll Cardiol. 2018; 71:875-886.

What is claimed herein is:

1. A method for treating a subject having, or at risk for, a HSC (hematopoietic stem cell) cardiometabolic driver gene mutation-mediated proinflammatory disease comprising: administering a therapeutically effective amount of a pharmaceutical composition comprising an inhibitor of HSC cardiometabolic driver gene mutation-mediated proinflammatory activity and a pharmaceutically acceptable carrier to a subject having one or more somatic mutations in one or more HSC cardiometabolic driver gene in a sub-population of peripheral blood hematopoietic cells.

2. The method of claim 1, wherein at least 2% of the peripheral blood hematopoietic cells have the one or more somatic mutations in the one or more HSC cardiometabolic driver genes.

3. The method of claim 1, wherein the one or more HSC cardiometabolic driver genes are selected from TP53, JAK2, DNMT3A, ASXL1, TET2, and PPM1D/WIP1.

4. The method of claim 3, wherein the one or more somatic mutations are in TP53 and are selected from a G743A mutation in SEQ ID NO:2 and a A659G mutation in SEQ ID NO: 2.

5. The method of claim 3, wherein the one or more somatic mutations are in JAK2 and is a G1849T in SEQ ID NO: 56.

6. The method of claim 3, wherein the one or more somatic mutations are in DNMT3A and are selected from a T1115C mutation in SEQ ID NO: 37; a C2711T mutation in SEQ ID NO: 37; a C1837G mutation in SEQ ID NO: 37; a A1666G mutation in SEQ ID NO: 37; a C1789T mutation in SEQ ID NO: 37; a G2719A mutation in SEQ ID NO: 37; a G1627T mutation in SEQ ID NO: 37; an A2723G mutation in SEQ ID NO: 37; a G1797T mutation in SEQ ID NO: 37; a T2252G mutation in SEQ ID NO: 37; a C1560A mutation in SEQ ID NO: 37; a T1031C mutation in SEQ ID NO: 37; a G2645A mutation in SEQ ID NO: 37; a C2043G mutation in SEQ ID NO: 37; a C2446T mutation in SEQ ID NO: 37; a A2198G mutation in SEQ ID NO: 37; A2281G mutation in SEQ ID NO: 37; a C920G mutation in SEQ ID NO: 37; a A2204G mutation in SEQ ID NO: 37; and a frameshift mutation in DNMT3A.

7. The method of claim 3, wherein the one or more somatic mutations are in ASXL 1 and are selected from a C2407T mutation in SEQ ID NO: 61; a C2893T mutation in SEQ ID NO: 61; a 1926_1926delinsAG mutation in SEQ ID NO: 61; and a frameshift mutation in ASXL1.

8. The method of claim 3, wherein the one or more somatic mutations are in PPM1D and are selected from a G1618T mutation in SEQ ID NO: 64; a C1372T mutation in SEQ ID NO: 64; and a frameshift mutation in PPM1D.

9. The method of claim 3, wherein one or more somatic mutations are in TET2 and are selected from an S282F mutation in SEQ ID NO: 68, an N312S mutation in SEQ ID NO: 68, an L346P mutation in SEQ ID NO: 68, an S460F mutation in SEQ ID NO: 68, a D666G mutation in SEQ ID NO: 68, a P941S mutation in SEQ ID NO: 68, and a C1135Y mutation in SEQ ID NO: 68.

10. The method of claim 1, wherein the inhibitor of HSC cardiometabolic driver gene mutation-mediated proinflammatory activity is an IL-1β inhibitor, an IL-1β inhibitor antibody or antigen-binding fragment thereof that binds to IL-1β and reduces IL-1β binding to its receptor(s), an IL-1 receptor antagonist, or a small molecule or microRNA inhibitor that inhibits IL-1β-mediated pro-inflammatory activity.

11. The method of claim 10, wherein the IL-1β inhibitor antibody or antigen-binding fragment thereof is selected from ABT981, an anti-interleukin-1β inhibitor antibody by ABZYME, APX002, Canakinumab/Ilaris, CDP48, immunereszumab, LY2189102, MEDI8968, and XOMA052.

12. The method of claim 10, wherein the IL-1 receptor antagonist is selected from CDP484, CP412245, CYT013 IL1bQb, XL 130, AMG108, HL 2351, IL1Hyl, AXXO, orthokine, PRT 1000, anakinra, and rilonacept.

13. The method of claim 10, wherein the small molecule or microRNA inhibitor is selected from AC201, CP412245, MCC950 or CRID3, inflabion, inflammasome modulator OPSONA, PGE3935199, PGE527667, TRK530, β-hydroxybutyrate (BHB), and microRNA-223.

14. The method of claim 1, wherein the inhibitor of HSC cardiometabolic driver gene mutation-mediated proinflammatory activity is an IL-6 inhibitor, an IL-6 inhibitor antibody or antigen-binding fragment thereof that binds to IL-6 and reduces IL-6 binding to its receptor(s), an IL-6 receptor antagonist, a small molecule or microRNA IL-6 inhibitor, or a JAK-STAT inhibitor.

15. The method of claim 14, wherein the IL-6 inhibitor antibody or antigen-binding fragment thereof is selected from Siltuximab, Olokizumab, Elsilimomab, mAb 1339, Sirukumab, Clazakizumab, ARGX-109, FM101, and C326.

16. The method of claim 14, wherein the IL-6 receptor antagonist is selected from tocilizumab, sarilumab, REGN88, FE301, and LMT-28.

17. The method of claim 14, wherein the small molecule IL-6 inhibitor is ALX-0061 or LMT-28.

18. The method of claim 14, wherein the JAK-STAT inhibitor is selected from baricitinib, decernotinid, filgotinib, INCB-039110, ruxolitinib, tofacitinib, Oclacitinib, Gandotinib, Lestaurtinib, Momelotinib, Pacritinib, PF-04965842, Upadacitinib, and Peficitinib.

19. The method of claim 1, wherein the inhibitor of HSC cardiometabolic driver gene mutation-mediated proinflammatory activity is a TNFα inhibitor, a TNFα inhibitor antibody or antigen-binding fragment thereof that binds to TNFα and reduces TNFα binding to its receptor(s), a TNFα receptor antagonist, or a small molecule or microRNA TNFα inhibitor.

20. The method of claim 19, wherein the TNFα inhibitor antibody or antigen-binding fragment thereof is selected from adalimumab, Adalimumab-atto, certolizumab pegol, golimumab, infliximab,

21. The method of claim 19, wherein the TNFα receptor antagonist is etanercept.

22. The method of claim 1, further comprising monitoring hematopoietic cell clonality, IL-1β proinflammatory activity, IL-6 proinflammatory activity, TNFα proinflammatory activity or any combination thereof following the administration of the inhibitor of HSC cardiometabolic driver gene mutation-mediated proinflammatory activity.

23. The method of claim 1, further comprising decreasing the number or percentage of hematopoietic cells comprising the one or more somatic mutations in the one or more HSC cardiometabolic driver genes in the subject by performing therapeutic cytapheresis on the subject.

24. The method of claim 1, further comprising administering one or more additional therapeutic agents to the subject.

25. The method of claim 1, wherein the HSC cardiometabolic driver gene mutation-mediated proinflammatory disease is a cardiometabolic or chronic kidney disease or disorder.

26. A method for treating a subject having, or at risk for, a HSC cardiometabolic driver gene mutation-mediated proinflammatory disease comprising:
(a) sequencing a hematopoietic cell sample from a subject to identify one or more somatic mutations in one or more HSC cardiometabolic driver genes in the hematopoietic cell sample; and
(b) administering a therapeutically effective amount of a pharmaceutical composition comprising an inhibitor of HSC cardiometabolic driver gene mutation-mediated proinflammatory activity and a pharmaceutically acceptable carrier if one or more somatic mutations in one or more HSC cardiometabolic driver genes are identified in the hematopoietic cell sample.

27. The method of claim 26, wherein the hematopoietic cell sample is a peripheral blood hematopoietic cell sample or enriched for myeloid-derived cells.
